(19) United States
(12) Patent Application Publication
(10) Pub. No.: US 2011/0294982 A1
(43) Pub. Date: Dec. 1, 2011

(54) AMINO ACID SEQUENCES DIRECTED AGAINST INTEGRINS AND USES THEREOF

(75) Inventors: Peter Vanlandschoot, Belgium (BE); Michael John Scott Saunders, Brussels (BE); Johannes Joseph Wilhelmus De Haard, Oudelande (NL); Christophe Blanchetot, Destelbergen (BE); Theo Verrips, Utrecht (NL); Marc Antoine Gijsbert Gilles Vooijs, Be Cadier en Keer (NL); Adrianus Johannes Groot, Je Maastricht (NL); Mohamed El Khattabi, Amersfoort (NL)

(73) Assignee: Ablynx N.V., Zwignaarde (BE)

(21) Appl. No.: 12/991,576

(22) PCT Filed: May 11, 2009
(86) PCT No.: PCT/EP2009/055679

§ 371 Date: Apr. 22, 2011

Related U.S. Application Data
(60) Provisional application No. 61/051,763, filed on May 9, 2008.

(51) Int. Cl. C07K 14/00

(52) U.S. Cl. 530/350

(57) ABSTRACT

The present invention relates to amino acid sequences that are directed against (as defined herein) Integrins, as well as to compounds or constructs, and in particular proteins and polypeptides, that comprise or essentially consist of one or more such amino acid sequences (also referred to herein as [symbol] □amino acid sequences of the invention□ com□pounds of the invention□, poly [symbol] [symbol] □peptides of the invention□, respectively).

The present invention relates to amino acid sequences that are directed against (as defined herein) Integrins, as well as to compounds or constructs, and in particular proteins and polypeptides, that comprise or essentially consist of one or more such amino acid sequences (also referred to herein as [symbol] □amino acid sequences of the invention□ com□pounds of the invention□, poly [symbol] [symbol] □peptides of the invention□, respectively).

The invention also relates to nucleic acids encoding such amino acid sequences and polypeptides (also referred to herein as □nucleic acids of the invention□ nucle□otide sequences of the invention□); to methods for preparing such amino acid sequences and polypeptides; to host cells expressing or capable of expressing such amino acid sequences or polypeptides; to compositions, and in particular to pharmaceutical compositions, that comprise such amino acid sequences, polypeptides, nucleic acids and/or host cells; and to uses of such amino acid sequences or polypeptides, nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

Integrins are cell adhesion molecules that mediate cell-cell, cell extracellular matrix, and cell-pathogen interactions. They play critical roles in development, wound healing, hemostasis, immunity and cancer.

Integrin adhesiveness can be dynamically regulated through a process termed inside-out signaling. In addition, ligand binding transduces signals from the extracellular domain to the cytoplasm in the classical outside-in direction. (Luo et al. Annu. Rev. Immunol. 2007. 25:619□47).

Integrins are noncovalently associated heterodimeric cell surface adhesion molecules composed of one alpha subunit and one beta subunit. In vertebrates, 18 α subunits and 8 β subunits form 24 known αβ pairs (see Luo et al., supra, page 620, FIG. 1). Half of integrin α-subunits contain inserted (I) domains, which are the principal ligand-binding domains when present. This diversity in subunit composition contributes to diversity in ligand recognition, binding to cytoskeletal components and coupling to downstream signaling pathways. Activation of integrin rely on a large change in conformation from a closed (low affinity) conformation to an open (high affinity) conformation (see Luo et al., supra, page 628, FIG. 6).

Dysregulation of integrins is involved in the pathogenesis of many disease states, from autoimmunity to thrombotic vascular diseases to cancer metastasis. Therefore, extensive efforts have been directed towards the discovery and development of integrin antagonists for clinical applications. Targeting aIIbb3 on platelets inhibits thrombosis, aVb3 and aVb5 blocks tumour metastasis, angiogenesis and bone resorption, and β2 integrins and a4 integrins on leukocytes for treating autoimmune diseases and other inflammatory disorders (see Shimaoka et al. Nature reviews in drug discovery 2003 pp 703-715 and reference therein).

Infiltration of leukocyte at the site of infection or inflammation depends on the adhesion of the leukocyte to the endothelial cell layer which is dependent integrins Inflammatory disease, auto-immune diseases, atherosclerosis. Efalizumab (Raptiva™) blocks LFA-1 (aLb2) and its use for the treatment of chronic plaque psoriasis. Natalizumab (Tysabri/Antegren™) blocks very late antigen-4 (VLA4/a4b1) for the treatment of relapsing-remitting multiple sclerosis. ReoPro/Abciximab target and blocks alphaIIbbeta3 (platelet integrin), Centocor/JandJ. FDA approved this anti-thrombotic agent in 1994. Blocking a5b1 blocks angiogenesis and consequently is used against cancer. Also anti alphaV therapies are efficient in blocking tumorigenesis. For example, Volociximab is an anti-a5b1 antibody inhibiting angiogenesis (PDL Biopharma/Biogen Idec) and CNTO95 is an anti-aV now in Phase I (Medarex/Centocor). Vitaxin/Abegrin/MEDI-522 (MedImmune) is in clinical trials (Phase 3) for metastatic melanoma and prostate cancer; blocks the interaction of alphaVbeta3 (the predominant integrin on osteoclasts) with various ligands such as osteopontin (see also e.g. table 1 from Sixt et al., Current Opinion in Cell Biology 2006, 18:482 [symbol] [symbol] 490. Virus infection. Several viruses use the integrin to bind and infect cells (see table 1 from Dunehoo et al., Journal of pharmaceutical sciences, vol. 95, (9), 2006, pp 1856-1872. The foot-and-mouth disease virus also use aVb6 as a receptor. In addition to the antibody therapies, many small-molecule antagonists have been developed: Tirofiban, and Epifibatide are two clinically approved antagonist. Other compounds also exist like BIRT0377, LFA703, and A-286982 (See Shimaoka and Springer, Nature reviews in Drug Discovery (2) pp 703-717, 2003 and reference therein). Many peptide therapies have been developed as well (see. Dunehoo et al., Journal of pharmaceutical sciences, vol. 95, (9), 2006, pp 1856-1872 and reference there in).

Agonistic compounds have also been found; see, A Small Molecule Agonist of an Integrin, alphaLbeta2 (Yang et al. JBC, (281) pp. 37904□387912, 2006). Although it stimulates ligand binding, this compound nonetheless inhibits lymphocyte transendothelial migration probably because of a de-adhesion defect.

The polypeptides and compositions of the present invention can generally be used to modulate, and in particular inhibit and/or prevent, binding of Integrin-Ligands to the Integrins, and thus to modulate, and in particular inhibit or prevent, the signalling that is mediated by Integrin-Ligands to Integrins, to modulate the biological pathways in which Integrin-Ligands and/or Integrins are involved, and/or to modulate the biological mechanisms, responses and effects associated with such signalling or these pathways.

As such, the polypeptides and compositions of the present invention can be used for the prevention and treatment (as defined herein) of autoimmune diseases, cancer metastasis and thrombotic vascular diseases. Generally, [symbol] [symbol] autoimmune diseases, cancer metastasis and thrombotic vascular diseases□ can be defined as diseases and disorders that can be prevented and/or treated, respectively, by suitably administering to a subject in need thereof (i.e. having the disease or disorder or at least one symptom thereof and/or at risk of attracting or developing the disease or disorder) of either a polypeptide or composition of the invention (and in particular, of a pharmaceutically active amount thereof) and/or of a known active principle active against Integrins or a biological pathway or mechanism in which Integrins is involved (and in particular, of a pharmaceutically active amount thereof). Examples of such autoimmune diseases, cancer metastasis and thrombotic vascular diseases will be clear to the skilled person based on the disclosure herein, and for example include the following diseases and disorders:

Inflammatory disease, auto-immune diseases, atherosclerosis: Infiltration of leukocyte at the site of infection or inflammation depends on the adhesion of the leukocyte to the endothelial cell layer which is dependent integrins.

Psoriasis: Efalizumab (Raptiva™) blocks LFA-1 (aLb2) and is use for the treatment of chronic plaque psoriasis

Multiple Sclerosis: Natalizumab (Tysabri/Antegren™) blocks very late antigen-4 (VLA4/a4b1) for the treatment of relapsing-remitting multiple sclerosis.

Thrombosis: ReoPro/Abciximab target and blocks alphaIIbbeta3 (platelet integrin), Centocor/JandJ FDA approved in 1994

Cancer: Blocking a5b1 blocks angiogenesis and consequently is used against cancer. Also anti alphaV therapies are efficient in blocking tumorigenesis. For example, Volociximab is an anti-a5b1 antibody inhibiting angiogenesis (PDL Biopharma/Biogen Idec) and CNTO95 is an anti-aV. now in Phase I (Medarex/Centocor). Vitaxin/Abegrin/MEDI-522 (MedImmune) is in clinical trials (Phase 3) for metastatic melanoma and prostate cancer; blocks the interaction of alphaVbeta3 (the predominant integrin on osteoclasts) with various ligands such as osteopontin.

Virus infection. Several virus use the integrin to bind and infect cells (see Table 1 from Dunehoo et al., Journal of pharmaceutical sciences, vol. 95(9), 2006, pp 1856-1872.

The foot-and-mouth disease virus also use aVb6 as a receptor.

### SUMMARY OF THE INVENTION

In particular, the polypeptides and compositions of the present invention can be used for the prevention and treatment of autoimmune diseases, cancer metastasis and thrombotic vascular diseases which are characterized by excessive and/or unwanted signalling mediated by Integrins or by the pathway(s) in which Integrins are involved. Examples of such autoimmune diseases, cancer metastasis and thrombotic vascular diseases will again be clear to the skilled person based on the disclosure herein.

Thus, without being limited thereto, the amino acid sequences and polypeptides of the invention can for example be used to prevent and/or to treat all diseases and disorders that are currently being prevented or treated with active principles that can modulate Integrins-mediated signalling, such as those mentioned in the prior art cited above. It is also envisaged that the polypeptides of the invention can be used to prevent and/or to treat all diseases and disorders for which treatment with such active principles is currently being developed, has been proposed, or will be proposed or developed in future. In addition, it is envisaged that, because of their favourable properties as further described herein, the polypeptides of the present invention may be used for the prevention and treatment of other diseases and disorders than those for which these known active principles are being used or will be proposed or developed; and/or that the polypeptides of the present invention may provide new methods and regimens for treating the diseases and disorders described herein.

Other applications and uses of the amino acid sequences and polypeptides of the invention will become clear to the skilled person from the further disclosure herein.

Generally, it is an object of the invention to provide pharmacologically active agents, as well as compositions comprising the same, that can be used in the diagnosis, prevention and/or treatment of autoimmune diseases, cancer metastasis and thrombotic vascular diseases and of the further diseases and disorders mentioned herein; and to provide methods for the diagnosis, prevention and/or treatment of such diseases and disorders that involve the administration and/or use of such agents and compositions.

In particular, it is an object of the invention to provide such pharmacologically active agents, compositions and/or methods that have certain advantages compared to the agents, compositions and/or methods that are currently used and/or known in the art. These advantages will become clear from the further description below.

More in particular, it is an object of the invention to provide therapeutic proteins that can be used as pharmacologically active agents, as well as compositions comprising the same, for the diagnosis, prevention and/or treatment of autoimmune diseases, cancer metastasis and thrombotic vascular diseases and of the further diseases and disorders mentioned herein; and to provide methods for the diagnosis, prevention and/or treatment of such diseases and disorders that involve the administration and/or the use of such therapeutic proteins and compositions.

Accordingly, it is a specific object of the present invention to provide amino acid sequences that are directed against (as defined herein) Integrins, in particular against Integrins from a warm-blooded animal, more in particular against Integrins from a mammal, and especially against human Integrins; and to provide proteins and polypeptides comprising or essentially consisting of at least one such amino acid sequence.

In particular, it is a specific object of the present invention to provide such amino acid sequences and such proteins and/or polypeptides that are suitable for prophylactic, therapeutic and/or diagnostic use in a warm-blooded animal, and in particular in a mammal, and more in particular in a human being.

More in particular, it is a specific object of the present invention to provide such amino acid sequences and such proteins and/or polypeptides that can be used for the prevention, treatment, alleviation and/or diagnosis of one or more diseases, disorders or conditions associated with Integrins and/or mediated by Integrins (such as the diseases, disorders and conditions mentioned herein) in a warm-blooded animal, in particular in a mammal, and more in particular in a human being.

It is also a specific object of the invention to provide such amino acid sequences and such proteins and/or polypeptides that can be used in the preparation of pharmaceutical or veterinary compositions for the prevention and/or treatment of one or more diseases, disorders or conditions associated with and/or mediated by Integrins (such as the diseases, disorders and conditions mentioned herein) in a warm-blooded animal, in particular in a mammal, and more in particular in a human being. In the invention, generally, these objects are achieved by the use of the amino acid sequences, proteins, polypeptides and compositions that are described herein.

In general, the invention provides amino acid sequences that are directed against (as defined herein) and/or can specifically bind (as defined herein) to Integrins; as well as compounds and constructs, and in particular proteins and polypeptides, that comprise at least one such amino acid sequence.

More in particular, the invention provides amino acid sequences that can bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such amino acid sequence.

In particular, amino acid sequences and polypeptides of the invention are preferably such that they:
bind to Integrins with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to Integrins with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷M⁻¹ s⁻¹;
   and/or such that they:
bind to Integrins with a k_{off} rate between 1s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a monovalent amino acid sequence of the invention (or a polypeptide that contains only one amino acid sequence of the invention) is preferably such that it will bind to Integrins with an affinity less than 500 nM, preferably less than 200 nM, more preferably less than 10 nM, such as less than 500 pM.

Some preferred IC50 values for binding of the amino acid sequences or polypeptides of the invention to Integrins will become clear from the further description and examples herein.

For binding to Integrins, an amino acid sequence of the invention will usually contain within its amino acid sequence one or more amino acid residues or one or more stretches of amino acid residues (i.e. with each□ stretch□ comprising two or amino acid residues that are adjacent to each other or in close proximity to each other, i.e. in the primary or tertiary structure of the amino acid sequence) via which the amino acid sequence of the invention can bind to Integrins, which amino acid residues or stretches of amino acid residues thus form the □site□ for binding to Integrins (also referred to herein [symbol] [symbol] binding site□).

The amino acid sequences provided by the invention are preferably in essentially isolated form (as defined herein), or form part of a protein or polypeptide of the invention (as defined herein), which may comprise or essentially consist of one or more amino acid sequences of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than Integrins), so as to provide a monovalent, multivalent or multispecific polypeptide of the invention, respectively, all as described herein. Such a protein or polypeptide may also be in essentially isolated form (as defined herein).

The amino acid sequences and polypeptides of the invention as such preferably essentially consist of a single amino acid chain that is not linked via disulphide bridges to any other amino acid sequence or chain (but that may or may not contain one or more intramolecular disulphide bridges. For example, it is known that Nanobodies □as described herein—may sometimes contain a disulphide bridge between CDR3 and CDR1 or FR2). However, it should be noted that one or more amino acid sequences of the invention may be linked to each other and/or to other amino acid sequences (e.g. via disulphide bridges) to provide peptide constructs that may also be useful in the invention (for example Fab□ fragments, F(ab□₂ fragments, ScFv constructs, □ diabodies□ and other multispecific constructs. Reference is for example made to the review by Holliger and Hudson, Nat. Biotechnol. 2005 September; 23(9):1126-36).

Generally, when an amino acid sequence of the invention (or a compound, construct or polypeptide comprising the same) is intended for administration to a subject (for example for therapeutic and/or diagnostic purposes as described herein), it is preferably either an amino acid sequence that does not occur naturally in said subject; or, when it does occur naturally in said subject, in essentially isolated form (as defined herein).

It will also be clear to the skilled person that for pharmaceutical use, the amino acid sequences of the invention (as well as compounds, constructs and polypeptides comprising the same) are preferably directed against human Integrins; whereas for veterinary purposes, the amino acid sequences and polypeptides of the invention are preferably directed against Integrins from the species to be treated, or at least cross-reactive with Integrins from the species to be treated.

Furthermore, an amino acid sequence of the invention may optionally, and in addition to the at least one binding site for binding against Integrins, contain one or more further binding sites for binding against other antigens, proteins or targets.

The efficacy of the amino acid sequences and polypeptides of the invention, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person, and for example include Biacore, FLIPR, cell based models such as adhesion of cell expressing relevant integrin on relevant coated ligands, binding of labelled ligand to cell expressing the relevant integrin, attachment of leukocyte to endothelial cells, cell survival text for cancer indication, animal models such as mouse models monitoring inflammatory cell recruitment, cancer models, as well as the assays and animal models used in the experimental part below and in the prior art cited herein.

Also, according to the invention, amino acid sequences and polypeptides that are directed against Integrins from a first species of warm-blooded animal may or may not show cross-reactivity with Integrins from one or more other species of warm-blooded animal. For example, amino acid sequences and polypeptides directed against human Integrins may or may not show cross reactivity with Integrins from one or more other species of primates (such as, without limitation, monkeys from the genus Macaca (such as, and in particular, cynomolgus monkeys (Macaca fascicularis) and/or rhesus monkeys (Macaca mulatta)) and baboon (Papio ursinus)) and/or with Integrins from one or more species of animals that are often used in animal models for diseases (for example mouse, rat, rabbit, pig or dog), and in particular in animal models for diseases and disorders associated with Integrins (such as the species and animal models mentioned herein). In this respect, it will be clear to the skilled person that such cross-reactivity, when present, may have advantages from a drug development point of view, since it allows the amino acid sequences and polypeptides against human Integrins to be tested in such disease models.

More generally, amino acid sequences and polypeptides of the invention that are cross-reactive with Integrins from multiple species of mammal will usually be advantageous for use in veterinary applications, since it will allow the same amino acid sequence or polypeptide to be used across multiple species. Thus, it is also encompassed within the scope of the invention that amino acid sequences and polypeptides directed against Integrins from one species of animal (such as amino acid sequences and polypeptides against human Integrins) can be used in the treatment of another species of animal, as long as the use of the amino acid sequences and/or polypeptides provide the desired effects in the species to be treated.

The present invention is in its broadest sense also not particularly limited to or defined by a specific antigenic determinant, epitope, part, domain, subunit or confirmation (where applicable) of Integrins against which the amino acid sequences and polypeptides of the invention are directed. For example, the amino acid sequences and polypeptides may or may not be directed against an □interaction site□ (as define herein [symbol] [symbol] ), it is generally assumed and preferred that the amino acid sequences and polypeptides of the invention are preferably directed against an interaction site (as defined herein), and in particular against the site of integrin activation, e.g. the amino acid sequences and polypeptides may or may not be directed against an epitope available only after activation, or site of integrin inactivation, e.g. the amino acid sequences and polypeptides may or may not be directed against an epitope available only when inactivated.

As further described herein, a polypeptide of the invention may contain two or more amino acid sequences of the invention that are directed against Integrins. Generally, such polypeptides will bind to Integrins with increased avidity compared to a single amino acid sequence of the invention. Such a polypeptide may for example comprise two amino acid sequences of the invention that are directed against the same antigenic determinant, epitope, part, domain, subunit or confirmation (where applicable) of Integrins (which may or may not be an interaction site); or comprise at least one □ first□ amino acid sequence of the invention that is directed against a first same antigenic determinant, epitope, part, domain, subunit or confirmation (where applicable) of Integrins (which may or may not be an interaction site); and at least one□second□ amino acid sequence of the invention [symbol] [symbol] inst a second antigenic determinant, epitope, part, domain, subunit or confirmation (where applicable) different from the first (and which again may or may not be an interaction site). Preferably, in such □biparatopic□ polypeptides of the [symbol] [symbol] amino acid sequence of the invention is directed against an interaction site (as defined herein), although the invention in its broadest sense is not limited thereto.

Also, when the target is part of a binding pair (for example, a receptor-ligand binding pair), the amino acid sequences and polypeptides may be such that they compete with the cognate binding partner (e.g. the ligand, receptor or other binding partner, as applicable) for binding to the target, and/or such that they (fully or partially) neutralize binding of the binding partner to the target.

It is also within the scope of the invention that, where applicable, an amino acid sequence of the invention can bind to two or more antigenic determinants, epitopes, parts, domains, subunits or confirmations of Integrins. In such a case, the antigenic determinants, epitopes, parts, domains or subunits of Integrins to which the amino acid sequences and/or polypeptides of the invention bind may be essentially the same (for example, if Integrins contains repeated structural motifs or occurs in a multimeric form) or may be different (and in the latter case, the amino acid sequences and polypeptides of the invention may bind to such different antigenic determinants, epitopes, parts, domains, subunits of Integrins with an affinity and/or specificity which may be the same or different). Also, for example, when Integrins exists in an activated conformation and in an inactive conformation, the amino acid sequences and polypeptides of the invention may bind to either one of these confirmation, or may bind to both these confirmations (i.e. with an affinity and/or specificity which may be the same or different). Also, for example, the amino acid sequences and polypeptides of the invention may bind to a conformation of Integrins in which it is bound to a pertinent ligand, may bind to a conformation of Integrins in which it not bound to a pertinent ligand, or may bind to both such conformations (again with an affinity and/or specificity which may be the same or different).

It is also expected that the amino acid sequences and polypeptides of the invention will generally bind to all naturally occurring or synthetic analogs, variants, mutants, alleles, parts and fragments of Integrins; or at least to those analogs, variants, mutants, alleles, parts and fragments of Integrins that contain one or more antigenic determinants or epitopes that are essentially the same as the antigenic determinant(s) or epitope(s) to which the amino acid sequences and polypeptides of the invention bind in Integrins (e.g. in wild-type Integrins). Again, in such a case, the amino acid sequences and polypeptides of the invention may bind to such analogs, variants, mutants, alleles, parts and fragments with an affinity and/or specificity that are the same as, or that are different from (i.e. higher than or lower than), the affinity and specificity with which the amino acid sequences of the invention bind to (wild-type) Integrins. It is also included within the scope of the invention that the amino acid sequences and polypeptides of the invention bind to some analogs, variants, mutants, alleles, parts and fragments of Integrins, but not to others.

When Integrins exists in a monomeric form and in one or more multimeric forms, it is within the scope of the invention that the amino acid sequences and polypeptides of the invention only bind to Integrins in monomeric form, only bind to Integrins in multimeric form, or bind to both the monomeric and the multimeric form. Again, in such a case, the amino acid sequences and polypeptides of the invention may bind to the monomeric form with an affinity and/or specificity that are the same as, or that are different from (i.e. higher than or lower than), the affinity and specificity with which the amino acid sequences of the invention bind to the multimeric form.

Also, when Integrins can associate with other proteins or polypeptides to form protein complexes (e.g. with multiple subunits), it is within the scope of the invention that the amino acid sequences and polypeptides of the invention bind to Integrins in its non-associated state, bind to Integrins in its associated state, or bind to both. In all these cases, the amino acid sequences and polypeptides of the invention may bind to such multimers or associated protein complexes with an affinity and/or specificity that may be the same as or different from (i.e. higher than or lower than) the affinity and/or specificity with which the amino acid sequences and polypeptides of the invention bind to Integrins in its monomeric and non-associated state.

Also, as will be clear to the skilled person, proteins or polypeptides that contain two or more amino acid sequences directed against Integrins may bind with higher avidity to Integrins than the corresponding monomeric amino acid sequence(s). For example, and without limitation, proteins or polypeptides that contain two or more amino acid sequences directed against different epitopes of Integrins may (and usually will) bind with higher avidity than each of the different monomers, and proteins or polypeptides that contain two or more amino acid sequences directed against Integrins may (and usually will) bind also with higher avidity to a multimer of Integrins.

Generally, amino acid sequences and polypeptides of the invention will at least bind to those forms of Integrins (including monomeric, multimeric and associated forms) that are the most relevant from a biological and/or therapeutic point of view, as will be clear to the skilled person.

It is also within the scope of the invention to use parts, fragments, analogs, mutants, variants, alleles and/or derivatives of the amino acid sequences and polypeptides of the invention, and/or to use proteins or polypeptides comprising or essentially consisting of one or more of such parts, fragments, analogs, mutants, variants, alleles and/or derivatives, as long as these are suitable for the uses envisaged herein. Such parts, fragments, analogs, mutants, variants, alleles and/or derivatives will usually contain (at least part of) a functional antigen-binding site for binding against Integrins; and more preferably will be capable of specific binding to Integrins, and even more preferably capable of binding to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Some non-limiting examples of such parts, fragments, analogs, mutants, variants, alleles, derivatives, proteins and/or polypeptides will become clear from the further description herein. Additional fragments or polypeptides of the invention may also be provided by suitably combining (i.e. by linking or genetic fusion) one or more (smaller) parts or fragments as described herein.

In one specific, but non-limiting aspect of the invention, which will be further described herein, such analogs, mutants, variants, alleles, derivatives have an increased half-life in serum (as further described herein) compared to the amino acid sequence from which they have been derived. For example, an amino acid sequence of the invention may be linked (chemically or otherwise) to one or more groups or moieties that extend the half-life (such as PEG), so as to provide a derivative of an amino acid sequence of the invention with increased half-life.

In one specific, but non-limiting aspect, the amino acid sequence of the invention may be an amino acid sequence that comprises an immunoglobulin fold or may be an amino acid sequence that, under suitable conditions (such as physiological conditions) is capable of forming an immunoglobulin fold (i.e. by folding). Reference is inter alia made to the review by Halaby et al., J. (1999) Protein Eng. 12, 563-71. Preferably, when properly folded so as to form an immunoglobulin fold, such an amino acid sequence is capable of specific binding (as defined herein) to Integrins; and more preferably capable of binding to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Also, parts, fragments, analogs, mutants, variants, alleles and/or derivatives of such amino acid sequences are preferably such that they comprise an immunoglobulin fold or are capable for forming, under suitable conditions, an immunoglobulin fold.

In particular, but without limitation, the amino acid sequences of the invention may be amino acid sequences that essentially consist of 4 framework regions (FR1 to FR4 respectively) and 3 complementarity determining regions (CDR1 to CDR3 respectively); or any suitable fragment of such an amino acid sequence (which will then usually contain at least some of the amino acid residues that form at least one of the CDR□s, as further described herein).

The amino acid sequences of the invention may in particular be an immunoglobulin sequence or a suitable fragment thereof, and more in particular be an immunoglobulin variable domain sequence or a suitable fragment thereof, such as light chain variable domain sequence (e.g. a V_{L}-sequence) or a suitable fragment thereof; or a heavy chain variable domain sequence (e.g. a V_{H}-sequence) or a suitable fragment thereof. When the amino acid sequence of the invention is a heavy chain variable domain sequence, it may be a heavy chain variable domain sequence that is derived from a conventional four-chain antibody (such as, without limitation, a V_{H} sequence that is derived from a human antibody) or be a so-called V_{HH}-sequence (as defined herein) that is derived from a so-called□ heavy chain antibody□ (as defined herein).

However, it should be noted that the invention is not limited as to the origin of the amino acid sequence of the invention (or of the nucleotide sequence of the invention used to express it), nor as to the way that the amino acid sequence or nucleotide sequence of the invention is (or has been) generated or obtained. Thus, the amino acid sequences of the invention may be naturally occurring amino acid sequences (from any suitable species) or synthetic or semi-synthetic amino acid sequences. In a specific but non-limiting aspect of the invention, the amino acid sequence is a naturally occurring immunoglobulin sequence (from any suitable species) or a synthetic or semi-synthetic immunoglobulin sequence, including but not limited to□ humanized□ (as defined herein) immunoglobulin sequences (such as partially or fully humanized mouse or rabbit immunoglobulin sequences, and in particular partially or fully humanized V_{HH} sequences or Nanobodies),□ camelized□ (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Reference is for example made to the standard handbooks, as well as to the further description and prior art mentioned herein.

Similarly, the nucleotide sequences of the invention may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

The amino acid sequence of the invention may in particular be a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody™ (as defined herein, and including but not limited to a V_{HH} sequence); other single variable domains, or any suitable fragment of any one thereof. For a general description of (single) domain antibodies, reference is also made to the prior art cited above, as well as to EP 0 368 684. For the term□ dAb□s□, reference is for example made to Ward et al. (Nature 1989 Oct 341 (6242): 544-6), to Holt et al., Trends Biotechnol., 2003, 21(11):484-490; as well as to for example WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called □ IgNAR domains□, see for example WO 05/18629).

In particular, the amino acid sequence of the invention may be a Nanobody® (as defined herein) or a suitable fragment thereof. [Note: Nanobody®, Nanobodies® and Nanoclone® are registered trademarks of Ablynx N. V] Such Nanobodies directed against Integrins will also be referred to herein as □Nanobodies of the invention□.

For a general description of Nanobodies, reference is made to the further description below, as well as to the prior art cited herein. In this respect, it should however be noted that this description and the prior art mainly described Nanobodies of the so-called □_{H}3 class □ (i.e. Nanobodies with a high degree of sequence homology to human germline sequences of the V_{H}3 class such as DP-47, DP-51 or DP-29), which Nanobodies form a preferred aspect of this invention. It should however be noted that the invention in its broadest sense generally covers any type of Nanobody directed against Integrins, and for example also covers the Nanobodies belonging to the so-called □_{H}4 class□ (i.e. Nanobodies with a high degree of sequence homology to human germline sequences of the V_{H}4 class such as DP-78), as for example described in WO 07/118,670.

Generally, Nanobodies (in particular V_{HH} sequences and partially humanized Nanobodies) can in particular be characterized by the presence of one or more □Hallmark residues□ as de(scribed herein) in one or more of the framework sequences (again as further described herein).

Thus, generally, a Nanobody can be defined as an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody can be an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody can be an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
i) preferably one or more of the amino acid residues at positions 11, 37, 44, 45, 47, 83, 84, 103, 104 and 108 according to the Kabat numbering are chosen from the Hallmark residues mentioned in Table A-3 below;
   and in which:
ii) said amino acid sequence has at least 80% amino acid identity with at least one of the amino acid sequences of SEQ ID NO□ s: 1 to 22, in which for the purposes of determining the degree of amino acid identity, the amino acid residues that form the CDR sequences (indicated with X in the sequences of SEQ ID NO□ s: 1 to 22) are disregarded.

In these Nanobodies, the CDR sequences are generally as further defined herein.

Thus, the invention also relates to such Nanobodies that can bind to (as defined herein) and/or are directed against Integrins, to suitable fragments thereof, as well as to polypeptides that comprise or essentially consist of one or more of such Nanobodies and/or suitable fragments.

SEQ ID NO□ s 1316 to 1487 (see Table 1) give the amino acid sequences of a number of V_{HH} sequences that have been raised against Integrins.

TABLE 1
Preferred VHH sequences or Nanobody sequences and their specificity (also
referred herein as a sequence with a particular name or SEQ ID NO: X,
wherein X is a number referring to the relevant amino acid sequence):

| | | SEQ | |
|---|---|---|---|
| | | ID | |
| | | NO: | |
| | | X, | |
| | | wherein | |
| Name | Specificity | X = | Amino acid sequence |
| 138-G2, | alphaL | 1316 | EVQLVESGGGLVQPGGSLRLSCAASGFTLDYYNIGWFRQAP |
| 235-E10 | | | GEEREGVSCVSSSGGSTYYADSVKGRFTISRDNAKNTVYLQ |
| | | | MNSLKPEDTAVYYCATLNLFTTCDGPWGYEYDYYGQGTQV |
| | | | TVSS |
| 138- | alphaL | 1317 | EVQLVESGGGLVQPGGSLRLSCAASGFTLDYYNIGWFRQAP |
| H5, 235A6, | | | GKEREGVSCVSSSGGSTYYADSVKGRFTISRDNAKNTVYLQ |
| 243B9 | | | MNSLKPEDTAVYYCATLNLFTTCDGPWGYEYDYYGQGTQV |
| | | | TVSS |
| 235-E9 | alphaL | 1318 | XVQLVESGGGLVQPGGSLRLSCAASGFTLDYYAIGWFRQAP |
| | | | GKEREGVSCISSSDGSTYYADSVKGRFTISRDNAKNTVYLQ |
| | | | MNSLKPEDTAVYYCATLNLFTTCDGPWGYEYDYYGQGTQV |
| | | | TVSS |
| 138-D2 | alphaL | 1319 | EVQLVESGGGLVQPGGSLRLSCAASGFTLDYYNIGWFRQAP |
| | | | GKEREGVSCVSDSGGSTYYADSVKGRFTISRDNAKNTVYLQ |
| | | | MNSLKPEDTAVYYCATLNLFTTCDGPWGYEYDYYGQGTQV |
| | | | TVSS |
| 138-E4 | alphaL | 1320 | EVQLVESGGGLVQPGGSLRLSCAASGFTLDSYNIGWFRQAP |
| | | | GEEREGVSCVSSSGGSTYYADSVKGRFTISRDNAKNTVYLQ |
| | | | MNSLKPEDTAVYYCATLNLFTTCDGPWGYEYDYYGQGTQV |
| | | | TVSS |
| 138-B10 | alphaL | 1321 | EVQLVESGGGLVQAGGSLRLSCAASGFTADDYAIGWFRQAP |
| | | | GKEREGVSCISSSDGSTFYADSVKGRFTISSDNAKNTVYLQM |
| | | | NSLKPEDTAVYYCAADPRFEPTLLCTDYDYEDYWGQGTQV |
| | | | TVSS |
| 138-G11 | alphaL | 1322 | EVQLVESGGGLVQPGGSLRLSCAASGFTFDDYAIGWFRQAP |
| | | | GKEREGVSCISSSDGSTFYANSVKDRFTVSSDNAKNTVYLQ |
| | | | MNSLKPEDTAVYYCAABPXLSPQTYCTDYDYSYWGQGTQV |
| | | | TVSS |
| 138-D7 | alphaL | 1323 | EVQLVESGGGLVQAGGSLRLSCAASGFTFDDYAIGWFRQAP |
| | | | GKEREGVSCISSSDGYTYYADSVKDRFTISSDDAKNTVYLQ |
| | | | MNSLKPEDTAVYYCAATPRRFGWCSDYBEYDYWGQGTQV |
| | | | TVSS |
| 138- | alphaL | 1324 | EVQLVESGGGLVQAGGSLRLSCAASGFTFDDYAIGWFRQAP |
| H7, 152- | | | GKEREGVSCISSSDGYSFYANSVKGRFTISSDNAKNTVYLQM |
| B7 | | | NSLKPEDTAVYYCAATPRRFGWCSDYDEYDYWGQGTQVT |
| | | | VSS |
| 138-D11 | alphaL | 1325 | EVQLVESGGGLVXAGGSLRLSCAXSGFXFDDYAIGWFRQAP |
| | | | GEEREGVSCISSSDGYTYYAHSVKDRFTISSDNAKNTMYLQ |
| | | | MNSLKPEDTAVYYCAATPRRFGWXSDYDXYDYWGQGTQV |
| | | | TVSS |
| 138-C10 | alphaL | 1326 | XVQLVESGGGLVQAGGSLRLSCAASGFTFDDYVIGWFXQAP |
| | | | GKEREGVSCISSSEGYTFYADSVKDRFTISXDNAKNTVSLQM |
| | | | NSLKPEDTAVYYCAAXRKIIGLWCSDYDNYDYWGQGTQVT |
| | | | VSS |
| 235-B7 | alphaL | 1327 | EVQLVESGGGLVRAGGSLRLSCTASGNILNIASMGWYRQAP |
| | | | GKQRTWVARIRSDGTTSYQDAVKGRFTIAKDNAKNAGYLQ |
| | | | MDNLKPEDTAVYYCNAAGSTIDGGFSSWGPGTQVTVSS |
| 248-F2 | alphaL | 1328 | EVQLVESGGGLVRAGGSLRLSCTASGNILNIASMGWYRQAL |
| | | | GKQRTWVARIRSDGTTSYQDAVKGRFTIAKDNAKNAGYLQ |
| | | | MDNLKPEDTAVYYCNAAGSTIDGGFSSWGPGTQVTVSS |
| 248- | alphaL | 1329 | EVQLVESGGGLVQAGGSLRLSCAASGSGFNIVNAGWYRQGP |
| F7, D8 | | | EKQRELVARITSGGTTNYAESVKGRFTISRDNAKNTVYLQM |
| | | | NSLKPEDTAVYSCNARVIAPGRLDDIWGQGTQVTVSS |
| 248-H7 | alphaL | 1330 | EVxLVESGGGLVQAGGSLRLSCAASGSGFNIVNAGWYRQGP |
| | | | GKQREFVARITSGGTTNYAESVKGRFTISRDNAKNTVYLQM |
| | | | NSLKPEDTAVYSCNARVIAPGRLDDIWGQGTQVTVSS |
| 248-D7 | alphaL | 1331 | EVQLVESGGGLVQPGGSLRLSCAASGFAFSSYAMSWVRQAP |
| | | | GKGVEWVSAINSGGGSTSYLNSVKGRFTISRDDAKNTLYLQ |
| | | | MNSLKPEDTAVYYCAKPIYYSPNTYPPTSRYDYRGQGTQVT |
| | | | VSS |
| 236- | alphaL | 1332 | KVQLVESGGGLVQPGGSLRLSCAASGFAFSSYVMTWVRQA |
| B4, A5, | | | PGKGLEWVSSITSGGGYTSYLNSVKGRFTISRDDAKNTLYLQ |
| 248-G8 | | | MNSLKPEDTAVYYCAKPTFYSPNMYPPTSRYDYRGQGTQV |
| | | | TVSS |
| 248-E8 | alphaL | 1333 | EVQLVESGGGLVQPGGSLRLSCAASGFAFSSYVMTWVRQAP |
| | | | GKGLEWVSSITSGGGYTSYVNSVKGRFTISRDDAKNTLYLQ |
| | | | MNSLKPEDTAVYYCAKPTFYSPNMYPPTSRYDYRGQGTQV |
| | | | TVSS |
| 248-C8 | alphaL | 1334 | EVQLVESGGGLVQAGGSLRLSCAASGFTLDAYAIGWFRQAP |
| | | | GKEREGVSCISSSDGSTFYADSVKDRFTISSDNAKNTVYLQM |
| | | | NSLKPEDTAVYYCAADRRGKSEMYCTDYSYSDAWGQGTQ |
| | | | VTVSS |
| 236-E11 | alphaL | 1335 | EVxLVESGGGLVQAGGSLRLSCAASGFTLDAYAIGWFRQAP |
| | | | GKEREGVSCISSSDGSRFYADSVKDRFTISSDNAKNTVYLQM |
| | | | NSLKPEDTAVYYCAADRRGKSEMYCTDYAYSDAWGQGTQ |
| | | | VTVSS |
| 235-F3 | alphaL | 1336 | EVQLVESGGGLVQAGGSLTLSCALSGGSSSIANSAWYRQAP |
| | | | GNQRELVARITSNDNTYYADSVKGRLTISKDNAKNTASLQM |
| | | | NSLKPEDTAVYYCFVRTVGTGSLFDYWGQGTQVTVSS |
| 248- | alphaL | 1337 | EVQLVESGGGLVQAGGSLTLSCALSGGSSSIANSAWYRQAP |
| F1, G1 | | | GNQRELVARITSNDNTYYADSVKGRFTISKDNAKNTASLQM |
| | | | NSLKPEDTAVYYCFVRTVGTGSLFDYWGQGTQVTVSS |
| 248-G2 | alphaL | 1338 | EVQPVESGGGLVQAGGSLTLSCALSGGSSSIANSAWYRQAP |
| | | | GNQRELVARITSNDNTYYADSVKGRFTISKDNAKNTASLQM |
| | | | NSLKPEDTAVYYCFVRTVGTGSLFDYWGQGTQVTVSS |
| 138- | alphaL | 1339 | EVQLVESGGGLVQAGGSLRLSCAASGFTFDDYAIGWFRQAP |
| G4, G5 | | | GKEREGVSCISSSHGSTFYADSVKDRFTISSDNAKNTVYLQM |
| | | | NSLKPEDTAVYYCAAALGGGSSWCTTYEYDAWGQGTQVT |
| | | | VSS |
| 235- | alphaL | 1340 | EVQLVESGGGLVQSGGSLRLSCAASGSIVGISDMRWYRQAP |
| A12, 248- | | | GNQRELAASISRGGSTNYGDFVKGRFTISRDNAKNTVSLQM |
| B1, C1, E1, | | | SSLEPEDTAVYYCNAAVAAGTVGAYTLRNYWGQGTQVTVS |
| H2 | | | S |
| 248-B7 | alphaL | 1341 | KVQLVESGGGLVQAGGSLRLTCAASGSILSVSTMTWYRQVL |
| | | | GTQRELVASITRSGGSNYADPVKGRFTIARDNAKNTMYLQM |
| | | | NSLKPEDTAIYYCNAVIASNSGRSYDLRNYWGQGTQVTVSS |
| 248-A1 | alphaL | 1342 | EVQLVESGGGLVQPGGSLRLSCTASGSVFSIGNMGWYRQAP |
| | | | GKQRELVATITQAGSPNYSQSAKGRFTISRDVPKNTVSLQMN |
| | | | SLKPEDTAVYYCNGNIVTYDRGRTTVKNYWGQGTQVTVSS |
| 248- | alphaL | 1343 | EVQLVESGGGLVQPGGSLRLYCAAPGTMYSFIEMGWYRQA |
| D2, D1, | | | PGKQRELVAHITSGGSTKYADSVKGRFTISRDLSLQMNNLNP |
| E2 | | | EDSAVYLCNMKGTDRRSYWGQGTQVTVSS |
| 248-A7 | alphaL | 1344 | XVQLVESGGGLVQAGGSLRLSCAASGFTFDDYAIGWFRQAP |
| | | | GKEREGVSCISRSAGSKYYADSVKDRFTISSDNAKNTVSLQM |
| | | | NSLKPEDTAVYYCAAYRAIGHFCTDYXDFVSWGQGTQVTV |
| | | | SS |
| 153-G1 | beta2 | 1345 | EVQLVESGGGLVQAGGSLRLSCTVSGSTGSINAMGWYRQAP |
| | | | GKQRELVAIIYSSGRIDYADSVKGRFTISRDNAKNTVYLQMN |
| | | | NLQPDDTAAYYCNAANPNTGWQRPHRASWGQGTQVTVSS |
| 138-C2 | beta2 | 1346 | EVQLVESGGGVVQAGGSLRLSCTVSGSTGSINAMGWYRQA |
| | | | PGKQRELVAIIYSSGTIBYADSVKGRFTISRDNAKNTVYLQM |
| | | | NSLKPDDTAAYYCNAANPNTGWQRPHRASWGQGTQVTVSS |
| 139-C2 | beta2 | 1347 | EVQLVESGRGLVQAGGSLRLSCTVSGSTGSINAMGWYRQAP |
| | | | GKQRELVAIIYSSGRIDYADSVKGRFTISRDNAKNTVBLQMN |
| | | | SLKPDDTAAYYCNAANPNTGWQRPHRASWGQGTQVTVSS |
| 235-E3 | beta2 | 1348 | EVQLVESGGGLVQAGGSLRLSCTVSGSTGSINVMGWYRQAP |
| | | | GKQRELVAIIYSSGTLDYADSVKGRFTISRDNAKNTVYLQM |
| | | | NSLKPDDTAAYYCNAAMQDSAWLRPHRASWGQGTQVTVS |
| | | | S |
| 152-C6 | beta2 | 1349 | EVQLVESGGGLVQAGGSLRLSCTVSGSTGSINAMGWYRQAP |
| | | | GR*RELVAIIYSSGRIDYADSVKGRFTISRDNAKNTVDLQMN |
| | | | SLKPDDTAAYYCNAANPNTGWQRPHRASWGQGTQVTVSS |
| 153-G1 | beta2 | 1350 | EVQLVESGGGLVQAGGSLRLSCTVSGSTGSINAMGWYRQAP |
| | | | GKQRELVAIIYSSGRIDYADSVKGRFTISRDNAKNTVYLQMN |
| | | | NLQPDDTAAYYCNAANPNTGWQRPHRASWGQGTQVTVSS |
| 138- | beta2 | 1351 | EVQLVESGGELVQAGGSLRLSCAASGSVSSINVMGWYRQAP |
| A2, E5, F5, | | | GKQRELVATISSSGYTDYSDSAKGRFTISRDNKNTVHLQMNS |
| 139- | | | LKPEDTAVYYCRASTLRTGWFTGWGQGTQVTVSS |
| G2, 152- | | | |
| A6, 153- | | | |
| B5, B6, F5, | | | |
| 253- | | | |
| D3, G3 | | | |
| 152- | beta2 | 1352 | EMQLVESGGELVQAGGSLRLSCAASGSVSSINVMGWYRQA |
| D4, E4 | | | PGKQRELVATISSSGYTDYSDSAKGRFTISRDNKNTVHLQMN |
| | | | SLKPEDTAVYYCRASTLRTGWFTGWGQGTQVTVSS |
| 152-F2 | beta2 | 1353 | EVQLVESGGELVQAGGSLRLSCAASGSVSSINVMGWYRQAP |
| | | | GKQRELVATISSSGCTDYSDSAKGRFTISRDNKNTVHLQMNS |
| | | | LKPEDTAVYYCRASTLRTGWFTGWGQGTQVTVSS |
| 138-B2 | beta2 | 1354 | AEVQLVESGGELVQAGGSLRLSCAAPGSVSSINVMGWYRQ |
| | | | APGKQRELVATISSSGYTDYADSAKGRFTISRDNKNTVHLQ |
| | | | MNSLKPEDTAVYYCRASTLRTGWFTGWGQGTQVTVSS |
| 153-D4 | beta2 | 1355 | EVQLVESGGGLVQAGRSLRLSCAASGSVSSINVMAWYRQAP |
| | | | GKQRELVATISSSGYTDYSDSAKGRFTISRDDXNTVHLQMNS |
| | | | LKPEDTAVYYCRASTARTGWLRAWGQGTQVTVSS |
| 153- | beta2 | 1356 | EVQLVESGGGLVQAGGSLRLSCAASGSVSSINVMGWYRQAP |
| G2,F4 | | | GKQRELVATISSSGYTDYSDSAKGRFTISRDNENTVHLQMNS |
| | | | LKPEDTGVYYCRASTARTGWLXPWGQGTQVTVSS |
| 153-G5 | beta2 | 1357 | EVQLVESGGGLVQAGGSLRLSCAASGSVSSTNVMAWYRQAP |
| | | | GKERELVATISTTGYTDYSXSAKGRFTISRDSKNTVHLQMNS |
| | | | LKPEDTAVYYCRASTLRTGWLMGWGQGTQVTVSS |
| 153-H2 | beta2 | 1358 | XVQLVESGGGLVQAGGSLRLSCAAXGSVSSINVMGWYRQA |
| | | | PGKQRELVATISTTGYTDYSXSAKGRFTISRDNKNTVHLQM |
| | | | NSLKPEDTAVYYCRASTLRTGWLKGWGQGTQVTVSS |
| 153-H4 | beta2 | 1359 | EVQLVESGGGLVQAGGSLRLSCAASGSVSSINVMAWYRQAP |
| | | | GKQRELVATISSTGYTDYSDSAKGRFTISRDNKNTVHLQMN |
| | | | SLKPEDTAVYYCRASTLRTGWFMGWGQGTQVTVSS |
| 152-C4 | beta2 | 1360 | EVQLVESGGGLVQAGGSLRLSCATSGSVSSINVMAWYRQAP |
| | | | GKQRELVATISTSGYTDYSDSAKGRFT1SRDNKNTVHLQMN |
| | | | SLKPEDTAVYYCRASTLRTGWLMGWGQGTQVTVSS |
| 152-F4 | beta2 | 1361 | EVQLVESGGGLVQAGGSLRLSCAASGSVSSINVMAWYRQAP |
| | | | GKQRELVATISTSGYTDYSDSAKGRFTISRDNKNTVHLQMN |
| | | | SLKPEDTAVYYCRASTLRTGWLMGWGQGTQVTVSS |
| 152-E1 | beta2 | 1362 | EVQLVESGGGLVQAGGSLRLSCAASGSVSAINVMAWYRQA |
| | | | PGKQRELVATISSTGYTDYSDSAKGRFTISRDNKNTVYLQM |
| | | | NSLKPEDTAIYYCRASTLRTGWLMGWGQGTQVTVSS |
| 152-H4 | beta2 | 1363 | EVxLVESGGGLVQAGGSLRLSCAASGSISSINLMGWYRQAPG |
| | | | KQRELVATISSTAYTDYSDSAKGRFTISRDNKNTVHLQMNSL |
| | | | KPEDTAVYYCRASTLRTGWLPGWGQGTQVTVSS |
| 235- | beta2 | 1364 | EV*LVESGGGLVQAGRSLRLSCAASGSVSSINVMAWYRQAP |
| C10, D10 | | | GKQRELVATISSSGYTDYSDSAKGRFTISRDDENTVHLQMNS |
| | | | LKPEDTAVYYCRASTARTGWLRAWGQGTQVTVSS |
| 235-C3 | beta2 | 1365 | XVQLVESGGGLVQAGGSLRLSCAASGSVSAINVMAWYRQA |
| | | | PGKQRELVATVSTTGYTDYSDSAKGRFTISRDNKNTVYLQM |
| | | | NSLKPEDTAIYYCRASTLRTGWLMGWGQGTQVTVSS |
| 139- | beta2 | 1366 | EVQLVESGGGLV*PGRSLXLSCAASGSIFGGNTMGWFRQAP |
| A10, 153- | | | GKRREMVATITSHGTGDYTGSVEGRFTISRDNAKNTVYLQM |
| D7, F7, E8, | | | NSLKPEDTAVYYCNSLIGWARNDYWGRGTQVTVSS |
| 152- | | | |
| A8, 236- | | | |
| A11, C5, | | | |
| F5, 248-F8 | | | |
| 139- | beta2 | 1367 | EVQLVESGGGLVQAGGSLRLSCAASGSRLRFELMGWYRQA |
| F10, 152- | | | PGKPRDLVALITSSGSANYADSVKGRFT1SRDNAKNTLYLQM |
| F11, H11, | | | NSLKPEDTGVYYCNAHTYTDNLWGQGTQVTVSS |
| C12, 153- | | | |
| G10 | | | |
| 153-B7, | beta2 | 1368 | XVQLVESGGGLVQAGGSLRLSCAASGSRLRFELMGWYRQA |
| E9 | | | PGKPRDLVALITSSGSANYADSVKGRFTISRDNAKNTLYLQM |
| | | | NSLKPEDTGVYYCNAHTYTDNLWGQGTQVTVSS |
| 152-H10 | beta2 | 1369 | EVQLVESGGGVVEAGGSLRLSCAATGSRFRFEIMGWYRQAP |
| | | | GKPRDLVALITRSGSANYADSVKGRFTISRDSAKNTLYLQM |
| | | | NSLKPEDTGVYYCNAHTYTDNLWGQGTQVTVSS |
| 152-B10 | beta2 | 1370 | EVQLVESGGGLVQAGGSLRLSCAASGSRFRFEIMGWYRQAP |
| | | | GKPRDLVALITSSGSANYADSVKGRFTISRDNAKNTLYLQM |
| | | | NSLKSEDTGVYYCNAHTYTDNLWGQGTQVTVSS |
| 139-D10 | beta2 | 1371 | EMQLVESGGGLVQAGGSLRLSCAASGSRLRFELMGWYRQA |
| | | | PGKPRDLVALITSSGSANYADSVKGRFTISRDNAKNTLYLQM |
| | | | NSLKPEDTGVYYCNAHTYTDNLWGQGTQVTVSS |
| 153-A10 | beta2 | 1372 | XVQLVESGGGLVQAGGSLRLSCAASGSRFRFEIMGWYRQAP |
| | | | GKPRDLVALITNSGSANYABSVKGRFTISRDNAKNTLYLQM |
| | | | NSLKPEDTGVYYCNAHTYTDSLWGQGTQVTVSS |
| 153-C7 | beta2 | 1373 | KVQLVESGGGLVQAGGSLRLSCAASGSRFRFELMGWYRQA |
| | | | PGKPRDLVALITSSGSANYAESVKGRFTISRDNAKNTLYLQM |
| | | | NSLKPEDTGVYYCNAHTYTDNLWGQGTQVTVSS |
| 153- | beta2 | 1374 | EVQLVESGGGLVQAGGSLRLSCAASGSRFRFELMGWYRQA |
| C9, 152- | | | PGKPRDLVALITSSGSANYADSVKGRFTISRDNAKNTLYLQM |
| A12, B9, | | | NSLKPEDTGVYYCNAHTYTDNLWGQGTQVTVSS |
| H7, H8, | | | |
| 236- | | | |
| D11, A12, | | | |
| C11, 248- | | | |
| B8, E7 | | | |
| 152-G9 | beta2 | 1375 | EVQLVESGGGLVQVGGSLRLSCAASGSRFRFELMGWYRQA |
| | | | PGKPRDLVALITSSGSANYADSVKGRFTISRDNAKNTLYLQM |
| | | | NSLKPEDTGVYYCNAHTYTDNLWGQGTQVTVSS |
| 153-F9 | beta2 | 1376 | EVQLVESGGGLVQAGGSLRLSCAASGSRLRFELMGWYRQA |
| | | | PGKPRDLVALITSSGSANYADSVKGRFTISRDNAKNTLYLQM |
| | | | NSLEPEDTGVYYCNAHTYTDNLWGQGTQVTVSS |
| 248-G7 | beta2 | 1377 | EVQLVESRGGLVQAGGSLRLSCAASGSRLRFELMGWYRQA |
| | | | PGKPRDLVALITSSGSANYADSVKGRFTISRDNAKNTLYLQM |
| | | | NSLKPEDTGVYYCNAHTYTDNLWGQGTQVTVSS |
| 236-B11 | beta2 | 1378 | EVQLVESGGGLVQAGGSLRLSCAASGSRFRFELMGWYRQA |
| | | | PGKPRDLVALITRSGSANYADSVKGRFTISRDNAKNTLYLQ |
| | | | MNSLKPEDTGVYYCNAHTYTDNLWGQGTQVTVSS |
| 236-D5 | beta2 | 1379 | EVQLVESGGGLVRAGGSLRLSCAASGSRFRFEIMGWYRQAP |
| | | | GSARDLVALITNSGSANYADSVKGRFTISRDNAKNTFYLQM |
| | | | NSLKPEDTGVYYCNAHTYTDNLWGQGTQVTVSS |
| 236-E12 | beta2 | 1380 | EVQLVESGGXLVQAEGSLRLSXAASGSRFRFELMGWYRXAP |
| | | | GKPRDLVALITXSGSANYADSVKGRFTISRXNAXNTLYLQM |
| | | | NSLKPEDTGVYYCNXHTYTDNLWGQGTQVTVSS |
| 236- | beta2 | 1381 | EVQLVESGGGLVQAGGSLRLSCAASGSRFRFEIMGWYRQAP |
| B5, F11, | | | GRMRDLVALITSSGSTNYADSVKGRFTISRDNAKNTLYLQM |
| 152-G10 | | | NSLKPEDTGVYYCNAHTYTDNLWGQGTQVTVSS |
| 153- | beta2 | 1382 | EVQLVESGGGLVQAGGSVRLSCAASGVTFRFVLMGWYRQA |
| A8, B11 | | | PGKQRELVAQITSSDATNYADSVKGRFTISRDNAKKTVDLQ |
| | | | MNSLKPEDTAVYYCLLARGPDVYWGQGTQVTVSS |
| 152- | beta2 | 1383 | KV*LVESGGGLVQAGGSVXLSCAASGVIFRFVLMGWYRXA |
| C9, 236- | | | PGKQRELVAQITSSDATNYADSVKGRFTISRDNAKKTVDLQ |
| E5 | | | MNSLKPEDTAVYYCLLARGPDVYWGQGTQVTVSS |
| 138- | beta2 | 1384 | EVQLVESGGGLVQAGGSQKLSCAASGSTLIYTMGWYRQAL |
| H2, 248- | | | GKKRVFVASISRDGSTIYGDSVKGRFTISRDNAKNTAYLQM |
| C2 | | | NSLKPEDTAVYVCKAEGWYQGYWGQGTQVTVSS |
| 138-B5 | beta2 | 1385 | EVQLVESGGGLVQAGGSLRLSCAASGSTEIYTMGWYRQAPG |
| | | | KQRVFVASISRDGSTIYGDSVKGRFTISRDSAKNTAYLQMNR |
| | | | LKPEDTAVYVCKAXGWYNGYWGQGTQVTVSS |
| 248-H1 | beta2 | 1386 | EVQLVESGGGLVQAGGSQKLSCAASGSTLIYTMGWYRQAL |
| | | | GKKRVFVASISRDGSTIYGDSVKGRFTISRDNAKNTAYLQM |
| | | | NNLKPEDTAVYVCKAEGWYQGYWGQGTQVTVSS |
| 138- | beta2 | 1387 | EVQLVESGGGLVQAGGSLRLSCAASGNTFSINAVGWYRQAP |
| A5, 139- | | | EKQRELVAIILSSGTTDYADSVKGRFTISRDNAKNIVYLQMN |
| E2, 235- | | | SLKPEDTAVYYCRVADREMGWAYWGQGTQVTVSS |
| E6 | | | |
| 152-G2 | beta2 | 1388 | EVQLVESGGGLVQAGGSLRLSCAASGNTFSINAVGWYRQAP |
| | | | EKQRELVAIIXSXGTTDYADSVKGRFTISRDNAKNTVYLQM |
| | | | NSLKPEDTAVYYCXXXDRXMGXAYWGQGTQVTVSS |
| 153- | beta2 | 1389 | EVQLVESGGGLVQTGGSLRLSCAASGSIFMILAMGWYRQAP |
| E10, D8, | | | GKQRELVTTIIRDGKTYYSDSVKDRFTISRDNAKNTAYLQM |
| G7 | | | NSLKPEDTAVYYCYTKVIVMGAGMDDNDFFGQGTQVTVSS |
| 235- | beta2 | 1390 | EVQLVESGGGLVQPGGSLRLSCAASGSILSRTDVDWYRQAP |
| D5, 153- | | | GKGREWVAIIAPFGTTNSRDSRFTISRDNAKNIVYLQMNSLE |
| A1 | | | PEDTAVYYCRIYWGGNVYWGQGTQVTVSS |
| 138-F2 | beta2 | 1391 | EVQLVESGGGLVQAGGSLRLSCAPSSSAVSIVHIQWHRQAPG |
| | | | KQRELVASVNSRGTTNYADSVKGRAIISRDNAKNTVYLQMN |
| | | | SLKPEDTAVYYCYARTLQLGALRDYWGQGTQVTVSS |
| 139-H10 | beta2 | 1392 | EVQLVESGGGLVQAGGSLRLSCAASASILSINTMDWFRRTPG |
| | | | KQRELVSTITRDGRTTYADSVKGRFTLSRDNTKNTVSLQMN |
| | | | SLKPEDTAVYYCLANVETTRGLTKNYWGQGIQVTVSS |
| 139- | beta2 | 1393 | EVQLVESGGGLVQTGGSLRLSCAASGSTFNINAWGWYRQAP |
| B2, 152- | | | GKQRELVAVISSSGSTDYSDAVKGRFTISTVNAGNTVYLEM |
| E2, 235- | | | NSLKPEDTAVYYCRAADSGPWRYWGQGSQVTVSS |
| C9D9 | | | |
| 248C7 | beta2 | 1394 | EVQLVESGGGKVQAGMSQRLSCAASGGIGTFSSVAWYRQA |
| | | | PGKQRELVAQITHEGTRNYADSVKGRFTISREFTLSRDGPKE |
| | | | MVHLQMVSLKPEDTAVYYCNAVQFGRNYWGQGTQVTVSS |
| 139- | alpha | 1395 | EVQLVESGGGLVQAGGSLRLSCAASGSIFSINYMGWYRQAP |
| B7, B10, | M | | GKQREAVAIIDRVGASTNYVDSVRGRFTISRSNAKNENMLY |
| C11 | | | LQMNSLKPEDTAVYYCNTVPTTSAYWGPGTQVTVSS |
| 139-C7 | alpha | 1396 | XVQLVESGGGLVQAGGSLRLSCAATGTIFSSNYMGWYRQA |
| | M | | PGLQRESVAIIDRGGASTNYVXSVRGRFTISRSNAKNQSMLY |
| | | | LQMNSLKPEDTAVYYCNTVPTTSAYWGPGTQVTVSS |
| 139-H9 | alpha | 1397 | EVQLVESGGGLVQAGGSLRLSCAASGSIFSISYMGWYRQAP |
| | M | | GKERESVAIIDRSGASTNYVDSVRGRFT1SRSNAKNKNMLYL |
| | | | QMNSLKPEDTAVYYCNTVPTTSAYWGPGTQVTVSS |
| 139-C10 | alpha | 1398 | EVQLVESGGGLVQAGGSLKLSCVASGLILSIHTMGWYRQAP |
| | M | | GKQREFVAVASNSGRTNYADSVKGRFTISRDNAKNTVYLQ |
| | | | MNNLKPEDTAVYYCNSASQFASWGQGTQVTVSS |
| 139-H11 | alpha | 1399 | EVQLVESGGGLVQAGGSLRLSCAASGIIFSTYTMGWYRQAPG |
| | M | | KQREFVAAATNAGTTSYAGSVKGRFTISRDNAKNTVILQMN |
| | | | NLKPEBTAVYYCRVLDYDYWGQGTQVTVSS |
| 139-D2 | alpha | 1400 | EVQLVESGGGLVQPGGSLRLSCAASRIIFSRNVMAWYRQAP |
| | M | | GKEREPVAIITTAHSTNYVDSVKGRFTISRDNAKNTLYLEMN |
| | | | SLKPEDTGVYYCNKLPHYPTDSWGQGTQVTVSS |
| 243-G9 | alpha | 1401 | EVQLVESGGGLVQPGGSLRLSCAASRSIFSRNAMAWYRQAP |
| | M | | GKQREPVAIITSNHGTNYVDPVKGRFTISRDNAKNTLYLQM |
| | | | NSLKPEDTGVYYCNK1PHYTVDSWGQGTQVTVSS |
| 243-C9 | alpha | 1402 | KVQLVESGGGLVQAGGSLRLSCTASGNIARITAFGWYRQAP |
| | M | | GRQRDFVAGIFSGALTNYADSVKGRFTISRDNAKNTVFLQM |
| | | | NSLKTEDTGTYYCRSDNNWGQGTQVTVSS |
| 243-A9 | alpha | 1403 | EVQLVESGGGLVQAGGSLRLSCTASGSDVRITAFAWYRQAP |
| | M | | GKQRDFVAGIFSGAITNYADSVKGRFTISRDNAKNTVFLQM |
| | | | NSLKTEDTATYYCRADNNWGQGTQVTVSS |
| 139-E9 | alpha | 1404 | EVQLVESGGGLVQAGGSLRLSCVASGFIFSIYGMAWYRQAP |
| | M | | GKQRELVASITGGYGPNYVDSVKGRFTISRDDAKNTLYLQM |
| | | | NSLKPEDTAVYYCNQLYSDYWGKGTQVTVSS |
| 139-H7 | alpha | 1405 | EVQLVASGGGLVQTGGSLRLSCAASGSGFSIDGMNWSRQAP |
| | M | | GKGRELVGY1TSSGSTDYADSVKGRFT+SRDNADNTVYLQM |
| | | | NSLKPEDTAVYYCAVATRSRLGLQQNYWGQGTQVTVSS |
| 139-H1 | alpha | 1406 | EVQLVESGGGLVQAGGSLRLSCAASGIIFTIYTMAWYRQAP |
| | M | | GKQRELVAAVTYAGNRYYVDSVKGRFTISRDDAKNKVYLQ |
| | | | MNSLKPEDTAVYYCAANPSDNPWLGQGTQVTVSS |
| 243-H9 | alpha | 1407 | EVQLVESGGGLVQPGGSLRLSCAASGSIRSIDAMAWYRQTP |
| | M | | GKQRDFVAAIFSGGLTHYADSVKGRFTISRDNAKNTLYLQM |
| | | | NSLKPEDTAVYYCKFRAPTGSDNWGQGTQVTVSS |
| 153-D3 | alpha | 1408 | EVQLVQSGGGLVQAGGSLRLSCAASGYIFSSNITGWYRQAP |
| | M | | GKQRELVAAISSDGLAHYTDSMKGRVIISRDNVENTVYLQM |
| | | | NSLKPEDTAVYYCAAPGAGRGQGTQVTVSS |
| 140-F10 | alphaV | 1409 | EVQLVESGGGSVQAGGSLRLSCAASQRTFSSDVMGWFRQAP |
| | beta6 | | GKERDFVAYIHRSGTTYYADSVKGRLTISRDNAKSTVYLQM |
| | | | NSLKPEDTAVYHCAAGRYGSTSDTLYDYWGQGTQVTVSS |
| 140-H10 | alphaV | 1410 | EVPLVESGGGSVQAGGSLRLSCAASQRTFSRDVTGWFRQAP |
| | beta6 | | GKERDFVAYIHRSGETTYYADSVKGRFTISRDNAKSTVYLQ |
| | | | MNSLKPEDTAVYHCAAGRYGSTSDTLYDYWGQGTQVTVSS |
| 140-B10 | alphaV | 1411 | EVQLVESGGGSV*AGGSLRLSCAASQRTFSSDVMGWFRQAP |
| | beta6 | | GKERDFVAYIHRSGTTYYADSVKGRFTISRDNAKSTVYLQM |
| | | | NSLKPEDTAVYHCAAGRYGSTADTLYDYWGQGTQVTVSS |
| 140-B8 | alphaV | 1412 | EVQLVESGGGSVQAGGSLrLSCAASQRTFSRDVMGWFRQAP |
| | beta6 | | GKERDFVAYSHRSGTTYYADSVKGRFTISRDNAKSTVYLQM |
| | | | NSLKPEDTAVYHCAAGRYGSTSDTLYDYWGQGTQVTVSS |
| 140-C8 | alphaV | 1413 | XVQLVESGGGSVQAGGSLrLSCAASQRTFSSDVMGWFRQAP |
| | beta6 | | GKEGDFVAYIHRSGTTYYADSVKGRFTISXDNAKSTVYLQM |
| | | | NSLKPEDTAVYHCAAGRYGSTSDTLYDYWGQGTQVTVSS |
| 140-D8 | alphaV | 1414 | EVQLVESGGGSVQAGGSLRLSCATSQRTFSSDVMGWFRQAP |
| | beta6 | | GKERDFVAYXHRSNTTYYADSVKGRFTISRDNAKSTVYLQ |
| | | | MNSLKPEDTAVYHCAAGRYGSTSDTLYDYWGQGTQVTVSS |
| 140-E5 | alphaV | 1415 | EVQLVESGVGLVQAGGSLrLSCAASGYTFNHNTMAWYRQA |
| | beta6 | | PGKQRELAASISSGGNTYYABSVKGRFTISRDNGNNTMYLQ |
| | | | MNNLKPEDTAVYYCNWKDWPPNYKNDYWGQGTQVTVSS |
| 140- | alphaV | 1416 | EVQLVESGGGLVQAGGSLRLSCAASGYTFNHNTMAWYRQA |
| F2, H5 | beta6 | | PVKQRELAASISSGGSTYYADSVKGRFTISRDNGNNTMYLQ |
| | | | MNNLKPEDTAVYYCNWKDWPPNYTNDYWGQGTQVTVSS |
| 140-E10 | alphaV | 1417 | EVQLVESGGGLVQAGESLKLSCVASGNILRVTSMGWGRQIP |
| | beta6 | | GKQRKLVAWITNEGRTEYADSVKGRFTISRDNAQNTLYLLM |
| | | | NSLKPEDTAVYYCYGFSPRESSGNTYWGQGTQVTVSS |
| 140-D10 | alphaV | 1418 | EVQLVESGGGLVQAGGSLRLSCAASGRIYSSNTMAWFRQAP |
| | beta6 | | GKEREFGSAIMWRGDATYTDYADSMKDRFTISRDNAKNTV |
| | | | YLQMNSLKPEDTATYYCAAGGRRWNTRKDSSQYDYWGQG |
| | | | TQVTVSS |
| 140-G8 | alphaV | 1419 | EVQLVESGGGSVQAGGSLRLSCTASGSIFSINDMGWYRQPPG |
| | beta6 | | EQRELVATLTSSDSTKYADSVKSRFTISRDNAKNTVYLQMN |
| | | | SLKPEDTAVYYCNAVINRRGDGRNWSREYWGQGTQVTVSS |
| 140-A10 | alphaV | 1420 | EVQLVESGGGLVQPGGSLrLSCAASGFTFSRMAMGWYRQAP |
| | beta6 | | GVERDFLAIISPGGGTNYADSVKGRFTISRDNAKNTVYLQM |
| | | | NSLKPEDTAVYYCNARNFEGRRVDYWGQGTQVTVSS |
| 140-A2 | alphaV | 1421 | EVQLVESGGGLVQAGGSLrLSCAASGSTYRLNNMAWYRQS |
| | beta6 | | PGKKRELVALISGITSDPSTYYLDSVRGRFSISRDNALNTVYL |
| | | | QMNSLKPEDTAVYYCKQAWAGVEYWGQGTQVTVSS |
| 140-H8 | alphaV | 1422 | EVQLVESGGGLVQAGGSLrLSCAASISANAIDVVSWYRQAPg |
| | beta6 | | KPRELAASITSAGRIKYAESVKGRFGISRDNAKNTVSLQMNS |
| | | | LKPEDTAVYYCNALYRNAIYWGQGTQVTVSS |
| 140-G10 | alphaV | 1423 | EVQLVESGGGLVQAGGSLXLSCAASGFTFDDYAIGWFRQAP |
| | beta6 | | GKEREGVSCIRNSDGRTYYADSVKGRFTISSDNAKNTVYLQ |
| | | | MNSLKPEDTALYYCAATQIGRPRGDKGANRYCSXSRDRGQ |
| | | | GTQVTVSS |
| 140-E2 | alphaV | 1424 | EVQLXESGGGLVQAGGSLXLSXAASGYIFSSNITGWYRQAP |
| | beta6 | | GKQRELVAAISSDGLAHYTDSMKGRVIISRXNVENTVYLQM |
| | | | NSLKPEDTAVYYCAAPGAGRGQGTQVTVSS |
| 140-D4 | alphaV | 1425 | EVQLVESGGGLVEAGGSLRLSCATSGSTFGIEAMAWYRQAP |
| | beta6 | | GKQRELVATINSASRTNYADSVKGRFTISRDTGKSILYLQMN |
| | | | NLEPEDTAVYYCKITTPLPYRRDFWGQGTQVTVSS |
| 140-A5 | alphaV | 1426 | EVQLVESGGGSVQAGGSLRLSCAASGTTATITVPGWYRQAP |
| | beta6 | | GKQRELVAVINSGGTKKYADSVKGRFTISIDNVKRTLYLEM |
| | | | NSLRPEDTAVYYCSTLKYWGQGTQVTVSS |
| 141-A10 | beta1 | 1427 | EVQLVESGGGLVQAGGSLRLSCAASGRTISRFTMGWFRQGP |
| | | | GKERDFVAAISWGGGRTNYEDSVKGRFTISRDNAQNTVYLQ |
| | | | MNSLKPEDTAVYYCAABSRGPYNSNWHQSSVSYDYWGQG |
| | | | TQVTVSS |
| 141- | beta1 | 1428 | EVQLVESGGGLVQAGGSLRLSCAASGRTISRFTMGWFRQAP |
| C10, 142- | | | GKERDFVAAISWGGGRTNYEDSVKGRFTISRDNAQNTVYLQ |
| C11, F7 | | | MNSLKPEDTAVYYCAABTRGPYNSNWAQSSVSYDGWGQG |
| | | | TQVTVSS |
| 141-E10 | beta1 | 1429 | EVQLVESGGGLVQAGGSLRLSCAASGRTISRFTMGWFRQAP |
| | | | GKEREFVAAISWGGGRTNYEDSVKGRFTISRDNAQNTVYLQ |
| | | | MNSLKPEDTAVYYCAABTRGPYNSNWAQSSVSYDYWGQG |
| | | | TQVTVSS |
| 141- | beta1 | 1430 | EVQLVESGGGLVQAGGSLRLSCAASGRTISRFTMGWFRQAP |
| F10, 142- | | | GKERDFVAAISWGGGRTNYEDSVKGRFTISRDNAQNTVYLQ |
| F10 | | | MNSLKPEDTAVYYCAABSRGPYNSNWHQSSVSYDYWGQG |
| | | | TQVTVSS |
| 141-A11, | beta1 | 1431 | EVQLVESGGGLVQAGGSLRLSCAASGRTISRFTMGWFRQAP |
| 242- | | | GKERDFVAAISWGGGRTNYEDSVKGRFTISRDNAQNTVYLQ |
| A12, B12, | | | MNSLKPEDTAVYYCAADTRGPYNSNWAQSSVSYDTWGQG |
| B4, C4, | | | TQVTVSS |
| D12, D4, E4, | | | |
| F12, F4, | | | |
| G12, B8, | | | |
| D8 | | | |
| 242-E8 | beta1 | 1432 | EVQLVESGGGLVQAGGSLRLSCAASGRTISRFTMGWFRQAP |
| | | | GKERDFVAAISWGGGRTNYEDSVKGRFT1SRDNAQNTVYLQ |
| | | | MNSLKPEDTAVYYCAADTRGPYNSNWAQSSVSYDGWGQG |
| | | | TQVTVSS |
| 141- | beta1 | 1433 | EVQLVESGGGLVQAGDSLRLSCAASGRTISRFTMGWFRQAP |
| C11, 242- | | | GKERDFVAAISWGGGRTNYEDSVKGRFTISRDNAQNTVYLQ |
| G4 | | | MNSLKPEDTAVYYCAADTRGPYNSNWAQSSVSYDGWGQG |
| | | | TQVTVSS |
| 142-C7 | beta1 | 1434 | EVQLVESGGGLVQAGGSLRLSCAASGRTISRFTMGWFRQAP |
| | | | GKERDFVAAISWGGGRTNYEDSVKGRFTISRDNAQNTVYLQ |
| | | | MNSLKPEDTAVYYCAABTRGPYNSNWHQSSVSYDAWGQG |
| | | | TQVTVSS |
| 142-B10 | beta1 | 1435 | EVQLVESGGGLVPAGGSLRLSCAASGRAISRFTMGWFRQAP |
| | | | GKERDFVAAISWGGGRTDYEDSVKGRFTISRDNARNTVYLQ |
| | | | MNSLKPEDTAVYYCAABTRGPYNSNWAQSSVSYNYWGQG |
| | | | TQVTVSS |
| 142-C9 | beta1 | 1436 | EVqLVESGGGLVQAGGSLRLSCAASGRTISRFTMGWFRQAP |
| | | | GKERDFVAAISWGGGRTKYEDSVTGRFTISRDNAQNTVYLQ |
| | | | MDSLKPEDTAVYYCAABSRGPYNSNWHQSSVSYDYWGQG |
| | | | TQVTVSS |
| 142- | beta1 | 1437 | EVQLVESGGGLVQAGGSLRLSCAASGRTISRFTMGWFRQAP |
| D8, A8, | | | GKERDFVAAISWGGGRTKYEDSVTGRFTISRDNAQNTVYLQ |
| H9 | | | MNSLKPEDTAVYYCAABSRGPYNSNWHQSSVSYDYWGQG |
| | | | TQVTVSS |
| 142-E10 | beta1 | 1438 | XVqLVESGGGLVQAGGSLRLSCAASGRTISRFTMGWERQAP |
| | | | GKERDFVAAISWGGGRTNYGDSVKGRSTISRDNAQNTVYLQ |
| | | | MNSLKPEDTAVYYCAABTRGPYNSNWAQSSVSYDYWGQG |
| | | | TQVTVSS |
| 142- | beta1 | 1439 | EVQLVESGGGLVQAGGSLRLSCAASGRTISRFTMGWFRQAP |
| E11, G11 | | | GKERDFVAAISWGGGRTNYEDSVKGRSTISRDNAQNTVYLQ |
| | | | MNSLKPEDTAVYYCAABTRGPYNSNWAQSSVSYDYWGQG |
| | | | TQVTVSS |
| 141-D11 | beta1 | 1440 | XVQLVESGGGLVQAGGSLRLSCAASGRTISRFTMGWFRQAP |
| | | | GKERDFVAAISWGGGRTNYEBSVKDRFTISRDNAQNTVYLQ |
| | | | MNSLKPEDTAVYYCAADSRGPYNSNWHQSSVSYDYWGQG |
| | | | TQVTVSS |
| 141- | beta1 | 1441 | EVQLVESGGGLVQAGGSLRLSCAASGRTISRFTMGWFRQAP |
| F11, 142- | | | GKERDFVAAISWGGGRTNYEDSVKDRFTISRDNAQNTVYLQ |
| B11 | | | MNSLKPEDTAVYYCAABSRGPYNSNWHQSSVSYDYWGQGT |
| | | | QVTVSS |
| 141-G11 | beta1 | 1442 | EV*LVESGGGLVQAGGSLRLSCAASGRTISRFTMGWFRQAP |
| | | | GKERDFVAAISWGGGRTKYEDSVTGRFTISRDNAQNTVYLQ |
| | | | MDSLKPEDTAVYYCAADSRGPYNSNWHQSSVSYDYWGQG |
| | | | TQVTVSS |
| 142-B7 | beta1 | 1443 | EV*LVESGGGLVQAGGSLRLSCAASGRTISRFTMGWFRQAP |
| | | | GKERDFVAAISWGGGRTKYEDSVTGRFTISRDNAQNTVYLQ |
| | | | MDSLKPEDTAVYYCAABSRGPYNSNWHQSSVSYDYWGQGT |
| | | | QVTVSS |
| 142-A7 | beta1 | 1444 | EVQLVESGGGLVQAGGSLSLSCAASGASGIIYSINAMGWYR |
| | | | QAPGKQREVVAVVTNGGSTEYADFVKGRFTVSREYAKNAV |
| | | | YLQMNSLKPEDTAVYYCYARGIIARWGSAPGNYWGQGTQV |
| | | | TVSS |
| 142-G7 | beta1 | 1445 | EVQLVESGGGLVQAGGSLRLSCAASGASGTIYSISSMGWYR |
| | | | QAPGKQREVVAVVTNGGSTEYADFVKGRFTVSREYAKNAV |
| | | | YLQMNSLKPEDTAVYYCYARGITARWGSAPGNYWGQGTQV |
| | | | TVSS |
| 141-H10 | beta1 | 1446 | EVQLVESGGGLVQPGGSLRLSCAVSGFTSDYYAIGWFRQAP |
| | | | GKEREGVSCISSSDGSTYYABSVKGRFTISRDNAKNTVYLQM |
| | | | NSLKPEDTAVYYCATTCVVNPEGYDFWGQGTQVTVSS |
| 141-E5 | beta1 | 1447 | EVQLVESGGGLVQAGGSLRLSCAASGSISSINSINIMGWYRQ |
| | | | APGKQRDLVAYITKRGSTKYADSVKGRFTISRDNAKNMATL |
| | | | QMNSLKPEDTAVYYCAAGPDGLGGQDDYWGQGTQVTVSS |
| 141-F5 | beta1 | 1448 | EVQLVESGGGLVQAGGSLRLACQASRAIERIVGWYRQAPGK |
| | | | QRELVAAITVPGITNYTDSVKDRFTISRDSAKNTVYLQMNKL |
| | | | KPEDTAVYYCAAPTYGRGQGTQVTVSS |
| 141-B6 | beta1 | 1449 | EVQLVESGGGMVQTGGSLRLSCAASGSTLNINNGEWYRQAP |
| | | | GKQREFVAAIGSGGTTDYADSVKGRFTISKANAKNTLYLQM |
| | | | NSLKPEDTAVYYCYVRSWRNYWGQGTQVTVSS |
| 245-D7 | beta1 | 1450 | EVQLVESGGGLVQAGGSLRLSCTASGRTASTYAMGWLRQA |
| | | | PGKEREFVAAISYSGATTYYADSVKGRFTISRDNAKSTAYLQ |
| | | | MNSLQPEDTAVYYCAASANRDLSLWVSTAYRSTGLVVYWG |
| | | | QGTQVTVSS |
| 242- | alpha5 | 1451 | EVQLVESGGGLVQAGGSLRLSCAASGGTFSTYAMGWFRQA |
| A4, A8, | | | PGEERQFVATITWTGYTYYTDSVKGRFT1SRDNAKKTVYLR |
| C12, C8, D7, | | | MDKLKPEDTAVYYCAADRRGYIETMSVNYDYWGQGTQVT |
| E12, F5, F8, | | | VSS |
| G7, E1l | | | |
| 241- | alpha5 | 1452 | EVQLVESGGGLVQPGGSLRLSCAASGSISIINFMNWYRQAPG |
| A10, A11, | | | KQRELVAQVTSGVTSGGTTYYDDSVKGRFTISRDNAKNMVS |
| B10, B11, | | | LQMNSLKPEDTAVYYCNVQGYFGSTWINYWGQGTQVTVSS |
| D10, D8, | | | |
| E10, E11, | | | |
| E4, F11, | | | |
| F8, G10, | | | |
| G11, H10, | | | |
| C11, xC8 | | | |
| 241-D11 | alpha5 | 1453 | EVQLVESGGGLVQPGGSLRLSCAAPGSISIINFMNWYRQAPG |
| | | | KQRELVAQVTSGVTSGGTTYYDDSVKGRFTISRDNAKNMVS |
| | | | LQMNSLKPEDTAVYYCNVQGYFGSTWINYWGQGTQVTVSS |
| 241- | alpha5 | 1454 | EVPLVESGGGLVQPGGSLRLSCAASGSISIINFMNWYRQAPG |
| C1, C2 | | | KQRELVAQVTSGVTSGGTTYYDDSVKGRFTISRDNAKNMVS |
| | | | LQMNSLKPEDTAVYYCNVQGYFGSTWINYWGQGTQVTVSS |
| 241-C10 | alpha5 | 1455 | EVRLVESGGGLVQPGGSLRLSCAASGSISIINFMNWYRQAPG |
| | | | KQRELVAQVTSGVTSGGTTYYDDSVKGRFTISRDNAKNMVS |
| | | | LQMNSLKPEDTAVYYCNVQGYFGSTWINYWGQGTQVTVSS |
| 241- | alpha5 | 1456 | EVQLVESGGGLVQPGGSLRLSCAASGFTFSRYTMTWVRQAP |
| E1, F10 | | | GKEPEWVSSITSRGSSTNYADSVKGRFTISRDNAKNTLYLQM |
| | | | NSLKPGDTAMYYCAKSGTETWYDRTYWGQGTQVTVSS |
| 241-E3 | alpha5 | 1457 | EVQLVESGGGLVQPGGSLRLSCAASGFTFSRYTMTWVRQAP |
| | | | GKEPEWVSSITSRGSSTNYADSAKGRFTISRDNAKNTLYLQM |
| | | | NSLKPGDTAMYYCAKSGTETWYDRTYWGQGTQVTVSS |
| 141-F1 | alpha3 | 1458 | EVQLVESGGGLVKAGGSLRLSCAASGSIFSINTMAWYRQAP |
| | | | GKQREWITSITSRGTTRYABSVKGRFTISTSNDKSTVYLQMN |
| | | | SLKPEDTAVYYCAADKDGVIGYSVGYWGQGTQVTVSS |
| 141-B4 | alpha3 | 1459 | EVQLVESGGGLVEAGGSLRLSCAASGSIFSINVMGWYRQAP |
| | | | GKQRELIGSITSRGTTRYTDSVKGRFTISRGNDKSTVYLQMN |
| | | | SLKPEDTAVYYCAABKGGVIGYSEGYWGQGTQVTVSS |
| 141- | alpha3 | 1460 | EVQLVESGGGLVEAGGSLRLSCAASGSIFSINVMGWYRQAP |
| F6, G1 | | | GKQRELIGSITSRGTTRYADSVKGRFTISRGNDMSTVYLQMN |
| | | | SLKPEDTAVYYCAABKGGVIGYSVGYWGQGTQVTVSS |
| 141-B11 | alpha3 | 1461 | EVQLVESGGGLVQAGGSLQLSCATSGESFSIKAMGWYRQAP |
| | | | GNQREMVATITGTGKTNYADSVKGRFTISRDIGTLYLQMNSL |
| | | | KPEDTAVYYCNLLSWPAGDYWGQGTQVTVSS |
| 141-H11 | alpha3 | 1462 | EVQLVESGGGLVQAGGSLRLSCTTSGRSFSIKAMGWYRQAP |
| | | | GNQRELVATITGTGSTTYADSAKGRFTISRXIGTLYLQMNSL |
| | | | KPEDTGVYYCNLLSWPAGDYWDQGTQVTVSS |
| 141-A5 | alpha3 | 1463 | EVQLVESGGALVQAGGSLRLSCAASGFAFSINTMAWYRQAP |
| | | | GNERDWVAIIFPGTGGSTVYEDSVKGRFTISRVNAKNTLYLQ |
| | | | MDSLRPEDTGVYYCARVRYIGGNYFPFDSWGQGTQVTVSS |
| 141-E6 | alpha3 | 1464 | EVQLVESGGALVQAGGSLRLSCAASGFXFSINTMAWYRQAP |
| | | | GKQRDWVAIIAPGTGGSTHYEDSVKGRFTISRVNAKNTLYL |
| | | | QMDSLRPEDTAVYYCARVRYTGGNYFPFDSWGQGTQVTVS |
| | | | S |
| 141-C2 | alpha3 | 1465 | XVQLVESGGGLVQPGGSLRLSCAASGFAFSNYPMGWVRQA |
| | | | PGKGLEWVSGISASSIRTSYADSVKGRFTISRDHAKNTLYLQ |
| | | | MNSLKVEDTAVYYCAQLRNYRYFGDMDYRGEGTLVTVSS |
| 245-G1 | alpha3 | 1466 | EVQLVESGGGLVQPGGSLRLSCAASGFAFSHYPMGWVRQAP |
| | | | GKGLEWVSGISASSIRTSYADSVKGRFTISRDHAKNTLYLQM |
| | | | NSLKVEDTAVYYCAQLRNYRYFGDMDYRGEGTLVTVSS |
| 141-D10 | alpha3 | 1467 | EVQLVESGGGLVQAGGSPRLSCVASGRTFSRCAMGWFRQAP |
| | | | GKEREEVAT1SANGELTYYANFVEGRFTISRDNAKNTVYLQM |
| | | | NSLKPEDTAVYFCAARRTFTRSSNRNEYADWGQGTQVTVSS |
| 141-A4 | alpha3 | 1468 | EVQLVESGGGLVQAGGSLTLSCAASGSVFSINAMGWYRQAP |
| | | | GKQRELVATITTRGTTNYVDAVKGRFTISKDNAKNTMYLQM |
| | | | NSLKPEDTAVYYCAABSPPYGMGSDLGYWGQGTQVTVSS |
| 141- | alpha3 | 1469 | EVQLVESGGGLVQAGGSLRLSCAASGRMFSPNVMGWFRQA |
| D4, 245- | | | PGDQREFVANIYSGGSTNYADTVKGRFTILSDNAKNTVYLQ |
| H1 | | | MTSLKPEDTGVYYCSVKRVGQSWFDSGYWGQGTQVTVSS |
| 141-B10 | alpha3 | 1470 | EVQLVESGGGLVQAGGSLNLSCAASGRILRINNMGWYRQAP |
| | | | GNKRDLVARITSGGSHRNYABSVKGRFTISIDNTRKTVYLQM |
| | | | NSLKPEDTAVYYCYGAIVSSRWGAEXTNDYWGQGTQVTVS |
| | | | S |
| 245-E7 | alpha3 | 1471 | EVQLVESGGGLVQAGGSLRLSCGASGSIGTFNIMGWYRQAP |
| | | | GKQREMVATMTSGGNTRYADSVKGRSTISRENAKKTITLQM |
| | | | NNLKPEDTGVYYCNLKTLTAWSTSTGDYWGQGTQVTVSS |
| 245-B1 | alpha3 | 1472 | EVQLVESGGGLVQAGGSLKLSCAASGFTFDDYAIGWFRQAP |
| | | | GKEREGVSCISSPDGSTYLVDSVKGRFTVSSDNAKNTAYLQ |
| | | | MNSLKPEDTAVYYCALRRGGSYYFCDPLTVYEYDYWGQGT |
| | | | QVTVSS |
| 245-D1 | alpha3 | 1473 | EVQLVESGGALVQPGGSLRLSCAASGFAFSNTVMSWARQAP |
| | | | GKGLEWVSTISASGVRTRYADSVTGRFTISRDYAKRTLYLQ |
| | | | MNSLSPEDTGVYYCVRRKSYTDYDPPRWDYDTWGQGTQVT |
| | | | VSS |
| 245- | alpha3 | 1474 | EVQLVESGGGLVQAGGSLRLSCAASGGTFRYQNMGWYRQA |
| A1, F1 | | | PGNEREWVASNWATGATAYADSVKGRFTISRDDAKNVVYL |
| | | | QMNNLKPEDTAVYYCNRLSRPWSWGQGTQVTVSS |
| 245-E1 | alpha3 | 1475 | EVQLVESGGGLVQAGGSLRLSCTVSITTYSFKRVAWHRQAP |
| | | | GNERELVAAIWNTDNTDYADSVKGRFTISRDNAKKTVYLQ |
| | | | MNRLKPEDTAVYYCSANRGSYGTYWGQGTQVTVSS |
| 245-F7 | alpha3 | 1476 | EVQLVDSGGRLVQPGDLLRLSCTTSGFASSGYVLGWFRQAP |
| | | | GKEREFVAAIIRSGGNTAYSDSVKGRFTISRDNAKNTVYLQM |
| | | | KSLKPEDTGIYYCARSSVLGRSPALYDLWGQGTQVTVSS |
| 248-A8 | not yet | 1477 | EVQLVESRGGLVQAGGSLRLSCTASERTFSRNVMAWFRQAP |
| | done | | GKEREFVAAIRWSGGTTSYADFVKGRFTMSRDNAKNTIYLE |
| | | | MNSLKPEDTAVYYCAADARLYSPLPRRSSAYDYWGQGTQV |
| | | | TVSS |
| 248-H8 | not yet | 1478 | EVQLVESGGGLVQPGGSLRLSCAASGFTFDDYDIGWFRQTP |
| | done | | GKEREGVSCISSSDGYKFYADSVKDRFTISSDNAKNTVYLQM |
| | | | NS LKPEDTAVYYCAAVKRAPKQYCSDYEAYDYWGQETQVT |
| | | | VSS |
| 248-A2 | not yet | 1479 | EVQLVESGGGLVQAGGSLRLSCAASGSISSLGLVQWHRQVS |
| | done | | RKQRGLVAQLNSGGTTTYADSVKGRFTISRDNAKSTVYLQM |
| | | | HSLKPEDTAVYYCFLRVIVPGGFRDYWGQGTQVTVSS |
| 248-B2 | not yet | 1480 | EVQLVESGGGLVQAGGSLRLSCVASGBTICIRAMDWYRQAP |
| | done | | GKERELVATITSDGSTYYADSVKGRFTISRDNAKNTLYLQM |
| | | | NSLKPEDTAAYYCKAPPYGSSCPLVWGQGTQVTVSS |
| 152-D2 | unknown | 1481 | EVQLVESGGGLVQAGGSLTLSCAASGNISSINIVNWYHQAPG |
| | | | KQRELVAFITNGEETNYAETVKGRFTVSRDNAKNTVSLQMN |
| | | | SLKPEDTGVYYCNLHIMWPTVRDYWGQGTQVTVSS |
| 152-E9 | unknown | 1482 | EVQLVESGGGLVQPGGSLRLSCAASGITLNYYAIGWFRQAPG |
| | | | KEREGVSCISSSAGSAYYADSVKGRFTISRDNAKNTLYLQMN |
| | | | SLKPEDTAVYYCAAQTAGTSIGCHISIGWYDYWGQGTQVTV |
| | | | SS |
| 152-E10 | unknown | 1483 | EVQLVESGGGLVQAGGSLRLSCAASGFTFDDYAIGWFRQAP |
| | | | GKEREGVSCISSSDGSIFYADSVKGRFTISSDNAKNTVYLQM |
| | | | NSLKPEDTAVYYCAAALRTVLAGTPTCDRYEYDYWGQGTQ |
| | | | VTVSS |
| 152-D7 | unknown | 1484 | EVQLVESGGGLVQAGGSLTLSCAASGNISSINIVNWYHQAPG |
| | | | KQRELVAFITNGEETNYAETVKGRFTVSRDNAKNTVSLQMN |
| | | | SLKPEDTGVYYCNLHIMWPTVRDYWGQGTQVTVSS |
| VHH-H6 | Alpha | 1485 | QVQLQDSGGGLVQAGGSLRLSCEASGRTFSSYAMGWFRQPP |
| | 3 | | GKEREWVSTISRSGSA1YAYPVKGRFTMSRDNAKNTVYLEM |
| | | | NSLKPEDTAVFYCAAARSGVPSSRPTDYDYWGQGTQVTVSS |
| VHH-5 | Alpha | 1486 | EVQLVESGGGLVQAGGSLRLSCAASGGTFRYQNMGWYRQA |
| bihead | 3 | | PGNEREVVVASNWATGATAYADSVKGRFTISRDDAKNVVYL |
| (GS15) | | | QMNNLKPEDTAVYYCNRLSRPWGWGQGTQVTVSSGGGGS |
| | | | GGGGSGGGGSEVQLVESGGGLVQAGGSLRLSCAASGGTFRY |
| | | | QNMGWYRQAPGNEREWVASNWATGATAYADSVKGRFTIS |
| | | | RDDAKNVVYLQMNNLKPEDTAVYYCNRLSRPWSWGQGTQ |
| | | | VTVSS |
| VHH-5 | Alpha | 1487 | EVQLVESGGGLVQAGGSLRLSCAASGGTFRYQNMGWYRQA |
| bihead | 3 | | PGNEREWVASNWATGATAYADSVKGRFTISRDDAKNVVYL |
| (GS5) | | | QMNNLKPEDTAVYYCNRLSRPWGWGQGTQVTVSSGGGGSE |
| | | | VQLVESGGGLVQAGGSLRLSCAASGGTFRYQNMGWYRQAP |
| | | | GNEREWVASNWATGATAYADSVKGRFTISRDDAKNVVYLQ |
| | | | MNNLKPEDTAVYYCNRLSRPWSWGQGTQVTVSS |

In particular, the invention in some specific aspects provides:
amino acid sequences that are directed against (as defined herein) Integrins and that have at least 80%, preferably at least 85%, such as 90% or 95% or more sequence identity with at least one of the amino acid sequences of SEQ ID NO□ 1316 to 1476, and SEQ ID NO: 1485, 1486, and 1487 (see Table 1). These amino acid sequences may further be such that they neutralize binding of the cognate ligand to Integrins; and/or compete with the cognate ligand for binding to Integrins; and/or are directed against an interaction site (as defined herein) on Integrins (such as the ligand binding site);
amino acid sequences that cross-block (as defined herein) the binding of at least one of the amino acid sequences of SEQ ID NO□ 1316 to 1476, and SEQ ID NO: 1485, 1486, and 1487 (see Table 1) to Integrins and/or that compete with at least one of the amino acid sequences of SEQ ID NO□ 1316 to 1476, and SEQ ID NO: 1485, 1486, and 1487 (see Table 1) for binding to Integrins. Again, these amino acid sequences may further be such that they neutralize binding of the cognate ligand to Integrins; and/or compete with the cognate ligand for binding to Integrins; and/or are directed against an interaction site (as defined herein) on Integrins (such as the ligand binding site);
   which amino acid sequences may be as further described herein (and may for example be Nanobodies); as well as polypeptides of the invention that comprise one or more of such amino acid sequences (which may be as further described herein, and may for example be bispecific and/or biparatopic polypeptides as described herein), and nucleic acid sequences that encode such amino acid sequences and polypeptides. Such amino acid sequences and polypeptides do not include any naturally occurring ligands.

Accordingly, some particularly preferred Nanobodies of the invention are Nanobodies which can bind (as further defined herein) to and/or are directed against to Integrins and which:
i) have at least 80% amino acid identity with at least one of the amino acid sequences of SEQ ID NO□s: 1316 to 1476, and SEQ ID NO: 1485, 1486, and 1487 (see Table 1), in which for the purposes of determining the degree of amino acid identity, the amino acid residues that form the CDR sequences are disregarded. In this respect, reference is also made to Table A-1, which lists the framework 1 sequences (SEQ ID NO□s: 1205, 2 framework 2 sequences (SEQ ID NO□s: 466 to 635), framework 3 sequences (SEQ ID NO□s: 806 to 975) and framework 4 sequences (SEQ ID NO□s: 1146 to 1315) of the Nanobodies of SEQ ID NO□s: 1316 to 1476, 1485, 1486, 1487 (see Table 1) (with respect to the amino acid residues at positions 1 to 4 and 27 to 30 of the framework 1 sequences, reference is also made to the comments made below. Thus, for determining the degree of amino acid identity, these residues are preferably disregarded);
   and in which:
ii) preferably one or more of the amino acid residues at positions 11, 37, 44, 45, 47, 83, 84, 103, 104 and 108 according to the Kabat numbering are chosen from the Hallmark residues mentioned in Table A-3 below.

In these Nanobodies, the CDR sequences are generally as further defined herein.

Again, such Nanobodies may be derived in any suitable manner and from any suitable source, and may for example be naturally occurring V_{HH} sequences (i.e. from a suitable species of Camelid) or synthetic or semi-synthetic amino acid sequences, including but not limited to □humanized□ (as defined herein) Nanobodies, □camelized□ (as defined herein immunoglobulin sequences (and in particular camelized heavy chain variable domain sequences), as well as Nanobodies that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein. Also, when a Nanobody comprises a V_{HH} sequence, said Nanobody may be suitably humanized, as further described herein, so as to provide one or more further (partially or fully) humanized Nanobodies of the invention. Similarly, when a Nanobody comprises a synthetic or semi-synthetic sequence (such as a partially humanized sequence), said Nanobody may optionally be further suitably humanized, again as described herein, again so as to provide one or more further (partially or fully) humanized Nanobodies of the invention.

In particular, humanized Nanobodies may be amino acid sequences that are as generally defined for Nanobodies in the previous paragraphs, but in which at least one amino acid residue is present (and in particular, in at least one of the framework residues) that is and/or that corresponds to a humanizing substitution (as defined herein). Some preferred, but non-limiting humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Thus, some other preferred Nanobodies of the invention are Nanobodies which can bind (as further defined herein) to Integrins and which:
i) are a humanized variant of one of the amino acid sequences of SEQ ID NO□s: 1316 to 1476, and SEQ ID NO: 1485, 1486, and 1487 (see Table 1); and/or
ii) have at least 80% amino acid identity with at least one of the amino acid sequences of SEQ ID NO□s: 1316 to 1476, and SEQ ID NO: 1485, 1486, and 1487 (see Table 1), in which for the purposes of determining the degree of amino acid identity, the amino acid residues that form the CDR sequences are disregarded;
   and in which:
i) preferably one or more of the amino acid residues at positions 11, 37, 44, 45, 47, 83, 84, 103, 104 and 108 according to the Kabat numbering are chosen from the Hallmark residues mentioned in Table A-3 below.

According to another specific aspect of the invention, the invention provides a number of stretches of amino acid residues (i.e. small peptides) that are particularly suited for binding to Integrins. These stretches of amino acid residues may be present in, and/or may be corporated into, an amino acid sequence of the invention, in particular in such a way that they form (part of) the antigen binding site of an amino acid sequence of the invention. As these stretches of amino acid residues were first generated as CDR sequences of heavy chain antibodies or V_{HH} sequences that were raised against Integrins (or may be based on and/or derived from such CDR sequences, as further described herein), they will also generally be referred to herein as □CDR sequences□ (i.e. as CDR1 sequences, CDR2 sequences and CDR3 sequences, respectively). It should however be noted that the invention in its broadest sense is not limited to a specific structural role or function that these stretches of amino acid residues may have in an amino acid sequence of the invention, as long as these stretches of amino acid residues allow the amino acid sequence of the invention to bind to Integrins. Thus, generally, the invention in its broadest sense comprises any amino acid sequence that is capable of binding to Integrins and that comprises one or more CDR sequences as described herein, and in particular a suitable combination of two or more such CDR sequences, that are suitably linked to each other via one or more further amino acid sequences, such that the entire amino acid sequence forms a binding domain and/or binding unit that is capable of binding to Integrins. It should however also be noted that the presence of only one such CDR sequence in an amino acid sequence of the invention may by itself already be sufficient to provide an amino acid sequence of the invention that is capable of binding to Integrins; reference is for example again made to the so-called□Expedite fragments□ described in WO 03/050531.

Thus, in another specific, but non-limiting aspect, the amino acid sequence of the invention may be an amino acid sequence that comprises at least one amino acid sequence that is chosen from the group consisting of the CDR1 sequences, CDR2 sequences and CDR3 sequences that are described herein (or any suitable combination thereof). In particular, an amino acid sequence of the invention may be an amino acid sequence that comprises at least one antigen binding site, wherein said antigen binding site comprises at least one amino acid sequence that is chosen from the group consisting of the CDR1 sequences, CDR2 sequences and CDR3 sequences that are described herein (or any suitable combination thereof).

Generally, in this aspect of the invention, the amino acid sequence of the invention may be any amino acid sequence that comprises at least one stretch of amino acid residues, in which said stretch of amino acid residues has an amino acid sequence that corresponds to the sequence of at least one of the CDR sequences described herein. Such an amino acid sequence may or may not comprise an immunoglobulin fold. For example, and without limitation, such an amino acid sequence may be a suitable fragment of an immunoglobulin sequence that comprises at least one such CDR sequence, but that is not large enough to form a (complete) immunoglobulin fold (reference is for example again made to the □Expedite fragments□ described in WO 03/050531). Alternatively, such an amino acid sequence may be a suitable□ protein scaffold□ that comprises least one stretch of amino acid residues that corresponds to such a CDR sequence (i.e. as part of its antigen binding site). Suitable scaffolds for presenting amino acid sequences will be clear to the skilled person, and for example comprise, without limitation, to binding scaffolds based on or derived from immunoglobulins (i.e. other than the immunoglobulin sequences already described herein), protein scaffolds derived from protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23:1257), and binding moieties based on DNA or RNA including but not limited to DNA or RNA aptamers (Ulrich et al., Comb Chem High Throughput Screen 2006 9(8):619-32).

Again, any amino acid sequence of the invention that comprises one or more of these CDR sequences is preferably such that it can specifically bind (as defined herein) to Integrins, and more in particular such that it can bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein), that is as defined herein.

More in particular, the amino acid sequences according to this aspect of the invention may be any amino acid sequence that comprises at least one antigen binding site, wherein said antigen binding site comprises at least two amino acid sequences that are chosen from the group consisting of the CDR1 sequences described herein, the CDR2 sequences described herein and the CDR3 sequences described herein, such that (i) when the first amino acid sequence is chosen from the CDR1 sequences described herein, the second amino acid sequence is chosen from the CDR2 sequences described herein or the CDR3 sequences described herein; (ii) when the first amino acid sequence is chosen from the CDR2 sequences described herein, the second amino acid sequence is chosen from the CDR1 sequences described herein or the CDR3 sequences described herein; or (iii) when the first amino acid sequence is chosen from the CDR3 sequences described herein, the second amino acid sequence is chosen from the CDR1 sequences described herein or the CDR3 sequences described herein.

Even more in particular, the amino acid sequences of the invention may be amino acid sequences that comprise at least one antigen binding site, wherein said antigen binding site comprises at least three amino acid sequences that are chosen from the group consisting of the CDR1 sequences described herein, the CDR2 sequences described herein and the CDR3 sequences described herein, such that the first amino acid sequence is chosen from the CDR1 sequences described herein, the second amino acid sequence is chosen from the CDR2 sequences described herein, and the third amino acid sequence is chosen from the CDR3 sequences described herein. Preferred combinations of CDR1, CDR2 and CDR3 sequences will become clear from the further description herein. As will be clear to the skilled person, such an amino acid sequence is preferably an immunoglobulin sequence (as further described herein), but it may for example also be any other amino acid sequence that comprises a suitable scaffold for presenting said CDR sequences.

Thus, in one specific, but non-limiting aspect, the invention relates to an amino acid sequence directed against Integrins, that comprises one or more stretches of amino acid residues chosen from the group consisting of:
a) the amino acid sequences of SEQ ID NO□s: 296 to 465;
b) amino acid sequences that have at least 80% amino acid identity with at least one of the amino acid sequences of SEQ ID NO□s: 296 to 465;
c) amino acid sequences that have 3, 2, or 1 amino acid difference with at least one of the amino acid sequences of SEQ ID NO□ 296s to 465;
d) the amino acid sequences of SEQ ID NO□s: 636 to 805;
e) amino acid sequences that have at least 80% amino acid identity with at least one of the amino acid sequences of SEQ ID NO□s: 636 to 805;
f) amino acid sequences that have 3, 2, or 1 amino acid difference with at least one of the amino acid sequences of SEQ ID NO□s: 636 to 805;
g) the amino acid sequences of SEQ ID NO□s: 976 to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□s: 976 to 1145;
   or any suitable combination thereof.

When an amino acid sequence of the invention contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
   and/or
ii) the amino acid sequence according to b) and/or c) preferably only contains amino acid substitutions, and no amino acid deletions or insertions, compared to the corresponding amino acid sequence according to a);
   and/or
iii) the amino acid sequence according to b) and/or c) may be an amino acid sequence that is derived from an amino acid sequence according to a) by means of affinity maturation using one or more techniques of affinity maturation known per se.

Similarly, when an amino acid sequence of the invention contains one or more amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
   and/or
ii) the amino acid sequence according to e) and/or f) preferably only contains amino acid substitutions, and no amino acid deletions or insertions, compared to the corresponding amino acid sequence according to d);
   and/or
iii) the amino acid sequence according to e) and/or f) may be an amino acid sequence that is derived from an amino acid sequence according to d) by means of affinity maturation using one or more techniques of affinity maturation known per se.

Also, similarly, when an amino acid sequence of the invention contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
   and/or
ii) the amino acid sequence according to h) and/or i) preferably only contains amino acid substitutions, and no amino acid deletions or insertions, compared to the corresponding amino acid sequence according to g);
   and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any amino acid sequences of the invention that comprise one or more amino acid sequences according to b), c), e), f), h) or i), respectively.

In this specific aspect, the amino acid sequence preferably comprises one or more stretches of amino acid residues chosen from the group consisting of:

i) the amino acid sequences of SEQ ID NO□ 296s to 465;
ii) the amino acid sequences of SEQ ID NO□s: 636 to 805; and
iii) the amino acid sequences of SEQ ID NO□s: 976 to 1145;
or any suitable combination thereof.

Also, preferably, in such an amino acid sequence, at least one of said stretches of amino acid residues forms part of the antigen binding site for binding against integrins.

In a more specific, but again non-limiting aspect, the invention relates to an amino acid sequence directed against Integrins, that comprises two or more stretches of amino acid residues chosen from the group consisting of:
a) the amino acid sequences of SEQ ID NO□s: 296 to 465;
b) amino acid sequences that have at least 80% amino acid identity with at least one of the amino acid sequences of SEQ ID NO□s: 296 to 465;
c) amino acid sequences that have 3, 2, or 1 amino acid difference with at least one of the amino acid sequences of SEQ ID NO□s: 296 to 465;
d) the amino acid sequences of SEQ ID NO□s: 636 to 805;
e) amino acid sequences that have at least 80% amino acid identity with at least one of the amino acid sequences of SEQ ID NO□s: 636 to 805;
f) amino acid sequences that have 3, 2, or 1 amino acid difference with at least one of the amino acid sequences of SEQ ID NO□s: 636 to 805;
g) the amino acid sequences of SEQ ID NO□s: 976 to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□ 976s to 1145;
   such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), e) or f).

In this specific aspect, the amino acid sequence preferably comprises two or more stretches of amino acid residues chosen from the group consisting of:

i) the amino acid sequences of SEQ ID NO□s: 296 to 465;
ii) the amino acid sequences of SEQ ID NO□s: 636 to 805; and
iii) the amino acid sequences of SEQ ID NO□s: 976 to 1145;
such that, (i) when the first stretch of amino acid residues corresponds to one of the amino acid sequences of SEQ ID NO□s: 296 to 165 the second stretch of amino acid residues corresponds to one of the amino acid sequences of SEQ ID NO□s: 636 to 805 or of SEQ ID NOD s: 976 to; [symbol] [symbol] when the first stretch of amino acid residues corresponds to one of the amino acid sequences of SEQ ID NO□s: 636 to 805 [symbol] [symbol] second stretch of amino acid residues corresponds to one of the amino acid sequences of SEQ ID NO□s: 296 to 465 or of SEQ ID NOD s: 976 to; [symbol] [symbol] (5ii) when the first stretch of amino acid residues corresponds to one of the amino acid sequences of SEQ ID NO□s: 976 to, [symbol] [symbol] second stretch of amino acid residues corresponds to one of the amino acid sequences of SEQ ID NO□s: 296 to 465 or of SEQ ID NO□s: 636 to 805

Also, in such an amino acid sequence, the at least two stretches of amino acid residues again preferably form part of the antigen binding site for binding against integrins.

In an even more specific, but non-limiting aspect, the invention relates to an amino acid sequence directed against Integrins, that comprises three or more stretches of amino acid residues, in which the first stretch of amino acid residues is chosen from the group consisting of:
a) the amino acid sequences of SEQ ID NO□s: 296 to 465;
b) amino acid sequences that have at least 80% amino acid identity with at least one of the amino acid sequences of SEQ ID NO□s: 296 to 465;
c) amino acid sequences that have 3, 2, or 1 amino acid difference with at least one of the amino acid sequences of SEQ ID NO□s: 296 to 465;
   the second stretch of amino acid residues is chosen from the group consisting of:
d) the amino acid sequences of SEQ ID NO□s: 636 to 805;
e) amino acid sequences that have at least 80% amino acid identity with at least one of the amino acid sequences of SEQ ID NO□s: 636 to 805;
f) amino acid sequences that have 3, 2, or 1 amino acid difference with at least one of the amino acid sequences of SEQ ID NO□s: 636 to 805;
   and the third stretch of amino acid residues is chosen from the group consisting of:
g) the amino acid sequences of SEQ ID NO□s: 976 to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□s: 976 to 1145.

Preferably, in this specific aspect, the first stretch of amino acid residues is chosen from the group consisting of the amino acid sequences of SEQ ID NO□s: 296 to 465; the second stretch of amino acid residues is chosen from the group consisting of the amino acid sequences of SEQ ID NO□s: 636 to 805; and the third stretch of amino acid residues is chosen from the group consisting of the amino acid sequences of SEQ ID NO□s: 976 to 1145.

Again, preferably, in such an amino acid sequence, the at least three stretches of amino acid residues forms part of the antigen binding site for binding against Integrins.

Preferred combinations of such stretches of amino acid sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO□131s6 to 1476, and SEQ ID NO: 1485, 1486, and 1487 (see Table 1). This degree of amino acid identity can for example be determined by determining the degree of amino acid identity (in a manner described herein) between said amino acid sequence and one or more of the sequences of SEQ ID NO□131s6 to 1476, and SEQ ID NO: 1485, 1486, and 1487 (see Table 1), in which the amino acid residues that form the framework regions are disregarded. Also, such amino acid sequences of the invention can be as further described herein.

Also, such amino acid sequences are preferably such that they can specifically bind (as defined herein) to Integrins; and more in particular bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:
CDR1 is chosen from the group consisting of:
a) the amino acid sequences of SEQ ID NO□s: 296 to 465;
b) amino acid sequences that have at least 80% amino acid identity with at least one of the amino acid sequences of SEQ ID NO□s: 296 to 465;
c) amino acid sequences that have 3, 2, or 1 amino acid difference with at least one of the amino acid sequences of SEQ ID NO□s: 296 to 465;
   and/or
   CDR2 is chosen from the group consisting of:
d) the amino acid sequences of SEQ ID NO□s: 636 to 805;
e) amino acid sequences that have at least 80% amino acid identity with at least one of the amino acid sequences of SEQ ID NO□s: 636 to 805;
f) amino acid sequences that have 3, 2, or 1 amino acid difference with at least one of the amino acid sequences of SEQ ID NO□s: 636 to 805;
   and/or
   CDR3 is chosen from the group consisting of:
g) the amino acid sequences of SEQ ID NO□s: 976 to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□s: 976 to 1145.

In particular, such an amino acid sequence of the invention may be such that CDR1 is chosen from the group consisting of the amino acid sequences of SEQ ID NO□s: 296 to 465; and/or CDR2 is chosen from the group consisting of the amino acid sequences of SEQ ID NO□s: 636 to 805; and/or CDR3 is chosen from the group consisting of the amino acid sequences of SEQ ID NO□s: 976 to 1145.

In particular, when the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO□s: 296 to 465;
b) amino acid sequences that have at least 80% amino acid identity with at least one of the amino acid sequences of SEQ ID NO□s: 296 to 465;
c) amino acid sequences that have 3, 2, or 1 amino acid difference with at least one of the amino acid sequences of SEQ ID NO□s: 296 to 465;
   and

CDR2 is chosen from the group consisting of:
d) the amino acid sequences of SEQ ID NO□s: 636 to 805;
e) amino acid sequences that have at least 80% amino acid identity with at least one of the amino acid sequences of SEQ ID NO□s: 636 to 805;
f) amino acid sequences that have 3, 2, or 1 amino acid difference with at least one of the amino acid sequences of SEQ ID NO□s: 636 to 805;
   and

CDR3 is chosen from the group consisting of:
g) the amino acid sequences of SEQ ID NO□s: 976 to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□s: 976 to 1145; or any suitable fragment of such an amino acid sequence

In particular, such an amino acid sequence of the invention may be such that CDR1 is chosen from the group consisting of the amino acid sequences of SEQ ID NO□s: 296 to 465; and CDR2 is chosen from the group consisting of the amino acid sequences of SEQ ID NO□s: 636 to 805; and CDR3 is chosen from the group consisting of the amino acid sequences of SEQ ID NO□s: 976 to 1145.

Again, preferred combinations of CDR sequences will become clear from the further description herein.

Also, such amino acid sequences are preferably such that they can specifically bind (as defined herein) to Integrins; and more in particular bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO□131s6 to 1476, and SEQ ID NO: 1485, 1486, and 1487 (see Table 1). This degree of amino acid identity can for example be determined by determining the degree of amino acid identity (in a mariner described herein) between said amino acid sequence and one or more of the sequences of SEQ ID NO□131s6 to 1476, and SEQ ID NO: 1485, 1486, and 1487 (see Table 1), in which the amino acid residues that form the framework regions are disregarded. Such amino acid sequences of the invention can be as further described herein.

In such an amino acid sequence of the invention, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

The framework sequences are preferably such that the amino acid sequence of the invention is a domain antibody (or an amino acid sequence that is suitable for use as a domain antibody); is a single domain antibody (or an amino acid sequence that is suitable for use as a single domain antibody); is a “dAb” (or an amino acid sequence that is suitable for use as a dAb); or is a Nanobody™ (including but not limited to V_{HH} sequence). Again, suitable framework sequences will be clear to the skilled person, for example on the basis the standard handbooks and the further disclosure and prior art mentioned herein.

In particular, the framework, sequences present in the amino acid sequences of the invention may contain one or more of Hallmark residues (as defined herein), such that the amino acid sequence of the invention is a Nanobody™. Some preferred, but non-limiting examples of (suitable combinations of) such framework sequences will become clear from the further disclosure herein.

Again, as generally described herein for the amino acid sequences of the invention, it is also possible to use suitable fragments (or combinations of fragments) of any of the foregoing, such as fragments that contain one or more CDR sequences, suitably flanked by and/or linked via one or more framework sequences (for example, in the same order as these CDR□s and framework sequences may occur in th [symbol] [symbol] immunoglobulin sequence from which the fragment has been derived). Such fragments may also again be such that they comprise or can form an immunoglobulin fold, or alternatively be such that they do not comprise or cannot form an immunoglobulin fold.

In one specific aspect, such a fragment comprises a single CDR sequence as described herein (and in particular a CDR3 sequence), that is flanked on each side by (part of) a framework sequence (and in particular, part of the framework sequence(s) that, in the immunoglobulin sequence from which the fragment is derived, are adjacent to said CDR sequence. For example, a CDR3 sequence may be preceded by (part of) a FR3 sequence and followed by (part of) a FR4 sequence). Such a fragment may also contain a disulphide bridge, and in particular a disulphide bridge that links the two framework regions that precede and follow the CDR sequence, respectively (for the purpose of forming such a disulphide bridge, cysteine residues that naturally occur in said framework regions may be used, or alternatively cysteine residues may be synthetically added to or introduced into said framework regions). For a further description of these □Expedite fragments□, reference [symbol] [symbol] WO 03/050531, as well as to the US provisional application of Ablynx N.V. entitled “Peptides capable of binding to serum proteins.” of Ablynx N.V. (inventors: Revets, Hilde Adi Pierrette; Kolkman, Joost Alexander; and Hoogenboom, Hendricus Renerus Jacobus Mattheus) filed on Dec. 5, 2006 (see also PCT/EP2007/063348).

In another aspect, the invention relates to a compound or construct, and in particular a protein or polypeptide (also referred to herein as a □compound of the invention□polypeptide of the invention□, respectively) that comprises or essentially consists of one or more amino acid sequences of the invention (or suitable fragments thereof), and optionally further comprises one or more other groups, residues, moieties or binding units. As will become clear to the skilled person from the further disclosure herein, such further groups, residues, moieties, binding units or amino acid sequences may or may not provide further functionality to the amino acid sequence of the invention (and/or to the compound or construct in which it is present) and may or may not modify the properties of the amino acid sequence of the invention.

For example, such further groups, residues, moieties or binding units may be one or more additional amino acid sequences, such that the compound or construct is a (fusion) protein or (fusion) polypeptide. In a preferred but non-limiting aspect, said one or more other groups, residues, moieties or binding units are immunoglobulin sequences. Even more preferably, said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”□s, amino acid sequences that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more amino acid sequences of the invention so as to provide a □derivative□ of an amino acid sequence or polypeptide of the invention, as further described herein.

Also within the scope of the present invention are compounds or constructs, that comprises or essentially consists of one or more derivatives as described herein, and optionally further comprises one or more other groups, residues, moieties or binding units, optionally linked via one or more linkers. Preferably, said one or more other groups, residues, moieties or binding units are amino acid sequences.

In the compounds or constructs described above, the one or more amino acid sequences of the invention and the one or more groups, residues, moieties or binding units may be linked directly to each other and/or via one or more suitable linkers or spacers. For example, when the one or more groups, residues, moieties or binding units are amino acid sequences, the linkers may also be amino acid sequences, so that the resulting compound or construct is a fusion (protein) or fusion (polypeptide).

As will be clear from the further description above and herein, this means that the amino acid sequences of the invention can be used as □ building blocks□ to form polypeptides of the invention, i.e. by suitably combining them with other groups, residues, moieties or binding units, in order to form compounds or constructs as described herein (such as, without limitations, the biparatopic. bi/multivalent and bi/multispecific polypeptides of the invention described herein) which combine within one molecule one or more desired properties or biological functions.

The compounds or polypeptides of the invention can generally be prepared by a method which comprises at least one step of suitably linking the one or more amino acid sequences of the invention to the one or more further groups, residues, moieties or binding units, optionally via the one or more suitable linkers, so as to provide the compound or polypeptide of the invention. Polypeptides of the invention can also be prepared by a method which generally comprises at least the steps of providing a nucleic acid that encodes a polypeptide of the invention, expressing said nucleic acid in a suitable manner, and recovering the expressed polypeptide of the invention. Such methods can be performed in a manner known per se, which will be clear to the skilled person, for example on the basis of the methods and techniques further described herein.

The process of designing/selecting and/or preparing a compound or polypeptide of the invention, starting from an amino acid sequence of the invention, is also referred to herein as □formatting□ id amino acid sequence of the invention; and an amino acid of the invention that is made part of a compound or polypeptide of the invention is said to be □formatted□ or to be □in the format of □ said compound or polypeptide of the invention. Examples of way which an amino acid sequence of the invention can be formatted and examples of such formats will be clear to the skilled person based on the disclosure herein; and such formatted amino acid sequences form a further aspect of the invention.

In one specific aspect of the invention, a compound of the invention or a polypeptide of the invention may have an increased half-life, compared to the corresponding amino acid sequence of the invention. Some preferred, but non-limiting examples of such compounds and polypeptides will become clear to the skilled person based on the further disclosure herein, and for example comprise amino acid sequences or polypeptides of the invention that have been chemically modified to increase the half-life thereof (for example, by means of pegylation); amino acid sequences of the invention that comprise at least one additional binding site for binding to a serum protein (such as serum albumin); or polypeptides of the invention that comprise at least one amino acid sequence of the invention that is linked to at least one moiety (and in particular at least one amino acid sequence) that increases the half-life of the amino acid sequence of the invention. Examples of polypeptides of the invention that comprise such half-life extending moieties or amino acid sequences will become clear to the skilled person based on the further disclosure herein; and for example include, without limitation, polypeptides in which the one or more amino acid sequences of the invention are suitable linked to one or more serum proteins or fragments thereof (such as (human) serum albumin or suitable fragments thereof) or to one or more binding units that can bind to serum proteins (such as, for example, domain antibodies, amino acid sequences that are suitable for use as a domain antibody, single domain antibodies, amino acid sequences that are suitable for use as a single domain antibody, “dAb”□s, amino acid sequences that are suitable for use as a dAb, or Nanobodies that can bind to serum proteins such as serum albumin (such as human serum albumin), serum immunoglobulins such as IgG, or transferrine; reference is made to the further description and references mentioned herein); polypeptides in which an amino acid sequence of the invention is linked to an Fc portion (such as a human Fc) or a suitable part or fragment thereof; or polypeptides in which the one or more amino acid sequences of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006 (see also PCT/EP2007/063348).

Generally, the compounds or polypeptides of the invention with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the compounds or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In a preferred, but non-limiting aspect of the invention, such compounds or polypeptides of the invention have a serum half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In another preferred, but non-limiting aspect of the invention, such compounds or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another aspect, the invention relates to a nucleic acid that encodes an amino acid sequence of the invention or a polypeptide of the invention (or a suitable fragment thereof). Such a nucleic acid will also be referred to herein as a □nucleic acid of the invention□ and may for example be in the form of a genetic construct, as further described herein.

In another aspect, the invention relates to a host or host cell that expresses (or that under suitable circumstances is capable of expressing) an amino acid sequence of the invention and/or a polypeptide of the invention; and/or that contains a nucleic acid of the invention. Some preferred but non-limiting examples of such hosts or host cells will become clear from the further description herein.

The invention further relates to a product or composition containing or comprising at least one amino acid sequence of the invention, at least one polypeptide of the invention (or a suitable fragment thereof) and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention also relates to the use of an amino acid sequence, Nanobody or polypeptide of the invention, or of a composition comprising the same, in (methods or compositions for) modulating Integrins, either in vitro (e.g. in an in vitro or cellular assay) or in vivo (e.g. in an a single cell or in a multicellular organism, and in particular in a mammal, and more in particular in a human being, such as in a human being that is at risk of or suffers from an autoimmune diseases, cancer metastasis and thrombotic vascular diseases).

The invention also relates to methods for modulating Integrins, either in vitro (e.g. in an in vitro or cellular assay) or in vivo (e.g. in an a single cell or multicellular organism, and in particular in a mammal, and more in particular in a human being, such as in a human being that is at risk of or suffers from an autoimmune diseases, cancer metastasis and thrombotic vascular diseases), which method comprises at least the step of contacting Integrins with at least one amino acid sequence, Nanobody or polypeptide of the invention, or with a composition comprising the same, in a manner and in an amount suitable to modulate Integrins, with at least one amino acid sequence, Nanobody or polypeptide of the invention.

The invention also relates to the use of an one amino acid sequence, Nanobody or polypeptide of the invention in the preparation of a composition (such as, without limitation, a pharmaceutical composition or preparation as further described herein) for modulating Integrins, either in vitro (e.g. in an in vitro or cellular assay) or in vivo (e.g. in an a single cell or multicellular organism, and in particular in a mammal, and more in particular in a human being, such as in a human being that is at risk of or suffers from an autoimmune diseases, cancer metastasis and thrombotic vascular diseases).

In the context of the present invention, □modulating□ or □to modulate□ generally means either reducing or inhibiting the activity of, or alternatively increasing the activity of, Integrins, as measured using a suitable in vitro, cellular or in vivo assay (such as those mentioned herein). In particular, □modulating□ or □ to modulate□ may mean either redu inhibiting the activity of, or alternatively increasing the activity of Integrins, as measured using a suitable in vitro, cellular or in vivo assay (such as those mentioned herein), by at least 1%, preferably at least 5%, such as at least 10% or at least 25%, for example by at least 50%, at least 60%, at least 70%, at least 80%, or 90% or more, compared to activity of Integrins in the same assay under the same conditions but without the presence of the amino acid sequence, Nanobody or polypeptide of the invention.

As will be clear to the skilled person, □modulating□ may also involve effecting a change (which may either be an increase or a decrease) in affinity, avidity, specificity and/or selectivity of Integrins for one or more of its targets, ligands or substrates; and/or effecting a change (which may either be an increase or a decrease) in the sensitivity of Integrins for one or more conditions in the medium or surroundings in which Integrins is present (such as pH, ion strength, the presence of co-factors, etc.), compared to the same conditions but without the presence of the amino acid sequence, Nanobody or polypeptide of the invention. As will be clear to the skilled person, this may again be determined in any suitable manner and/or using any suitable assay known per se, such as the assays described herein or in the prior art cited herein.

□Modulating□ also mean effecting a change (i.e. an activity as an agonist or as an antagonist, respectively) with respect to one or more biological or physiological mechanisms, effects, responses, functions, pathways or activities in which Integrins (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, such as the assays described herein or in the prior art cited herein. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the amino acid sequence, Nanobody or polypeptide of the invention.

Modulating may for example involve reducing or inhibiting the binding of Integrins to one of its substrates or ligands and/or competing with a natural ligand, substrate for binding to Integrins. Modulating may also involve activating Integrins or the mechanism or pathway in which it is involved. Modulating may be reversible or irreversible, but for pharmaceutical and pharmacological purposes will usually be in a reversible manner.

The invention further relates to methods for preparing or generating the amino acid sequences, polypeptides, nucleic acids, host cells, products and compositions described herein. Some preferred but non-limiting examples of such methods will become clear from the further description herein.

Generally, these methods may comprise the steps of:
a) providing a set, collection or library of amino acid sequences; and
b) screening said set, collection or library of amino acid sequences for amino acid sequences that can bind to and/or have affinity for Integrins; and
c) isolating the amino acid sequence(s) that can bind to and/or have affinity for Integrins.

In such a method, the set, collection or library of amino acid sequences may be any suitable set, collection or library of amino acid sequences. For example, the set, collection or library of amino acid sequences may be a set, collection or library of immunoglobulin sequences (as described herein), such as a naïve set, collection or library of immunoglobulin sequences; a synthetic or semi-synthetic set, collection or library of immunoglobulin sequences; and/or a set, collection or library of immunoglobulin sequences that have been subjected to affinity maturation.

Also, in such a method, the set, collection or library of amino acid sequences may be a set, collection or library of heavy chain variable domains (such as V_{H} domains or V_{HH} domains) or of light chain variable domains. For example, the set, collection or library of amino acid sequences may be a set, collection or library of domain antibodies or single domain antibodies, or may be a set, collection or library of amino acid sequences that are capable of functioning as a domain antibody or single domain antibody.

In a preferred aspect of this method, the set, collection or library of amino acid sequences may be an immune set, collection or library of immunoglobulin sequences, for example derived from a mammal that has been suitably immunized with Integrins or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

In another aspect, the method for generating amino acid sequences comprises at least the steps of:
a) providing a collection or sample of cells expressing amino acid sequences;
b) screening said collection or sample of cells for cells that express an amino acid sequence that can bind to and/or have affinity for Integrins;
   and
c) either (i) isolating said amino acid sequence; or (ii) isolating from said cell a nucleic acid sequence that encodes said amino acid sequence, followed by expressing said amino acid sequence.

For example, when the desired amino acid sequence is an immunoglobulin sequence, the collection or sample of cells may for example be a collection or sample of B-cells. Also, in this method, the sample of cells may be derived from a mammal that has been suitably immunized with Integrins or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820 (2001).

In another aspect, the method for generating an amino acid sequence directed against Integrins may comprise at least the steps of:
a) providing a set, collection or library of nucleic acid sequences encoding amino acid sequences;
b) screening said set, collection or library of nucleic acid sequences for nucleic acid sequences that encode an amino acid sequence that can bind to and/or has affinity for Integrins;
   and
c) isolating said nucleic acid sequence, followed by expressing said amino acid sequence.

In such a method, the set, collection or library of nucleic acid sequences encoding amino acid sequences may for example be a set, collection or library of nucleic acid sequences encoding a naïve set, collection or library of immunoglobulin sequences; a set, collection or library of nucleic acid sequences encoding a synthetic or semi-synthetic set, collection or library of immunoglobulin sequences; and/or a set, collection or library of nucleic acid sequences encoding a set, collection or library of immunoglobulin sequences that have been subjected to affinity maturation.

Also, in such a method, the set, collection or library of nucleic acid sequences may encode a set, collection or library of heavy chain variable domains (such as V_{H} domains or V_{HH} domains) or of light chain variable domains. For example, the set, collection or library of nucleic acid sequences may encode a set, collection or library of domain antibodies or single domain antibodies, or a set, collection or library of amino acid sequences that are capable of functioning as a domain antibody or single domain antibody.

In a preferred aspect of this method, the set, collection or library of amino acid sequences may be an immune set, collection or library of nucleic acid sequences, for example derived from a mammal that has been suitably immunized with Integrins or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The set, collection or library of nucleic acid sequences may for example encode an immune set, collection or library of heavy chain variable domains or of light chain variable domains. In one specific aspect, the set, collection or library of nucleotide sequences may encode a set, collection or library of V_{HH} sequences.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to the review by Hoogenboom in Nature Biotechnology, 23, 9, 1105-1116 (2005).

In another aspect, the method for generating an amino acid sequence directed against Integrins may comprise at least the steps of:
a) providing a set, collection or library of nucleic acid sequences encoding amino acid sequences;
b) screening said set, collection or library of nucleic acid sequences for nucleic acid sequences that encode an amino acid sequence that can bind to and/or has affinity for Integrins and that is cross-blocked or is cross blocking a Nanobody of the invention, e.g. SEQ ID NO: 1316 to 1476, and SEQ ID NO: 1485, 1486, and 1487 (Table-1); and
c) isolating said nucleic acid sequence, followed by expressing said amino acid sequence.

The invention also relates to amino acid sequences that are obtained by the above methods, or alternatively by a method that comprises the one of the above methods and in addition at least the steps of determining the nucleotide sequence or amino acid sequence of said immunoglobulin sequence; and of expressing or synthesizing said amino acid sequence in a manner known per se, such as by expression in a suitable host cell or host organism or by chemical synthesis.

Also, following the steps above, one or more amino acid sequences of the invention may be suitably humanized (or alternatively camelized); and/or the amino acid sequence(s) thus obtained may be linked to each other or to one or more other suitable amino acid sequences (optionally via one or more suitable linkers) so as to provide a polypeptide of the invention. Also, a nucleic acid sequence encoding an amino acid sequence of the invention may be suitably humanized (or alternatively camelized) and suitably expressed; and/or one or more nucleic acid sequences encoding an amino acid sequence of the invention may be linked to each other or to one or more nucleic acid sequences that encode other suitable amino acid sequences (optionally via nucleotide sequences that encode one or more suitable linkers), after which the nucleotide sequence thus obtained may be suitably expressed so as to provide a polypeptide of the invention.

The invention further relates to applications and uses of the amino acid sequences, compounds, constructs, polypeptides, nucleic acids, host cells, products and compositions described herein, as well as to methods for the prevention and/or treatment for diseases and disorders associated with Integrins. Some preferred but non-limiting applications and uses will become clear from the further description herein.

The invention also relates to the amino acid sequences, compounds, constructs, polypeptides, nucleic acids, host cells, products and compositions described herein for use in therapy. In particular, the invention also relates to the amino acid sequences, compounds, constructs, polypeptides, nucleic acids, host cells, products and compositions described herein for use in therapy of a disease or disorder that can be prevented or treated by administering, to a subject in need thereof, of (a pharmaceutically effective amount of) an amino acid sequence, compound, construct or polypeptide as described herein.

More in particular, the invention relates to the amino acid sequences, compounds, constructs, polypeptides, nucleic acids, host cells, products and compositions described herein for use in therapy of autoimmune diseases, cancer metastasis and thrombotic vascular diseases. Other aspects, embodiments, advantages and applications of the invention will also become clear from the further description herein, in which the invention will be described and discussed in more detail with reference to the Nanobodies of the invention and polypeptides of the invention comprising the same, which form some of the preferred aspects of the invention.

As will become clear from the further description herein, Nanobodies generally offer certain advantages (outlined herein) compared to □dAb□s□ or similar (single) domain antibodies or immunoglobulin sequences, which advantages are also provided by the Nanobodies of the invention. However, it will be clear to the skilled person that the more general aspects of the teaching below can also be applied (either directly or analogously) to other amino acid sequences of the invention.

### DETAILED DESCRIPTION OF THE INVENTION

In the present description, examples and claims:
a) Unless indicated or defined otherwise, all terms used have their usual meaning in the art, which will be clear to the skilled person. Reference is for example made to the standard handbooks mentioned in paragraph a) on page 46 of WO 08/020,079.
b) Unless indicated otherwise, the terms □immunoglobulin sequence□, □sequence□, □nucleotide sequence□ and □nucleic acid□ are as described in paragraph b) on pa of WO 08/020,079.
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2(1): 49-57; Irving et al., J. Immunol. Methods, 2001, 248(1-2), 31-45; Schmitz et al., Placenta, 2000, 21 Suppl. A, S106-12, Gonzales et al., Tumour Biol., 2005, 26(1), 31-43, which describe techniques for protein engineering, such as affinity maturation and other techniques for improving the specificity and other desired properties of proteins such as immunoglobulins.
d) Amino acid residues will be indicated according to the standard three-letter or one-letter amino acid code. Reference is made to Table A-2 on page 48 of the International application WO 08/020,079 of Ablynx N.V. entitled □Amino acid sequences directed against IL-6R and polypeptides comprising the same for the treatment of diseases and disorders associated with 11-6 mediated signalling□.
e) For the purposes of comparing two or more nucleotide sequences, the percentage of □sequence identity□ between a first nucleotide sequence and a second nucleotide sequence may be calculated or determined as described in paragraph c) on page 49 of WO 08/020,079 (incorporated herein by reference), such as by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position); or using a suitable computer algorithm or technique, again as described in paragraph c) on pages 49 of WO 08/020,079 (incorporated herein by reference).
f) For the purposes of comparing two or more amino acid sequences, the percentage of □sequence identity□ between a first amino acid sequence [symbol] [symbol] second amino acid sequence (also referred to herein as □amino acid identity □may be calculated or determined as described in paragraph 0 on pages 49 and 50 of WO 08/020,079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an □amino acid difference□ as defined herein; or using a suitable computer algorithm or technique, again as described in paragraph f) on pages 49 and 50 of WO 08/020,079 (incorporated herein by reference). Also, in determining the degree of sequence identity between two amino acid sequences, the skilled person may take into account so-called □conservative□ amino acid substitutions, as described on ps WO [symbol] [symbol] /020079. Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Natl. Acad. Sci. USA 81: 140-144, 1984; Kyte & Doolittle; J. Molcc. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
g) Amino acid sequences and nucleic acid sequences are said to be □exactly the same□ if they have 100% sequence identity (as defined herein) over their entire length.
h) When comparing two amino acid sequences, the term □amino acid difference□ refers to an insertion, deletion or substitution of a single amino acid residue on a position of the first sequence, compared to the second sequence; it being understood that two amino acid sequences can contain one, two or more such amino acid differences.
i) When a nucleotide sequence or amino acid sequence is said to □comprise□ another nucleotide sequence or amino acid sequence, respectively, or to □essentially consist of□ another nucleotide sequence or amino acid sequence, this has the meaning given in paragraph i) on pages 51-52 of WO 08/020,079.
j) The term □essentially isolated form□ has the meaning given to it in paragraph j) on pages 52 and 53 of WO 08/020,079.
k) The terms □domain□ and □binding domain□ have the meanings [symbol] [symbol] iven to it in pare k) on page 53 of WO 08/020,079.
l) The terms □antigenic determinant□ [symbol] [symbol] □, which may also be used interchangeably herein have the meanings given to it in paragraph l) on page 53 of WO 08/020,079.
m) As further described in paragraph m) on page 53 of WO 08/020,079, an amino acid sequence (such as a Nanobody, an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be □against□ directed against□ said antigenic determinant, epitope, antigen or protein.
n) The term □specificity□ has the meaning given to it in paragraph n) on page [symbol] [symbol] f WO 08/020,079; and as mentioned therein refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity, as described on pages 53-56 of WO 08/020,079 (incorporated herein by reference), which also describes some preferred techniques for measuring binding between an antigen-binding molecule (such as a Nanobody or polypeptide of the invention) and the pertinent antigen. Typically, antigen-binding proteins (such as the amino acid sequences, Nanobodies and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. As will be clear to the skilled person, and as described on pages 53-56 of WO 08/020,079, the dissociation constant may be the actual or apparent dissociation constant. Methods for determining the dissociation constant will be clear to the skilled person, and for example include the techniques mentioned on pages 53-56 of WO 08/020,079. The half-life of an amino acid sequence, compound or polypeptide of the invention can generally be defined as described in paragraph o) on page 57 of WO 08/020,079 and as mentioned therein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally be as described in paragraph o) on page 57 of WO 08/020,079. As also mentioned in paragraph o) on page 57 of WO 08/020,079, the half-life can be expressed using parameters such as the t1/2-alpha, t1/2-beta and the area under the curve (AUC). Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982). The terms □increase or [symbol] [symbol] or □ [symbol] [symbol] half as also as defined in paragraph o) on page 57 of WO 08/020,079 and in particular refer to an increase in the t1/2-beta, either with or without an increase in the t1/2-alpha and/or the AUC or both.
o) In the context of the present invention, □modulating□ or □to modulate□ generally either reducing or inhibiting the activity of, or alternatively increasing the activity of, a target or antigen, as measured using a suitable in vitro, cellular or in vivo assay. In particular, □modulating□ or □to modulate□ may mean either reducing or inhibiting activity of, or alternatively increasing a (relevant or intended) biological activity of, a target or antigen, as measured using a suitable in vitro, cellular or in vivo assay (which will usually depend on the target or antigen involved), by at least 1%, preferably at least 5%, such as at least 10% or at least 25%, for example by at least 50%, at least 60%, at least 70%, at least 80%, or 90% or more, compared to activity of the target or antigen in the same assay under the same conditions but without the presence of the construct of the invention. As will be clear to the skilled person, □modulating□ may also involve effecting a change (which may either be an increase or a decrease) in affinity, avidity, specificity and/or selectivity of a target or antigen for one or more of its ligands, binding partners, partners for association into a homomultimeric or heteromultimeric form, or substrates; and/or effecting a change (which may either be an increase or a decrease) in the sensitivity of the target or antigen for one or more conditions in the medium or surroundings in which the target or antigen is present (such as pH, ion strength, the presence of co-factors, etc.), compared to the same conditions but without the presence of the construct of the invention. As will be clear to the skilled person, this may again be determined in any suitable manner and/or using any suitable assay known per se, depending on the target or antigen involved. □Modulating□ may also mean effecting a change (i.e. an activity as an agonist, as an antagonist or as a reverse agonist, respectively, depending on the target or antigen and the desired biological or physiological effect) with respect to one or more biological or physiological mechanisms, effects, responses, functions, pathways or activities in which the target or antigen (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, depending on the target or antigen involved. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the construct of the invention. Modulating may for example also involve allosteric modulation of the target or antigen; and/or reducing or inhibiting the binding of the target or antigen to one of its substrates or ligands and/or competing with a natural ligand, substrate for binding to the target or antigen. Modulating may also involve activating the target or antigen or the mechanism or pathway in which it is involved. Modulating may for example also involve effecting a change in respect of the folding or confirmation of the target or antigen, or in respect of the ability of the target or antigen to fold, to change its confirmation (for example, upon binding of a ligand), to associate with other (sub)units, or to disassociate. Modulating may for example also involve effecting a change in the ability of the target or antigen to transport other compounds or to serve as a channel for other compounds (such as ions). Modulating may be reversible or irreversible, but for pharmaceutical and pharmacological purposes will usually be in a reversible manner.
p) In respect of a target or antigen, the term □interaction site□ on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen. More generally, an □interaction site□ can be any site, epitope, antigenic determinant, part, domain or stretch of amino acid residues on the target or antigen to which an amino acid sequence or polypeptide of the invention can bind such that the target or antigen (and/or any pathway, interaction, signalling, biological mechanism or biological effect in which the target or antigen is involved) is modulated (as defined herein).
q) An amino acid sequence or polypeptide is said to be □specific for□ a first target or antigen compared to a second target or antigen when is binds to the first antigen with an affinity (as described above, and suitably expressed as a K_{D} value, K_{A} value, K_{off} rate and/or Kₒₙ rate) that is at least 10 times, such as at least 100 times, and preferably at least 1000 times, and up to 10.000 times or more better than the affinity with which said amino acid sequence or polypeptide binds to the second target or polypeptide. For example, the first antigen may bind to the target or antigen with a K_{D} value that is at least 10 times less, such as at least 100 times less, and preferably at least 1000 times less, such as 10.000 times less or even less than that, than the K_{D} with which said amino acid sequence or polypeptide binds to the second target or polypeptide. Preferably, when an amino acid sequence or polypeptide is □specific for□ a first target or antigen compared to a second target or antigen, it is directed against (as defined herein) said first target or antigen, but not directed against said second target or antigen.
r) The terms □cross-block□ cross-blocked□ and cross-blocking□ are used interchangeably herein to mean the ability of an amino acid sequence or other binding agents (such as a polypeptide of the invention) to interfere with the binding of other amino acid sequences or binding agents of the invention to a given target. The extend to which an amino acid sequence or other binding agents of the invention is able to interfere with the binding of another, and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between amino acid sequence or another binding agents in terms of their binding to the target. The following generally describes a suitable Biacore assay for determining whether an amino acid sequence or other binding agent cross-blocks or is capable of cross-blocking according to the invention. It will be appreciated that the assay can be used with any of the amino acid sequence or other binding agents described herein. The Biacore machine (for example the Biacore 3000) is operated in line with the manufacturer's recommendations. Thus in one cross-blocking assay, the target protein is coupled to a CM5 Biacore chip using standard amine coupling chemistry to generate a surface that is coated with the target. Typically 200-800 resonance units of the target would be coupled to the chip (an amount that gives easily measurable levels of binding but that is readily saturable by the concentrations of test reagent being used). Two test amino acid sequences (termed A* and B*) to be assessed for their ability to cross-block each other are mixed at a one to one molar ratio of binding sites in a suitable buffer to create the test mixture. When calculating the concentrations on a binding site basis the molecular weight of an amino acid sequence is assumed to be the total molecular weight of the amino acid sequence divided by the number of target binding sites on that amino acid sequence. The concentration of each amino acid sequence in the test mix should be high enough to readily saturate the binding sites for that amino acid sequence on the target molecules captured on the Biacore chip. The amino acid sequences in the mixture are at the same molar concentration (on a binding basis) and that concentration would typically be between 1.00 and 1.5 micromolar (on a binding site basis). Separate solutions containing A* alone and B* alone are also prepared. A* and B* in these solutions should be in the same buffer and at the same concentration as in the test mix. The test mixture is passed over the target-coated Biacore chip and the total amount of binding recorded. The chip is then treated in such a way as to remove the bound amino acid sequences without damaging the chip-bound target. Typically this is done by treating the chip with 30 mM HCl for 60 seconds. The solution of A* alone is then passed over the target-coated surface and the amount of binding recorded. The chip is again treated to remove all of the bound amino acid sequences without damaging the chip-bound target. The solution of B* alone is then passed over the target-coated surface and the amount of binding recorded. The maximum theoretical binding of the mixture of A* and B* is next calculated, and is the sum of the binding of each amino acid sequence when passed over the target surface alone. If the actual recorded binding of the mixture is less than this theoretical maximum then the two amino acid sequences are cross-blocking each other. Thus, in general, a cross-blocking amino acid sequence or other binding agent according to the invention is one which will bind to the target in the above Biacore cross-blocking assay such that during the assay and in the presence of a second amino acid sequence or other binding agent of the invention the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two amino acid sequences or binding agents in combination. The Biacore assay described above is a primary assay used to determine if amino acid sequences or other binding agents cross-block each other according to the invention. On rare occasions particular amino acid sequences or other binding agents may not bind to target coupled via amine chemistry to a CM5 Biacore chip (this usually occurs when the relevant binding site on target is masked or destroyed by the coupling to the chip). In such cases cross-blocking can be determined using a tagged version of the target, for example a N-terminal His-tagged version (R & D Systems, Minneapolis, Minn., USA; 2005 cat# 1406-ST-025). In this particular format, an anti-His amino acid sequence would be coupled to the Biacore chip and then the His-tagged target would be passed over the surface of the chip and captured by the anti-His amino acid sequence. The cross blocking analysis would be carried out essentially as described above, except that after each chip regeneration cycle, new His-tagged target would be loaded back onto the anti-His amino acid sequence coated surface. In addition to the example given using N-terminal His-tagged target, C-terminal His-tagged target could alternatively be used. Furthermore, various other tags and tag binding protein combinations that are known in the art could be used for such a cross-blocking analysis (e.g. HA tag with anti-FIA antibodies; FLAG tag with anti-FLAG antibodies; biotin tag with streptavidin). The following generally describes an ELISA assay for determining whether an amino acid sequence or other binding agent directed against a target cross-blocks or is capable of cross-blocking as defined herein. It will be appreciated that the assay can be used with any of the amino acid sequences (or other binding agents such as polypeptides of the invention) described herein. The general principal of the assay is to have an amino acid sequence or binding agent that is directed against the target coated onto the wells of an ELISA plate. An excess amount of a second, potentially cross-blocking, anti-target amino acid sequence is added in solution (i.e. not bound to the ELISA plate). A limited amount of the target is then added to the wells. The coated amino acid sequence and the amino acid sequence in solution compete for binding of the limited number of target molecules. The plate is washed to remove excess target that has not been bound by the coated amino acid sequence and to also remove the second, solution phase amino acid sequence as well as any complexes formed between the second, solution phase amino acid sequence and target. The amount of bound target is then measured using a reagent that is appropriate to detect the target. An amino acid sequence in solution that is able to cross-block the coated amino acid sequence will be able to cause a decrease in the number of target molecules that the coated amino acid sequence can bind relative to the number of target molecules that the coated amino acid sequence can bind in the absence of the second, solution phase, amino acid sequence. In the instance where the first amino acid sequence, e.g. an Ab-X, is chosen to be the immobilized amino acid sequence, it is coated onto the wells of the ELISA plate, after which the plates are blocked with a suitable blocking solution to minimize non-specific binding of reagents that are subsequently added. An excess amount of the second amino acid sequence, i.e. Ab-Y, is then added to the ELISA plate such that the moles of Ab-Y target binding sites per well are at least 10 fold higher than the moles of Ab-X target binding sites that were used, per well, during the coating of the ELISA plate. Target is then added such that the moles of target added per well are at least 25-fold lower than the moles of Ab-X target binding sites that were used for coating each well. Following a suitable incubation period the ELISA plate is washed and a reagent for detecting the target is added to measure the amount of target specifically bound by the coated anti-target amino acid sequence (in this case Ab-X). The background signal for the assay is defined as the signal obtained in wells with the coated amino acid sequence (in this case Ab-X), second solution phase amino acid sequence (in this case Ab-Y), [target] buffer only (i.e. no target) and target detection reagents. The positive control signal for the assay is defined as the signal obtained in wells with the coated amino acid sequence (in this case Ab-X), second solution phase amino acid sequence buffer only (i.e. no second solution phase amino acid sequence), target and target detection reagents. The ELISA assay may be run in such a manner so as to have the positive control signal be at least 6 times the background signal. To avoid any artefacts (e.g. significantly different affinities between Ab-X and Ab-Y for target) resulting from the choice of which amino acid sequence to use as the coating amino acid sequence and which to use as the second (competitor) amino acid sequence, the cross-blocking assay may to be run in two formats: 1) format 1 is where Ab-X is the amino acid sequence that is coated onto the ELISA plate and Ab-Y is the competitor amino acid sequence that is in solution and 2) format 2 is where Ab-Y is the amino acid sequence that is coated onto the ELISA plate and Ab-X is the competitor amino acid sequence that is in solution. Ab-X and Ab-Y are defined as cross-blocking if, either in format 1 or in format 2, the solution phase anti-target amino acid sequence is able to cause a reduction of between 60% and 100%, specifically between 70% and 100%, and more specifically between 80% and 100%, of the target detection signal (i.e. the amount of target bound by the coated amino acid sequence) as compared to the target detection signal obtained in the absence of the solution phase anti-target amino acid sequence (i.e. the positive control wells).
s) As further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein. As further described in paragraph q) on pages 58 and 59 of WO 08/020,079 (incorporated herein by reference), the amino acid residues of a Nanobody are numbered according to the general numbering for V_{H} domains given by Kabat et al. (□Sequence of proteins immunological interest□, US Public Health Service NIH Bethesda, Md., Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication), and accordingly FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113.
t) The Figures, Sequence Listing and the Experimental Part/Examples are only given to further illustrate the invention and should not be interpreted or construed as limiting the scope of the invention and/or of the appended claims in any way, unless explicitly indicated otherwise herein.

For a general description of heavy chain antibodies and the variable domains thereof, reference is inter alia made to the prior art cited herein, as well as to the prior art mentioned on page 59 of WO 08/020,079 and to the list of references mentioned on pages 41-43 of the International application WO 06/040153, which prior art and references are incorporated herein by reference.

In accordance with the terminology used in the art (see the above references), the variable domains present in naturally occurring heavy chain antibodies will also be referred to as □V_{HH} domains□, in order to distinguish them from the heavy chain variable domains that are present in conventional 4-chain antibodies (which will be referred to hereinbelow as □V_{H} domains□) [symbol] [symbol] the light chain variable domains that are present in conventional 4-chain antibodies (which will be referred to hereinbelow as □V_{L}, domains□).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been □designed□ by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{I}, domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv□s fragments, which consist of [symbol] [symbol] domain covalently linked to a V_{L} domain).

Because of these unique properties, the use of V_{HH} domains and Nanobodies as single antigen-binding proteins or as antigen-binding domains (i.e. as part of a larger protein or polypeptide) offers a number of significant advantages over the use of conventional V_{H} and V_{L} domains, scFv□s or conventional antibody fragments (such a [symbol] [symbol] (ab□₂-fragments), including the advantages that are listed on pages 60 and 61 of WO 08/020,079. In a specific and preferred aspect, the invention provides Nanobodies against Integrins, and in particular Nanobodies against Integrins from a warm-blooded animal, and more in particular Nanobodies against Integrins from a mammal, and especially Nanobodies against human Integrins; as well as proteins and/or polypeptides comprising at least one such Nanobody.

In particular, the invention provides Nanobodies against Integrins, and proteins and/or polypeptides comprising the same, that have improved therapeutic and/or pharmacological properties and/or other advantageous properties (such as, for example, improved ease of preparation and/or reduced costs of goods), compared to conventional antibodies against Integrins or fragments thereof, compared to constructs that could be based on such conventional antibodies or antibody fragments (such as Fab□fragments, F₂(fragments), ScFv constructs, □diabodies□ and other multispecific constructs (see for example the review by Holliger and Hudson, Nat Biotechnol. 2005 Scp;23(9):1126-36)), and also compared to the so-called □ dAb□s□ or similar (single) domain antibodies that may be derived from varial domains of conventional antibodies. These improved and advantageous properties will become clear from the further description herein, and for example include, without limitation, one or more of:
increased affinity and/or avidity for Integrins, either in a monovalent format, in a multivalent format (for example in a bivalent format) and/or in a multispecific format (for example one of the multispecific formats described hereinbelow);
better suitability for formatting in a multivalent format (for example in a bivalent format);
better suitability for formatting in a multispecific format (for example one of the multispecific formats described hereinbelow);
improved suitability or susceptibility for □humanizing□substitutions (as defined herein);
less immunogenicity, either in a monovalent format, in a multivalent format (for example in a bivalent format) and/or in a multispecific format (for example one of the multispecific formats described hereinbelow);
increased stability, either in a monovalent format, in a multivalent format (for example in a bivalent format) and/or in a multispecific format (for example one of the multispecific formats described hereinbelow);
increased specificity towards Integrins, either in a monovalent format, in a multivalent format (for example in a bivalent format) and/or in a multispecific format (for example one of the multispecific formats described hereinbelow);
decreased or where desired increased cross-reactivity with Integrins from different species;
   and/or
one or more other improved properties desirable for pharmaceutical use (including prophylactic use and/or therapeutic use) and/or for diagnostic use (including but not limited to use for imaging purposes), either in a monovalent format, in a multivalent format (for example in a bivalent format) and/or in a multispecific format (for example one of the multispecific formats described hereinbelow).

As generally described herein for the amino acid sequences of the invention, the Nanobodies of the invention are preferably in essentially isolated form (as defined herein), or form part of a protein or polypeptide of the invention (as defined herein), which may comprise or essentially consist of one or more Nanobodies of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than Integrins), so as to provide a monovalent, multivalent or multispecific polypeptide of the invention, respectively, all as described herein. In particular, such a protein or polypeptide may comprise or essentially consist of one or more Nanobodies of the invention and optionally one or more (other) Nanobodies (i.e. directed against other targets than Integrins), all optionally linked via one or more suitable linkers, so as to provide a monovalent, multivalent or multispecific Nanobody construct, respectively, as further described herein. Such proteins or polypeptides may also be in essentially isolated form (as defined herein).

In a Nanobody of the invention, the binding site for binding against Integrins is preferably formed by the CDR sequences. Optionally, a Nanobody of the invention may also, and in addition to the at least one binding site for binding against Integrins, contain one or more further binding sites for binding against other antigens, proteins or targets. For methods and positions for introducing such second binding sites, reference is for example made to Keck and Huston, Biophysical Journal, 71, October 1996, 2002-2011; EP 0 640 130; and WO 06/07260.

As generally described herein for the amino acid sequences of the invention, when a Nanobody of the invention (or a polypeptide of the invention comprising the same) is intended for administration to a subject (for example for therapeutic and/or diagnostic purposes as described herein), it is preferably directed against human Integrins; whereas for veterinary purposes, it is preferably directed against Integrins from the species to be treated. Also, as with the amino acid sequences of the invention, a Nanobody of the invention may or may not be cross-reactive (i.e. directed against Integrins from two or more species of mammal, such as against human Integrins and Integrins from at least one of the species of mammal mentioned herein).

Also, again as generally described herein for the amino acid sequences of the invention, the Nanobodies of the invention may generally be directed against any antigenic determinant, epitope, part, domain, subunit or confirmation (where applicable) of Integrins.

As already described herein, the amino acid sequence and structure of a Nanobody can be considered—without however being limited thereto—to be comprised of four framework regions or □FR□s (or sometimes also referred to as□FW□s), w [symbol] [symbol] to in the art and herein as □Framework region□□ FR [symbol] [symbol] d□; as □Framework region 2□ [symbol] [symbol] □FR2□; as □Framework region 3□ or □FR3□; and as □ Framework region respectively; which framework regions are interrupted by three complementary determining regions or □CDR□s□, which are referred□ to in [symbol] Complementarity Determining Region 1□ or □CDR1□; as □Complementarity Determining Region 2□ or□ CD □Complementarity Determining Region 3□ or □CDR3□, respectively. Some preferred framework sequences and CDR s (and combinations thereof) that present in the Nanobodies of the invention are as described herein. Other suitable CDR sequences can be obtained by the methods described herein.

According to a non-limiting but preferred aspect of the invention, (the CDR sequences present in) the Nanobodies of the invention are such that:
the Nanobodies can bind to Integrins with a dissociation constant (Kₙ) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that:
the Nanobodies can bind to Integrins with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁻⁵ M⁻¹s⁻¹ and 10⁷ M⁻¹ s⁻¹;
   and/or such that they:
the Nanobodies can bind to Integrins with a k_{off} rate between (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹

Preferably, (the CDR sequences present in) the Nanobodies of the invention are such that: a monovalent Nanobody of the invention (or a polypeptide that contains only one Nanobody of the invention) is preferably such that it will bind to Integrins with an affinity less than 500 nM, preferably less than 200 nM, more preferably less than 10 nM, such as less than 500 pM.

The affinity of the Nanobody of the invention against Integrins can be determined in a manner known per se, for example using the general techniques for measuring Kᵣ). K_{A}, k_{off} or kₒₙ mentioned herein, as well as some of the specific assays described herein.

Some preferred IC50 values for binding of the Nanobodies of the invention (and of polypeptides comprising the same) to Integrins will become clear from the further description and examples herein.

In a preferred but non-limiting aspect, the invention relates to a Nanobody (as defined herein) against Integrins, which consists of 4 framework regions (FR1 to FR4 respectively) and 3 complementarity determining regions (CDR1 to CDR3 respectively), in which:
CDR1 is chosen from the group consisting of:
a) the amino acid sequences of SEQ ID NO□s: 296 to 465;
b) amino acid sequences that have at least 80% amino acid identity with at least one of the amino acid sequences of SEQ ID NO□s: 296 to 465;
c) amino acid sequences that have 3, 2, or 1 amino acid difference with at least one of the amino acid sequences of SEQ ID NO□s: 296 to 465;
   and/or
   CDR2 is chosen from the group consisting of:
d) the amino acid sequences of SEQ ID NO□s: 636 to 805;
e) amino acid sequences that have at least 80% amino acid identity with at least one of the amino acid sequences of SEQ ID NO□s: 636 to 805;
f) amino acid sequences that have 3, 2, or 1 amino acid difference with at least one of the amino acid sequences of SEQ ID NO□s: [symbol] ;
   and/or
   CDR3 is chosen from the group consisting of:
g) the amino acid sequences of SEQ ID NO□s: 976 to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□s: 976 to 1145;
   or any suitable fragment of such an amino acid sequence.

In particular, according to this preferred but non-limiting aspect, the invention relates to a Nanobody (as defined herein) against Integrins, which consists of 4 framework regions (FR1 to FR4 respectively) and 3 complementarity determining regions (CDR1 to CDR3 respectively), in which:
CDR1 is chosen from the group consisting of:
a) the amino acid sequences of SEQ ID NO□s: 296 to 465;
b) amino acid sequences that have at least 80% amino acid identity with at least one of the amino acid sequences of SEQ ID NO□s: 296 to 465;
c) amino acid sequences that have 3, 2, or 1 amino acid difference with at least one of the amino acid sequences of SEQ ID NO□s: 296 to 465;
   and
   CDR2 is chosen from the group consisting of:
d) the amino acid sequences of SEQ ID NO□s: 636 to 805;
e) amino acid sequences that have at least 80% amino acid identity with at least one of the amino acid sequences of SEQ ID NO□s: 636 to 805;
f) amino acid sequences that have 3, 2, or 1 amino acid difference with at least one of the amino acid sequences of SEQ ID NO□s: 636 to 805;
   and
   CDR3 is chosen from the group consisting of:
g) the amino acid sequences of SEQ ID NO□s: 976 to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□s: 976 to 1145;
   or any suitable fragment of such an amino acid sequences.

As generally mentioned herein for the amino acid sequences of the invention, when a Nanobody of the invention contains one or more CDR1 sequences according to b) and/or c):
i) any amino acid substitution in such a CDR according to b) and/or c) is preferably, and compared to the corresponding CDR according to a), a conservative amino acid substitution (as defined herein);
   and/or
ii) the CDR according to b) and/or c) preferably only contains amino acid substitutions, and no amino acid deletions or insertions, compared to the corresponding CDR according to a);
   and/or
iii) the CDR according to b) and/or c) may be a CDR that is derived from a CDR according to a) by means of affinity maturation using one or more techniques of affinity maturation known per se.

Similarly, when a Nanobody of the invention contains one or more CDR2 sequences according to e) and/or f):
i) any amino acid substitution in such a CDR according to e) and/or f) is preferably, and compared to the corresponding CDR according to d), a conservative amino acid substitution (as defined herein);
   and/or
ii) the CDR according to e) and/or f) preferably only contains amino acid substitutions, and no amino acid deletions or insertions, compared to the corresponding CDR according to d);
   and/or
iii) the CDR according to e) and/or f) may be a CDR that is derived from a CDR according to d) by means of affinity maturation using one or more techniques of affinity maturation known per se.

Also, similarly, when a Nanobody of the invention contains one or more CDR3 sequences according to h) and/or i):
i) any amino acid substitution in such a CDR according to h) and/or i) is preferably, and compared to the corresponding CDR according to g), a conservative amino acid substitution (as defined herein);
   and/or
ii) the CDR according to h) and/or i) preferably only contains amino acid substitutions, and no amino acid deletions or insertions, compared to the corresponding CDR according to g);
   and/or
iii) the CDR according to h) and/or i) may be a CDR that is derived from a CDR according to g) by means of affinity maturation using one or more techniques of affinity maturation known per se.

It should be understood that the last three paragraphs generally apply to any Nanobody of the invention that comprises one or more CDR1 sequences, CDR2 sequences and/or CDR3 sequences according to b), c), e), h) or i), respectively.

Of the Nanobodies of the invention, Nanobodies comprising one or more of the CDR□s explicitly listed above are particularly preferred; Nanobodies comprising two or more of the CDR□s explicitly listed above are more particularly preferred; and Nanobodies comprising three of the CDR□s explicitly listed above [symbol] particularly preferred. Some particularly preferred, but non-limiting combinations of CDR sequences, as well as preferred combinations of CDR sequences and framework sequences, are mentioned in Table A-1 below, which lists the CDR sequences and framework sequences that are present in a number of preferred (but non-limiting) Nanobodies of the invention. As will be clear to the skilled person, a combination of CDR1, CDR2 and CDR3 sequences that occur in the same clone (i.e. CDR1, CDR2 and CDR3 sequences that are mentioned on the same line in Table A-1) will usually be preferred (although the invention in its broadest sense is not limited thereto, and also comprises other suitable combinations of the CDR sequences mentioned in Table A-1). Also, a combination of CDR sequences and framework sequences that occur in the same clone (i.e. CDR sequences and framework sequences that are mentioned on the same line in Table A-1) will usually be preferred (although the invention in its broadest sense is not limited thereto, and also comprises other suitable combinations of the CDR sequences and framework sequences mentioned in Table A-1, as well as combinations of such CDR sequences and other suitable framework sequences, e.g. as further described herein). Also, in the Nanobodies of the invention that comprise the combinations of CDR□s mentioned in Table A-1, each CDR can be replaced by a CDR chosen from the group consisting of amino acid sequences that have at least 80%, preferably at least 90%, more preferably at least 95%, even more preferably at least 99% sequence identity (as defined herein) with the mentioned CDR□s; in which:
i) any amino acid substitution in such a CDR is preferably, and compared to the corresponding CDR sequence mentioned in Table A-1, a conservative amino acid substitution (as defined herein);
   and/or
ii) any such CDR sequence preferably only contains amino acid substitutions, and no amino acid deletions or insertions, compared to the corresponding CDR sequence mentioned in Table A-1;
   and/or
iii) any such CDR sequence is a CDR that is derived by means of a technique for affinity maturation known per se, and in particular starting from the corresponding CDR sequence mentioned in Table A-1.

However, as will be clear to the skilled person, the (combinations of) CDR sequences, as well as (the combinations of) CDR sequences and framework sequences mentioned in Table A-1 will generally be preferred.

TABLE A-1
Preferred combinations of CDR sequences, preferred combinations of framework sequences,
and preferred combinations of framework and CDR sequences.

| ID | FR1 | ID | CDR1 | ID | FR2 | ID | CDR2 | ID | FR3 | ID | CDR3 | ID | FR4 |
|---|---|---|---|---|---|---|---|---|---|---|---|---|---|
| 126 | EVQLVES | 296 | YYNIG | 466 | WFRQAPGE | 636 | CVSSSG | 806 | RFTISRDNAKNTV | 976 | LNLFTTCDGPWGYEY | 1146 | YGQGTQ |
| | GGGLVQP | | | | EREGVS | | GSTYYA | | YLQMNSLKPEDTA | | DY | | VTVSS |
| | GGSLRLS | | | | | | DSVKG | | VYYCAT | | | | |
| | CAASGFT | | | | | | | | | | | | |
| | LD | | | | | | | | | | | | |
| 127 | EVQLVES | 297 | YYNIG | 467 | WFRQAPGK | 637 | CVSSSG | 807 | RFTISRDNAKNTV | 977 | LNLFTTCDGPWGYEY | 1147 | YGQGTQ |
| | GGGLVQP | | | | EREGVS | | GSTYYA | | YLQMNSLKPEDTA | | DY | | VTVSS |
| | GGSLRLS | | | | | | DSVKG | | VYYCAT | | | | |
| | CAASGFT | | | | | | | | | | | | |
| | LD | | | | | | | | | | | | |
| 128 | XVQLVES | 298 | YYAIG | 468 | WFRQAPGK | 638 | CISSSDG | 808 | RFTISRDNAKNTV | 978 | LNLFTTCDGPWGYEY | 1148 | YGQGTQ |
| | GGGLVQP | | | | EREGVS | | STYYAD | | YLQMNSLKPEDTA | | DY | | VTVSS |
| | GGSLRLS | | | | | | SVKG | | VYYCAT | | | | |
| | CAASGFT | | | | | | | | | | | | |
| | LD | | | | | | | | | | | | |
| 129 | EVQLVES | 299 | YYNIG | 469 | WFRQAPGK | 639 | CVSDSG | 809 | RFTISRDNAKNTV | 979 | LNLFTTCDGPWGYEY | 1149 | YGQGTQ |
| | GGGLVQP | | | | EREGVS | | GSTYYA | | YLQMNSLKPEDTA | | DY | | VTVSS |
| | GGSLRLS | | | | | | DSVKG | | VYYCAT | | | | |
| | CAASGFT | | | | | | | | | | | | |
| | LD | | | | | | | | | | | | |
| 130 | EVQLVES | 300 | SYNIG | 470 | WFRQAPGE | 640 | CVSSSG | 810 | RFTISRDNAKNTV | 980 | LNLFTTCDGPWGYEY | 1150 | YGQGTQ |
| | GGGLVQP | | | | EREGVS | | GSTYYA | | YLQMNSLKPEDTA | | DY | | VTVSS |
| | GGSLRLS | | | | | | DSVKG | | VYYCAT | | | | |
| | CAASGFT | | | | | | | | | | | | |
| | LD | | | | | | | | | | | | |
| 131 | EVQLVES | 301 | DYAIG | 471 | WFRQAPGK | 641 | CISSSDG | 811 | RFTISSDNAKNTV | 981 | DPRFEPTLLCTDYDYE | 1151 | WGQGT |
| | GGGLVQA | | | | EREGVS | | STFYAD | | YLQMNSLKPEDTA | | DY | | QVTVSS |
| | GGSLRLS | | | | | | SVKG | | VYYCAA | | | | |
| | CAASGFT | | | | | | | | | | | | |
| | AD | | | | | | | | | | | | |
| 132 | EVQLVES | 302 | DYAIG | 472 | WFRQAPGK | 642 | CISSSDG | 812 | RFTVSSDNAKNTV | 982 | BPXLSPQTYCTDYDY | 1152 | WGQGT |
| | GGGLVQP | | | | EREGVS | | STFYAN | | YLQMNSLKPEDTA | | SY | | QVTVSS |
| | GGSLRLS | | | | | | SVKD | | VYYCAA | | | | |
| | CAASGFT | | | | | | | | | | | | |
| | FD | | | | | | | | | | | | |
| 133 | EVQLVES | 303 | DYAIG | 473 | WFRQAPGK | 643 | CISSSDG | 813 | RFTISSDDAKNTV | 983 | TPRRFGWCSDYBEYD | 1153 | WGQGT |
| | GGGLVQA | | | | EREGVS | | YTYYAD | | YLQMNSLKPEDTA | | Y | | QVTVSS |
| | GGSLRLS | | | | | | SVKD | | VYYCAA | | | | |
| | CAASGFT | | | | | | | | | | | | |
| | FD | | | | | | | | | | | | |
| 134 | EVQLVES | 304 | DYAIG | 474 | WFRQAPGK | 644 | CISSSDG | 814 | RFTISSDNAKNTV | 984 | TPRRFGWCSDYDEYD | 1154 | WGQGT |
| | GGGLVQA | | | | EREGVS | | YSFYAN | | YLQMNSLKPEDTA | | Y | | QVTVSS |
| | GGSLRLS | | | | | | SVKG | | VYYCAA | | | | |
| | CAASGFT | | | | | | | | | | | | |
| | FD | | | | | | | | | | | | |
| 135 | EVQLVES | 305 | DYAIG | 475 | WFRQAPGE | 645 | CISSSDG | 815 | RFTISSDNAKNTM | 985 | TPRRFGWXSDYDXYD | 1155 | WGQGT |
| | GGGLVXA | | | | EREGVS | | YTYYAH | | YLQMNSLKPEDTA | | Y | | QVTVSS |
| | GGSLRLS | | | | | | SVKD | | VYYCAA | | | | |
| | CAXSGFX | | | | | | | | | | | | |
| | FD | | | | | | | | | | | | |
| 136 | XVQLVES | 306 | DYVIG | 476 | WFXQAPGK | 646 | CISSSEG | 816 | RFTISXDNAKNTV | 986 | XRKIIGLWCSDYDNY | 1156 | WGQGT |
| | GGGLVQA | | | | EREGVS | | YTFYAD | | SLQMNSLKPEDTA | | DY | | QVTVSS |
| | GGSLRLS | | | | | | SVKD | | VYYCAA | | | | |
| | CAASGFT | | | | | | | | | | | | |
| | FD | | | | | | | | | | | | |
| 137 | EVQLVES | 307 | IASMG | 477 | WYRQAPGK | 647 | RIRSDGT | 817 | RFTIAKDNAKNAG | 987 | AGSTIDGGFSS | 1157 | WGPGTQ |
| | GGGLVRA | | | | QRTWVA | | TSYQDA | | YLQMDNLKPEDT | | | | VTVSS |
| | GGSLRLS | | | | | | VKG | | AVYYCNA | | | | |
| | CTASGNIL | | | | | | | | | | | | |
| | N | | | | | | | | | | | | |
| 138 | EVQLVES | 308 | IASMG | 478 | WYRQALGK | 648 | RIRSDGT | 818 | RFTIAKDNAKNAG | 988 | AGSTIDGGFSS | 1158 | WGPGTQ |
| | GGGLVRA | | | | QRTWVA | | TSYQDA | | YLQMDNLKPEDT | | | | VTVSS |
| | GGSLRLS | | | | | | VKG | | AVYYCNA | | | | |
| | CTASGNIL | | | | | | | | | | | | |
| | N | | | | | | | | | | | | |
| 139 | EVQLVES | 309 | IVNAG | 479 | WYRQGPEK | 649 | RITSGGT | 819 | RFTISRDNAKNTV | 989 | RVIAPGRLDDI | 1159 | WGQGT |
| | GGGLVQA | | | | QRELVA | | TNYAES | | YLQMNSLKPEDTA | | | | QVTVSS |
| | GGSLRLS | | | | | | VKG | | VYSCNA | | | | |
| | CAASGSG | | | | | | | | | | | | |
| | FN | | | | | | | | | | | | |
| 140 | EVxLVESG | 310 | IVNAG | 480 | WYRQGPGK | 650 | RITSGGT | 820 | RFTISRDNAKNTV | 990 | RVIAPGRLDDI | 1160 | WGQGT |
| | GGLVQAG | | | | QREFVA | | TNYAES | | YLQMNSLKPEDTA | | | | QVTVSS |
| | GSLRLSC | | | | | | VKG | | VYSCNA | | | | |
| | AASGSGF | | | | | | | | | | | | |
| | N | | | | | | | | | | | | |
| 141 | EVQLVES | 311 | SYAMS | 481 | WVRQAPGK | 651 | AINSGG | 821 | RFTISRDDAKNTL | 991 | PIYYSPNTYPPTSRYD | 1161 | RGQGTQ |
| | GGGLVQP | | | | GVEWVS | | GSTSYL | | YLQMNSLKPEDTA | | Y | | VTVSS |
| | GGSLRLS | | | | | | NSVKG | | VYYCAK | | | | |
| | CAASGFA | | | | | | | | | | | | |
| | FS | | | | | | | | | | | | |
| 142 | KVQLVES | 312 | SYVMT | 482 | WVRQAPGK | 652 | SITSGGG | 822 | RFTISRDDAKNTL | 992 | PTFYSPNMYPPTSRYD | 1162 | RGQGTQ |
| | GGGLVQP | | | | GLEWVS | | YTSYLN | | YLQMNSLKPEDTA | | Y | | VTVSS |
| | GGSLRLS | | | | | | SVKG | | VYYCAK | | | | |
| | CAASGFA | | | | | | | | | | | | |
| | FS | | | | | | | | | | | | |
| 143 | EVQLVES | 313 | SYVMT | 483 | WVRQAPGK | 653 | SITSGGG | 823 | RFTISRDDAKNTL | 993 | PTFYSPNMYPPTSRYD | 1163 | RGQGTQ |
| | GGGLVQP | | | | GLEWVS | | YTSYVN | | YLQMNSLKPEDTA | | Y | | VTVSS |
| | GGSLRLS | | | | | | SVKG | | VYYCAK | | | | |
| | CAASGFA | | | | | | | | | | | | |
| | FS | | | | | | | | | | | | |
| 144 | EVQLVES | 314 | AYAIG | 484 | WFRQAPGK | 654 | CISSSDG | 824 | RFTISSDNAKNTV | 994 | DRRGKSEMYCTDYSY | 1164 | WGQGT |
| | GGGLVQA | | | | EREGVS | | STFYAD | | YLQMNSLKPEDTA | | SDA | | QVTVSS |
| | GGSLRLS | | | | | | SVKD | | VYYCAA | | | | |
| | CAASGFT | | | | | | | | | | | | |
| | LD | | | | | | | | | | | | |
| 145 | EVXLVESG | 315 | AYAIG | 485 | WFRQAPGK | 655 | CISSSDG | 825 | RFTISSDNAKNTV | 995 | DRRGKSEMYCTDYA | 1165 | WGQGT |
| | GGLVQAG | | | | EREGVS | | SRFYAD | | YLQMNSLKPEDTA | | YSDA | | QVTVSS |
| | GSLRLSC | | | | | | SVKD | | VYYCAA | | | | |
| | AASGFTL | | | | | | | | | | | | |
| | D | | | | | | | | | | | | |
| 146 | EVQLVES | 316 | IANSA | 486 | WYRQAPGN | 656 | RITSNDN | 826 | RLTISKDNAKNTA | 996 | RTVGTGSLFDY | 1166 | WGQGT |
| | GGGLVQA | | | | QRELVA | | TYYADS | | SLQMNSLKPEDTA | | | | QVTVSS |
| | GGSLTLSC | | | | | | VKG | | VYYCFV | | | | |
| | ALSGGSSS | | | | | | | | | | | | |
| 147 | EVQLVES | 317 | IANSA | 487 | WYRQAPGN | 657 | RITSNDN | 827 | RFTISKDNAKNTA | 997 | RTVGTGSLFDY | 1167 | WGQGT |
| | GGGLVQA | | | | QRELVA | | TYYADS | | SLQMNSLKPEDTA | | | | QVTVSS |
| | GGSLTLSC | | | | | | VKG | | VYYCFV | | | | |
| | ALSGGSSS | | | | | | | | | | | | |
| 148 | EVQPVES | 318 | IANSA | 488 | WYRQAPGN | 658 | RITSNDN | 828 | RFTISKDNAKNTA | 998 | RTVGTGSLFDY | 1168 | WGQGT |
| | GGGLVQA | | | | QRELVA | | TYYADS | | SLQMNSLKPEDTA | | | | QVTVSS |
| | GGSLTLSC | | | | | | VKG | | VYYCFV | | | | |
| | ALSGGSSS | | | | | | | | | | | | |
| 149 | EVQLVES | 319 | DYAIG | 489 | WFRQAPGK | 659 | CISSSHG | 829 | RFTISSDNAKNTV | 999 | ALGGGSSWCTTYEYD | 1169 | WGQGT |
| | GGGLVQA | | | | EREGVS | | STFYAD | | YLQMNSLKPEDTA | | A | | QVTVSS |
| | GGSLRLS | | | | | | SVKD | | VYYCAA | | | | |
| | CAASGFT | | | | | | | | | | | | |
| | FD | | | | | | | | | | | | |
| 150 | EVQLVES | 320 | ISDMR | 490 | WYRQAPGN | 660 | SISRGGS | 830 | RFTISRDNAKNTV | 1000 | AVAAGTVGAYTLRN | 1170 | WGQGT |
| | GGGLVQS | | | | QRELAA | | TNYGDF | | SLQMSSLEPEDTA | | Y | | QVTVSS |
| | GGSLRLS | | | | | | VKG | | VYYCNA | | | | |
| | CAASGSIV | | | | | | | | | | | | |
| | G | | | | | | | | | | | | |
| 151 | KVQLVES | 321 | VSTMT | 491 | WYRQVLGT | 661 | SITRSGG | 831 | RFTIARDNAKNTM | 1001 | VIASNSGRSYDLRNY | 1171 | WGQGT |
| | GGGLVQA | | | | QRELVA | | SNYADP | | YLQMNSLKPEDTA | | | | QVTVSS |
| | GGSLRLT | | | | | | VKG | | IYYCNA | | | | |
| | CAASGSIL | | | | | | | | | | | | |
| | S | | | | | | | | | | | | |
| 152 | EVQLVES | 322 | IGNMG | 492 | WYRQAPGK | 662 | TITQAGS | 832 | RFTISRDVPKNTVS | 1002 | NIVTYDRGRTTVKNY | 1172 | WGQGT |
| | GGGLVQP | | | | QRELVA | | PNYSQS | | LQMNSLKPEDTAV | | | | QVTVSS |
| | GGSLRLS | | | | | | AKG | | YYCNG | | | | |
| | CTASGSV | | | | | | | | | | | | |
| | FS | | | | | | | | | | | | |
| 153 | EVQLVES | 323 | FIEMG | 493 | WYRQAPGK | 663 | HITSGGS | 833 | RFTISRDLSLQMN | 1003 | KGTDRRSY | 1173 | WGQGT |
| | GGGLVQP | | | | QRELVA | | TKYADS | | NLNPEDSAVYLCN | | | | QVTVSS |
| | GGSLRLY | | | | | | VKG | | M | | | | |
| | CAAPGTM | | | | | | | | | | | | |
| | YS | | | | | | | | | | | | |
| 154 | XVQLVES | 324 | DYAIG | 494 | WFRQAPGK | 664 | CISRSAG | 834 | RFTISSDNAKNTVS | 1004 | YRAIGHFCTDYXDFV | 1174 | WGQGT |
| | GGGLVQA | | | | EREGVS | | SKYYAD | | LQMNSLKPEDTAV | | S | | QVTVSS |
| | GGSLRLS | | | | | | SVKD | | YYCAA | | | | |
| | CAASGFT | | | | | | | | | | | | |
| | FD | | | | | | | | | | | | |
| 155 | EVQLVES | 325 | INAMG | 495 | WYRQAPGK | 665 | IIYSSGRI | 835 | RFTISRDNAKNTV | 1005 | ANPNTGWQRPHRAS | 1175 | WGQGT |
| | GGGLVQA | | | | QRELVA | | DYADSV | | YLQMNNLQPDDT | | | | QVTVSS |
| | GGSLRLS | | | | | | KG | | AAYYCNA | | | | |
| | CTVSGST | | | | | | | | | | | | |
| | GS | | | | | | | | | | | | |
| 156 | EVQLVES | 326 | INAMG | 496 | WYRQAPGK | 666 | IIYSSGTI | 836 | RFTISRDNAKNTV | 1006 | ANPNTGWQRPHRAS | 1176 | WGQGT |
| | GGGVVQA | | | | QRELVA | | BYADSV | | YLQMNSLKPDDT | | | | QVTVSS |
| | GGSLRLS | | | | | | KG | | AAYYCNA | | | | |
| | CTVSGST | | | | | | | | | | | | |
| | GS | | | | | | | | | | | | |
| 157 | EVQLVES | 327 | INAMG | 497 | WYRQAPGK | 667 | IIYSSGRI | 837 | RFTISRDNAKNTV | 1007 | ANPNTGWQRPHRAS | 1177 | WGQGT |
| | GRGLVQA | | | | QRELVA | | DYADSV | | BLQMNSLKPDDTA | | | | QVTVSS |
| | GGSLRLS | | | | | | KG | | AYYCNA | | | | |
| | CTVSGST | | | | | | | | | | | | |
| | GS | | | | | | | | | | | | |
| 158 | EVQLVES | 328 | INVMG | 498 | WYRQAPGK | 668 | IIYSSGT | 838 | RFTISRDNAKNTV | 1008 | AMQDSAWLRPHRAS | 1178 | WGQGT |
| | GGGLVQA | | | | QRELVA | | LDYADS | | YLQMNSLKPDDT | | | | QVTVSS |
| | GGSLRLS | | | | | | VKG | | AAYYCNA | | | | |
| | CTVSGST | | | | | | | | | | | | |
| | GS | | | | | | | | | | | | |
| 159 | EVQLVES | 329 | INAMG | 499 | WYRQAPGR | 669 | IIYSSGRI | 839 | RFTISRDNAKNTV | 1009 | ANPNTGWQRPHRAS | 1179 | WGQGT |
| | GGGLVQA | | | | *RELVA | | DYADSV | | DLQMNSLKPDDT | | | | QVTVSS |
| | GGSLRLS | | | | | | KG | | AAYYCNA | | | | |
| | CTVSGST | | | | | | | | | | | | |
| | GS | | | | | | | | | | | | |
| 160 | EVQLVES | 330 | INAMG | 500 | WYRQAPGK | 670 | IIYSSGRI | 840 | RFTISRDNAKNTV | 1010 | ANPNTGWQRPHRAS | 1180 | WGQGT |
| | GGGLVQA | | | | QRELVA | | DYADSV | | YLQMNNLQPDDT | | | | QVTVSS |
| | GGSLRLS | | | | | | KG | | AAYYCNA | | | | |
| | CTVSGST | | | | | | | | | | | | |
| | GS | | | | | | | | | | | | |
| 161 | EVQLVES | 331 | INVMG | 501 | WYRQAPGK | 671 | TISSSGY | 841 | RFTISRDNKNTVH | 1011 | STLRTGWFTG | 1181 | WGQGT |
| | GGELVQA | | | | QRELVA | | TDYSDS | | LQMNSLKPEDTAV | | | | QVTVSS |
| | GGSLRLS | | | | | | AKG | | YYCRA | | | | |
| | CAASGSV | | | | | | | | | | | | |
| | SS | | | | | | | | | | | | |
| 162 | EMQLVES | 332 | INVMG | 502 | WYRQAPGK | 672 | TISSSGY | 842 | RFTISRDNKNTVH | 1012 | STLRTGWFTG | 1182 | WGQGT |
| | GGELVQA | | | | QRELVA | | TDYSDS | | LQMNSLKPEDTAV | | | | QVTVSS |
| | GGSLRLS | | | | | | AKG | | YYCRA | | | | |
| | CAASGSV | | | | | | | | | | | | |
| | SS | | | | | | | | | | | | |
| 163 | EVQLVES | 333 | INVMG | 503 | WYRQAPGK | 673 | TISSSGC | 843 | RFTISRDNKNTVH | 1013 | STLRTGWFTG | 1183 | WGQGT |
| | GGELVQA | | | | QRELVA | | TDYSDS | | LQMNSLKPEDTAV | | | | QVTVSS |
| | GGSLRLS | | | | | | AKG | | YYCRA | | | | |
| | CAASGSV | | | | | | | | | | | | |
| | SS | | | | | | | | | | | | |
| 164 | AEVQLVE | 334 | INVMG | 504 | WYRQAPGK | 674 | TISSSGY | 844 | RFTISRDNKNTVH | 1014 | STLRTGWFTG | 1184 | WGQGT |
| | SGGELVQ | | | | QRELVA | | TDYADS | | LQMNSLKPEDTAV | | | | QVTVSS |
| | AGGSLRL | | | | | | AKG | | YYCRA | | | | |
| | SCAAPGS | | | | | | | | | | | | |
| | VSS | | | | | | | | | | | | |
| 165 | EVQLVES | 335 | INVMA | 505 | WYRQAPGK | 675 | TISSSGY | 845 | RFTISRDDXNTVH | 1015 | STARTGWLRA | 1185 | WGQGT |
| | GGGLVQA | | | | QRELVA | | TDYSDS | | LQMNSLKPEDTAV | | | | QVTVSS |
| | GRSLRLSC | | | | | | AKG | | YYCRA | | | | |
| | AASGSVS | | | | | | | | | | | | |
| | S | | | | | | | | | | | | |
| 166 | EVQLVES | 336 | INVMG | 506 | WYRQAPGK | 676 | TISSSGY | 846 | RFTISRDNENTVHL | 1016 | STARTGWLXP | 1186 | WGQGT |
| | GGGLVQA | | | | QRELVA | | TDYSDS | | QMNSLKPEDTGV | | | | QVTVSS |
| | GGSLRLS | | | | | | AKG | | YYCRA | | | | |
| | CAASGSV | | | | | | | | | | | | |
| | SS | | | | | | | | | | | | |
| 167 | EVQLVES | 337 | INVMA | 507 | WYRQAPGK | 677 | TISTTGY | 847 | RFTISRDSKNTVHL | 1017 | STLRTGWLMG | 1187 | WGQGT |
| | GGGLVQA | | | | ERELVA | | TDYSXS | | QMNSLKPEDTAV | | | | QVTVSS |
| | GGSLRLS | | | | | | AKG | | YYCRA | | | | |
| | CAASGSV | | | | | | | | | | | | |
| | SS | | | | | | | | | | | | |
| 168 | XVQLVES | 338 | INVMG | 508 | WYRQAPGK | 678 | TISTTGY | 848 | RFTISRDNKNTVH | 1018 | STLRTGWLKG | 1188 | WGQGT |
| | GGGLVQA | | | | QRELVA | | TDYSXS | | LQMNSLKPEDTAV | | | | QVTVSS |
| | GGSLRLS | | | | | | AKG | | YYCRA | | | | |
| | CAAXGSV | | | | | | | | | | | | |
| | SS | | | | | | | | | | | | |
| 169 | EVQLVES | 339 | INVMA | 509 | WYRQAPGK | 679 | TISSTGY | 849 | RFTISRDNKNTVH | 1019 | STLRTGWFMG | 1189 | WGQGT |
| | GGGLVQA | | | | QRELVA | | TDYSDS | | LQMNSLKPEDTAV | | | | QVTVSS |
| | GGSLRLS | | | | | | AKG | | YYCRA | | | | |
| | CAASGSV | | | | | | | | | | | | |
| | SS | | | | | | | | | | | | |
| 170 | EVQLVES | 340 | INVMA | 510 | WYRQAPGK | 680 | TISTSGY | 850 | RFTISRDNKNTVH | 1020 | STLRTGWLMG | 1190 | WGQGT |
| | GGGLVQA | | | | QRELVA | | TDYSDS | | LQMNSLKPEDTAV | | | | QVTVSS |
| | GGSLRLS | | | | | | AKG | | YYCRA | | | | |
| | CATSGSV | | | | | | | | | | | | |
| | SS | | | | | | | | | | | | |
| 171 | EVQLVES | 341 | INVMA | 511 | WYRQAPGK | 681 | TISTSGY | 851 | RFTISRDNKNTVH | 1021 | STLRTGWLMG | 1191 | WGQGT |
| | GGGLVQA | | | | QRELVA | | TDYSDS | | LQMNSLKPEDTAV | | | | QVTVSS |
| | GGSLRLS | | | | | | AKG | | YYCRA | | | | |
| | CAASGSV | | | | | | | | | | | | |
| | SS | | | | | | | | | | | | |
| 172 | EVQLVES | 342 | INVMA | 512 | WYRQAPGK | 682 | TISSTGY | 852 | RFTISRDNKNTVY | 1022 | STLRTGWLMG | 1192 | WGQGT |
| | GGGLVQA | | | | QRELVA | | TDYSDS | | LQMNSLKPEDTAI | | | | QVTVSS |
| | GGSLRLS | | | | | | AKG | | YYCRA | | | | |
| | CAASGSV | | | | | | | | | | | | |
| | SA | | | | | | | | | | | | |
| 173 | EVxLVESG | 343 | INLMG | 513 | WYRQAPGK | 683 | TISSTAY | 853 | RFTISRDNKNTVH | 1023 | STLRTGWLPG | 1193 | WGQGT |
| | GGLVQAG | | | | QRELVA | | TDYSDS | | LQMNSLKPEDTAV | | | | QVTVSS |
| | GSLRLSC | | | | | | AKG | | YYCRA | | | | |
| | AASGSISS | | | | | | | | | | | | |
| 174 | EV*LVESG | 344 | INVMA | 514 | WYRQAPGK | 684 | TISSSGY | 854 | RFTISRDDENTVHL | 1024 | STARTGWLRA | 1194 | WGQGT |
| | GGLVQAG | | | | QRELVA | | TDYSDS | | QMNSLKPEDTAV | | | | QVTVSS |
| | RSLRLSCA | | | | | | AKG | | YYCRA | | | | |
| | ASGSVSS | | | | | | | | | | | | |
| 175 | XVQLVES | 345 | INVMA | 515 | WYRQAPGK | 685 | TVSTTG | 855 | RFTISRDNKNTVY | 1025 | STLRTGWLMG | 1195 | WGQGT |
| | GGGLVQA | | | | QRELVA | | YTDYSD | | LQMNSLKPEDTAI | | | | QVTVSS |
| | GGSLRLS | | | | | | SAKG | | YYCRA | | | | |
| | CAASGSV | | | | | | | | | | | | |
| | SA | | | | | | | | | | | | |
| 176 | EVQLVES | 346 | FELMG | 516 | WYRQAPGK | 686 | LITSSGS | 856 | RFTISRDVAKNTL | 1026 | HTYTDNL | 1196 | WGQGT |
| | GGGLVQA | | | | PRDLVA | | TNYADS | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 177 | EVQLVES | 347 | FELMG | 517 | WYRQAPGK | 687 | LITSSGS | 857 | RFTISRDNAKNTL | 1027 | HTYTDNL | 1197 | WGQGT |
| | GGGLVQA | | | | PRDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | LR | | | | | | | | | | | | |
| 178 | XVQLVES | 348 | FELMG | 518 | WYRQAPGK | 688 | LITSSGS | 858 | RFTISRDNAKNTL | 1028 | HTYTDNL | 1198 | WGQGT |
| | GGGLVQA | | | | PRDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | LR | | | | | | | | | | | | |
| 179 | EVQLVES | 349 | FEIMG | 519 | WYRQAPGK | 689 | LITRSGS | 859 | RFTISRDSAKNTLY | 1029 | HTYTDNL | 1199 | WGQGT |
| | GGGVVEA | | | | PRDLVA | | ANYADS | | LQMNSLKPEDTGV | | | | QVTVSS |
| | GGSLRLS | | | | | | VKG | | YYCNA | | | | |
| | CAATGSR | | | | | | | | | | | | |
| | FR | | | | | | | | | | | | |
| 180 | EVQLVES | 350 | FEIMG | 520 | WYRQAPGK | 690 | LITSSGS | 860 | RFTISRDNAKNTL | 1030 | HTYTDNL | 1200 | WGQGT |
| | GGGLVQA | | | | PRDLVA | | ANYADS | | YLQMNSLKSEDTG | | | | QVTVSS |
| | GGSLRLS | | | | | | VKG | | VYYCNA | | | | |
| | CAASGSR | | | | | | | | | | | | |
| | LAASG | | | | | | | | | | | | |
| | FR | | | | | | | | | | | | |
| 181 | EMQLVES | 351 | FELMG | 521 | WYRQAPGK | 691 | LITSSGS | 861 | RFTISRDNAKNTL | 1031 | HTYTDNL | 1201 | WGQGT |
| | GGGLVQA | | | | PRDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | LR | | | | | | | | | | | | |
| 182 | XVQLVES | 352 | FEIMG | 522 | WYRQAPGK | 692 | LITNSGS | 862 | RFTISRDNAKNTL | 1032 | HTYTDSL | 1202 | WGQGT |
| | GGGLVQA | | | | PRDLVA | | ANYABS | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 183 | KVQLVES | 353 | FELMG | 523 | WYRQAPGK | 693 | LITSSGS | 863 | RFTISRDNAKNTL | 1033 | HTYTDNL | 1203 | WGQGT |
| | GGGLVQA | | | | PRDLVA | | ANYAES | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 184 | EVQLVES | 354 | FELMG | 524 | WYRQAPGK | 694 | LITSSGS | 864 | RFTISRDNAKNTL | 1034 | HTYTDNL | 1204 | WGQGT |
| | GGGLVQA | | | | PRDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 185 | EVQLVES | 355 | FELMG | 525 | WYRQAPGK | 695 | LITSSGS | 865 | RFTISRDNAKNTL | 1035 | HTYTDNL | 1205 | WGQGT |
| | GGGLVQV | | | | PRDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 186 | EVQLVES | 356 | FELMG | 526 | WYRQAPGK | 696 | LITSSGS | 866 | RFTISRDNAKNTL | 1036 | HTYTDNL | 1206 | WGQGT |
| | GGGLVQA | | | | PRDLVA | | ANYADS | | YLQMNSLEPEDTG | | | | QVTVSS |
| | GGSLRLS | | | | | | VKG | | VYYCNA | | | | |
| | CAASGSR | | | | | | | | | | | | |
| | LR | | | | | | | | | | | | |
| 187 | EVQLVES | 357 | FELMG | 527 | WYRQAPGK | 697 | LITSSGS | 867 | RFTISRDNAKNTL | 1037 | HTYTDNL | 1207 | WGQGT |
| | RGGLVQA | | | | PRDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | LR | | | | | | | | | | | | |
| 188 | EVQLVES | 358 | FELMG | 528 | WYRQAPGK | 698 | LITRSGS | 868 | RFTISRDNAKNTL | 1038 | HTYTDNL | 1208 | WGQGT |
| | GGGLVQA | | | | PRDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 189 | EVQLVES | 359 | FEIMG | 529 | WYRQAPGS | 699 | LITNSGS | 869 | RFTISRDNAKNTF | 1039 | HTYTDNL | 1209 | WGQGT |
| | GGGLVRA | | | | ARDLVA | | ANYADS | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 190 | EVQLVES | 360 | FELMG | 530 | WYRXAPGK | 700 | LITXSGS | 870 | RFTISRXNAXNTL | 1040 | HTYTDNL | 1210 | WGQGT |
| | GGXLVQA | | | | PRDLVA | | ANYADS | | YLQMNSLKPEDTG | | | | QVTVSS |
| | EGSLRLSX | | | | | | VKG | | VYYCNX | | | | |
| | AASGSRF | | | | | | | | | | | | |
| | R | | | | | | | | | | | | |
| 191 | EVQLVES | 361 | FEIMG | 531 | WYRQAPGR | 701 | LITSSGS | 871 | RFTISRDNAKNTL | 1041 | HTYTDNL | 1211 | WGQGT |
| | GGGLVQA | | | | MRDLVA | | TNYADS | | YLQMNSLKPEDTG | | | | QVTVSS |
| | GGSLRLS | | | | | | VKG | | VYYCNA | | | | |
| | CAASGSR | | | | | | | | | | | | |
| | FR | | | | | | | | | | | | |
| 192 | EVQLVES | 362 | FVLMG | 532 | WYRQAPGK | 702 | QITSSDA | 872 | RFTISRDNAKKTV | 1042 | ARGPDVY | 1212 | WGQGT |
| | GGGLVQA | | | | QRELVA | | TNYADS | | DLQMNSLKPEDTA | | | | QVTVSS |
| | GGSVRLS | | | | | | VKG | | VYYCLL | | | | |
| | CAASGVIF | | | | | | | | | | | | |
| | R | | | | | | | | | | | | |
| 193 | KV*LVES | 363 | FVLMG | 533 | WYRXAPGK | 703 | QITSSDA | 873 | RFTISRDNAKKTV | 1043 | ARGPDVY | 1213 | WGQGT |
| | GGGLVQA | | | | QRELVA | | TNYADS | | DLQMNSLKPEDTA | | | | QVTVSS |
| | GGSVRLS | | | | | | VKG | | VYYCLL | | | | |
| | CAASGVIF | | | | | | | | | | | | |
| | R | | | | | | | | | | | | |
| 194 | EVQLVES | 364 | IYTMG | 534 | WYRQALGK | 704 | SISRDGS | 874 | RFTISRDNAKNTA | 1044 | EGWYQGY | 1214 | WGQGT |
| | GGGLVQA | | | | KRVFVA | | TIYGDS | | YLQMNSLKPEDTA | | | | QVTVSS |
| | GGSQKLS | | | | | | VKG | | VYVCKA | | | | |
| | CAASGST | | | | | | | | | | | | |
| | L | | | | | | | | | | | | |
| 195 | EVQLVES | 365 | IYTMG | 535 | WYRQAPGK | 705 | SISRDGS | 875 | RFTISRDSAKNTA | 1045 | XGWYNGY | 1215 | WGQGT |
| | GGGLVQA | | | | QRVFVA | | TIYGDS | | YLQMNRLKPEDT | | | | QVTVSS |
| | GGSLRLS | | | | | | VKG | | AVYVCKA | | | | |
| | CAASGST | | | | | | | | | | | | |
| | E | | | | | | | | | | | | |
| 196 | EVQLVES | 366 | IYTMG | 536 | WYRQALGK | 706 | SISRDGS | 876 | RFTISRDNAKNTA | 1046 | EGWYQGY | 1216 | WGQGT |
| | GGGLVQA | | | | KRVFVA | | TIYGDS | | YLQMNNLKPEDT | | | | QVTVSS |
| | GGSQKLS | | | | | | VKG | | AVYVCKA | | | | |
| | CAASGST | | | | | | | | | | | | |
| | L | | | | | | | | | | | | |
| 197 | EVQLVES | 367 | INAVG | 537 | WYRQAPEK | 707 | IILSSGTT | 877 | RFTISRDNAKNIVY | 1047 | ADREMGWAY | 1217 | WGQGT |
| | GGGLVQA | | | | QRELVA | | DYADSV | | LQMNSLKPEDTAV | | | | QVTVSS |
| | GGSLRLS | | | | | | KG | | YYCRV | | | | |
| | CAASGNT | | | | | | | | | | | | |
| | FS | | | | | | | | | | | | |
| 198 | EVQLVES | 368 | INAVG | 538 | WYRQAPEK | 708 | IIXSXGT | 878 | RFTISRDNAKNTV | 1048 | XDRXMGXAY | 1218 | WGQGT |
| | GGGLVQA | | | | QRELVA | | TDYADS | | YLQMNSLKPEDTA | | | | QVTVSS |
| | GGSLRLS | | | | | | VKG | | VYYCXX | | | | |
| | CAASGNT | | | | | | | | | | | | |
| | FS | | | | | | | | | | | | |
| 199 | EVQLVES | 369 | ILAMG | 539 | WYRQAPGK | 709 | TIIRDGK | 879 | RFTISRDNAKNTA | 1049 | KVIVMGAGMDDNDF | 1219 | FGQGTQ |
| | GGGLVQT | | | | QRELVT | | TYYSDS | | YLQMNSLKPEDTA | | | | VTVSS |
| | GGSLRLS | | | | | | VKD | | VYYCYT | | | | |
| | CAASGSIF | | | | | | | | | | | | |
| | M | | | | | | | | | | | | |
| 200 | EVQLVES | 370 | RTDVD | 540 | WYRQAPGK | 710 | IIAPFGT | 880 | RFTISRDNAKNIVY | 1050 | YWGGNVY | 1220 | WGQGT |
| | GGGLVQP | | | | GREWVA | | TNSRDS | | LQMNSLEPEDTAV | | | | QVTVSS |
| | GGSLRLS | | | | | | | | YYCRI | | | | |
| | CAASGSIL | | | | | | | | | | | | |
| | S | | | | | | | | | | | | |
| 201 | EVQLVES | 371 | IVHIQ | 541 | WHRQAPGK | 711 | SVNSRG | 881 | RAIISRDNAKNTV | 1051 | RTLQLGALRDY | 1221 | WGQGT |
| | GGGLVQA | | | | QRELVA | | TTNYAD | | YLQMNSLKPEDTA | | | | QVTVSS |
| | GGSLRLS | | | | | | SVKG | | VYYCYA | | | | |
| | CAPSSSAV | | | | | | | | | | | | |
| | S | | | | | | | | | | | | |
| 202 | EVQLVES | 372 | INTMD | 542 | WFRRTPGK | 712 | TITRDGR | 882 | RFTLSRDNTKNTV | 1052 | NVETTRGLTKNY | 1222 | WGQGIQ |
| | GGGLVQA | | | | QRELVS | | TTYADS | | SLQMNSLKPEDTA | | | | VTVSS |
| | GGSLRLS | | | | | | VKG | | VYYCLA | | | | |
| | CAASASIL | | | | | | | | | | | | |
| | S | | | | | | | | | | | | |
| 203 | EVQLVES | 373 | INAWG | 543 | WYRQAPGK | 713 | VISSSGS | 883 | RFTISTVNAGNTV | 1053 | ADSGPWRY | 1223 | WGQGS |
| | GGGLVQT | | | | QRELVA | | TDYSDA | | YLEMNSLKPEDTA | | | | QVTVSS |
| | GGSLRLS | | | | | | VKD | | VYYCAR | | | | |
| | CAASGST | | | | | | | | | | | | |
| | FN | | | | | | | | | | | | |
| 204 | EVQLVES | 374 | FSSVA | 544 | WYRQAPGK | 714 | QITHEGT | 884 | RFTISREFTLSRDG | 1054 | VQFGRNY | 1224 | WGQGT |
| | GGGKVQA | | | | QRELVA | | RNYADS | | PKEMVHLQMVSL | | | | QVTVSS |
| | GMSQRLS | | | | | | VKG | | | | | | |
| | CAASGGI | | | | | | | | | | | | |
| | GT | | | | | | | | | | | | |
| 205 | EVQLVES | 375 | INYMG | 545 | WYRQAPGK | 715 | IIDRVGA | 885 | RFTISRSNAKNEN | 1055 | VPTTSAY | 1225 | WGPGTQ |
| | GGGLVQA | | | | QREAVA | | STNYVD | | MLYLQMNSLKPE | | | | VTVSS |
| | GGSLRLS | | | | | | SVRG | | DTAVYYCNT | | | | |
| | CAASGSIF | | | | | | | | | | | | |
| | S | | | | | | | | | | | | |
| 206 | XVQLVES | 376 | SNYMG | 546 | WYRQAPGL | 716 | IIDRGGA | 886 | RFTISRSNAKNQS | 1056 | VPTTSAY | 1226 | WGPGTQ |
| | GGGLVQA | | | | QRESVA | | STNYVX | | MLYLQMNSLKPE | | | | VTVSS |
| | GGSLRLS | | | | | | SVRG | | DTAVYYCNT | | | | |
| | CAATGTIF | | | | | | | | | | | | |
| | S | | | | | | | | | | | | |
| 207 | EVQLVES | 377 | ISYMG | 547 | WYRQAPGK | 717 | IIDRSGA | 887 | RFTISRSNAKNKN | 1057 | VPTTSAY | 1227 | WGPGTQ |
| | GGGLVQA | | | | ERESVA | | STNYVD | | MLYLQMNSLKPE | | | | VTVSS |
| | GGSLRLS | | | | | | SVRG | | DTAVYYCNT | | | | |
| | CAASGSIF | | | | | | | | | | | | |
| | S | | | | | | | | | | | | |
| 208 | EVQLVES | 378 | IHTMG | 548 | WYRQAPGK | 718 | VASNSG | 888 | RFTISRDNAKNTV | 1058 | ASQFAS | 1228 | WGQGT |
| | GGGLVQA | | | | QREFVA | | RTNYAD | | YLQMNNLKPEDT | | | | QVTVSS |
| | GGSLKLS | | | | | | SVKG | | AVYYCNS | | | | |
| | CVASGLIL | | | | | | | | | | | | |
| | S | | | | | | | | | | | | |
| 209 | EVQLVES | 379 | IYTMG | 549 | WYRQAPGK | 719 | AATNAG | 889 | RFTISRDNAKNTVI | 1059 | LDYDY | 1229 | WGQGT |
| | GGGLVQA | | | | QREFVA | | TTSYAG | | LQMNNLKPEBTA | | | | QVTVSS |
| | GGSLRLS | | | | | | SVKG | | VYYCRV | | | | |
| | CAASGIIF | | | | | | | | | | | | |
| | S | | | | | | | | | | | | |
| 210 | EVQLVES | 380 | RNVMA | 550 | WYRQAPGK | 720 | IITTAHS | 890 | RFTISRDNAKNTL | 1060 | LPHYPTDS | 1230 | WGQGT |
| | GGGLVQP | | | | EREPVA | | TNYVDS | | YLEMNSLKPEDTG | | | | QVTVSS |
| | GGSLRLS | | | | | | VKG | | VYYCNK | | | | |
| | CAASRIIF | | | | | | | | | | | | |
| | S | | | | | | | | | | | | |
| 211 | EVQLVES | 381 | RNAMA | 551 | WYRQAPGK | 721 | IITSNHG | 891 | RFTISRDNAKNTL | 1061 | IPHYTVDS | 1231 | WGQGT |
| | GGGLVQP | | | | QREPVA | | TNYVDP | | YLQMNSLKPEDTG | | | | QVTVSS |
| | GGSLRLS | | | | | | VKG | | VYYCNK | | | | |
| | CAASRSIF | | | | | | | | | | | | |
| | S | | | | | | | | | | | | |
| 212 | KVQLVES | 382 | RITAFG | 552 | WYRQAPGR | 722 | GIFSGAL | 892 | RFTISRDNAKNTV | 1062 | DNN | 1232 | WGQGT |
| | GGGLVQA | | | | QREFVA | | TNYADS | | FLQMNSLKTEDTG | | | | QVTVSS |
| | GGSLRLS | | | | | | VKG | | TYYCRS | | | | |
| | CTASGNIA | | | | | | | | | | | | |
| 213 | EVQLVES | 383 | ITAFA | 553 | WYRQAPGK | 723 | GIFSGAI | 893 | RFTISRDNAKNTV | 1063 | DNN | 1233 | WGQGT |
| | GGGLVQA | | | | QRDFVA | | TNYADS | | FLQMNSLKTEDTA | | | | QVTVSS |
| | GGSLRLS | | | | | | VKG | | TYYCRA | | | | |
| | CTASGSD | | | | | | | | | | | | |
| | VR | | | | | | | | | | | | |
| 214 | EVQLVES | 384 | IYGMA | 554 | WYRQAPGK | 724 | SITGGY | 894 | RFTISRDDAKNTL | 1064 | LYSDY | 1234 | WGKGT |
| | GGGLVQA | | | | QRELVA | | GPNYVD | | YLQMNSLKPEDTA | | | | QVTVSS |
| | GGSLRLS | | | | | | SVKG | | VYYCNQ | | | | |
| | CVASGFIF | | | | | | | | | | | | |
| | S | | | | | | | | | | | | |
| 215 | EVQLVAS | 385 | IDGMN | 555 | WSRQAPGK | 725 | YITSSGS | 895 | RFTISRDNADNTV | 1065 | ATRSRLGLQQNY | 1235 | WGQGT |
| | GGGLVQT | | | | GRELVG | | TDYADS | | YLQMNSLKPEDTA | | | | QVTVSS |
| | GGSLRLS | | | | | | VKG | | VYYCAV | | | | |
| | CAASGSG | | | | | | | | | | | | |
| | FS | | | | | | | | | | | | |
| 216 | EVQLVES | 386 | IYTMA | 556 | WYRQAPGK | 726 | AVTYAG | 896 | RFTISRDDAKNKV | 1066 | NPSDNPW | 1236 | LGQGTQ |
| | GGGLVQA | | | | QRELVA | | NRYYVD | | YLQMNSLKPEDTA | | | | VTVSS |
| | GGSLRLS | | | | | | SVKG | | VYYCAA | | | | |
| | CAASGIIF | | | | | | | | | | | | |
| | T | | | | | | | | | | | | |
| 217 | EVQLVES | 387 | IDAMA | 557 | WYRQTPGK | 727 | AIFSGGL | 897 | RFTISRDNAKNTL | 1067 | RAPTGSDN | 1237 | WGQGT |
| | GGGLVQP | | | | QRDFVA | | THYADS | | YLQMNSLKPEDTA | | | | QVTVSS |
| | GGSLRLS | | | | | | VKG | | VYYCKF | | | | |
| | CAASGSIR | | | | | | | | | | | | |
| | S | | | | | | | | | | | | |
| 218 | EVQLVQS | 388 | SNITG | 558 | WYRQAPGK | 728 | AISSDGL | 898 | RVIISRDNVENTVY | 1068 | PGAG | 1238 | RGQGTQ |
| | GGGLVQA | | | | QRELVA | | AHYTDS | | LQMNSLKPEDTAV | | | | VTVSS |
| | GGSLRLS | | | | | | MKG | | YYCAA | | | | |
| | CAASGYIF | | | | | | | | | | | | |
| | S | | | | | | | | | | | | |
| 219 | EVQLVES | 389 | SDVMG | 559 | WFRQAPGK | 729 | YIHRSGT | 899 | RLTISRDNAKSTV | 1069 | GRYGSTSDTLYDY | 1239 | WGQGT |
| | GGGSVQA | | | | ERDFVA | | TYYADS | | YLQMNSLKPEDTA | | | | QVTVSS |
| | GGSLRLS | | | | | | VKG | | VYHCAA | | | | |
| | CAASQRT | | | | | | | | | | | | |
| | FS | | | | | | | | | | | | |
| 220 | EVPLVES | 390 | RDVTG | 560 | WFRQAPGK | 730 | YIHRSGE | 900 | RFTISRDNAKSTV | 1070 | GRYGSTSDTLYDY | 1240 | WGQGT |
| | GGGSVQA | | | | ERDFVA | | TTYYAD | | YLQMNSLKPEDTA | | | | QVTVSS |
| | GGSLRLS | | | | | | SVKG | | VYHCAA | | | | |
| | CAASQRT | | | | | | | | | | | | |
| | FS | | | | | | | | | | | | |
| 221 | EVQLVES | 391 | SDVMG | 561 | WFRQAPGK | 731 | YIHRSGT | 901 | RFTISRDNAKSTV | 1071 | GRYGSTADTLYDY | 1241 | WGQGT |
| | GGGSV*A | | | | ERDFVA | | TYYADS | | YLQMNSLKPEDTA | | | | QVTVSS |
| | GGSLRLS | | | | | | VKG | | VYHCAA | | | | |
| | CAASQRT | | | | | | | | | | | | |
| | FS | | | | | | | | | | | | |
| 222 | EVQLVES | 392 | RDVMG | 562 | WFRQAPGK | 732 | YSHRSG | 902 | RFTISRDNAKSTV | 1072 | GRYGSTSDTLYDY | 1242 | WGQGT |
| | GGGSVQA | | | | ERDFVA | | TTYYAD | | YLQMNSLKPEDTA | | | | QVTVSS |
| | GGSLrLSC | | | | | | SVKG | | VYHCAA | | | | |
| | AASQRTF | | | | | | | | | | | | |
| | S | | | | | | | | | | | | |
| 223 | XVQLVES | 393 | SDVMG | 563 | WFRQAPGK | 733 | YIHRSGT | 903 | RFTISXDNAKSTV | 1073 | GRYGSTSDTLYDY | 1243 | WGQGT |
| | GGGSVQA | | | | EGDFVA | | TYYADS | | YLQMNSLKPEDTA | | | | QVTVSS |
| | GGSLrLSC | | | | | | VKG | | VYHCAA | | | | |
| | AASQRTF | | | | | | | | | | | | |
| | S | | | | | | | | | | | | |
| 224 | EVQLVES | 394 | SDVMG | 564 | WFRQAPGK | 734 | YXHRSN | 904 | RFTISRDNAKSTV | 1074 | GRYGSTSDTLYDY | 1244 | WGQGT |
| | GGGSVQA | | | | ERDFVA | | TTYYAD | | YLQMNSLKPEDTA | | | | QVTVSS |
| | GGSLRLS | | | | | | SVKG | | VYHCAA | | | | |
| | CATSQRT | | | | | | | | | | | | |
| | FS | | | | | | | | | | | | |
| 225 | EVQLVES | 395 | HNTMA | 565 | WYRQAPGK | 735 | SISSGGN | 905 | RFTISRDNGNNTM | 1075 | KDWPPNYKNDY | 1245 | WGQGT |
| | GVGLVQA | | | | QRELAA | | TYYABS | | YLQMNNLKPEDT | | | | QVTVSS |
| | GGSLrLSC | | | | | | VKG | | AVYYCNW | | | | |
| | AASGYTF | | | | | | | | | | | | |
| | N | | | | | | | | | | | | |
| 226 | EVQLVES | 396 | HNTMA | 566 | WYRQAPVK | 736 | SISSGGS | 906 | RFTISRDNGNNTM | 1076 | KDWPPNYTNDY | 1246 | WGQGT |
| | GGGLVQA | | | | QRELAA | | TYYADS | | YLQMNNLKPEDT | | | | QVTVSS |
| | GGSLRLS | | | | | | VKG | | AVYYCNW | | | | |
| | CAASGYT | | | | | | | | | | | | |
| | FN | | | | | | | | | | | | |
| 227 | EVQLVES | 397 | VTSMG | 567 | WGRQIPGK | 737 | WITNEG | 907 | RFTISRDNAQNTL | 1077 | FSPRESSGNTY | 1247 | WGQGT |
| | GGGLVQA | | | | QRKLVA | | RTEYAD | | YLLMNSLKPEDTA | | | | QVTVSS |
| | GESLKLSC | | | | | | SVKG | | VYYCYG | | | | |
| | VASGNILR | | | | | | | | | | | | |
| 228 | EVQLVES | 398 | SNTMA | 568 | WFRQAPGK | 738 | AIMWRG | 908 | RFTISRDNAKNTV | 1078 | GGRRWNTRKDSSQY | 1248 | WGQGT |
| | GGGLVQA | | | | EREFGS | | DATYTD | | YLQMNSLKPEDTA | | DY | | QVTVSS |
| | GGSLRLS | | | | | | YADSMK | | IYYCAA | | | | |
| | CAASGRI | | | | | | D | | | | | | |
| | YS | | | | | | | | | | | | |
| 229 | EVQLVES | 399 | INDMG | 569 | WYRQPPGE | 739 | TLTSSDS | 909 | RFTISRDNAKNTV | 1079 | VINRRGDGRNWSREY | 1249 | WGQGT |
| | GGGSVQA | | | | QRELVA | | TKYADS | | YLQMNSLKPEDTA | | | | QVTVSS |
| | GGSLRLS | | | | | | VKS | | VYYCNA | | | | |
| | CTASGSIF | | | | | | | | | | | | |
| | S | | | | | | | | | | | | |
| 230 | EVQLVES | 400 | RMAMG | 570 | WYRQAPGV | 740 | IISPGGG | 910 | RFTISRDNAKNTV | 1080 | RNFEGRRVDY | 1250 | WGQGT |
| | GGGLVQP | | | | ERDFLA | | TNYADS | | YLQMNSLKPEDTA | | | | QVTVSS |
| | GGSLrLSC | | | | | | VKG | | VYYCNA | | | | |
| | AASGFTFS | | | | | | | | | | | | |
| 231 | EVQLVES | 401 | LNNMA | 571 | WYRQSPGK | 741 | LISGITS | 911 | RFSISRDNALNTV | 1081 | AWAGVEY | 1251 | WGQGT |
| | GGGLVQA | | | | KRELVA | | DPSTYY | | YLQMNSLKPEDTA | | | | QVTVSS |
| | GGSLrLSC | | | | | | LDSVRG | | VYYCKQ | | | | |
| | AASGSTY | | | | | | | | | | | | |
| | R | | | | | | | | | | | | |
| 232 | EVQLVES | 402 | IDVVS | 572 | WYRQAPgK | 742 | SITSAGR | 912 | RFGISRDNAKNTV | 1082 | LYRNAIY | 1252 | WGQGT |
| | GGGLVQA | | | | PRELAA | | IKYAES | | SLQMNSLKPEDTA | | | | QVTVSS |
| | GGSLrLSC | | | | | | VKG | | VYYCNA | | | | |
| | AASISANA | | | | | | | | | | | | |
| 233 | EVQLVES | 403 | DYAIG | 573 | WFRQAPGK | 743 | CIRNSD | 913 | RFTISSDNAKNTV | 1083 | TQIGRPRGDKGANRY | 1253 | RGQGTQ |
| | GGGLVQA | | | | EREGVS | | GRTYYA | | YLQMNSLKPEDTA | | CSXSRD | | VTVSS |
| | GGSLXLS | | | | | | DSVKG | | LYYCAA | | | | |
| | CAASGFT | | | | | | | | | | | | |
| | FD | | | | | | | | | | | | |
| 234 | EVQLXES | 404 | SNITG | 574 | WYRQAPGK | 744 | AISSDGL | 914 | RVIISRXNVENTVY | 1084 | PGAG | 1254 | RGQGTQ |
| | GGGLVQA | | | | QRELVA | | AHYTDS | | LQMNSLKPEDTAV | | | | VTVSS |
| | GGSLXLS | | | | | | MKG | | YYCAA | | | | |
| | XAASGYIF | | | | | | | | | | | | |
| | S | | | | | | | | | | | | |
| 235 | EVQLVES | 405 | IEAMA | 575 | WYRQAPGK | 745 | TINSASR | 915 | RFTISRDTGKSILY | 1085 | TTPLPYRRDF | 1255 | WGQGT |
| | GGGLVEA | | | | QRELVA | | TNYADS | | LQMNNLEPEDTAV | | | | QVTVSS |
| | GGSLRLS | | | | | | VKG | | YYCKI | | | | |
| | CATSGSTF | | | | | | | | | | | | |
| | G | | | | | | | | | | | | |
| 236 | EVQLVES | 406 | ITVPG | 576 | WYRQAPGK | 746 | VINSGG | 916 | RFTISIDNVKRTLY | 1086 | LKY | 1256 | WGQGT |
| | GGGSVQA | | | | QRELVA | | TKKYAD | | LEMNSLRPEDTAV | | | | QVTVSS |
| | GGSLRLS | | | | | | SVKG | | YYCST | | | | |
| | CAASGTT | | | | | | | | | | | | |
| | AT | | | | | | | | | | | | |
| 237 | EVQLVES | 407 | RFTMG | 577 | WFRQGPGK | 747 | AISWGG | 917 | RFTISRDNAQNTV | 1087 | BSRGPYNSNWHQSSV | 1257 | WGQGT |
| | GGGLVQA | | | | ERDFVA | | GRTNYE | | YLQMNSLKPEDTA | | SYDY | | QVTVSS |
| | GGSLRLS | | | | | | DSVKG | | VYYCAA | | | | |
| | CAASGRTI | | | | | | | | | | | | |
| | S | | | | | | | | | | | | |
| 238 | EVQLVES | 408 | RFTMG | 578 | WFRQAPGK | 748 | AISWGG | 918 | RFTISRDNAQNTV | 1088 | BTRGPYNSNWAQSSV | 1258 | WGQGT |
| | GGGLVQA | | | | ERDFVA | | GRTNYE | | YLQMNSLKPEDTA | | SYDG | | QVTVSS |
| | GGSLRLS | | | | | | DSVKG | | VYYCAA | | | | |
| | CAASGRTI | | | | | | | | | | | | |
| | S | | | | | | | | | | | | |
| 239 | EVQLVES | 409 | RFTMG | 579 | WFRQAPGK | 749 | AISWGG | 919 | RFTISRDNAQNTV | 1089 | BTRGPYNSNWAQSSV | 1259 | WGQGT |
| | GGGLVQA | | | | EREFVA | | GRTNYE | | YLQMNSLKPEDTA | | SYDY | | QVTVSS |
| | GGSLRLS | | | | | | DSVKG | | VYYCAA | | | | |
| | CAASGRTI | | | | | | | | | | | | |
| | S | | | | | | | | | | | | |
| 240 | EVQLVES | 410 | RFTMG | 580 | WFRQAPGK | 750 | AISWGG | 920 | RFTISRDNAQNTV | 1090 | BSRGPYNSNWHQSSV | 1260 | WGQGT |
| | GGGLVQA | | | | ERDFVA | | GRTNYE | | YLQMNSLKPEDTA | | SYDY | | QVTVSS |
| | GGSLRLS | | | | | | DSVKG | | VYYCAA | | | | |
| | CAASGRTI | | | | | | | | | | | | |
| | S | | | | | | | | | | | | |
| 241 | EVQLVES | 411 | RFTMG | 581 | WFRQAPGK | 751 | AISWGG | 921 | RFTISRDNAQNTV | 1091 | DTRGPYNSNWAQSSV | 1261 | WGQGT |
| | GGGLVQA | | | | ERDFVA | | GRTNYE | | YLQMNSLKPEDTA | | SYDT | | QVTVSS |
| | GGSLRLS | | | | | | DSVKG | | VYYCAA | | | | |
| | CAASGRTI | | | | | | | | | | | | |
| | S | | | | | | | | | | | | |
| 242 | EVQLVES | 412 | RFTMG | 582 | WFRQAPGK | 752 | AISWGG | 922 | RFTISRDNAQNTV | 1092 | DTRGPYNSNWAQSSV | 1262 | WGQGT |
| | GGGLVQA | | | | ERDFVA | | GRTNYE | | YLQMNSLKPEDTA | | SYDG | | QVTVSS |
| | GGSLRLS | | | | | | DSVKG | | VYYCAA | | | | |
| | CAASGRTI | | | | | | | | | | | | |
| | S | | | | | | | | | | | | |
| 243 | EVQLVES | 413 | RFTMG | 583 | WFRQAPGK | 753 | AISWGG | 923 | RFTISRDNAQNTV | 1093 | DTRGPYNSNWAQSSV | 1263 | WGQGT |
| | GGGLVQA | | | | ERDFVA | | GRTNYE | | YLQMNSLKPEDTA | | SYDG | | QVTVSS |
| | GDSLRLS | | | | | | DSVKG | | VYYCAA | | | | |
| | CAASGRTI | | | | | | | | | | | | |
| | S | | | | | | | | | | | | |
| 244 | EVQLVES | 414 | RFTMG | 584 | WFRQAPGK | 754 | AISWGG | 924 | RFTISRDNAQNTV | 1094 | BTRGPYNSNWHQSSV | 1264 | WGQGT |
| | GGGLVQA | | | | ERDFVA | | GRTNYE | | YLQMNSLKPEDTA | | SYDA | | QVTVSS |
| | GGSLRLS | | | | | | DSVKG | | VYYCAA | | | | |
| | CAASGRTI | | | | | | | | | | | | |
| | S | | | | | | | | | | | | |
| 245 | EVQLVES | 415 | RFTMG | 585 | WFRQAPGK | 755 | AISWGG | 925 | RFTISRDNARNTV | 1095 | BTRGPYNSNWAQSSV | 1265 | WGQGT |
| | GGGLVPA | | | | ERDFVA | | GRTDYE | | YLQMNSLKPEDTA | | SYNY | | QVTVSS |
| | GGSLRLS | | | | | | DSVKG | | VYYCAA | | | | |
| | CAASGRA | | | | | | | | | | | | |
| | IS | | | | | | | | | | | | |
| 246 | EVqLVESG | 416 | RFTMG | 586 | WFRQAPGK | 756 | AISWGG | 926 | RFTISRDNAQNTV | 1096 | BSRGPYNSNWHQSSV | 1266 | WGQGT |
| | GGLVQAG | | | | ERDFVA | | GRTKYE | | YLQMDSLKPEDTA | | SYDY | | QVTVSS |
| | GSLRLSC | | | | | | DSVTG | | VYYCAA | | | | |
| | AASGRTIS | | | | | | | | | | | | |
| 247 | EVQLVES | 417 | RFTMG | 587 | WFRQAPGK | 757 | AISWGG | 927 | RFTISRDNAQNTV | 1097 | BSRGPYNSNWHQSSV | 1267 | WGQGT |
| | GGGLVQA | | | | ERDFVA | | GRTKYE | | YLQMNSLKPEDTA | | SYDY | | QVTVSS |
| | GGSLRLS | | | | | | DSVTG | | VYYCAA | | | | |
| | CAASGRTI | | | | | | | | | | | | |
| | S | | | | | | | | | | | | |
| 248 | XVqLVES | 418 | RFTMG | 588 | WFRQAPGK | 758 | AISWGG | 928 | RSTISRDNAQNTV | 1098 | BTRGPYNSNWAQSSV | 1268 | WGQGT |
| | GGGLVQA | | | | ERDFVA | | GRTNYG | | YLQMNSLKPEDTA | | SYDY | | QVTVSS |
| | GGSLRLS | | | | | | DSVKG | | VYYCAA | | | | |
| | CAASGRTI | | | | | | | | | | | | |
| | S | | | | | | | | | | | | |
| 249 | EVQLVES | 419 | RFTMG | 589 | WFRQAPGK | 759 | AISWGG | 929 | RSTISRDNAQNTV | 1099 | BTRGPYNSNWAQSSV | 1269 | WGQGT |
| | GGGLVQA | | | | ERDFVA | | GRTNYE | | YLQMNSLKPEDTA | | SYDY | | QVTVSS |
| | GGSLRLS | | | | | | DSVKG | | VYYCAA | | | | |
| | CAASGRTI | | | | | | | | | | | | |
| | S | | | | | | | | | | | | |
| 250 | XVQLVES | 420 | RFTMG | 590 | WFRQAPGK | 760 | AISWGG | 930 | RFTISRDNAQNTV | 1100 | DSRGPYNSNWHQSSV | 1270 | WGQGT |
| | GGGLVQA | | | | ERDFVA | | GRTNYE | | YLQMNSLKPEDTA | | SYDY | | QVTVSS |
| | GGSLRLS | | | | | | BSVKD | | VYYCAA | | | | |
| | CAASGRTI | | | | | | | | | | | | |
| | S | | | | | | | | | | | | |
| 251 | EVQLVES | 421 | RFTMG | 591 | WFRQAPGK | 761 | AISWGG | 931 | RFTISRDNAQNTV | 1101 | BSRGPYNSNWHQSSV | 1271 | WGQGT |
| | GGGLVQA | | | | ERDFVA | | GRTNYE | | YLQMNSLKPEDTA | | SYDY | | QVTVSS |
| | GGSLRLS | | | | | | DSVKD | | VYYCAA | | | | |
| | CAASGRTI | | | | | | | | | | | | |
| | S | | | | | | | | | | | | |
| 252 | EV*LVESG | 422 | RFTMG | 592 | WFRQAPGK | 762 | AISWGG | 932 | RFTISRDNAQNTV | 1102 | DSRGPYNSNWHQSSV | 1272 | WGQGT |
| | GGLVQAG | | | | ERDFVA | | GRTKYE | | YLQMDSLKPEDTA | | SYDY | | QVTVSS |
| | GSLRLSC | | | | | | DSVTG | | VYYCAA | | | | |
| | AASGRTIS | | | | | | | | | | | | |
| 253 | EV*LVESG | 423 | RFTMG | 593 | WFRQAPGK | 763 | AISWGG | 933 | RFTISRDNAQNTV | 1103 | BSRGPYNSNWHQSSV | 1273 | WGQGT |
| | GGLVQAG | | | | ERDFVA | | GRTKYE | | YLQMDSLKPEDTA | | SYDY | | QVTVSS |
| | GSLRLSC | | | | | | DSVTG | | VYYCAA | | | | |
| | AASGRTIS | | | | | | | | | | | | |
| 254 | EVQLVES | 424 | INAMG | 594 | WYRQAPGK | 764 | VVTNGG | 934 | RFTVSREYAKNAV | 1104 | RGIIARWGSAPGNY | 1274 | WGQGT |
| | GGGLVQA | | | | QREVVA | | STEYAD | | YLQMNSLKPEDTA | | | | QVTVSS |
| | GGSLSLSC | | | | | | FVKG | | VYYCYA | | | | |
| | AASGASGI | | | | | | | | | | | | |
| | IYS | | | | | | | | | | | | |
| 255 | EVQLVES | 425 | ISSMG | 595 | WYRQAPGK | 765 | VVTNGG | 935 | RFTVSREYAKNAV | 1105 | RGIIARWGSAPGNY | 1275 | WGQGT |
| | GGGLVQA | | | | QREVVA | | STEYAD | | YLQMNSLKPEDTA | | | | QVTVSS |
| | GGSLRLS | | | | | | FVKG | | VYYCYA | | | | |
| | CAASGAS | | | | | | | | | | | | |
| | GTIYS | | | | | | | | | | | | |
| 256 | EVQLVES | 426 | YYAIG | 596 | WFRQAPGK | 766 | CISSSDG | 936 | RFTISRDNAKNTV | 1106 | TCVVNPEGYDF | 1276 | WGQGT |
| | GGGLVQP | | | | EREGVS | | STYYAB | | YLQMNSLKPEDTA | | | | QVTVSS |
| | GGSLRLS | | | | | | SVKG | | VYYCAT | | | | |
| | CAVSGFT | | | | | | | | | | | | |
| | SD | | | | | | | | | | | | |
| 257 | EVQLVES | 427 | INIMG | 597 | WYRQAPGK | 767 | YITKRGS | 937 | RFTISRDNAKNMA | 1107 | GPDGLGGQDDY | 1277 | WGQGT |
| | GGGLVQA | | | | QRDLVA | | TKYADS | | TLQMNSLKPEDTA | | | | QVTVSS |
| | GGSLRLS | | | | | | VKG | | VYYCAA | | | | |
| | CAASGSIS | | | | | | | | | | | | |
| | SINS | | | | | | | | | | | | |
| 258 | EVQLVES | 428 | ERIVG | 598 | WYRQAPGK | 768 | AITVPGI | 938 | RFTISRDSAKNTV | 1108 | PTYG | 1278 | RGQGTQ |
| | GGGLVQA | | | | QRELVA | | TNYTDS | | YLQMNKLKPEDT | | | | VTVSS |
| | GGSLRLA | | | | | | VKD | | AVYYCAA | | | | |
| | CQASRAI | | | | | | | | | | | | |
| 259 | EVQLVES | 429 | INNGE | 599 | WYRQAPGK | 769 | AIGSGG | 939 | RFTISKANAKNTL | 1109 | RSWRNY | 1279 | WGQGT |
| | GGGMVQ | | | | QREFVA | | TTDYAD | | YLQMNSLKPEDTA | | | | QVTVSS |
| | TGGSLRLS | | | | | | SVKG | | VYYCYV | | | | |
| | CAASGST | | | | | | | | | | | | |
| | LN | | | | | | | | | | | | |
| 260 | EVQLVES | 430 | TYAMG | 600 | WLRQAPGK | 770 | AISYSGA | 940 | RFTISRDNAKSTA | 1110 | SANRDLSLWVSTAYR | 1280 | WGQGT |
| | GGGLVQA | | | | ERQFVA | | TTYYAD | | YLQMNSLQPEDTA | | STGLWY | | QVTVSS |
| | GGSLRLS | | | | | | SVKG | | VYYCAA | | | | |
| | CTASGRT | | | | | | | | | | | | |
| | AS | | | | | | | | | | | | |
| 261 | EVQLVES | 431 | TYAMG | 601 | WFRQAPGE | 771 | TITWTG | 941 | RFTISRDNAKKTV | 1111 | DRRGYIETMSVNYDY | 1281 | WGQGT |
| | GGSLRLS | | | | ERQFVA | | YTYYTD | | YLRMDKLKPEDT | | | | QVTVSS |
| | GGGLVQA | | | | | | SVKG | | AVYYCAA | | | | |
| | CAASGGT | | | | | | | | | | | | |
| | FS | | | | | | | | | | | | |
| 262 | EVQLVES | 432 | INFMN | 602 | WYRQAPGK | 772 | QVTSGV | 942 | RFTISRDNAKNMV | 1112 | QGYFGSTWINY | 1282 | WGQGT |
| | GGGLVQP | | | | QRELVA | | TSGGTT | | SLQMNSLKPEDTA | | | | QVTVSS |
| | GGSLRLS | | | | | | YYDDSV | | VYYCNV | | | | |
| | CAASGSIS | | | | | | KG | | | | | | |
| | I | | | | | | | | | | | | |
| 263 | EVQLVES | 433 | INFMN | 603 | WYRQAPGK | 773 | QVTSGV | 943 | RFTISRDNAKNMV | 1113 | QGYFGSTWINY | 1283 | WGQGT |
| | GGGLVQP | | | | QRELVA | | TSGGTT | | SLQMNSLKPEDTA | | | | QVTVSS |
| | GGSLRLS | | | | | | YYDDSV | | VYYCNV | | | | |
| | CAAPGSIS | | | | | | KG | | | | | | |
| | I | | | | | | | | | | | | |
| 264 | EVPLVES | 434 | INFMN | 604 | WYRQAPGK | 774 | QVTSGV | 944 | RFTISRDNAKNMV | 1114 | QGYFGSTWINY | 1284 | WGQGT |
| | GGGLVQP | | | | QRELVA | | TSGGTT | | SLQMNSLKPEDTA | | | | QVTVSS |
| | GGSLRLS | | | | | | YYDDSV | | VYYCNV | | | | |
| | CAASGSIS | | | | | | KG | | | | | | |
| | I | | | | | | | | | | | | |
| 265 | EVRLVES | 435 | INFMN | 605 | WYRQAPGK | 775 | QVTSGV | 945 | RFTISRDNAKNMV | 1115 | QGYFGSTWINY | 1285 | WGQGT |
| | GGGLVQP | | | | QRELVA | | TSGGTT | | SLQMNSLKPEDTA | | | | QVTVSS |
| | GGSLRLS | | | | | | YYDDSV | | VYYCNV | | | | |
| | CAASGSIS | | | | | | KG | | | | | | |
| | I | | | | | | | | | | | | |
| 266 | EVQLVES | 436 | RYTMT | 606 | WVRQAPGK | 776 | SITSRGS | 946 | RFTISRDNAKNTL | 1116 | SGTETWYDRTY | 1286 | WGQGT |
| | GGGLVQP | | | | EPEWVS | | STNYAD | | YLQMNSLKPGDT | | | | QVTVSS |
| | GGSLRLS | | | | | | SVKG | | AMYYCAK | | | | |
| | CAASGFT | | | | | | | | | | | | |
| | FS | | | | | | | | | | | | |
| 267 | EVQLVES | 437 | RYTMT | 607 | WVRQAPGK | 777 | SITSRGS | 947 | RFTISRDNAKNTL | 1117 | SGTETWYDRTY | 1287 | WGQGT |
| | GGGLVQP | | | | EPEWVS | | STNYAD | | YLQMNSLKPGDT | | | | QVTVSS |
| | GGSLRLS | | | | | | SAKG | | AMYYCAK | | | | |
| | CAASGFT | | | | | | | | | | | | |
| | FS | | | | | | | | | | | | |
| 268 | EVQLVES | 438 | INTMA | 608 | WYRQAPGK | 778 | SITSRGT | 948 | RFTISTSNDKSTVY | 1118 | DKDGVIGYSVGY | 1288 | WGQGT |
| | GGGLVKA | | | | QREWIT | | TRYABS | | LQMNSLKPEDTAV | | | | QVTVSS |
| | GGSLRLS | | | | | | VKG | | YYCAA | | | | |
| | CAASGSIF | | | | | | | | | | | | |
| | S | | | | | | | | | | | | |
| 269 | EVQLVES | 439 | INVMG | 609 | WYRQAPGK | 779 | SITSRGT | 949 | RFTISRGNDKSTV | 1119 | BKGGVIGYSEGY | 1289 | WGQGT |
| | GGGLVEA | | | | QRELIG | | TRYTDS | | YLQMNSLKPEDTA | | | | QVTVSS |
| | GGSLRLS | | | | | | VKG | | VYYCAA | | | | |
| | CAASGSIF | | | | | | | | | | | | |
| | S | | | | | | | | | | | | |
| 270 | EVQLVES | 440 | INVMG | 610 | WYRQAPGK | 780 | SITSRGT | 950 | RFTISRGNDMSTV | 1120 | BKGGVIGYSVGY | 1290 | WGQGT |
| | GGGLVEA | | | | QRELIG | | TRYADS | | YLQMNSLKPEDTA | | | | QVTVSS |
| | GGSLRLS | | | | | | VKG | | VYYCAA | | | | |
| | CAASGSIF | | | | | | | | | | | | |
| | S | | | | | | | | | | | | |
| 271 | EVQLVES | 441 | IKAMG | 611 | WYRQAPGN | 781 | TITGTGK | 951 | RFTISRDIGTLYLQ | 1121 | LSWPAGDY | 1291 | WGQGT |
| | GGGLVQA | | | | QREMVA | | TNYADS | | MNSLKPEDTAVY | | | | QVTVSS |
| | GGSLQLS | | | | | | VKG | | YCNL | | | | |
| | CATSGESF | | | | | | | | | | | | |
| | S | | | | | | | | | | | | |
| 272 | EVQLVES | 442 | IKAMG | 612 | WYRQAPGN | 782 | TITGTGS | 952 | RFTISRXIGTLYLQ | 1122 | LSWPAGDY | 1292 | WDQGT |
| | GGGLVQA | | | | QRELVA | | TTYADS | | MNSLKPEDTGVY | | | | QVTVSS |
| | GGSLRLS | | | | | | AKG | | YCNL | | | | |
| | CTTSGRSF | | | | | | | | | | | | |
| | S | | | | | | | | | | | | |
| 273 | EVQLVES | 443 | INTMA | 613 | WYRQAPGN | 783 | IIFPGTG | 953 | RFTISRVNAKNTL | 1123 | VRYIGGNYFPFDS | 1293 | WGQGT |
| | GGALVQA | | | | ERDWVA | | GSTVYE | | YLQMDSLRPEDTG | | | | QVTVSS |
| | GGSLRLS | | | | | | DSVKG | | VYYCAR | | | | |
| | CAASGFA | | | | | | | | | | | | |
| | FS | | | | | | | | | | | | |
| 274 | EVQLVES | 444 | INTMA | 614 | WYRQAPGK | 784 | IIAPGTG | 954 | RFTISRVNAKNTL | 1124 | VRYTGGNYFPFDS | 1294 | WGQGT |
| | GGALVQA | | | | QRDWVA | | GSTHYE | | YLQMDSLRPEDTA | | | | QVTVSS |
| | GGSLRLS | | | | | | DSVKG | | VYYCAR | | | | |
| | CAASGFX | | | | | | | | | | | | |
| | FS | | | | | | | | | | | | |
| 275 | XVQLVES | 445 | NYPMG | 615 | WVRQAPGK | 785 | GISASSI | 955 | RFTISRDHAKNTL | 1125 | LRNYRYFGDMDY | 1295 | RGEGTL |
| | GGGLVQP | | | | GLEWVS | | RTSYAD | | YLQMNSLKVEDT | | | | VTVSS |
| | GGSLRLS | | | | | | SVKG | | AVYYCAQ | | | | |
| | CAASGFA | | | | | | | | | | | | |
| | FS | | | | | | | | | | | | |
| 276 | EVQLVES | 446 | HYPMG | 616 | WVRQAPGK | 786 | GISASSI | 956 | RFTISRDHAKNTL | 1126 | LRNYRYFGDMDY | 1296 | RGEGTL |
| | GGGLVQP | | | | GLEWVS | | RTSYAD | | YLQMNSLKVEDT | | | | VTVSS |
| | GGSLRLS | | | | | | SVKG | | AVYYCAQ | | | | |
| | CAASGFA | | | | | | | | | | | | |
| | FS | | | | | | | | | | | | |
| 277 | EVQLVES | 447 | RCAMG | 617 | WFRQAPGK | 787 | TISANGE | 957 | RFTISRDNAKNTV | 1127 | RRTFTRSSNRNEYAD | 1297 | WGQGT |
| | GGGLVQA | | | | EREFVA | | LIYYAN | | YLQMNSLKPEDTA | | | | QVTVSS |
| | GGSPRLSC | | | | | | FVEG | | VYFCAA | | | | |
| | VASGRTF | | | | | | | | | | | | |
| | S | | | | | | | | | | | | |
| 278 | EVQLVES | 448 | INAMG | 618 | WYRQAPGK | 788 | TITTRGT | 958 | RFTISKDNAKNTM | 1128 | BSPPYGMGSDLGY | 1298 | WGQGT |
| | GGGLVQA | | | | QRELVA | | TNYVDA | | YLQMNSLKPEDTA | | | | QVTVSS |
| | GGSLTLSC | | | | | | VKG | | VYYCAA | | | | |
| | AASGSVF | | | | | | | | | | | | |
| | S | | | | | | | | | | | | |
| 279 | EVQLVES | 449 | PNVMG | 619 | WFRQAPGD | 789 | NIYSGGS | 959 | RFTILSDNAKNTV | 1129 | KRVGQSWFDSGY | 1299 | WGQGT |
| | GGGLVQA | | | | QREFVA | | TNYADT | | YLQMTSLKPEDTG | | | | QVTVSS |
| | GGSLRLS | | | | | | VKG | | VYYCSV | | | | |
| | CAASGRM | | | | | | | | | | | | |
| | FS | | | | | | | | | | | | |
| 280 | EVQLVES | 450 | INNMG | 620 | WYRQAPGN | 790 | RITSGGS | 960 | RFTISIDNTRKTVY | 1130 | AIVSSRWGAEXTNDY | 1300 | WGQGT |
| | GGGLVQA | | | | KRDLVA | | HRNYAB | | LQMNSLKPEDTAV | | | | QVTVSS |
| | GGSLNLS | | | | | | SVKG | | YYCYG | | | | |
| | CAASGRIL | | | | | | | | | | | | |
| | R | | | | | | | | | | | | |
| 281 | EVQLVES | 451 | FNIMG | 621 | WYRQAPGK | 791 | TMTSGG | 961 | RSTISRENAKKTIT | 1131 | KTLTAWSTSTGDY | 1301 | WGQGT |
| | GGGLVQA | | | | QREMVA | | NTRYAD | | LQMNNLKPEDTG | | | | QVTVSS |
| | GGSLRLS | | | | | | SVKG | | VYYCNL | | | | |
| | CGASGSIG | | | | | | | | | | | | |
| | T | | | | | | | | | | | | |
| 282 | EVQLVES | 452 | DYAIG | 622 | WFRQAPGK | 792 | CISSPDG | 962 | RFTVSSDNAKNTA | 1132 | RRGGSYYFCDPLTVY | 1302 | WGQGT |
| | GGGLVQA | | | | EREGVS | | STYLVD | | YLQMNSLKPEDTA | | EYDY | | QVTVSS |
| | GGSLKLS | | | | | | SVKG | | VYYCAL | | | | |
| | CAASGFT | | | | | | | | | | | | |
| | FD | | | | | | | | | | | | |
| 283 | EVQLVES | 453 | NTVMS | 623 | WARQAPGK | 793 | TISASGV | 963 | RFTISRDYAKRTL | 1133 | RKSYTDYDPPRWDYD | 1303 | WGQGT |
| | GGALVQP | | | | GLEWVS | | RTRYAD | | YLQMNSLSPEDTG | | T | | QVTVSS |
| | GGSLRLS | | | | | | SVTG | | VYYCVR | | | | |
| | CAASGFA | | | | | | | | | | | | |
| | FS | | | | | | | | | | | | |
| 284 | EVQLVES | 454 | YQNMG | 624 | WYRQAPGN | 794 | SNWATG | 964 | RFTISRDDAKNVV | 1134 | LSRPWS | 1304 | WGQGT |
| | GGGLVQA | | | | EREWVA | | ATAYAD | | YLQMNNLKPEDT | | | | QVTVSS |
| | GGSLRLS | | | | | | SVKG | | AVYYCNR | | | | |
| | CAASGGT | | | | | | | | | | | | |
| | FR | | | | | | | | | | | | |
| 285 | EVQLVES | 455 | FKRVA | 625 | WHRQAPGN | 795 | AIWNTD | 965 | RFTISRDNAKKTV | 1135 | NRGSYGTY | 1305 | WGQGT |
| | GGGLVQA | | | | ERELVA | | NTDYAD | | YLQMNRLKPEDT | | | | QVTVSS |
| | GGSLRLS | | | | | | SVKG | | AVYYCSA | | | | |
| | CTVSITTY | | | | | | | | | | | | |
| | S | | | | | | | | | | | | |
| 286 | EVQLVDS | 456 | GYVLG | 626 | WFRQAPGK | 796 | AIIRSGG | 966 | RFTISRDNAKNTV | 1136 | SSVLGRSPALYDL | 1306 | WGQGT |
| | GGRLVQP | | | | EREFVA | | NTAYSD | | YLQMKSLKPEDTG | | | | QVTVSS |
| | GDLLRLS | | | | | | SVKG | | IYYCAR | | | | |
| | CTTSGFAS | | | | | | | | | | | | |
| | S | | | | | | | | | | | | |
| 287 | EVQLVES | 457 | RNVMA | 627 | WFRQAPGK | 797 | AIRWSG | 967 | RFTMSRDNAKNTI | 1137 | DARLYSPLPRRSSAYD | 1307 | WGQGT |
| | RGGLVQA | | | | EREFVA | | GTTSYA | | YLEMNSLKPEDTA | | Y | | QVTVSS |
| | GGSLRLS | | | | | | DFVKG | | VYYCAA | | | | |
| | CTASERTF | | | | | | | | | | | | |
| | S | | | | | | | | | | | | |
| 288 | EVQLVES | 458 | DYDIG | 628 | WFRQTPGK | 798 | CISSSDG | 968 | RFTISSDNAKNTV | 1138 | VKRAPKQYCSDYEAY | 1308 | WGQETQ |
| | GGGLVQP | | | | EREGVS | | YKFYAD | | YLQMNSLKPEDTA | | DY | | VTVSS |
| | GGSLRLS | | | | | | SVKD | | VYYCAA | | | | |
| | CAASGFT | | | | | | | | | | | | |
| | FD | | | | | | | | | | | | |
| 289 | EVQLVES | 459 | LGLVQ | 629 | WHRQVSRK | 799 | QLNSGG | 969 | RFTISRDNAKSTV | 1139 | RVIVPGGFRDY | 1309 | WGQGT |
| | GGGLVQA | | | | QRGLVA | | TTTYAD | | YLQMHSLKPEDTA | | | | QVTVSS |
| | GGSLRLS | | | | | | SVKG | | VYYCFL | | | | |
| | CAASGSIS | | | | | | | | | | | | |
| | S | | | | | | | | | | | | |
| 290 | EVQLVES | 460 | IRAMD | 630 | WYRQAPGK | 800 | TITSDGS | 970 | RFTISRDNAKNTL | 1140 | PPYGSSCPLV | 1310 | WGQGT |
| | GGGLVQA | | | | ERELVA | | TYYADS | | YLQMNSLKPEDTA | | | | QVTVSS |
| | GGSLRLS | | | | | | VKG | | AYYCKA | | | | |
| | CVASGBTI | | | | | | | | | | | | |
| | C | | | | | | | | | | | | |
| 291 | EVQLVES | 461 | INIVN | 631 | WYHQAPGK | 801 | FITNGEE | 971 | RFTVSRDNAKNTV | 1141 | HIMWPTVRDY | 1311 | WGQGT |
| | GGGLVQA | | | | QRELVA | | TNYAET | | SLQMNSLKPEDTG | | | | QVTVSS |
| | GGSLTLSC | | | | | | VKG | | VYYCNL | | | | |
| | AASGNISS | | | | | | | | | | | | |
| 292 | EVQLVES | 462 | YYAIG | 632 | WFRQAPGK | 802 | CISSSAG | 972 | RFTISRDNAKNTL | 1142 | QTAGTSIGCHISIGWY | 1312 | WGQGT |
| | GGGLVQP | | | | EREGVS | | SAYYAD | | YLQMNSLKPEDTA | | DY | | QVTVSS |
| | GGSLRLS | | | | | | SVKG | | VYYCAA | | | | |
| | CAASGITL | | | | | | | | | | | | |
| | N | | | | | | | | | | | | |
| 293 | EVQLVES | 463 | DYAIG | 633 | WFRQAPGK | 803 | CISSSDG | 973 | RFTISSDNAKNTV | 1143 | ALRTVLAGTPTCDRY | 1313 | WGQGT |
| | GGGLVQA | | | | EREGVS | | SIFYADS | | YLQMNSLKPEDTA | | EYDY | | QVTVSS |
| | GGSLRLS | | | | | | VKG | | VYYCAA | | | | |
| | CAASGFT | | | | | | | | | | | | |
| | FD | | | | | | | | | | | | |
| 294 | EVQLVES | 464 | INIVN | 634 | WYHQAPGK | 804 | FITNGEE | 974 | RFTVSRDNAKNTV | 1144 | HIMWPTVRDY | 1314 | WGQGT |
| | GGGLVQA | | | | QRELVA | | TNYAET | | SLQMNSLKPEDTG | | | | QVTVSS |
| | GGSLTLSC | | | | | | VKG | | VYYCNL | | | | |
| | AASGNISS | | | | | | | | | | | | |
| | FD | | | | | | | | | | | | |
| 295 | EVQLVES | 465 | INIVN | 635 | WYHQAPGK | 805 | FITNGEE | 975 | RFTVSRDNAKNTV | 1145 | HIMWPTVRDY | 1315 | WGQGT |
| | GGGLVQA | | | | QRELVA | | TNYAET | | SLQMNSLKPEDTG | | | | QVTVSS |
| | GGSLTLSC | | | | | | VKG | | VYYCNL | | | | |
| | AASGNISS | | | | | | | | | | | | |

(□ID□ refers to the SEQ ID NO as used herein)

Thus, in the Nanobodies of the invention, at least one of the CDR1, CDR2 and CDR3 sequences present is suitably chosen from the group consisting of the CDR1, CDR2 and CDR3 sequences, respectively, listed in Table A-1; or from the group of CDR1, CDR2 and CDR3 sequences, respectively, that have at least 80%, preferably at least 90%, more preferably at least 95%, even more preferably at least 99% □sequence identity □ (as defined herein) with at least one of the CDR1, CDR2 and CDR3 sequences, respectively, listed in Table A-1; and/or from the group consisting of the CDR1, CDR2 and CDR3 sequences, respectively, that have 3, 2 or only 1 □amino acid difference(s)□ (as defined herein) with at least one of the CDR1, CDR2 and CDR3 sequences, respectively, listed in Table A-1.

In this context, by □suitably chosen□ is meant that, as applicable, a [symbol] seq chosen from suitable CDR1 sequences (i.e. as defined herein), a CDR2 sequence is chosen from suitable CDR2 sequences (i.e. as defined herein), and a CDR3 sequence is chosen from suitable CDR3 sequence (i.e. as defined herein), respectively. More in particular, the CDR sequences are preferably chosen such that the Nanobodies of the invention bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In particular, in the Nanobodies of the invention, at least the CDR3 sequence present is suitably chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1; and/or from the group consisting of the CDR3 sequences that have 3, 2 or only 1 amino acid difference(s) with at least one of the CDR3 sequences listed in Table A-1.

Preferably, in the Nanobodies of the invention, at least two of the CDR1, CDR2 and CDR3 sequences present are suitably chosen from the group consisting of the CDR1, CDR2 and CDR3 sequences, respectively, listed in Table A-1 or from the group consisting of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 □amino acid difference(s)□ with at least one of the CDR1, CDR2 and CDR3 sequences, respectively, listed in Table A-1.

In particular, in the Nanobodies of the invention, at least the CDR3 sequence present is suitably chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1, respectively; and at least one of the CDR1 and CDR2 sequences present is suitably chosen from the group consisting of the CDR1 and CDR2 sequences, respectively, listed in Table A-1 or from the group of CDR1 and CDR2 sequences, respectively, that have at least 80%, preferably at least 90%, more preferably at least 95%, even more preferably at least 99% sequence identity with at least one of the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Most preferably, in the Nanobodies of the invention, all three CDR1, CDR2 and CDR3 sequences present are suitably chosen from the group consisting of the CDR1, CDR2 and CDR3 sequences, respectively, listed in Table A-1 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table A-I.

Even more preferably, in the Nanobodies of the invention, at least one of the CDR1, CDR2 and CDR3 sequences present is suitably chosen from the group consisting of the CDR1, CDR2 and CDR3 sequences, respectively, listed in Table A-1. Preferably, in this aspect, at least one or preferably both of the other two CDR sequences present are suitably chosen from CDR sequences that have at least 80%, preferably at least 90%, more preferably at least 95%, even more preferably at least 99% sequence identity with at least one of the corresponding CDR sequences, respectively, listed in Table A-1; and/or from the group consisting of the CDR sequences that have 3, 2 or only 1 amino acid difference(s) with at least one of the corresponding sequences, respectively, listed in Table A-1.

In particular, in the Nanobodies of the invention, at least the CDR3 sequence present is suitably chosen from the group consisting of the CDR3 listed in Table A-1. Preferably, in this aspect, at least one and preferably both of the CDR1 and CDR2 sequences present are suitably chosen from the groups of CDR1 and CDR2 sequences, respectively, that have at least 80%, preferably at least 90%, more preferably at least 95%, even more preferably at least 99% sequence identity with the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Even more preferably, in the Nanobodies of the invention, at least two of the CDR1, CDR2 and CDR3 sequences present are suitably chosen from the group consisting of the CDR1, CDR2 and CDR3 sequences, respectively, listed in Table A-1. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with at least one of the corresponding sequences listed in Table A-1.

In particular, in the Nanobodies of the invention, at least the CDR3 sequence is suitably chosen from the group consisting of the CDR3 sequences listed in Table A-1, and either the CDR1 sequence or the CDR2 sequence is suitably chosen from the group consisting of the CDR1 and CDR2 sequences, respectively, listed in Table A-1. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with the corresponding CDR sequences listed in Table A-1

Even more preferably, in the Nanobodies of the invention, all three CDR1, CDR2 and CDR3 sequences present are suitably chosen from the group consisting of the CDR1, CDR2 and CDR3 sequences, respectively, listed in Table A-1.

Also, generally, the combinations of CDR□s listed in Table A (i.e. those mentioned on the same line in Table A-1) are preferred. Thus, it is generally preferred that, when a CDR in a Nanobody of the invention is a CDR sequence mentioned in Table A-1 or is suitably chosen from the group of CDR sequences that have at least 80%, preferably at least 90%, more preferably at least 95%, even more preferably at least 99% sequence identity with a CDR sequence listed in Table A-1; and/or from the group consisting of CDR sequences that have 3, 2 or only 1 amino acid difference(s) with a CDR sequence listed in Table A-1, that at least one and preferably both of the other CDR D s are suitably chosen from the CDR sequences that belong to the same combination in Table A-1 (i.e. mentioned on the same line in Table A-1) or are suitably chosen from the group of CDR sequences that have at least 80%, preferably at least 90%, more preferably at least 95%, even more preferably at least 99% sequence identity with the CDR sequence(s) belonging to the same combination and/or from the group consisting of CDR sequences that have 3, 2 or only 1 amino acid difference(s) with the CDR sequence(s) belonging to the same combination. The other preferences indicated in the above paragraphs also apply to the combinations of CDR□s mentioned in Table A-1.

Thus, by means of non-limiting examples, a Nanobody of the invention can for example comprise a CDR1 sequence that has more than 80% sequence identity with one of the CDR1 sequences mentioned in Table A-1, a CDR2 sequence that has 3, 2 or 1 amino acid difference with one of the CDR2 sequences mentioned in Table A-1 (but belonging to a different combination), and a CDR3 sequence.

Some preferred Nanobodies of the invention may for example comprise: (I) a CDR1 sequence that has more than 80% sequence identity with one of the CDR1 sequences mentioned in Table A-1; a CDR2 sequence that has 3, 2 or 1 amino acid difference with one of the CDR2 sequences mentioned in Table A-1 (but belonging to a different combination); and a CDR3 sequence that has more than 80% sequence identity with one of the CDR3 sequences mentioned in Table A-1 (but belonging to a different combination); or (2) a CDR1 sequence that has more than 80% sequence identity with one of the CDR1 sequences mentioned in Table A-1; a CDR2 sequence, and one of the CDR3 sequences listed in Table A-1; or (3) a CDR1 sequence; a CDR2 sequence that has more than 80% sequence identity with one of the CDR2 sequence listed in Table A-1; and a CDR3 sequence that has 3, 2 or 1 amino acid differences with the CDR3 sequence mentioned in Table A-1 that belongs to the same combination as the CDR2 sequence.

Some particularly preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with the CDR2 sequence mentioned in Table A-1 that belongs to the same combination; and a CDR3 sequence that has more than 80% sequence identity with the CDR3 sequence mentioned in Table A-1 that belongs to the same combination; (2) a CDR1 sequence; a CDR 2 listed in Table A-1 and a CDR3 sequence listed in Table A-1 (in which the CDR2 sequence and CDR3 sequence may belong to different combinations).

Some even more preferred Nanobodies of the invention may for example comprise: (1) a CDR1 sequence that has more than 80% sequence identity with one of the CDR1 sequences mentioned in Table A-1; the CDR2 sequence listed in Table A-1 that belongs to the same combination; and a CDR3 sequence mentioned in Table A-1 that belongs to a different combination; or (2) a CDR1 sequence mentioned in Table A-1; a CDR2 sequence that has 3, 2 or 1 amino acid differences with the CDR2 sequence mentioned in Table A-1 that belongs to the same combination; and a CDR3 sequence that has more than 80% sequence identity with the CDR3 sequence listed in Table A-1 that belongs to the same or a different combination.

Particularly preferred Nanobodies of the invention may for example comprise a CDR1 sequence mentioned in Table A-1, a CDR2 sequence that has more than 80% sequence identity with the CDR2 sequence mentioned in Table A-1 that belongs to the same combination; and the CDR3 sequence mentioned in Table A-1 that belongs to the same combination.

In the most preferred Nanobodies of the invention, the CDR1, CDR2 and CDR3 sequences present are suitably chosen from one of the combinations of CDR1, CDR2 and CDR3 sequences, respectively, listed in Table A-1.

According to another preferred, but non-limiting aspect of the invention (a) CDR1 has a length of between 1 and 12 amino acid residues, and usually between 2 and 9 amino acid residues, such as 5, 6 or 7 amino acid residues; and/or (b) CDR2 has a length of between 13 and 24 amino acid residues, and usually between 15 and 21 amino acid residues, such as 16 and 17 amino acid residues; and/or (c) CDR3 has a length of between 2 and 35 amino acid residues, and usually between 3 and 30 amino acid residues, such as between 6 and 23 amino acid residues.

In another preferred, but non-limiting aspect, the invention relates to a Nanobody in which the CDR sequences (as defined herein) have more than 80%, preferably more than 90%, more preferably more than 95%, such as 99% or more sequence identity (as defined herein) with the CDR sequences of at least one of the amino acid sequences of SEQ ID NO□s: 1316 to 1476, and SEQ ID NO: 1485, 1486, and 1487 (see Table 1).

Generally, Nanobodies with the above CDR sequences may be as further described herein, and preferably have framework sequences that are also as further described herein. Thus, for example and as mentioned herein, such Nanobodies may be naturally occurring Nanobodies (from any suitable species), naturally occurring V_{HH} sequences (i.e. from a suitable species of Camelid) or synthetic or semi-synthetic amino acid sequences or Nanobodies, including but not limited to partially humanized Nanobodies or V_{HH} sequences, fully humanized Nanobodies or V_{HH} sequences, camelized heavy chain variable domain sequences, as well as Nanobodies that have been obtained by the techniques mentioned herein.

Thus, in one specific, but non-limiting aspect, the invention relates to a humanized Nanobody, which consists of 4 framework regions (FR1 to FR4 respectively) and 3 complementarity determining regions (CDR1 to CDR3 respectively), in which CDR1 to CDR3 are as defined herein and in which said humanized Nanobody comprises at least one humanizing substitution (as defined herein), and in particular at least one humanizing substitution in at least one of its framework sequences (as defined herein).

In another preferred, but non-limiting aspect, the invention relates to a Nanobody in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO□131s6 to 1476, and SEQ ID NO: 1485, 1486, and 1487 (see Table 1). This degree of amino acid identity can for example be determined by determining the degree of amino acid identity (in a manner described herein) between said Nanobody and one or more of the sequences of SEQ ID NO□131s6 to 1476, and SEQ ID NO: 1485, 1486, and 1487 (see Table 1), in which the amino acid residues that form the framework regions are disregarded. Such Nanobodies can be as further described herein.

In another preferred, but non-limiting aspect, the invention relates to a Nanobody with an amino acid sequence that is chosen from the group consisting of SEQ ID NO□s: 1316 to 1476, and SEQ ID NO: 1485, 1486, and 1487 (see Table 1) or from the group consisting of from amino acid sequences that have more than 80%, preferably more than 90%, more preferably more than 95%, such as 99% or more sequence identity (as defined herein) with at least one of the amino acid sequences of SEQ ID NO□s: 1316 to 1476, and SEQ ID NO: 1485, 1486, and 1487 (see Table 1).

Another preferred, but non-limiting aspect of the invention relates to humanized variants of the Nanobodies of SEQ ID NO□s: 1316 to 1476, and SEQ ID NO: 1485, 1486, and 1487 (see Table 1), that comprise, compared to the corresponding native V_{HH} sequence, at least one humanizing substitution (as defined herein), and in particular at least one humanizing substitution in at least one of its framework sequences (as defined herein).

The polypeptides of the invention comprise or essentially consist of at least one Nanobody of the invention.

It will be clear to the skilled person that the Nanobodies that are mentioned herein as □preferred□ (or □more preferred□, □even more preferred□, etc.) are also pre [symbol] preferred, or even more preferred, etc.) for use in the polypeptides described herein. Thus, polypeptides that comprise or essentially consist of one or more □preferred□ Nanobodies of the invention will generally be preferred, and polypeptides that comprise or essentially consist of one or more □more preferred□ Nanobodies of the invention will generally be more preferred, etc.

Generally, proteins or polypeptides that comprise or essentially consist of a single Nanobody (such as a single Nanobody of the invention) will be referred to herein as □monovalent□ proteins or polypeptides or as □monovalent constructs□. Proteins and polypeptides that comprise or essentially consist of two or more Nanobodies (such as at least two Nanobodies of the invention or at least one Nanobody of the invention and at least one other Nanobody) will be referred to herein as □multivalent□ proteins or polypeptides or multivalent constructs□, and these may provide certain advantages compared to the corresponding monovalent Nanobodies of the invention. Some non-limiting examples of such multivalent constructs will become clear from the further description herein.

According to one specific, but non-limiting aspect, a polypeptide of the invention comprises or essentially consists of at least two Nanobodies of the invention, such as two or three Nanobodies of the invention. As further described herein, such multivalent constructs can provide certain advantages compared to a protein or polypeptide comprising or essentially consisting of a single Nanobody of the invention, such as a much improved avidity for Integrins. Such multivalent constructs will be clear to the skilled person based on the disclosure herein.

According to another specific, but non-limiting aspect, a polypeptide of the invention comprises or essentially consists of at least one Nanobody of the invention and at least one other binding unit (i.e. directed against another epitope, antigen, target, protein or polypeptide), which is preferably also a Nanobody. Such proteins or polypeptides are also referred to herein as □multispecific□ proteins or polypeptides or as □multispecific constructs and these may provide certain advantages compared to the corresponding monovalent Nanobodies of the invention (as will become clear from the further discussion herein of some preferred, but-nonlimiting multispecific constructs). Such multispecific constructs will be clear to the skilled person based on the disclosure herein.

According to yet another specific, but non-limiting aspect, a polypeptide of the invention comprises or essentially consists of at least one Nanobody of the invention, optionally one or more further Nanobodies, and at least one other amino acid sequence (such as a protein or polypeptide) that confers at least one desired property to the Nanobody of the invention and/or to the resulting fusion protein. Again, such fusion proteins may provide certain advantages compared to the corresponding monovalent Nanobodies of the invention. Some non-limiting examples of such amino acid sequences and of such fusion constructs will become clear from the further description herein.

It is also possible to combine two or more of the above aspects, for example to provide a trivalent bispecific construct comprising two Nanobodies of the invention and one other Nanobody, and optionally one or more other amino acid sequences. Further non-limiting examples of such constructs, as well as some constructs that are particularly preferred within the context of the present invention, will become clear from the further description herein. In the above constructs, the one or more Nanobodies and/or other amino acid sequences may be directly linked to each other and/or suitably linked to each other via one or more linker sequences. Some suitable but non-limiting examples of such linkers will become clear from the further description herein.

In one specific aspect of the invention, a Nanobody of the invention or a compound, construct or polypeptide of the invention comprising at least one Nanobody of the invention may have an increased half-life, compared to the corresponding amino acid sequence of the invention. Some preferred, but non-limiting examples of such Nanobodies, compounds and polypeptides will become clear to the skilled person based on the further disclosure herein, and for example comprise Nanobodies sequences or polypeptides of the invention that have been chemically modified to increase the half-life thereof (for example, by means of pegylation); amino acid sequences of the invention that comprise at least one additional binding site for binding to a serum protein (such as serum albumin, see for example EP 0 368 684 B1, page 4); or polypeptides of the invention that comprise at least one Nanobody of the invention that is linked to at least one moiety (and in particular at least one amino acid sequence) that increases the half-life of the Nanobody of the invention. Examples of polypeptides of the invention that comprise such half-life extending moieties or amino acid sequences will become clear to the skilled person based on the further disclosure herein; and for example include, without limitation, polypeptides in which the one or more Nanobodies of the invention are suitable linked to one or more serum proteins or fragments thereof (such as serum albumin or suitable fragments thereof) or to one or more binding units that can bind to serum proteins (such as, for example, Nanobodies or (single) domain antibodies that can bind to serum proteins such as serum albumin, serum immunoglobulins such as IgG, or transferrine); polypeptides in which a Nanobody of the invention is linked to an Fc portion (such as a human Fc) or a suitable part or fragment thereof; or polypeptides in which the one or more Nanobodies of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006 (see also PCT/EP/2007/063348). Again, as will be clear to the skilled person, such Nanobodies, compounds, constructs or polypeptides may contain one or more additional groups, residues, moieties or binding units, such as one or more further amino acid sequences and in particular one or more additional Nanobodies (i.e. not directed against Integrins), so as to provide a tri- of multispecific Nanobody construct.

Generally, the Nanobodies of the invention (or compounds, constructs or polypeptides comprising the same) with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the Nanobodies, compounds, constructs or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In a preferred, but non-limiting aspect of the invention, such Nanobodies, compound, constructs or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another one aspect of the invention, a polypeptide of the invention comprises one or more (such as two or preferably one) Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) Nanobodies, such as the Nanobodies described in WO 02/057445, of which FC44 (SEQ ID NO: 189 of WO 06/040153) and FC5 (SEQ ID NO: 190 of WO 06/040154) are preferred examples.

In particular, polypeptides comprising one or more Nanobodies of the invention are preferably such that they:
bind to Integrins with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to Integrins with a kₒₙ-rate of between 10² M⁻¹s⁻¹ to about 10⁷ M⁻¹s⁻¹, preferably between 10³ M¹s⁻¹ and 10⁷ M⁻¹s⁻¹, more preferably between 10⁴ M⁻¹s¹ and 10⁷ M⁻¹s⁻¹, such as between 10⁵ M⁻¹s⁻¹ and 10⁷ M⁻¹s⁻¹;
   and/or such that they:
bind to Integrins with a k_{off} rate between 1 s⁻¹ (t_{1/2}=0.69 s) and 10⁻⁶ s¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a polypeptide that contains only one amino acid sequence of the invention is preferably such that it will bind to Integrins with an affinity less than 500 nM, preferably less than 200 nM, more preferably less than 10 nM, such as less than 500 pM. In this respect, it will be clear to the skilled person that a polypeptide that contains two or more Nanobodies of the invention may bind to Integrins with an increased avidity, compared to a polypeptide that contains only one amino acid sequence of the invention.

Some preferred IC₅₀ values for binding of the amino acid sequences or polypeptides of the invention to Integrins will become clear from the further description and examples herein.

Other polypeptides according to this preferred aspect of the invention may for example be chosen from the group consisting of amino acid sequences that have more than 80%, preferably more than 90%, more preferably more than 95%, such as 99% or more □sequence identity □ (as defined herein) with [symbol] the amino acid sequences of SEQ ID NO□s: [polypeptides of the invention] (see Table 3), in which the Nanobodies comprised within said amino acid sequences are preferably as further defined herein.

Another aspect of this invention relates to a nucleic acid that encodes an amino acid sequence of the invention (such as a Nanobody of the invention) or a polypeptide of the invention comprising the same. Again, as generally described herein for the nucleic acids of the invention, such a nucleic acid may be in the form of a genetic construct, as defined herein.

In another aspect, the invention relates to host or host cell that expresses or that is capable of expressing an amino acid sequence (such as a Nanobody) of the invention and/or a polypeptide of the invention comprising the same; and/or that contains a nucleic acid of the invention. Some preferred but non-limiting examples of such hosts or host cells will become clear from the further description herein.

Another aspect of the invention relates to a product or composition containing or comprising at least one amino acid sequence of the invention, at least one polypeptide of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention further relates to methods for preparing or generating the amino acid sequences, compounds, constructs, polypeptides, nucleic acids, host cells, products and compositions described herein. Some preferred but non-limiting examples of such methods will become clear from the further description herein.

The invention further relates to applications and uses of the amino acid sequences, compounds, constructs, polypeptides, nucleic acids, host cells, products and compositions described herein, as well as to methods for the prevention and/or treatment for diseases and disorders associated with Integrins. Some preferred but non-limiting applications and uses will become clear from the further description herein.

Other aspects, embodiments, advantages and applications of the invention will also become clear from the further description hereinbelow.

Generally, it should be noted that the term Nanobody as used herein in its broadest sense is not limited to a specific biological source or to a specific method of preparation. For example, as will be discussed in more detail below, the Nanobodies of the invention can generally be obtained by any of the techniques (1) to (8) mentioned on pages 61 and 62 of WO 08/020,079, or any other suitable technique known per se. One preferred class of Nanobodies corresponds to the V_{HH} domains of naturally occurring heavy chain antibodies directed against Integrins. As further described herein, such V_{HH} sequences can generally be generated or obtained by suitably immunizing a species of Camelid with Integrins (i.e. so as to raise an immune response and/or heavy chain antibodies directed against Integrins), by obtaining a suitable biological sample from said Camelid (such as a blood sample, serum sample or sample of B-cells), and by generating V_{HH} sequences directed against Integrins, starting from said sample, using any suitable technique known per se. Such techniques will be clear to the skilled person and/or are further described herein.

Alternatively, such naturally occurring V_{HH} domains against Integrins, can be obtained from naïve libraries of Camelid V_{HH} sequences, for example by screening such a library using Integrins, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from naïve Vim libraries may be used, such as V_{HH} libraries obtained from naïve V_{HH} libraries by techniques such as random mutagenesis and/or CDR shuffling, as for example described in WO 00/43507.

Thus, in another aspect, the invention relates to a method for generating Nanobodies, that are directed against Integrins. In one aspect, said method at least comprises the steps of:
a) providing a set, collection or library of Nanobody sequences; and
b) screening said set, collection or library of Nanobody sequences for Nanobody sequences that can bind to and/or have affinity for Integrins;
   and
c) isolating the amino acid sequence(s) that can bind to and/or have affinity for Integrins.

In such a method, the set, collection or library of Nanobody sequences may be a naïve set, collection or library of Nanobody sequences; a synthetic or semi-synthetic set, collection or library of Nanobody sequences; and/or a set, collection or library of Nanobody sequences that have been subjected to affinity maturation.

In a preferred aspect of this method, the set, collection or library of Nanobody sequences may be an immune set, collection or library of Nanobody sequences, and in particular an immune set, collection or library of V_{HH} sequences, that have been derived from a species of Camelid that has been suitably immunized with Integrins or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of Nanobody or V_{HH} sequences may be displayed on a phage, phagemid, ribosome or suitable micro-organism (such as yeast), such as to facilitate screening. Suitable methods, techniques and host organisms for displaying and screening (a set, collection or library of) Nanobody sequences will be clear to the person skilled in the art, for example on the basis of the further disclosure herein. Reference is also made toWO 03/054016 and to the review by Hoogenboom in Nature Biotechnology, 23, 9, 1105-1116 (2005).

In another aspect, the method for generating Nanobody sequences comprises at least the steps of:
a) providing a collection or sample of cells derived from a species of Camelid that express immunoglobulin sequences;
b) screening said collection or sample of cells for (i) cells that express an immunoglobulin sequence that can bind to and/or have affinity for Integrins; and (ii) cells that express heavy chain antibodies, in which substeps (i) and (ii) can be performed essentially as a single screening step or in any suitable order as two separate screening steps, so as to provide at least one cell that expresses a heavy chain antibody that can bind to and/or has affinity for Integrins;
   and
c) either (i) isolating from said cell the V_{HH} sequence present in said heavy chain antibody; or (ii) isolating from said cell a nucleic acid sequence that encodes the V_{HH} sequence present in said heavy chain antibody, followed by expressing said V_{HH} domain.

In the method according to this aspect, the collection or sample of cells may for example be a collection or sample of B-cells. Also, in this method, the sample of cells may be derived from a Camelid that has been suitably immunized with Integrins or a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820. Particular reference is made to the so-called □Nanoclone™ technique described in International application WO 06/079372 by Ablynx N.V.

In another aspect, the method for generating an amino acid sequence directed against Integrins may comprise at least the steps of:
a) providing a set, collection or library of nucleic acid sequences encoding heavy chain antibodies or Nanobody sequences;
b) screening said set, collection or library of nucleic acid sequences for nucleic acid sequences that encode a heavy chain antibody or a Nanobody sequence that can bind to and/or has affinity for Integrins;
   and
c) isolating said nucleic acid sequence, followed by expressing the V_{HH} sequence present in said heavy chain antibody or by expressing said Nanobody sequence, respectively.

In such a method, the set, collection or library of nucleic acid sequences encoding heavy chain antibodies or Nanobody sequences may for example be a set, collection or library of nucleic acid sequences encoding a naïve set, collection or library of heavy chain antibodies or V_{HH} sequences; a set, collection or library of nucleic acid sequences encoding a synthetic or semi-synthetic set, collection or library of Nanobody sequences; and/or a set, collection or library of nucleic acid sequences encoding a set, collection or library of Nanobody sequences that have been subjected to affinity maturation.

In a preferred aspect of this method, the set, collection or library of amino acid sequences may be an immune set, collection or library of nucleic acid sequences encoding heavy chain antibodies or VHH sequences derived from a Camelid that has been suitably immunized with Integrins or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to WO 03/054016 and to the review by Hoogenboom in Nature Biotechnology, 23, 9, 1105-1116 (2005).

As will be clear to the skilled person, the screening step of the methods described herein can also be performed as a selection step. Accordingly the term □screening□ as used in the present description can comprise selection, screening or any suitable combination of selection and/or screening techniques. Also, when a set, collection or library of sequences is used, it may contain any suitable number of sequences, such as 1, 2, 3 or about 5, 10, 50, 100, 500, 1000, 5000, 10⁴, 10⁵, 10⁶, 10⁷, 10⁸ or more sequences.

Also, one or more or all of the sequences in the above set, collection or library of amino acid sequences may be obtained or defined by rational, or semi-empirical approaches such as computer modelling techniques or biostatics or datamining techniques.

Furthermore, such a set, collection or library can comprise one, two or more sequences that are variants from one another (e.g. with designed point mutations or with randomized positions), compromise multiple sequences derived from a diverse set of naturally diversified sequences (e.g. an immune library)), or any other source of diverse sequences (as described for example in Hoogenboom et al, Nat Biotechnol 23:1105, 2005 and Binz et al, Nat Biotechnol 2005, 23:1247). Such set, collection or library of sequences can be displayed on the surface of a phage particle, a ribosome, a bacterium, a yeast cell, a mammalian cell, and linked to the nucleotide sequence encoding the amino acid sequence within these carriers. This makes such set, collection or library amenable to selection procedures to isolate the desired amino acid sequences of the invention. More generally, when a sequence is displayed on a suitable host or host cell, it is also possible (and customary) to first isolate from said host or host cell a nucleotide sequence that encodes the desired sequence, and then to obtain the desired sequence by suitably expressing said nucleotide sequence in a suitable host organism. Again, this can be performed in any suitable manner known per se, as will be clear to the skilled person.

Yet another technique for obtaining V_{HH} sequences or Nanobody sequences directed against Integrins involves suitably immunizing a transgenic mammal that is capable of expressing heavy chain antibodies (i.e. so as to raise an immune response and/or heavy chain antibodies directed against Integrins), obtaining a suitable biological sample from said transgenic mammal that contains (nucleic acid sequences encoding) said VHH sequences or

Nanobody sequences (such as a blood sample, serum sample or sample of B-cells), and then generating V_{HH} sequences directed against Integrins, starting from said sample, using any suitable technique known per se (such as any of the methods described herein or a hybridoma technique). For example, for this purpose, the heavy chain antibody-expressing mice and the further methods and techniques described in WO 02/085945, WO 04/049794 and WO 06/008548 and Janssens et al., Proc. Natl. Acad. Sci. USA. 2006 Oct. 10; 103(41):15130-5 can be used. For example, such heavy chain antibody expressing mice can express heavy chain antibodies with any suitable (single) variable domain, such as (single) variable domains from natural sources (e.g. human (single) variable domains, Camelid (single) variable domains or shark (single) variable domains), as well as for example synthetic or semi-synthetic (single) variable domains.

The invention also relates to the V_{HH} sequences or Nanobody sequences that are obtained by the above methods, or alternatively by a method that comprises the one of the above methods and in addition at least the steps of determining the nucleotide sequence or amino acid sequence of said V_{HH} sequence or Nanobody sequence; and of expressing or synthesizing said Vim sequence or Nanobody sequence in a manner known per se, such as by expression in a suitable host cell or host organism or by chemical synthesis.

As mentioned herein, a particularly preferred class of Nanobodies of the invention comprises Nanobodies with an amino acid sequence that corresponds to the amino acid sequence of a naturally occurring V_{HH} domain, but that has been □humanized□, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above), as further described on, and using the techniques mentioned on, page 63 of WO 08/020,079. Another particularly preferred class of Nanobodies of the invention comprises Nanobodies with an amino acid sequence that corresponds to the amino acid sequence of a naturally occurring V_{H} domain, but that has been □camelized□, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody, as further described on, and using the techniques mentioned on, page 63 of WO 08/020,079. Other suitable methods and techniques for obtaining the Nanobodies of the invention and/or nucleic acids encoding the same, starting from naturally occurring V_{H} sequences or preferably V_{HH} sequences, will be clear from the skilled person, and may for example include the techniques that are mentioned on page 64 of WO 08/00279As mentioned herein, Nanobodies may in particular be characterized by the presence of one or more □Hallmark residues□ (as described herein) in one or more of the framework sequences.

Thus, according to one preferred, but non-limiting aspect of the invention, a Nanobody in its broadest sense can be generally defined as a polypeptide comprising:
a) an amino acid sequence that is comprised of four framework regions/sequences interrupted by three complementarity determining regions/sequences, in which the amino acid residue at position 108 according to the Kabat numbering is Q;
   and/or:
b) an amino acid sequence that is comprised of four framework regions/sequences interrupted by three complementarity determining regions/sequences, in which the amino acid residue at position 45 according to the Kabat numbering is a charged amino acid (as defined herein) or a cysteine residue, and position 44 is preferably an E;
   and/or:
c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, in a first preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q;
   and/or in which:
b) the amino acid residue at position 45 according to the Kabat numbering is a charged amino acid or a cysteine and the amino acid residue at position 44 according to the Kabat numbering is preferably E;
   and/or in which:
c) the amino acid residue at position 103 according to the Kabat numbering is chosen from the group consisting of P, R and S, and is in particular chosen from the group consisting of R and S;
   and in which:
d) CDR1, CDR2 and CDR3 are as defined herein, and are preferably as defined according to one of the preferred aspects herein, and are more preferably as defined according to one of the more preferred aspects herein.

In particular, a Nanobody in its broadest sense can be generally defined as a polypeptide comprising:
a) an amino acid sequence that is comprised of four framework regions/sequences interrupted by three complementarity determining regions/sequences, in which the amino acid residue at position 108 according to the Kabat numbering is Q;
   and/or:
b) an amino acid sequence that is comprised of four framework regions/sequences interrupted by three complementarity determining regions/sequences, in which the amino acid residue at position 44 according to the Kabat numbering is E and in which the amino acid residue at position 45 according to the Kabat numbering is an R;
   and/or:
c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, according to a preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q;
   and/or in which:
b) the amino acid residue at position 44 according to the Kabat numbering is E and in which the amino acid residue at position 45 according to the Kabat numbering is an R;
   and/or in which:
c) the amino acid residue at position 103 according to the Kabat numbering is chosen from the group consisting of P, R and S, and is in particular chosen from the group consisting of R and S;
   and in which:
d) CDR1, CDR2 and CDR3 are as defined herein, and are preferably as defined according to one of the preferred aspects herein, and are more preferably as defined according to one of the more preferred aspects herein.

In particular, a Nanobody against Integrins according to the invention may have the structure:
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q;
   and/or in which:
b) the amino acid residue at position 44 according to the Kabat numbering is E and in which the amino acid residue at position 45 according to the Kabat numbering is an R;
   and/or in which:
c) the amino acid residue at position 103 according to the Kabat numbering is chosen from the group consisting of P, R and S, and is in particular chosen from the group consisting of R and S;
   and in which:
d) CDR1, CDR2 and CDR3 are as defined herein, and are preferably as defined according to one of the preferred aspects herein, and are more preferably as defined according to one of the more preferred aspects herein.

In particular, according to one preferred, but non-limiting aspect of the invention, a Nanobody can generally be defined as a polypeptide comprising an amino acid sequence that is comprised of four framework regions/sequences interrupted by three complementarity determining regions/sequences, in which;
a-1) the amino acid residue at position 44 according to the Kabat numbering is chosen from the group consisting of A, G, E, D, G, Q, R, S, L; and is preferably chosen from the group consisting of G, E or Q; and
a-2) the amino acid residue at position 45 according to the Kabat numbering is chosen from the group consisting of L, R or C; and is preferably chosen from the group consisting of L or R; and
a-3) the amino acid residue at position 103 according to the Kabat numbering is chosen from the group consisting of W, R or S; and is preferably W or R, and is most preferably W;
a-4) the amino acid residue at position 108 according to the Kabat numbering is Q;
   or in which:
b-1) the amino acid residue at position 44 according to the Kabat numbering is chosen from the group consisting of E and Q; and
b-2) the amino acid residue at position 45 according to the Kabat numbering is R; and
b-3) the amino acid residue at position 103 according to the Kabat numbering is chosen from the group consisting of W, R and S; and is preferably W;
b-4) the amino acid residue at position 108 according to the Kabat numbering is chosen from the group consisting of Q and L; and is preferably Q;
   or in which:
c-1) the amino acid residue at position 44 according to the Kabat numbering is chosen from the group consisting of A, G, E, D, Q, R, S and L; and is preferably chosen from the group consisting of G, E and Q; and
c-2) the amino acid residue at position 45 according to the Kabat numbering is chosen from the group consisting of L, R and C; and is preferably chosen from the group consisting of L and R; and
c-3) the amino acid residue at position 103 according to the Kabat numbering is chosen from the group consisting of P, R and S; and is in particular chosen from the group consisting of R and S; and
c-4) the amino acid residue at position 108 according to the Kabat numbering is chosen from the group consisting of Q and L; is preferably Q;
   and in which
d) CDR1, CDR2 and CDR3 are as defined herein, and are preferably as defined according to one of the preferred aspects herein, and are more preferably as defined according to one of the more preferred aspects herein.

Thus, in another preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
a-1) the amino acid residue at position 44 according to the Kabat numbering is chosen from the group consisting of A, G, E, D, G, Q, R, S, L; and is preferably chosen from the group consisting of G, E or Q; and in which:
a-2) the amino acid residue at position 45 according to the Kabat numbering is chosen from the group consisting of L, R or C; and is preferably chosen from the group consisting of L or R;
   and in which:
a-3) the amino acid residue at position 103 according to the Kabat numbering is chosen from the group consisting of W, R or S; and is preferably W or R, and is most preferably W;
   and in which
a-4) the amino acid residue at position 108 according to the Kabat numbering is Q;
   and in which:
d) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
b-1) the amino acid residue at position 44 according to the Kabat numbering is chosen from the group consisting of E and Q;
   and in which:
b-2) the amino acid residue at position 45 according to the Kabat numbering is R;
   and in which:
b-3) the amino acid residue at position 103 according to the Kabat numbering is chosen from the group consisting of W, R and S; and is preferably W;
   and in which:
b-4) the amino acid residue at position 108 according to the Kabat numbering is chosen from the group consisting of Q and L; and is preferably Q;
   and in which:
d) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
c-1) the amino acid residue at position 44 according to the Kabat numbering is chosen from the group consisting of A, G, E, D, Q, R, S and L; and is preferably chosen from the group consisting of G, E and Q;
   and in which:
c-2) the amino acid residue at position 45 according to the Kabat numbering is chosen from the group consisting of L, R and C; and is preferably chosen from the group consisting of L and R;
   and in which:
c-3) the amino acid residue at position 103 according to the Kabat numbering is chosen from the group consisting of P, R and S; and is in particular chosen from the group consisting of R and S;
   and in which:
c-4) the amino acid residue at position 108 according to the Kabat numbering is chosen from the group consisting of Q and L; is preferably Q;
   and in which:
d) CDR1, CDR2 and CDR3 are as defined herein, and are preferably as defined according to one of the preferred aspects herein, and are more preferably as defined according to one of the more preferred aspects herein.

Two particularly preferred, but non-limiting groups of the Nanobodies of the invention are those according to a) above; according to (a-1) to (a-4) above; according to b) above; according to (b-1) to (b-4) above; according to (c) above; and/or according to (c-1) to (c-4) above, in which either:
i) the amino acid residues at positions 44-47 according to the Kabat numbering form the sequence GLEW (or a GLEW-like sequence as described herein) and the amino acid residue at position 108 is Q;
   or in which:
ii) the amino acid residues at positions 43-46 according to the Kabat numbering form the sequence KERE or KQRE (or a KERE-like sequence as described) and the amino acid residue at position 108 is Q or L, and is preferably Q.

Thus, in another preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
i) the amino acid residues at positions 44-47 according to the Kabat numbering form the sequence GLEW (or a GLEW-like sequence as defined herein) and the amino acid residue at position 108 is Q;
   and in which:
ii) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
i) the amino acid residues at positions 43-46 according to the Kabat numbering form the sequence KERE or KQRE (or a KERE-like sequence) and the amino acid residue at position 108 is Q or L, and is preferably Q;
   and in which:
ii) CDR1, CDR2 and CDR3 are as defined herein, and are preferably as defined according to one of the preferred aspects herein, and are more preferably as defined according to one of the more preferred aspects herein.

In the Nanobodies of the invention in which the amino acid residues at positions 43-46 according to the Kabat numbering form the sequence KERE or KQRE, the amino acid residue at position 37 is most preferably F. In the Nanobodies of the invention in which the amino acid residues at positions 44-47 according to the Kabat numbering form the sequence GLEW, the amino acid residue at position 37 is chosen from the group consisting of Y, H, I, L, V or F, and is most preferably V.

Thus, without being limited hereto in any way, on the basis of the amino acid residues present on the positions mentioned above, the Nanobodies of the invention can generally be classified on the basis of the following three groups:
i) The □GELEW-group□: Nanobodies with the amino acid sequence GLEW at positions 44 47 according to the Kabat numbering and Q at position 108 according to the Kabat numbering. As further described herein, Nanobodies within this group usually have a V at position 37, and can have a W, P, R or S at position 103, and preferably have a W at position 103. The GLEW group also comprises some GLEW-like sequences such as those mentioned in Table A-3 below. More generally, and without limitation, Nanobodies belonging to the GLEW-group can be defined as Nanobodies with a G at position 44 and/or with a W at position 47, in which position 46 is usually E and in which preferably position 45 is not a charged amino acid residue and not cysteine;
ii) The □KERE-group□: Nanobodies with the amino acid sequence KERE or KQRE (or another KERE-like sequence) at positions 43-46 according to the Kabat numbering and Q or L at position 108 according to the Kabat numbering. As further described herein, Nanobodies within this group usually have a F at position 37, an L or F at position 47; and can have a W, P, R or S at position 103, and preferably have a W at position 103. More generally, and without limitation, Nanobodies belonging to the KERE-group can be defined as Nanobodies with a K, Q or R at position 44 (usually K) in which position 45 is a charged amino acid residue or cysteine, and position 47 is as further defined herein;
iii) The □103 P, R, S-group□: Nanobodies with a P, R or S at position 103. These

Nanobodies can have either the amino acid sequence GLEW at positions 44-47 according to the Kabat numbering or the amino acid sequence KERE or KQRE at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

Also, where appropriate, Nanobodies may belong to (i.e. have characteristics of) two or more of these classes. For example, one specifically preferred group of Nanobodies has GLEW or a GLEW-like sequence at positions 44-47; P, R or S (and in particular R) at position 103; and Q at position 108 (which may be humanized to L).

More generally, it should be noted that the definitions referred to above describe and apply to Nanobodies in the form of a native (i.e. non-humanized) V_{HH} sequence, and that humanized variants of these Nanobodies may contain other amino acid residues than those indicated above (i.e. one or more humanizing substitutions as defined herein). For example, and without limitation, in some humanized Nanobodies of the GLEW-group or the 103 P, R, S-group, Q at position 108 may be humanized to 108 L. As already mentioned herein, other humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring VHH sequence with the corresponding framework sequence of one or more closely related human V H sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Thus, in another preferred, but non-limiting aspect, a Nanobody of the invention may be a Nanobody belonging to the GLEW-group (as defined herein), and in which CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may be a Nanobody belonging to the KERE-group (as defined herein), and CDR1, CDR2 and CDR3 are as defined herein, and are preferably as defined according to one of the preferred aspects herein, and are more preferably as defined according to one of the more preferred aspects herein.

Thus, in another preferred, but non-limiting aspect, a Nanobody of the invention may be a Nanobody belonging to the 103 P, R, S-group (as defined herein), and in which CDR1, CDR2 and CDR3 are as defined herein, and are preferably as defined according to one of the preferred aspects herein, and are more preferably as defined according to one of the more preferred aspects herein.

Also, more generally and in addition to the 108Q, 43E/44R and 103 P,R,S residues mentioned above, the Nanobodies of the invention can contain, at one or more positions that in a conventional V_{H} domain would form (part of) the V_{H}/V_{L} interface, one or more amino acid residues that are more highly charged than the amino acid residues that naturally occur at the same position(s) in the corresponding naturally occurring V_{H} sequence, and in particular one or more charged amino acid residues (as mentioned in Table A-2). Such substitutions include, but are not limited to, the GLEW-like sequences mentioned in Table A-3 below; as well as the substitutions that are described in the International Application WO 00/29004 for so-called □microbodies□, e.g. so as to obtain a Nanobody with Q at position 108 in combination with KLEW at positions 44-47. Other possible substitutions at these positions will be clear to the skilled person based upon the disclosure herein.

In one aspect of the Nanobodies of the invention, the amino acid residue at position 83 is chosen from the group consisting of L, M, S, V and W; and is preferably L.

Also, in one aspect of the Nanobodies of the invention, the amino acid residue at position 83 is chosen from the group consisting of R, K, N, E, G, I, T and Q; and is most preferably either K or E (for Nanobodies corresponding to naturally occurring V_{HH} domains) or R (for □humanized□ Nanobodies, as described herein). The amino acid resi [symbol] pos 84 is chosen from the group consisting of P, A, R, S, D T, and V in one aspect, and is most preferably P (for Nanobodies corresponding to naturally occurring V_{HH} domains) or R (for □humanized□ Nanobodies, as described herein).

Furthermore, in one aspect of the Nanobodies of the invention, the amino acid residue at position 104 is chosen from the group consisting of G and D; and is most preferably G.

Collectively, the amino acid residues at positions 11, 37, 44, 45, 47, 83, 84, 103, 104 and 108, which in the Nanobodies are as mentioned above, will also be referred to herein as the □Hallmark Residues□. The Hallmark Residues and the amino acid residues at the corresponding positions of the most closely related human V_{H} domain, V_{H}3, are summarized in Table A-3.

Some especially preferred but non-limiting combinations of these Hallmark Residues as occur in naturally occurring VHH domains are mentioned in Table A-4. For comparison, the corresponding amino acid residues of the human V_{H}3 called DP-47 have been indicated in italics.

TABLE A-3
Hallmark Residues in Nanobodies

| Position | Human V_{H}3 | Hallmark Residues |
|---|---|---|
| 11 | L, V; | L, M, S, V, W; preferably L |
| | predominantly L | |
| 37 | V, I, F; usually V | F⁽¹⁾, Y, H, I, L or V, preferably |
| | | F⁽¹⁾ or Y |
| 44⁽⁸⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾. |
| 45⁽⁸⁾ | L | L⁽²⁾, R⁽³⁾, C, I, L, P, Q, V; |
| | | preferably L⁽²⁾ or R⁽³⁾ |
| 47⁽⁸⁾ | W, Y | W⁽²⁾, L⁽¹⁾ or F⁽¹⁾, A, G, I, M, R, S, V |
| | | or Y; preferably W⁽²⁾, L⁽¹⁾, F⁽¹⁾ or R |
| 83 | R or K; usually R | R, K⁽⁵⁾, N, E⁽⁵⁾, G, I, M, Q or T; |
| | | preferably K or R; most preferably K |
| 84 | A, T, D; | P⁽⁵⁾, A, L, R, S, T, D, V; preferably P |
| | predominantly A | |
| 103 | W | W⁽⁴⁾, P⁽⁶⁾, R⁽⁶⁾, S; preferably W |
| 104 | G | G or D; preferably G |
| 108 | L, M or T; | Q, L⁽⁷⁾ or R; preferably Q or L⁽⁷⁾ |
| | predominantly L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at position 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

TABLE A-4
Some preferred but non-limiting combinations of
Hallmark Residues in naturally occurring Nanobodies.
For humanization of these combinations,
reference is made to the specification.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| □ KERE□ group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| □ GLEW□ group | L | V | G | L | W | K | S | W | G | Q |
| | M | V | G | L | W | K | P | R | G | Q |

In the Nanobodies, each amino acid residue at any other position than the Hallmark Residues can be any amino acid residue that naturally occurs at the corresponding position (according to the Kabat numbering) of a naturally occurring V_{HH} domain.

Such amino acid residues will be clear to the skilled person. Tables A-5 to A-8 mention some non-limiting residues that can be present at each position (according to the Kabat numbering) of the FR1, FR2, FR3 and FR4 of naturally occurring V_{HH} domains. For each position, the amino acid residue that most frequently occurs at each position of a naturally occurring V_{HH} domain (and which is the most preferred amino acid residue for said position in a Nanobody) is indicated in bold; and other preferred amino acid residues for each position have been underlined (note: the number of amino acid residues that are found at positions 26-30 of naturally occurring V_{HH} domains supports the hypothesis underlying the numbering by Chothia (supra) that the residues at these positions already form part of CDR1.)

In Tables A-5 □A-8, some of the non-limiting residues that can be present at each position of a human V_{H}3 domain have also been mentioned. Again, for each position, the amino acid residue that most frequently occurs at each position of a naturally occurring human V_{H}3 domain is indicated in bold; and other preferred amino acid residues have been underlined.

For reference only, Tables A-5-A-8 also contain data on the V_{HH} entropy (□V_{HH}Ent. □) and V_{HH} variability (□V_{HH} Var.□) at each amino acid position for a representative sample of 1118 VHH sequences (data kindly provided by David Lutje Hulsing and Prof. Theo Verrips of Utrecht University). The values for the V_{HH} entropy and the V_{HH} variability provide a measure for the variability and degree of conservation of amino acid residues between the 1118 V_{HH} sequences analyzed: low values (i.e. <1, such as <0.5) indicate that an amino acid residue is highly conserved between the V_{HH} sequences (i.e. little variability). For example, the G at position 8 and the G at position 9 have values for the V_{HH} entropy of 0.1 and 0 respectively, indicating that these residues are highly conserved and have little variability (and in case of position 9 is G in all 1118 sequences analysed), whereas for residues that form part of the CDR□s generally values of 1.5 or more are found (data not shown). Note that (1) the amino acid residues listed in the second column of Tables A-5-A-8 are based on a bigger sample than the 1118 V_{HH} sequences that were analysed for determining the V_{HH} entropy and V_{HH} variability referred to in the last two columns; and (2) the data represented below support the hypothesis that the amino acid residues at positions 27-30 and maybe even also at positions 93 and 94 already form part of the CDR□s (although the invention is not limited to any specific hypothesis or explanation, and as mentioned above, herein the numbering according to Kabat is used). For a general explanation of sequence entropy, sequence variability and the methodology for determining the same, see Oliveira et al., PROTEINS: Structure, Function and Genetics, 52: 544-552 (2003).

TABLE A-5
Non-limiting examples of amino acid residues in FR1
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, V, W; preferably L | | 0.8 | 2 |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, R, F, Y | 1.7 | 11 |
| 29 | F, V | F, L, D, S, I, G, V, A | 1.9 | 11 |
| 30 | S, D, G | N, S, E, G, A, D, M, T | 1.8 | 11 |

TABLE A-6
Non-limiting examples of amino acid residues in FR2
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y | | 1.1 | 6 |
| | or V, preferably F⁽¹⁾ or Y | | | |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, D, Q, | | 1.3 | 5 |
| | R, S, L; preferably G⁽²⁾, E⁽³⁾ or | | | |
| | Q; most preferably G⁽²⁾ or E⁽³⁾. | | | |
| 45 | Hallmark residue: L⁽²⁾, R⁽³⁾, C, I, L, P, Q, V; | | 0.6 | 4 |
| | preferably L⁽²⁾ or R⁽³⁾ | | | |
| 46 | E, V | E, D, K, Q, V | 0.4 | 2 |
| 47 | Hallmark residue: W⁽²⁾, L⁽¹⁾ or F⁽¹⁾, A, G, I, | | 1.9 | 9 |
| | M, R, S, V or Y; preferably W⁽²⁾, L⁽¹⁾, F⁽¹⁾ or R | | | |
| 48 | V | V, I, L | 0.4 | 3 |
| 49 | S, A, G | A, S, G, T, V | 0.8 | 3 |

TABLE A-7
Non-limiting examples of amino acid residues in FR3
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, M, Q or T; | | 0.9 | 7 |
| | preferably K or R; most preferably K | | | |
| 84 | Hallmark residue: P⁽⁵⁾, A, D, L, R, S, T, V; | | 0.7 | 6 |
| | preferably P | | | |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

TABLE A-8
Non-limiting examples of amino acid residues in FR4
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 103 | Hallmark residue: W⁽⁴⁾, P⁽⁶⁾, R⁽⁶⁾, S; preferably W | | 0.4 | 2 |
| 104 | Hallmark residue: G or D; preferably G | | 0.1 | 1 |
| 105 | Q, R | Q, E, K, P, R | 0.6 | 4 |
| 106 | G | G | 0.1 | 1 |
| 107 | T | T, A, I | 0.3 | 2 |
| 108 | Hallmark residue: Q, L⁽⁷⁾ or R: preferably Q or L⁽⁷⁾ | | 0.4 | 3 |
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

Thus, in another preferred, but not limiting aspect, a Nanobody of the invention can be defined as an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
i) one or more of the amino acid residues at positions 11, 37, 44, 45, 47, 83, 84, 103, 104 and 108 according to the Kabat numbering are chosen from the Hallmark residues mentioned in Table A-3;
   and in which:
ii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In particular, a Nanobody of the invention can be an amino acid sequence with the (general) structure
FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) (preferably) one or more of the amino acid residues at positions 11, 37, 44, 45, 47, 83, 84, 103, 104 and 108 according to the Kabat numbering are chosen from the Hallmark residues mentioned in Table A-3 (it being understood that VHH sequences will contain one or more Hallmark residues; and that partially humanized Nanobodies will usually, and preferably, [still] contain one or more Hallmark residues [although it is also within the scope of the invention to provide—where suitable in accordance with the invention—partially humanized Nanobodies in which all Hallmark residues, but not one or more of the other amino acid residues, have been humanized]; and that in fully humanized Nanobodies, where suitable in accordance with the invention, all amino acid residues at the positions of the Hallmark residues will be amino acid residues that occur in a human V_{H}3 sequence. As will be clear to the skilled person based on the disclosure herein that such V_{HH} sequences, such partially humanized Nanobodies with at least one Hallmark residue, such partially humanized Nanobodies without Hallmark residues and such fully humanized Nanobodies all form aspects of this invention);
   and in which:
ii) said amino acid sequence has at least 80% amino acid identity with at least one of the amino acid sequences of SEQ ID NO□s: 1 to 22, in which for the purposes of determining the degree of amino acid identity, the amino acid residues that form the CDR sequences (indicated with X in the sequences of SEQ ID NOL s: 1 to 22) are disregarded;
   and in which:
iii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

TABLE A-9
Representative amino acid sequences for Nanobodies of the KERE, GLEW and P, R, S 103 group.

| | | |
|---|---|---|
| KERE sequence no. 1 | SEQ ID NO: 1 | EVQLVESGGGLVQPGGSLRLSCAASGIPFSXXXXXWFRQAPGKQRDSVAXXXXXRFTI |
| | | SRDNAKNTVYLQMNSLKPEDTAVYRCYFXXXXXWGQGTQVTVSS |
| KERE sequence no. 2 | SEQ ID NO: 2 | QVKLEESGGGLVQAGGSLRLSCVGSGRTFSXXXXXWFRLAPGKEREFVAXXXXXRFTI |
| | | SRDTASNRGYLHMNNLTPEDTAVYYCAAXXXXXWGQGTQVTVSS |
| KERE sequence no. 3 | SEQ ID NO: 3 | AVQLVDSGGGLVQAGDSLKLSCALTGGAFTXXXXXWFRQTPGREREFVAXXXXXRFTI |
| | | SRDNAKNMVYLRMNSLIPEDAAVYSCAAXXXXXWGQGTLVTVSS |
| KERE sequence no. 4 | SEQ ID NO: 4 | QVQLVESGGGLVEAGGSLRLSCTASESPFRXXXXXWFRQTSGQEREFVAXXXXXRFTI |
| | | SRDDAKNTVWLHGSTLKPEDTAVYYCAAXXXXXWGQGTQVTVSS |
| KERE sequence no. 5 | SEQ ID NO: 5 | AVQLVESGGGLVQGGGSLRLACAASERIFDXXXXXWYRQGPGNERELVAXXXXXRFTI |
| | | SMDYTKQTVYLHMNSLRPEDTGLYYCKIXXXXXWGQGTQVTVSS |
| KERE sequence no. 6 | SEQ ID NO: 6 | DVKFVESGGGLVQAGGSLRLSCVASGFNFDXXXXXWFRQAPGKEREEVAXXXXXRFT |
| | | ISSEKDKNSVYLQMNSLKPEDTALYICAGXXXXXWGRGTQVTVSS |
| KERE sequence no. 7 | SEQ ID NO: 7 | QVRLAESGGGLVQSGGSLRLSCVASGSTYTXXXXXWYRQYPGKQRALVAXXXXXRFT |
| | | IARDSTKDTFCLQMNNLKPEDTAVYYCYAXXXXXWGQGTQVTVSS |
| KERE sequence no. 8 | SEQ ID NO: 8 | EVQLVESGGGLVQAGGSLRLSCAASGFTSDXXXXXWFRQAPGKPREGVSXXXXXRFT |
| | | ISTDNAKNTVHLLMNRVNAEDTALYYCAVXXXXXWGRGTRVTVSS |
| KERE sequence no. 9 | SEQ ID NO: 9 | QVQLVESGGGLVQPGGSLRLSCQASGDISTXXXXXWYRQVPGKLREFVAXXXXXRFTI |
| | | SGDNAKRAIYLQMNNLKPDDTAVYYCNRXXXXXWGQGTQVTVSP |
| KERE sequence no. 10 | SEQ ID NO: 10 | QVPVVESGGGLVQAGDSLRLFCAVPSFTSTXXXXXWFRQAPGKEREFVAXXXXXRFTI |
| | | SRNATKNTLTLRMDSLKPEDTAVYYCAAXXXXXWGQGTQVTVSS |
| KERE sequence no. 11 | SEQ ID NO: 11 | EVQLVESGGGLVQAGDSLRLFCTVSGGTASXXXXXWFRQAPGEKREFVAXXXXXRFTI |
| | | ARENAGNMVYLQMNNLKPDDTALYTCAAXXXXXWGRGTQVTVSS |
| KERE sequence no. 12 | SEQ ID NO: 12 | AVQLVESGGDSVQPGDSQTLSCAASGRTNSXXXXXWFRQAPGKERVFLAXXXXXRFT |
| | | ISRDSAKNMMYLQMNNLKPQDTAVYYCAAXXXXXWGQGTQVTVSS |
| KERE sequence no. 13 | SEQ ID NO: 13 | AVQLVESGGGLVQAGGSLRLSCVVSGLTSSXXXXXWFRQTPWQERDFVAXXXXXRFT |
| | | ISRDNYKDTVLLEMNFLKPEDTAIYYCAAXXXXXWGQGTQVTVSS |
| KERE sequence no. 14 | SEQ ID NO: 14 | AVQLVESGGGLVQAGASLRLSCATSTRTLDXXXXXWFRQAPGRDREFVAXXXXXRFT |
| | | VSRDSAENTVALQMNSLKPEDTAVYYCAAXXXXXWGQGTRVTVSS |
| KERE sequence no. 15 | SEQ ID NO: 15 | QVQLVESGGGLVQPGGSLRLSCTVSRLTAHXXXXXWFRQAPGKEREAVSXXXXXRFTI |
| | | SRDYAGNTAFLQMDSLKPEDTGVYYCATXXXXXWGQGTQVTVSS |
| KERE sequence no. 16 | SEQ ID NO: 16 | EVQLVESGGELVQAGGSLKLSCTASGRNFVXXXXXWFRRAPGKEREFVAXXXXXRFT |
| | | VSRDNGKNTAYLRMNSLKPEDTADYYCAVXXXXXLGSGTQVTVSS |
| GLEW sequence no. 1 | SEQ ID NO: 17 | AVQLVESGGGLVQPGGSLRLSCAASGFTFSXXXXXWVRQAPGKVLEWVSXXXXXRFT |
| | | ISRDNAKNTLYLQMNSLKPEDTAVYYCVKXXXXXGSQGTQVTVSS |
| GLEW sequence no. 2 | SEQ ID NO: 18 | EVQLVESGGGLVQPGGSLRLSCVCVSSGCTXXXXXWVRQAPGKAEEWVSXXXXXRF |
| | | KISRDNAKKTLYLQMNSLGPEDTAMYYCQRXXXXXRGQGTQVTVSS |
| GLEW sequence no. 3 | SEQ ID NO: 19 | EVQLVESGGGLALPGGSLTLSCVFSGSTFSXXXXXWVRHTPGKAEEWVSXXXXXRFTI |
| | | SRDNAKNTLYLEMNSLSPEDTAMYYCGRXXXXXRSKGIQVTVSS |
| P, R, S 103 | SEQ ID NO: 20 | AVQLVESGGGLVQAGGSLRLSCAASGRTFSXXXXXWFRQAPGKEREFVAXXXXXRFTI |
| sequence no. 1 | | SRDNAKNTVYLQMNSLKPEDTAVYYCAAXXXXXRGQGTQVTVSS |
| P, R, S 103 | SEQ ID NO: 21 | DVQLVESGGDLVQPGGSLRLSCAASGFSFDXXXXXWLRQTPGKGLEWVGXXXXXRFT |
| sequence no. 2 | | ISRDNAKNMLYLHLNNLKSEDTAVYYCRRXXXXXLGQGTQVTVSS |
| P, R, S 103 | SEQ ID NO: 22 | EVQLVESGGGLVQPGGSLRLSCVCVSSGCTXXXXXWVRQAPGKAEEWVSXXXXXRF |
| sequence no. 3 | | KISRDNAKKTLYLQMNSLGPEDTAMYYCQRXXXXXRGQGTQVTVSS |

The CDR□ s are indicated with XXXX

In particular, a Nanobody of the invention of the KERE group can be an amino acid sequence with the (general) structure
FR1-CDR1-FR2-CDR2-FR3-CDR3-FR4
   in which:
i) the amino acid residue at position 45 according to the Kabat numbering is a charged amino acid (as defined herein) or a cysteine residue, and position 44 is preferably an E;
   and in which:
ii) FR1 is an amino acid sequence that has at least 80% amino acid identity with at least one of the following amino acid sequences:

TABLE A-10
Representative FW1 sequences for Nanobodies of the KERE-group.

| | | |
|---|---|---|
| KERE FW1 sequence no. 1 | SEQ ID NO: 23 | QVQRVESGGGLVQAGGSLRLSCAASGRTSS |
| KERE FW1 sequence no. 2 | SEQ ID NO: 24 | QVQLVESGGGLVQTGDSLSLSCSASGRTFS |
| KERE FW1 sequence no. 3 | SEQ ID NO: 25 | QVKLEESGGGLVQAGDSLRLSCAATGRAFG |
| KERE FW1 sequence no. 4 | SEQ ID NO: 26 | AVQLVESGGGLVQPGESLGLSCVASGRDFV |
| KERE FW1 sequence no. 5 | SEQ ID NO: 27 | EVQLVESGGGLVQAGGSLRLSCEVLGRTAG |
| KERE FW1 sequence no. 6 | SEQ ID NO: 28 | QVQLVESGGGWVQPGGSLRLSCAASETILS |
| KERE FW1 sequence no. 7 | SEQ ID NO: 29 | QVQLVESGGGTVQPGGSLNLSCVASGNTFN |
| KERE FW1 sequence no. 8 | SEQ ID NO: 30 | EVQLVESGGGLAQPGGSLQLSCSAPGFTLD |
| KERE FW1 sequence no. 9 | SEQ ID NO: 31 | AQELEESGGGLVQAGGSLRLSCAASGRTFN |

and in which:
iii) FR2 is an amino acid sequence that has at least 80% amino acid identity with at least one of the following amino acid sequences:

TABLE A-11
Representative FW2 sequences for Nanobodies of the KERE-group.

| | | |
|---|---|---|
| KERE FW2 sequence no. 1 | SEQ ID NO: 41 | WFRQAPGKEREFVA |
| KERE FW2 sequence no. 2 | SEQ ID NO: 42 | WFRQTPGREREFVA |
| KERE FW2 sequence no. 3 | SEQ ID NO: 43 | WYRQAPGKQREMVA |
| KERE FW2 sequence no. 4 | SEQ ID NO: 44 | WYRQGPGKQRELVA |
| KERE FW2 sequence no. 5 | SEQ ID NO: 45 | WIRQAPGKEREGVS |
| KERE FW2 sequence no. 6 | SEQ ID NO: 46 | WFREAPGKEREGIS |
| KERE FW2 sequence no. 7 | SEQ ID NO: 47 | WYRQAPGKERDLVA |
| KERE FW2 sequence no. 8 | SEQ ID NO: 48 | WFRQAPGKQREEVS |
| KERE FW2 sequence no. 9 | SEQ ID NO: 49 | WFRQPPGKVREFVG |

and in which:
iv) FR3 is an amino acid sequence that has at least 80% amino acid identity with at least one of the following amino acid sequences:

TABLE A-12
Representative FW3 sequences for Nanobodies of the KERE-group.

| | | |
|---|---|---|
| KERE FW3 sequence no. 1 | SEQ ID NO: 50 | RFTISRDNAKNTVYLQMNSLKPEDTAVYRCYF |
| KERE FW3 sequence no. 2 | SEQ ID NO: 51 | RFAISRDNNKNTGYLQMNSLEPEDTAVYYCAA |
| KERE FW3 sequence no. 3 | SEQ ID NO: 52 | RFTVARNNAKNTVNLEMNSLKPEDTAVYYCAA |
| KERE FW3 sequence no. 4 | SEQ ID NO: 53 | RFTISRDIAKNTVDLLMNNLEPEDTAVYYCAA |
| KERE FW3 sequence no. 5 | SEQ ID NO: 54 | RLTISRDNAVDTMYLQMNSLKPEDTAVYYCAA |
| KERE FW3 sequence no. 6 | SEQ ID NO: 55 | RFTISRDNAKNTVYLQMDNVKPEDTAIYYCAA |
| KERE FW3 sequence no. 7 | SEQ ID NO: 56 | RFTISKDSGKNTVYLQMTSLKPEDTAVYYCAT |
| KERE FW3 sequence no. 8 | SEQ ID NO: 57 | RFTISRDSAKNMMYLQMNNLKPQDTAVYYCAA |
| KERE FW3 sequence no. 9 | SEQ ID NO: 58 | RFTISRENDKSTVYLQLNSLKPEDTAVYYCAA |
| KERE FW3 sequence no. 10 | SEQ ID NO: 59 | RFTISRDYAGNTAYLQMNSLKPEDTGVYYCAT |

and in which:
v) FR4 is an amino acid sequence that has at least 80% amino acid identity with at least one of the following amino acid sequences:

TABLE A-13
Representative FW4 sequences for Nanobodies of the KERE-group.

| | | |
|---|---|---|
| KERE FW4 sequence no. 1 | SEQ ID NO: 60 | WGQGTQVTVSS |
| KERE FW4 sequence no. 2 | SEQ ID NO: 61 | WGKGTLVTVSS |
| KERE FW4 sequence no. 3 | SEQ ID NO: 62 | RGQGTRVTVSS |
| KERE FW4 sequence no. 4 | SEQ ID NO: 63 | WGLGTQVTISS |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

Also, the above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

With regard to framework 1, it will be clear to the skilled person that, when an amino acid sequence as outlined above is generated by expression of a nucleotide sequence, the first four amino acid sequences (i.e. amino acid residues 1-4 according to the Kabat numbering) may often be determined by the primer(s) that have been used to generate said nucleic acid. Thus, for determining the degree of amino acid identity, the first four amino acid residues are preferably disregarded.

Also, with regard to framework 1, and although amino acid positions 27 to 30 are according to the Kabat numbering considered to be part of the framework regions (and not the CDR□s), it has been found by analysis of a database of more than 1000 V_{HH} sequences that the positions 27 to 30 have a variability (expressed in terms of VHH entropy and V_{HH} variability □see Tables A-5 to A-8) that is much greater than the variability on positions 1 to 26. Because of this, for determining the degree of amino acid identity, the amino acid residues at positions 27 to 30 are preferably also disregarded.

In view of this, a Nanobody of the KERE class may be an amino acid sequence that is comprised of four framework regions/sequences interrupted by three complementarity determining regions/sequences, in which:
i) the amino acid residue at position 45 according to the Kabat numbering is a charged amino acid (as defined herein) or a cysteine residue, and position 44 is preferably an E;
   and in which:
ii) FR1 is an amino acid sequence that, on positions 5 to 26 of the Kabat numbering, has at least 80% amino acid identity with at least one of the following amino acid sequences:

TABLE A-14
Representative FW1 sequences (amino acid residues 5 to 26) for
Nanobodies of the KERE-group.

| | | |
|---|---|---|
| KERE FW1 sequence no. 10 | SEQ ID NO: 32 | VESGGGLVQPGGSLRLSCAASG |
| KERE FW1 sequence no. 11 | SEQ ID NO: 33 | VDSGGGLVQAGDSLKLSCALTG |
| KERE FW1 sequence no. 12 | SEQ ID NO: 34 | VDSGGGLVQAGDSLRLSCAASG |
| KERE FW1 sequence no. 13 | SEQ ID NO: 35 | VDSGGGLVEAGGSLRLSCQVSE |
| KERE FW1 sequence no. 14 | SEQ ID NO: 36 | QDSGGGSVQAGGSLKLSCAASG |
| KERE FW1 sequence no. 15 | SEQ ID NO: 37 | VQSGGRLVQAGDSLRLSCAASE |
| KERE FW1 sequence no. 16 | SEQ ID NO: 38 | VESGGTLVQSGDSLKLSCASST |
| KERE FW1 sequence no. 17 | SEQ ID NQ: 39 | MESGGDSVQSGGSLTLSCVASG |
| KERE FW1 sequence no. 18 | SEQ ID NO: 40 | QASGGGLVQAGGSLRLSCSASV |

and in which:
iii) FR2, FR3 and FR4 are as mentioned herein for FR2, FR3 and FR4 of Nanobodies of the KERE-class;
   and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

A Nanobody of the GLEW class may be an amino acid sequence that is comprised of four framework regions/sequences interrupted by three complementarity determining regions/sequences, in which
i) preferably, when the Nanobody of the GLEW-class is a non-humanized Nanobody, the amino acid residue in position 108 is Q;
ii) FR1 is an amino acid sequence that has at least 80% amino acid identity with at least one of the following amino acid sequences:

TABLE A-15
Representative FW1 sequences for Nanobodies of the GLEW-group.

| | | |
|---|---|---|
| GLEW FW1 sequence no. 1 | SEQ ID NO: 64 | QVQLVESGGGLVQPGGSLRLSCAASGFTFS |
| GLEW FW1 sequence no. 2 | SEQ ID NO: 65 | EVHLVESGGGLVRPGGSLRLSCAAFGFIFK |
| GLEW FW1 sequence no. 3 | SEQ ID NO: 66 | QVKLEESGGGLAQPGGSLRLSCVASGFTFS |
| GLEW FW1 sequence no. 4 | SEQ ID NO: 67 | EVQLVESGGGLVQPGGSLRLSCVCVSSGCT |
| GLEW FW1 sequence no. 5 | SEQ ID NO: 68 | EVQLVESGGGLALPGGSLTLSCVFSGSTFS |

and in which:
iii) FR2 is an amino acid sequence that has at least 80% amino acid identity with at least one of the following amino acid sequences:

TABLE A-16
Representative FW2 sequences for Nanobodies of the GLEW-group.

| | | |
|---|---|---|
| GLEW FW2 sequence no. 1 | SEQ ID NO: 72 | WVRQAPGKVLEWVS |
| GLEW FW2 sequence no. 2 | SEQ ID NO: 73 | WVRRPPGKGLEWVS |
| GLEW FW2 sequence no. 3 | SEQ ID NO: 74 | WVRQAPGMGLEWVS |
| GLEW FW2 sequence no. 4 | SEQ ID NO: 75 | WVRQAPGKEPEWVS |
| GLEW FW2 sequence no. 5 | SEQ ID NO: 76 | WVRQAPGKDQEWVS |
| GLEW FW2 sequence no. 6 | SEQ ID NO: 77 | WVRQAPGKAEEWVS |
| GLEW FW2 sequence no. 7 | SEQ ID NO: 78 | WVRQAPGKGLEWVA |
| GLEW FW2 sequence no. 8 | SEQ ID NO: 79 | WVRQAPGRATEWVS |

and in which:
iv) FR3 is an amino acid sequence that has at least 80% amino acid identity with at least one of the following amino acid sequences:

TABLE A-17
Representative FW3 sequences for Nanobodies of the GLEW-group.

| | | |
|---|---|---|
| GLEW FW3 sequence no. 1 | SEQ ID NO: 80 | RFTISRDNAKNTLYLQMNSLKPEDTAVYYCVK |
| GLEW FW3 sequence no. 2 | SEQ ID NO: 81 | RFTISRDNARNTLYLQMDSLIPEDTALYYCAR |
| GLEW FW3 sequence no. 3 | SEQ ID NO: 82 | RFTSSRDNAKSTLYLQMNDLKPEDTALYYCAR |
| GLEW FW3 sequence no. 4 | SEQ ID NO: 83 | RFIISRDNAKNTLYLQMNSLGPEDTAMYYCQR |
| GLEW FW3 sequence no. 5 | SEQ ID NO: 84 | RFTASRDNAKNTLYLQMNSLKSEDTARYYCAR |
| GLEW FW3 sequence no. 6 | SEQ ID NO: 85 | RFTISRDNAKNTLYLQMDDLQSEDTAMYYCGR |

and in which:
v) FR4 is an amino acid sequence that has at least 80% amino acid identity with at least one of the following amino acid sequences:

TABLE A-18
Representative FW4 sequences for
Nanobodies of the GLEW-group.

| | | |
|---|---|---|
| GLEW FW4 sequence no. 1 | SEQ ID NO: 86 | GSQGTQVTVSS |
| GLEW FW4 sequence no. 2 | SEQ ID NO: 87 | LRGGTQVTVSS |
| GLEW FW4 sequence no. 3 | SEQ ID NO: 88 | RGQGTLVTVSS |
| GLEW FW4 sequence no. 4 | SEQ ID NO: 89 | RSRGIQVTVSS |
| GLEW FW4 sequence no. 5 | SEQ ID NO: 90 | WGKGTQVTVSS |
| GLEW FW4 sequence no. 6 | SEQ ID NO: 91 | WGQGTQVTVSS |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are Vim sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the GLEW class may be an amino acid sequence that is comprised of four framework regions/sequences interrupted by three complementarity determining regions/sequences, in which:
i) preferably, when the Nanobody of the GLEW-class is a non-humanized Nanobody, the amino acid residue in position 108 is Q;
   and in which:
ii) FR1 is an amino acid sequence that, on positions 5 to 26 of the Kabat numbering, has at least 80% amino acid identity with at least one of the following amino acid sequences:

TABLE A-19
Representative FW1 sequences (amino acid residues 5 to 26) for Nanobodies
of the KERE-group.

| | | |
|---|---|---|
| GLEW FW1 sequence no. 6 | SEQ ID NO: 69 | VESGGGLVQPGGSLRLSCAASG |
| GLEW FW1 sequence no. 7 | SEQ ID NO: 70 | EESGGGLAQPGGSLRLSCVASG |
| GLEW FW1 sequence no. 8 | SEQ ID NO: 71 | VESGGGLALPGGSLTLSCVFSG |

and in which:
iii) FR2, FR3 and FR4 are as mentioned herein for FR2, FR3 and FR4 of Nanobodies of the GLEW-class;
   and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein. In the above Nanobodies, one or more of the further Hallmark residues are preferably as described herein (for example, when they are V_{HH} sequences or partially humanized Nanobodies).

A Nanobody of the P, R, S 103 class may be an amino acid sequence that is comprised of four framework regions/sequences interrupted by three complementarity determining regions/sequences, in which
i) the amino acid residue at position 103 according to the Kabat numbering is different from W;
   and in which:
ii) preferably the amino acid residue at position 103 according to the Kabat numbering is P, R or S, and more preferably R;
   and in which:
iii) FR1 is an amino acid sequence that has at least 80% amino acid identity with at least one of the following amino acid sequences:

TABLE A-20
Representative FW1 sequences for Nanobodies of the P, R, S 103-group.

| | | |
|---|---|---|
| P, R, S 103 FW1 sequence no. 1 | SEQ ID NO: 92 | AVQLVESGGGLVQAGGSLRLSCAASGRTFS |
| P, R, S 103 FW1 sequence no. 2 | SEQ ID NO: 93 | QVQLQESGGGMVQPGGSLRLSCAASGFDFG |
| P, R, S 103 FW1 sequence no. 3 | SEQ ID NO: 94 | EVHLVESGGGLVRPGGSLRLSCAAFGFIFK |
| P, R, S 103 FW1 sequence no. 4 | SEQ ID NO: 95 | QVQLAESGGGLVQPGGSLKLSCAASRTIVS |
| P, R, S 103 FW1 sequence no. 5 | SEQ ID NO: 96 | QEHLVESGGGLVDIGGSLRLSCAASERIFS |
| P, R, S 103 FW1 sequence no. 6 | SEQ ID NO: 97 | QVKLEESGGGLAQPGGSLRLSCVASGFTFS |
| P, R, S 103 FW1 sequence no. 7 | SEQ ID NO: 98 | EVQLVESGGGLVQPGGSLRLSCVCVSSGCT |
| P, R, S 103 FW1 sequence no. 8 | SEQ ID NO: 99 | EVQLVESGGGLALPGGSLTLSCVFSGSTFS |

and in which
iv) FR2 is an amino acid sequence that has at least 80% amino acid identity with at least one of the following amino acid sequences:

TABLE A-21
Representative FW2 sequences for Nanobodies of the P, R, S 103-group.

| | | |
|---|---|---|
| P, R, S 103 FW2 sequence no. 1 | SEQ ID NO: 102 | WFRQAPGKEREFVA |
| P, R, S 103 FW2 sequence no. 2 | SEQ ID NO: 103 | WVRQAPGKVLEWVS |
| P, R, S 103 FW2 sequence no. 3 | SEQ ID NO: 104 | WVRRPPGKGLEWVS |
| P, R, S 103 FW2 sequence no. 4 | SEQ ID NO: 105 | WIRQAPGKEREGVS |
| P, R, S 103 FW2 sequence no. 5 | SEQ ID NO: 106 | WVRQYPGKEPEWVS |
| P, R, S 103 FW2 sequence no. 6 | SEQ ID NO: 107 | WFRQPPGKEHEFVA |
| P, R, S 103 FW2 sequence no. 7 | SEQ ID NO: 108 | WYRQAPGKRTELVA |
| P, R, S 103 FW2 sequence no. 8 | SEQ ID NO: 109 | WLRQAPGQGLEWVS |
| P, R, S 103 FW2 sequence no. 9 | SEQ ID NO: 110 | WLRQTPGKGLEWVG |
| P, R, S 103 FW2 sequence no. 10 | SEQ ID NO: 111 | WVRQAPGKAEEFVS |

and in which:
v) FR3 is an amino acid sequence that has at least 80% amino acid identity with at least one of the following amino acid sequences:

TABLE A-22
Representative FW3 sequences for Nanobodies of the P, R, S 103-group.

| | | |
|---|---|---|
| P, R, S 103 FW3 sequence no. 1 | SEQ ID NO: 112 | RFTISRDNAKNTVYLQMNSLKPEDTAVYYCAA |
| P, R, S 103 FW3 sequence no. 2 | SEQ ID NO: 113 | RFTISRDNARNTLYLQMDSLIPEDTALYYCAR |
| P, R, S 103 FW3 sequence no. 3 | SEQ ID NO: 114 | RFTISRDNAKNEMYLQMNNLKTEDTGVYWCGA |
| P, R, S 103 FW3 sequence no. 4 | SEQ ID NO: 115 | RFTISSDSNRNMIYLQMNNLKPEDTAVYYCAA |
| P, R, S 103 FW3 sequence no. 5 | SEQ ID NO: 116 | RFTISRDNAKNMLYLHLNNLKSEDTAVYYCRR |
| P, R, S 103 FW3 sequence no. 6 | SEQ ID NO: 117 | RFTISRDNAKKTVYLRLNSLNPEDTAVYSCNL |
| P, R, S 103 FW3 sequence no. 7 | SEQ ID NO: 118 | RFKISRDNAKKTLYLQMNSLGPEDTAMYYCQR |
| P, R, S 103 FW3 sequence no. 8 | SEQ ID NO: 119 | RFTVSRDNGKNTAYLRMNSLKPEDTADYYCAV |

and in which:
vi) FR4 is an amino acid sequence that has at least 80% amino acid identity with at least one of the following amino acid sequences:

TABLE A-23
Representative FW4 sequences for Nanobodies of the
P, R, S 103-group.

| | | |
|---|---|---|
| P, R, S 103 FW4 sequence no. 1 | SEQ ID NO: 120 | RGQGTQVTVSS |
| P, R, S 103 FW4 sequence no. 2 | SEQ ID NO: 121 | LRGGTQVTVSS |
| P, R, S 103 FW4 sequence no. 3 | SEQ ID NO: 122 | GNKGTLVTVSS |
| P, R, S 103 FW4 sequence no. 4 | SEQ ID NO: 123 | SSPGTQVTVSS |
| P, R, S 103 FW4 sequence no. 5 | SEQ ID NO: 124 | SSQGTLVTVSS |
| P, R, S 103 FW4 sequence no. 6 | SEQ ID NO: 125 | RSRGIQVTVSS |

and in which:
vii) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the P,R,S 103 class may be an amino acid sequence that is comprised of four framework regions/sequences interrupted by three complementarity determining regions/sequences, in which:
i) the amino acid residue at position 103 according to the Kabat numbering is different from W;
   and in which:
ii) preferably the amino acid residue at position 103 according to the Kabat numbering is P, R or S, and more preferably R;
   and in which:
iii) FR1 is an amino acid sequence that, on positions 5 to 26 of the Kabat numbering, has at least 80% amino acid identity with at least one of the following amino acid sequences:

TABLE A-24
Representative FW1 sequences (amino acid residues 5 to 26) for
Nanobodies of the P, R, S 103-group.

| | | |
|---|---|---|
| P, R, S 103 FW1 sequence no. 9 | SEQ ID NO: 100 | VESGGGLVQAGGSLRLSCAASG |
| P, R, S 103 FW1 sequence no. 10 | SEQ ID NO: 101 | AESGGGLVQPGGSLKLSCAASR |

and in which:
iv) FR2, FR3 and FR4 are as mentioned herein for FR2, FR3 and FR4 of Nanobodies of the P,R,S 103 class;
   and in which:
v) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In the above Nanobodies, one or more of the further Hallmark residues are preferably as described herein (for example, when they are V_{HH} sequences or partially humanized Nanobodies).

In another preferred, but non-limiting aspect, the invention relates to a Nanobody as described above, in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO□s 1316 to 1476, and SEQ ID NO: 1485, 1486, and 1487 (see Table 1). This degree of amino acid identity can for example be determined by determining the degree of amino acid identity (in a manner described herein) between said Nanobody and one or more of the sequences of SEQ ID NO□s 1316 to 1476, and SEQ ID NO: 1485, 1486, and 1487 (see Table 1), in which the amino acid residues that form the framework regions are disregarded. Such Nanobodies can be as further described herein.

As already mentioned herein, another preferred but non-limiting aspect of the invention relates to a Nanobody with an amino acid sequence that is chosen from the group consisting of SEQ ID NO□s 1316 to 1476, and SEQ ID NO: 1485, 1486, and 1487 (see Table 1) or from the group consisting of from amino acid sequences that have more than 80%, preferably more than 90%, more preferably more than 95%, such as 99% or more sequence identity (as defined herein) with at least one of the amino acid sequences of SEQ ID NO□s: 1316 to 1476, and SEQ ID NO: 1485, 1486, and 1487 (see Table 1).

Also, in the above Nanobodies:
i) any amino acid substitution (when it is not a humanizing substitution as defined herein) is preferably, and compared to the corresponding amino acid sequence of SEQ ID NO□s 1316 to 1476, and SEQ ID NO: 1485, 1486, and 1487 (see Table 1), a conservative amino acid substitution, (as defined herein);
   and/or:
ii) its amino acid sequence preferably contains either only amino acid substitutions, or otherwise preferably no more than 5, preferably no more than 3, and more preferably only 1 or 2 amino acid deletions or insertions, compared to the corresponding amino acid sequence of SEQ ID NOD□s 1316 to 1476, and SEQ ID NO: 1485, 1486, and 1487 (see Table 1);
   and/or
iii) the CDR□s may be CDR□s that are derived by means of affinity maturation, for example starting from the CDR□s of to the corresponding amino acid sequence of SEQ ID NO□s: 1316 to 1476, and SEQ ID NO: 1485, 1486, and 1487 (see Table 1).

Preferably, the CDR sequences and FR sequences in the Nanobodies of the invention are such that the Nanobodies of the invention (and polypeptides of the invention comprising the same):
bind to Integrins with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to Integrins with a kₒₙ-rate of between 10² M⁻¹s⁻¹ to about 10⁷ M⁻¹ s⁻¹, preferably between 10³ M⁻¹s⁻¹ and 10⁷ s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to Integrins with a k_{off}-rate between 1s⁻¹ (t_{1/2}=0.69 s) and 10⁻⁶ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, CDR sequences and FR sequences present in the Nanobodies of the invention are such that the Nanobodies of the invention will bind to Integrins with an affinity less than 500 nM, preferably less than 200 nM, more preferably less than 10 nM, such as less than 500 pM.

According to one non-limiting aspect of the invention, a Nanobody may be as defined herein, but with the proviso that it has at least □one amino acid difference□ (as defined herein) in at least one of the framework regions compared to the corresponding framework region of a naturally occurring human V_{H} domain, and in particular compared to the corresponding framework region of DP-47. More specifically, according to one non-limiting aspect of the invention, a Nanobody may be as defined herein, but with the proviso that it has at least □one amino acid difference□ (as defined herein) at least [symbol] Hallmark residues (including those at positions 108, 103 and/or 45) compared to the corresponding framework region of a naturally occurring human V_{H} domain, and in particular compared to the corresponding framework region of DP-47. Usually, a Nanobody will have at least one such amino acid difference with a naturally occurring V_{H} domain in at least one of FR2 and/or FR4, and in particular at least one of the Hallmark residues in FR2 and/or FR4 (again, including those at positions 108, 103 and/or 45).

Also, a humanized Nanobody of the invention may be as defined herein, but with the proviso that it has at least □one amino acid difference□ (as defined herein) in at least one of the framework regions compared to the corresponding framework region of a naturally occurring V_{HH} domain. More specifically, according to one non-limiting aspect of the invention, a humanized Nanobody may be as defined herein, but with the proviso that it has at least □one amino acid difference □ (as defined herein) at [symbol] mark residues (including those at positions 108, 103 and/or 45) compared to the corresponding framework region of a naturally occurring V_{HH} domain. Usually, a humanized Nanobody will have at least one such amino acid difference with a naturally occurring V_{HH} domain in at least one of FR2 and/or FR4, and in particular at least one of the Hallmark residues in FR2 and/or FR4 (again, including those at positions 108, 103 and/or 45).

As will be clear from the disclosure herein, it is also within the scope of the invention to use natural or synthetic analogs, mutants, variants, alleles, homologs and orthologs (herein collectively referred to as □analogs□) of the Nanobodies of the invention as defined herein, and in particular analogs of the Nanobodies of SEQ ID NO□s 1316 to 1476, and SEQ ID NO: 1485, 1486, and 1487 (see Table 1). Thus, according to one aspect of the invention, the term □Nanobody of the invention□ in its broadest sense also covers such analogs.

Generally, in such analogs, one or more amino acid residues may have been replaced, deleted and/or added, compared to the Nanobodies of the invention as defined herein. Such substitutions, insertions or deletions may be made in one or more of the framework regions and/or in one or more of the CDR□s. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

By means of non-limiting examples, a substitution may for example be a conservative substitution (as described herein) and/or an amino acid residue may be replaced by another amino acid residue that naturally occurs at the same position in another V_{HH} domain (see Tables A-5 to A-8 for some non-limiting examples of such substitutions), although the invention is generally not limited thereto. Thus, any one or more substitutions, deletions or insertions, or any combination thereof, that either improve the properties of the Nanobody of the invention or that at least do not detract too much from the desired properties or from the balance or combination of desired properties of the Nanobody of the invention (i.e. to the extent that the Nanobody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may for example involve introducing a limited number of possible substitutions and determining their influence on the properties of the Nanobodies thus obtained.

For example, and depending on the host organism used to express the Nanobody or polypeptide of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation (again as described herein).

As can be seen from the data on the V_{HH} entropy and V_{HH} variability given in Tables A-5 to A-8 above, some amino acid residues in the framework regions are more conserved than others. Generally, although the invention in its broadest sense is not limited thereto, any substitutions, deletions or insertions are preferably made at positions that are less conserved. Also, generally, amino acid substitutions are preferred over amino acid deletions or insertions.

The analogs are preferably such that they can bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

The analogs are preferably also such that they retain the favourable properties the Nanobodies, as described herein.

Also, according to one preferred aspect, the analogs have a degree of sequence identity of at least 70%, preferably at least 80%, more preferably at least 90%, such as at least 95% or 99% or more; and/or preferably have at most 20, preferably at most 10, even more preferably at most 5, such as 4, 3, 2 or only 1 amino acid difference (as defined herein), with one of the Nanobodies of SEQ ID NOs: 1316 to 1476, and SEQ ID NO: 1485, 1486, and 1487 (see Table 1).

Also, the framework sequences and CDR□s of the analogs are preferably such that they are in accordance with the preferred aspects defined herein. More generally, as described herein, the analogs will have (a) a Q at position 108; and/or (b) a charged amino acid or a cysteine residue at position 45 and preferably an E at position 44, and more preferably E at position 44 and R at position 45; and/or (c) P, R or S at position 103.

One preferred class of analogs of the Nanobodies of the invention comprise Nanobodies that have been humanized (i.e. compared to the sequence of a naturally occurring Nanobody of the invention). As mentioned in the background art cited herein, such humanization generally involves replacing one or more amino acid residues in the sequence of a naturally occurring V_{HH} with the amino acid residues that occur at the same position in a human V_{H} domain, such as a human V_{H}3 domain. Examples of possible humanizing substitutions or combinations of humanizing substitutions will be clear to the skilled person, for example from the Tables herein, from the possible humanizing substitutions mentioned in the background art cited herein, and/or from a comparison between the sequence of a Nanobody and the sequence of a naturally occurring human V_{H} domain.

The humanizing substitutions should be chosen such that the resulting humanized Nanobodies still retain the favourable properties of Nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may for example involve introducing a limited number of possible humanizing substitutions and determining their influence on the properties of the Nanobodies thus obtained.

Generally, as a result of humanization, the Nanobodies of the invention may become more □ [symbol] □, while still retaining the favorable properties of the Nanobodies of the invention as described herein. As a result, such humanized Nanobodies may have several advantages, such as a reduced immunogenicity, compared to the corresponding naturally occurring V_{HH} domains. Again, based on the disclosure herein and optionally after a limited degree of routine experimentation, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring V_{HH} domains on the other hand.

The Nanobodies of the invention may be suitably humanized at any framework residue(s), such as at one or more Hallmark residues (as defined herein) or at one or more other framework residues (i.e. non-Hallmark residues) or any suitable combination thereof. One preferred humanizing substitution for Nanobodies of the □P [symbol] group□ or the □ KERE group□ is Q108 into L108. Nanobodies of the □GLEW class□ may also be hurr [symbol] by a Q108 into L108 substitution, provided at least one of the other Hallmark residues contains a camelid (camelizing) substitution (as defined herein). For example, as mentioned above, one particularly preferred class of humanized Nanobodies has GLEW or a GLEW-like sequence at positions 44-47; P, R or S (and in particular R) at position 103, and an L at position 108.

The humanized and other analogs, and nucleic acid sequences encoding the same, can be provided in any manner known per se, for example using one or more of the techniques mentioned on pages 103 and 104 of WO 08/020,079.

As mentioned there, it will be also be clear to the skilled person that the Nanobodies of the invention (including their analogs) can be designed and/or prepared starting from human V_{H} sequences (i.e. amino acid sequences or the corresponding nucleotide sequences), such as for example from human V_{H}3 sequences such as DP-47, DP-51 or DP-29, i.e. by introducing one or more camelizing substitutions (i.e. changing one or more amino acid residues in the amino acid sequence of said human V_{H} domain into the amino acid residues that occur at the corresponding position in a V_{HH} domain), so as to provide the sequence of a Nanobody of the invention and/or so as to confer the favourable properties of a Nanobody to the sequence thus obtained. Again, this can generally be performed using the various methods and techniques referred to in the previous paragraph, using an amino acid sequence and/or nucleotide sequence for a human V_{H} domain as a starting point.

Some preferred, but non-limiting camelizing substitutions can be derived from Tables A-5□A-8. It will also be clear that camelizing substitutions at one or more of the Hallmark residues will generally have a greater influence on the desired properties than substitutions at one or more of the other amino acid positions, although both and any suitable combination thereof are included within the scope of the invention. For example, it is possible to introduce one or more camelizing substitutions that already confer at least some the desired properties, and then to introduce further camelizing substitutions that either further improve said properties and/or confer additional favourable properties. Again, the skilled person will generally be able to determine and select suitable camelizing substitutions or suitable combinations of camelizing substitutions, based on the disclosure herein and optionally after a limited degree of routine experimentation, which may for example involve introducing a limited number of possible camelizing substitutions and determining whether the favourable properties of Nanobodies are obtained or improved (i.e. compared to the original V_{H} domain). Generally, however, such camelizing substitutions are preferably such that the resulting an amino acid sequence at least contains (a) a Q at position 108; and/or (b) a charged amino acid or a cysteine residue at position 45 and preferably also an E at position 44, and more preferably E at position 44 and R at position 45; and/or (c) P, R or S at position 103; and optionally one or more further camelizing substitutions. More preferably, the camelizing substitutions are such that they result in a Nanobody of the invention and/or in an analog thereof (as defined herein), such as in a humanized analog and/or preferably in an analog that is as defined in the preceding paragraphs.

As will also be clear from the disclosure herein, it is also within the scope of the invention to use parts or fragments, or combinations of two or more parts or fragments, of the Nanobodies of the invention as defined herein, and in particular parts or fragments of the Nanobodies of SEQ ID NO□s: 1316 to 1476, and SEQ ID NO: 1485, 1486, and 1487 (see Table 1). Thus, according to one aspect of the invention, the term □Nanobody of the invention□in its broadest [symbol] covers such parts or fragments.

Generally, such parts or fragments of the Nanobodies of the invention (including analogs thereof) have amino acid sequences in which, compared to the amino acid sequence of the corresponding full length Nanobody of the invention (or analog thereof), one or more of the amino acid residues at the N-terminal end, one or more amino acid residues at the C-terminal end, one or more contiguous internal amino acid residues, or any combination thereof, have been deleted and/or removed.

The parts or fragments are preferably such that they can bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

Any part or fragment is preferably such that it comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody of the invention.

Also, any part or fragment is such preferably that it comprises at least one of CDR1, CDR2 and/or CDR3 or at least part thereof (and in particular at least CDR3 or at least part thereof). More preferably, any part or fragment is such that it comprises at least one of the CDR□s (and preferably at least CDR3 part thereof) and at least one other CDR (i.e. CDR1 or CDR2) or at least part thereof, preferably connected by suitable framework sequence(s) or at least part thereof. More preferably, any part or fragment is such that it comprises at least one of the CDR□s (and preferably at least CDR3 or part thereof) and at least part of the two remaining CDR□s, again preferably connected by suitable framework sequence(s) or at least part thereof.

According to another particularly preferred, but non-limiting aspect, such a part or fragment comprises at least CDR3, such as FR3, CDR3 and FR4 of the corresponding full length Nanobody of the invention, i.e. as for example described in the International application WO 03/050531 (Lasters et al.).

As already mentioned above, it is also possible to combine two or more of such parts or fragments (i.e. from the same or different Nanobodies of the invention), i.e. to provide an analog (as defined herein) and/or to provide further parts or fragments (as defined herein) of a Nanobody of the invention. It is for example also possible to combine one or more parts or fragments of a Nanobody of the invention with one or more parts or fragments of a human V_{H} domain.

According to one preferred aspect, the parts or fragments have a degree of sequence identity of at least 50%, preferably at least 60%, more preferably at least 70%, even more preferably at least 80%, such as at least 90%, 95% or 99% or more with one of the Nanobodies of SEQ ID NOs 1316 to 1476, and SEQ ID NO: 1485, 1486, and 1487 (see Table 1).

The parts and fragments, and nucleic acid sequences encoding the same, can be provided and optionally combined in any manner known per se. For example, such parts or fragments can be obtained by inserting a stop codon in a nucleic acid that encodes a full-sized Nanobody of the invention, and then expressing the nucleic acid thus obtained in a manner known per se (e.g. as described herein). Alternatively, nucleic acids encoding such parts or fragments can be obtained by suitably restricting a nucleic acid that encodes a full-sized Nanobody of the invention or by synthesizing such a nucleic acid in a manner known per se. Parts or fragments may also be provided using techniques for peptide synthesis known per se.

The invention in its broadest sense also comprises derivatives of the Nanobodies of the invention. Such derivatives can generally be obtained by modification, and in particular by chemical and/or biological (e.g. enzymatical) modification, of the Nanobodies of the invention and/or of one or more of the amino acid residues that form the Nanobodies of the invention.

Examples of such modifications, as well as examples of amino acid residues within the Nanobody sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the Nanobody of the invention, and in particular of one or more functional groups, residues or moieties that confer one or more desired properties or functionalities to the Nanobody of the invention. Example of such functional groups will be clear to the skilled person.

For example, such modification may comprise the introduction (e.g. by covalent binding or in any other suitable manner) of one or more functional groups that increase the half-life, the solubility and/or the absorption of the Nanobody of the invention, that reduce the immunogenicity and/or the toxicity of the Nanobody of the invention, that eliminate or attenuate any undesirable side effects of the Nanobody of the invention, and/or that confer other advantageous properties to and/or reduce the undesired properties of the Nanobodies and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFv□s and single domain antibodies), for which reference is for example made to Remington's Pharmaceutical Sciences, 16th ed., Mack Publishing Co., Easton, Pa. (1980). Such functional groups may for example be linked directly (for example covalently) to a Nanobody of the invention, or optionally via a suitable linker or spacer, as will again be clear to the skilled person.

One of the most widely used techniques for increasing the half-life and/or reducing the immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFv s); reference is made to for example Chapman, Nat. Biotcchnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in a Nanobody of the invention, a Nanobody of the invention may be modified so as to suitably introduce one or more cysteine residues for attachment of PEG, or an amino acid sequence comprising one or more cysteine residues for attachment of PEG may be fused to the N- and/or C-terminus of a Nanobody of the invention, all using techniques of protein engineering known per se to the skilled person.

Preferably, for the Nanobodies and proteins of the invention, a PEG is used with a molecular weight of more than 5000, such as more than 10,000 and less than 200,000, such as less than 100,000; for example in the range of 20,000-80,000.

Another, usually less preferred modification comprises N-linked or O-linked glycosylation, usually as part of co-translational and/or post-translational modification, depending on the host cell used for expressing the Nanobody or polypeptide of the invention.

Yet another modification may comprise the introduction of one or more detectable labels or other signal-generating groups or moieties, depending on the intended use of the labelled Nanobody. Suitable labels and techniques for attaching, using and detecting them will be clear to the skilled person, and for example include, but are not limited to, the fluorescent labels, phosphorescent labels, chemiluminescent labels, bioluminescent labels, radio-isotopes, metals, metal chelates, metallic cations, chromophores and enzymes, such as those mentioned on page 109 of WO 08/020,079—Other suitable labels will be clear to the skilled person, and for example include moieties that can be detected using NMR or ESR spectroscopy.

Such labelled Nanobodies and polypeptides of the invention may for example be used for in vitro, in vivo or in situ assays (including immunoassays known per se such as ELISA, RIA, EIA and other □sandwich assays□, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the Nanobody of the invention to another protein, polypeptide or chemical compound that is bound to the other half of the binding pair, i.e. through formation of the binding pair. For example, a Nanobody of the invention may be conjugated to biotin, and linked to another protein, polypeptide, compound or carrier conjugated to avidin or streptavidin. For example, such a conjugated Nanobody may be used as a reporter, for example in a diagnostic system where a detectable signal-producing agent is conjugated to avidin or streptavidin. Such binding pairs may for example also be used to bind the Nanobody of the invention to a carrier, including carriers suitable for pharmaceutical purposes. One non-limiting example are the liposomal formulations described by Cao and Suresh, Journal of Drug Targetting, 8, 4, 257 (2000). Such binding pairs may also be used to link a therapeutically active agent to the Nanobody of the invention.

For some applications, in particular for those applications in which it is intended to kill a cell that expresses the target against which the Nanobodies of the invention are directed (e.g. in the treatment of cancer), or to reduce or slow the growth and/or proliferation such a cell, the Nanobodies of the invention may also be linked to a toxin or to a toxic residue or moiety. Examples of toxic moieties, compounds or residues which can be linked to a Nanobody of the invention to provide □for example □a cytotoxic compound will be clear to the skilled person and can for example be found in the prior art cited above and/or in the further description herein. One example is the so-called ADEPT™ technology described in WO 03/055527.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

Preferably, the derivatives are such that they bind to Integrins with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

As mentioned above, the invention also relates to proteins or polypeptides that essentially consist of or comprise at least one Nanobody of the invention. By □essentially consist of□ is meant that the amino acid sequence of the polypeptide of the invention either is exactly the same as the amino acid sequence of a Nanobody of the invention or corresponds to the amino acid sequence of a Nanobody of the invention which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the Nanobody.

Said amino acid residues may or may not change, alter or otherwise influence the (biological) properties of the Nanobody and may or may not add further functionality to the Nanobody. For example, such amino acid residues:
can comprise an N-terminal Met residue, for example as result of expression in a heterologous host cell or host organism.
may form a signal sequence or leader sequence that directs secretion of the Nanobody from a host cell upon synthesis. Suitable secretory leader peptides will be clear to the skilled person, and may be as further described herein. Usually, such a leader sequence will be linked to the N-terminus of the Nanobody, although the invention in its broadest sense is not limited thereto;
may form a sequence or signal that allows the Nanobody to be directed towards and/or to penetrate or enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such amino acid sequences will be clear to the skilled person and include those mentioned in paragraph c) on page 112 of WO 08/020,079
may form a □tag□, for example an amino [symbol] residue that allows or facilitates the purification of the Nanobody, for example using affinity techniques directed against said sequence or residue. Thereafter, said sequence or residue may be removed (e.g. by chemical or enzymatical cleavage) to provide the Nanobody sequence (for this purpose, the tag may optionally be linked to the Nanobody sequence via a cleavable linker sequence or contain a cleavable motif). Some preferred, but non-limiting examples of such residues are multiple histidine residues, glutathione residues and a myc-tag (see for example SEQ ID NO:31 of WO 06/12282).
may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the Nanobodies of the invention.

According to another aspect, a polypeptide of the invention comprises a Nanobody of the invention, which is fused at its amino terminal end, at its carboxy terminal end, or both at its amino terminal end and at its carboxy terminal end to at least one further amino acid sequence, i.e. so as to provide a fusion protein comprising said Nanobody of the invention and the one or more further amino acid sequences. Such a fusion will also be referred to herein as a □Nanobody fusion □.

The one or more further amino acid sequence may be any suitable and/or desired amino acid sequences. The further amino acid sequences may or may not change, alter or otherwise influence the (biological) properties of the Nanobody, and may or may not add further functionality to the Nanobody or the polypeptide of the invention. Preferably, the further amino acid sequence is such that it confers one or more desired properties or functionalities to the Nanobody or the polypeptide of the invention.

For example, the further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody of the invention is directed, or a different protein, polypeptide, antigen, antigenic determinant or epitope).

Example of such amino acid sequences will be clear to the skilled person, and may generally comprise all amino acid sequences that are used in peptide fusions based on conventional antibodies and fragments thereof (including but not limited to ScFv□s and single domain antibodies). Reference is for example made to the review by Holliger and Hudson, Nature Biotechnology, 23, 9, 1126-1136 (2005).

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides of the invention, compared to the Nanobody of the invention per se. Some non-limiting examples of such amino acid sequences are serum proteins, such as humanserum albumin (see for example WO 00/27435) or haptenic molecules (for example haptens that are recognized by circulating antibodies, see for example WO 98/22141).

In particular, it has been described in the art that linking fragments of immunoglobulins (such as V_{H} domains) to serum albumin or to fragments thereof can be used to increase the half-life. Reference is for made to WO 00/27435 and WO 01/077137). According to the invention, the Nanobody of the invention is preferably either directly linked to serum albumin (or to a suitable fragment thereof) or via a suitable linker, and in particular via a suitable peptide linked so that the polypeptide of the invention can be expressed as a genetic fusion (protein). According to one specific aspect, the Nanobody of the invention may be linked to a fragment of serum albumin that at least comprises the domain III of serum albumin or part thereof. Reference is for example made to WO 07/112,940 of Ablynx N.V

Alternatively, the further amino acid sequence may provide a second binding site or binding unit that is directed against a serum protein (such as, for example, human serum albumin or another serum protein such as IgG), so as to provide increased half-life in serum. Such amino acid sequences for example include the Nanobodies described below, as well as the small peptides and binding proteins described in WO 91/01743, WO 01/45746 and WO 02/076489 and the dAb□s described in WO 03/002609 and WO 04/003019. Reference is also made to Harmsen et al., Vaccine, 23 (41); 4926-42, 2005, as well as to EP 0 368 684, as well as to the following the U.S. provisional applications 60/843,349 (see also PCT/EP2007/059475), 60/850,774 (see also PCT/EP2007/060849), 60/850,775 (see also PCT/EP2007/060850) by Ablynx N.V. mentioned herein and US provisional application of Ablynx N.V. entitled “Peptides capable of binding to serum proteins □ filed on Dec. 5, 2006 ((see also PCT/EP2007/063348).

Such amino acid sequences may in particular be directed against serum albumin (and more in particular human serum albumin) and/or against IgG (and more in particular human IgG). For example, such amino acid sequences may be amino acid sequences that are directed against (human) serum albumin and amino acid sequences that can bind to amino acid residues on (human) serum albumin that are not involved in binding of serum albumin to FcRn (see for example WO 06/0122787) and/or amino acid sequences that are capable of binding to amino acid residues on serum albumin that do not form part of domain III of serum albumin (see again for example WO 06/0122787); amino acid sequences that have or can provide an increased half-life (see for example WO 08/028,977 by Ablynx N.V.); amino acid sequences against human serum albumin that are cross-reactive with scrum albumin from at least one species of mammal, and in particular with at least one species of primate (such as, without limitation, monkeys from the genus Macaca (such as, and in particular, cynomologus monkeys (Macaca fascicularis) and/or rhesus monkeys (Macaca mulatta)) and baboon (Papio ursinus), reference is again made to the U.S. provisional application 60/843,349 and PCT/EP2007/059475); amino acid sequences that can bind to serum albumin in a pH independent manner (see for example the U.S. provisional application 60/850,774 by Ablynx N.V. entitled □Amino acid sequences that bind to serum proteins in a manner that is essentially independent of the pH, compounds comprising the same, and uses thereof□, filed on Oct. 11, 2006; see also and PCT/EP2007/059475) and/or amino acid sequences that are conditional binders (see for example the U.S. provisional application 60/850,775 by Ablynx N.V. entitled □Amino acid sequences that bind to a desired molecule in a conditional manner□, filed on Oct. 11, 2006; see also PCT/EP2007/060850).

According to another aspect, the one or more further amino acid sequences may comprise one or more parts, fragments or domains of conventional 4-chain antibodies (and in particular human antibodies) and/or of heavy chain antibodies. For example, although usually less preferred, a Nanobody of the invention may be linked to a conventional (preferably human) V_{H} or V_{L}, domain or to a natural or synthetic analog of a V_{H} or V_{L} domain, again optionally via a linker sequence (including but not limited to other (single) domain antibodies, such as the dAb□s described by Ward et al.).

The at least one Nanobody may also be linked to one or more (preferably human) C_{H}1, C_{H}2 and/or C_{H}3 domains, optionally via a linker sequence. For instance, a Nanobody linked to a suitable C_{H}1 domain could for example be used—together with suitable light chains—to generate antibody fragments/structures analogous to conventional Fab fragments or F(ab□₂ fragments, but in which one or (in case of an F(ab □₂ fragment) one or both of the conventional V_{H} domains have been replaced by a Nanobody of the invention. Also, two Nanobodies could be linked to a C_{H}3 domain (optionally via a linker) to provide a construct with increased half-life in vivo.

According to one specific aspect of a polypeptide of the invention, one or more Nanobodies of the invention may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more C_{H}2 and/or C_{H}3 domains of an antibody, such as from a heavy chain antibody (as described herein) and more preferably from a conventional human 4-chain antibody; and/or may form (part of) and Fc region, for example from IgG (e.g. from IgG1, IgG2, IgG3 or IgG4), from IgE or from another human Ig such as IgA, IgD or IgM. For example, WO 94/04678 describes heavy chain antibodies comprising a Camelid V_{HH} domain or a humanized derivative thereof (i.e. a Nanobody), in which the Camelidae C_{H}2 and/or C_{H}3 domain have been replaced by human C_{H}2 and C_{H}3 domains, so as to provide an immunoglobulin that consists of 2 heavy chains each comprising a Nanobody and human C_{H}2 and C_{H}3 domains (but no C_{H}1 domain), which immunoglobulin has the effector function provided by the C_{H}2 and C_{H}3 domains and which immunoglobulin can function without the presence of any light chains. Other amino acid sequences that can be suitably linked to the Nanobodies of the invention so as to provide an effector function will be clear to the skilled person, and may be chosen on the basis of the desired effector function(s). Reference is for example made to WO 04/058820, WO 99/42077, WO 02/056910 and WO 05/017148, as well as the review by Holliger and Hudson, supra; and to the non-prepublished US provisional application by Ablynx N.V. entitled □Constructs comprising single variable domains and an Fc portion derived from IgE□ which has a filing date of Dec. 4, 2007. Coupling of a Nanobody of the invention to an Fc portion may also lead to an increased half-life, compared to the corresponding Nanobody of the invention. For some applications, the use of an Fc portion and/or of constant domains (i.e. C_{H}2 and/or C_{H}3 domains) that confer increased half-life without any biologically significant effector function may also be suitable or even preferred. Other suitable constructs comprising one or more Nanobodies and one or more constant domains with increased half-life in vivo will be clear to the skilled person, and may for example comprise two Nanobodies linked to a C_{H}3 domain, optionally via a linker sequence. Generally, any fusion protein or derivatives with increased half-life will preferably have a molecular weight of more than 50 kD, the cut-off value for renal absorption.

In another one specific, but non-limiting, aspect, in order to form a polypeptide of the invention, one or more amino acid sequences of the invention may be linked (optionally via a suitable linker or hinge region) to naturally occurring, synthetic or semisynthetic constant domains (or analogs, variants, mutants, parts or fragments thereof) that have a reduced (or essentially no) tendency to self-associate into dimers (i.e. compared to constant domains that naturally occur in conventional 4-chain antibodies). Such monomeric (i.e. not self-associating) Fc chain variants, or fragments thereof, will be clear to the skilled person. For example, Helm et al., J Biol Chem 1996 271 7494, describe monomeric Fee chain variants that can be used in the polypeptide chains of the invention.

Also, such monomeric Fc chain variants are preferably such that they are still capable of binding to the complement or the relevant Fc receptor(s) (depending on the Fc portion from which they are derived), and/or such that they still have some or all of the effector functions of the Fc portion from which they are derived (or at a reduced level still suitable for the intended use). Alternatively, in such a polypeptide chain of the invention, the monomeric Fc chain may be used to confer increased half-life upon the polypeptide chain, in which case the monomeric Fc chain may also have no or essentially no effector functions.

Bivalent/multivalent, bispecific/multispecific or biparatopic/multiparatopic polypeptides of the invention may also be linked to Fc portions, in order to provide polypeptide constructs of the type that is described in the non-prepublished U.S. provisional application US 61/005,331 entitled □immunoglobulin construct □ filed Dec. 4, 2007.

The further amino acid sequences may also form a signal sequence or leader sequence that directs secretion of the Nanobody or the polypeptide of the invention from a host cell upon synthesis (for example to provide a pre-, pro- or prepro-form of the polypeptide of the invention, depending on the host cell used to express the polypeptide of the invention).

The further amino acid sequence may also form a sequence or signal that allows the Nanobody or polypeptide of the invention to be directed towards and/or to penetrate or enter into specific organs, tissues, cells, or parts or compartments of cells, and/or that allows the Nanobody or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, those mentioned on page 118 of WO 08/020,079. For some applications, in particular for those applications in which it is intended to kill a cell that expresses the target against which the Nanobodies of the invention are directed (e.g. in the treatment of cancer), or to reduce or slow the growth and/or proliferation of such a cell, the Nanobodies of the invention may also be linked to a (cyto)toxic protein or polypeptide. Examples of such toxic proteins and polypeptides which can be linked to a Nanobody of the invention to provide □for example□ a cytotoxic polypeptide of the invention will be clear to the skilled person and can for example be found in the prior art cited above and/or in the further description herein. One example is the so-called ADEPT™ technology described in WO 03/055527.

According to one preferred, but non-limiting aspect, said one or more further amino acid sequences comprise at least one further Nanobody, so as to provide a polypeptide of the invention that comprises at least two, such as three, four, five or more Nanobodies, in which said Nanobodies may optionally be linked via one or more linker sequences (as defined herein). As described on pages 119 and 120 of WO 08/020,079, polypeptides of the invention that comprise two or more Nanobodies, of which at least one is a Nanobody of the invention, will also be referred to herein as □multivalent□ polypeptides of the invention, and the Nanobodies present in such polypeptides will also be referred to herein as being in a □multivalent format□. For example □□ [symbol] trivalent□ polypeptides of the invention may be as further described on pages 119 and 120 of WO 08/020,079.

Polypeptides of the invention that contain at least two Nanobodies, in which at least one Nanobody is directed against a first antigen (i.e. against Integrins,) and at least one Nanobody is directed against a second antigen (i.e. different from Integrins,), will also be referred to as □multispecific□ polypeptides of the invention, and the Nanobodies present in such polypeptides will also be referred to herein as being in a □multispecific format□. Thus, for example, a □bispecific□ polypeptide of the invention is a polypeptide that comprises at least one Nanobody directed against a first antigen (i.e. Integrins,) and at least one further Nanobody directed against a second antigen (i.e. different from Integrins,), whereas a □trispecific□ polypeptide of the invention is a polypeptide that comprises at least one Nanobody directed against a first antigen (i.e. Integrins,), at least one further Nanobody directed against a second antigen (i.e. different from Integrins,) and at least one further Nanobody directed against a third antigen (i.e. different from both Integrins, and the second antigen); etc.

Accordingly, in its simplest form, a bispecific polypeptide of the invention is a bivalent polypeptide of the invention (as defined herein), comprising a first Nanobody directed against Integrins, and a second Nanobody directed against a second antigen, in which said first and second Nanobody may optionally be linked via a linker sequence (as defined herein); whereas a trispecific polypeptide of the invention in its simplest form is a trivalent polypeptide of the invention (as defined herein), comprising a first Nanobody directed against Integrins, a second Nanobody directed against a second antigen and a third Nanobody directed against a third antigen, in which said first, second and third Nanobody may optionally be linked via one or more, and in particular one and more, in particular two, linker sequences.

However, as will be clear from the description hereinabove, the invention is not limited thereto, in the sense that a multispecific polypeptide of the invention may comprise at least one Nanobody against Integrins, and any number of Nanobodies directed against one or more antigens different from Integrins.

Furthermore, although it is encompassed within the scope of the invention that the specific order or arrangement of the various Nanobodies in the polypeptides of the invention may have some influence on the properties of the final polypeptide of the invention (including but not limited to the affinity, specificity or avidity for Integrins, or against the one or more other antigens), said order or arrangement is usually not critical and may be suitably chosen by the skilled person, optionally after some limited routine experiments based on the disclosure herein. Thus, when reference is made to a specific multivalent or multispccific polypeptide of the invention, it should be noted that this encompasses any order or arrangements of the relevant Nanobodies, unless explicitly indicated otherwise.

Finally, it is also within the scope of the invention that the polypeptides of the invention contain two or more Nanobodies and one or more further amino acid sequences (as mentioned herein).

For multivalent and multispecific polypeptides containing one or more V_{HH} domains and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 96/34103 and WO 99/23221. Some other examples of some specific multispecific and/or multivalent polypeptide of the invention can be found in the applications by Ablynx N.V. referred to herein.

One preferred, but non-limiting example of a multispecific polypeptide of the invention comprises at least one Nanobody of the invention and at least one Nanobody that provides for an increased half-life. Such Nanobodies may for example be Nanobodies that are directed against a serum protein, and in particular a human serum protein, such as human serum albumin, thyroxine-binding protein, (human) transferrin, fibrinogen, an immunoglobulin such as IgG, IgE or IgM, or against one of the serum proteins listed in WO 04/003019. Of these, Nanobodies that can bind to serum albumin (and in particular human serum albumin) or to IgG (and in particular human IgG, see for example Nanobody VH-1 described in the review by Muyldermans, supra) are particularly preferred (although for example, for experiments in mice or primates, Nanobodies against or cross-reactive with mouse serum albumin (MSA) or serum albumin from said primate, respectively, can be used. However, for pharmaceutical use, Nanobodies against human serum albumin or human IgG will usually be preferred). Nanobodies that provide for increased half-life and that can be used in the polypeptides of the invention include the Nanobodies directed against serum albumin that are described in WO 04/041865, in WO 06/122787 and in the further patent applications by Ablynx N.V., such as those mentioned above.

For example, the some preferred Nanobodies that provide for increased half-life for use in the present invention include Nanobodies that can bind to amino acid residues on (human) serum albumin that are not involved in binding of serum albumin to FcRn (see for example WO 06/0122787); Nanobodies that are capable of binding to amino acid residues on serum albumin that do not form part of domain III of serum albumin (see for example WO 06/0122787); Nanobodies that have or can provide an increased half-life (see for example the U.S. provisional application 60/843,349 by Ablynx N.V mentioned herein; see also PCT/EP2007/059475); Nanobodies against human serum albumin that are cross-reactive with serum albumin from at least one species of mammal, and in particular with at least one species of primate (such as, without limitation, monkeys from the genus Macaca (such as, and in particular, cynomologus monkeys (Macaca fascicularis) and/or rhesus monkeys (Macaca mulatta)) and baboon (Papio ursinus)) (see for example the U.S. provisional application 60/843,349 by Ablynx N.V; see also PCT/EP2007/059475)); Nanobodies that can bind to serum albumin in a pH independent manner (see for example the U.S. provisional application 60/850,774 by Ablynx N.V. mentioned herein) and/or Nanobodies that are conditional binders (see for example the U.S. provisional application 60/850,775 by Ablynx N.V.; see also PCT/EP2007/060850).

Some particularly preferred Nanobodies that provide for increased half-life and that can be used in the polypeptides of the invention include the Nanobodies ALB-1 to ALB-10 disclosed in WO 06/122787 (see Tables II and III) of which ALB-8 (SEQ ID NO: 62 in WO 06/122787) is particularly preferred.

According to a specific, but non-limiting aspect of the invention, the polypeptides of the invention contain, besides the one or more Nanobodies of the invention, at least one Nanobody against human serum albumin.

Generally, any polypeptides of the invention with increased half-life that contain one or more Nanobodies of the invention, and any derivatives of Nanobodies of the invention or of such polypeptides that have an increased half-life, preferably have a half-life that is at least 1.5 times, preferably at least 2 times, such as at least 5 times, for example at least 10 times or more than 20 times, greater than the half-life of the corresponding Nanobody of the invention per se. For example, such a derivative or polypeptides with increased half-life may have a half-life that is increased with more than 1 hours, preferably more than 2 hours, more preferably more than 6 hours, such as more than 12 hours, or even more than 24, 48 or 72 hours, compared to the corresponding Nanobody of the invention per se.

In a preferred, but non-limiting aspect of the invention, such derivatives or polypeptides may exhibit a serum half-life in human of at least about 12 hours, preferably at least 24 hours, more preferably at least 48 hours, even more preferably at least 72 hours or more. For example, such derivatives or polypeptides may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

According to one aspect of the invention the polypeptides are capable of binding to one or more molecules which can increase the half-life of the polypeptide in vivo.

The polypeptides of the invention are stabilised in vivo and their half-life increased by binding to molecules which resist degradation and/or clearance or sequestration. Typically, such molecules are naturally occurring proteins which themselves have a long half-life in vivo.

Another preferred, but non-limiting example of a multispecific polypeptide of the invention comprises at least one Nanobody of the invention and at least one Nanobody that directs the polypeptide of the invention towards, and/or that allows the polypeptide of the invention to penetrate or to enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such Nanobodies include Nanobodies that are directed towards specific cell-surface proteins, markers or epitopes of the desired organ, tissue or cell (for example cell-surface markers associated with tumor cells), and the single-domain brain targeting antibody fragments described in WO 02/057445 and WO 06/040153, of which FC44 (SEQ ID NO: 189 of WO 06/040153) and FC5 (SEQ ID NO: 190 of WO 06/040154) are preferred examples.

In the polypeptides of the invention, the one or more Nanobodies and the one or more polypeptides may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each Nanobody by itself forms a complete antigen-binding site).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y})_{z}, such as (for example (gly₄ser)₃ or (gly₃ser₂)₃, as described in WO 99/42077 and the GS30, GS15, GS9 and GS7 linkers described in the applications by Ablynx mentioned herein (see for example WO 06/040153 and WO 06/122825), as well as hinge-like regions, such as the hinge regions of naturally occurring heavy chain antibodies or similar sequences (such as described in WO 94/04678).

Some other particularly preferred linkers are poly-alanine (such as AAA), as well as the linkers GS30 (SEQ ID NO: 85 in WO 06/122825) and GS9 (SEQ ID NO: 84 in WO 06/122825).

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for Integrins, or for one or more of the other antigens. Based on the disclosure herein, the skilled person will be able to determine the optimal linker(s) for use in a specific polypeptide of the invention, optionally after some limited routine experiments.

For example, in multivalent polypeptides of the invention that comprise Nanobodies directed against a multimeric antigen (such as a multimeric receptor or other protein), the length and flexibility of the linker are preferably such that it allows each Nanobody of the invention present in the polypeptide to bind to the antigenic determinant on each of the subunits of the multimer. Similarly, in a multispecific polypeptide of the invention that comprises Nanobodies directed against two or more different antigenic determinants on the same antigen (for example against different epitopes of an antigen and/or against different subunits of a multimeric receptor, channel or protein), the length and flexibility of the linker are preferably such that it allows each Nanobody to bind to its intended antigenic determinant. Again, based on the disclosure herein, the skilled person will be able to determine the optimal linker(s) for use in a specific polypeptide of the invention, optionally after some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the Nanobodies of the invention). For example, linkers containing one or more charged amino acid residues (see Table A-2 above) can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after some limited routine experiments.

Finally, when two or more linkers are used in the polypeptides of the invention, these linkers may be the same or different. Again, based on the disclosure herein, the skilled persdn will be able to determine the optimal linkers for use in a specific polypeptide of the invention, optionally after some limited routine experiments.

Usually, for easy of expression and production, a polypeptide of the invention will be a linear polypeptide. However, the invention in its broadest sense is not limited thereto. For example, when a polypeptide of the invention comprises three of more Nanobodies, it is possible to link them by use of a linker with three or more □arms□, which each□ arm□ linked to a Nanobody, so as to provide a □shaped□ construct. It is also possible, although usually less preferred, to use circular constructs.

The invention also comprises derivatives of the polypeptides of the invention, which may be essentially analogous to the derivatives of the Nanobodies of the invention, i.e. as described herein.

The invention also comprises proteins or polypeptides that □essentially consist□ of a polypeptide of the invention (in which the wording □essentially consist of essentially the same meaning as indicated hereinabove).

According to one aspect of the invention, the polypeptide of the invention is in essentially isolated from, as defined herein.

The amino acid sequences, Nanobodies, polypeptides and nucleic acids of the invention can be prepared in a manner known per se, as will be clear to the skilled person from the further description herein. For example, the Nanobodies and polypeptides of the invention can be prepared in any manner known per se for the preparation of antibodies and in particular for the preparation of antibody fragments (including but not limited to (single) domain antibodies and ScFv fragments). Some preferred, but non-limiting methods for preparing the amino acid sequences, Nanobodies, polypeptides and nucleic acids include the methods and techniques described herein.

As will be clear to the skilled person, one particularly useful method for preparing an amino acid sequence, Nanobody and/or a polypeptide of the invention generally comprises the steps of:
i) the expression, in a suitable host cell or host organism (also referred to herein as a □host of the invention□) or in another suitable expression system of a nucleic acid that encodes said amino acid sequence, Nanobody or polypeptide of the invention (also referred to herein as a □nucleic acid of the invention□), optionally followed by:
ii) isolating and/or purifying the amino acid sequence, Nanobody or polypeptide of the invention thus obtained.

In particular, such a method may comprise the steps of:
i) cultivating and/or maintaining a host of the invention under conditions that are such that said host of the invention expresses and/or produces at least one amino acid sequence, Nanobody and/or polypeptide of the invention; optionally followed by:
ii) isolating and/or purifying the amino acid sequence, Nanobody or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one aspect of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more □mismatched□primers, using for example a sequence of a naturally [symbol] Integrins as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art and as described on pages 131-134 of WO 08/020,079 (incorporated herein by reference). Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as □genetic constructs of the invention□.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting aspect, a genetic construct of the invention comprises
i) at least one nucleic acid of the invention; operably connected to
ii) one or more regulatory elements, such as a promoter and optionally a suitable terminator;
   and optionally also
iii) one or more further elements of genetic constructs known per se;

in which the terms □operably connected□ and □operably linked□ have the mean given on pages 131-134 of WO 08/020,079; and in which the □regulatory elements□, □promoter□, □terminator□ and □ [symbol] WO 08/020,079; and in which the genetic constructs may further be as described on pages 131-134 of WO 08/020,079.

The nucleic acids of the invention and/or the genetic constructs of the invention may be used to transform a host cell or host organism, i.e. for expression and/or production of the amino acid sequence, Nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example those described on pages 134 and 135 of WO 08/020,079.; as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al., (1998), supra; Riechmann and Muyldermans, (1999), supra; van der Linden, (2000), supra; Thomassen et al., (2002), supra; Joosten et al., (2003), supra; Joosten et al., (2005), supra; and the further references cited herein.

The amino acid sequences, Nanobodies and polypeptides of the invention can also be introduced and expressed in one or more cells, tissues or organs of a multicellular organism, for example for prophylactic and/or therapeutic purposes (e.g. as a gene therapy), as further described on pages 135 and 136 of in WO 08/020,079 and in the further references cited in WO 08/020,079.

For expression of the Nanobodies in a cell, they may also be expressed as so-called □intrabodies□, as for example [symbol] 02610, WO 95/22618 and U.S. Pat. No. 7,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

The amino acid sequences, Nanobodies and polypeptides of the invention can for example also be produced in the milk of transgenic mammals, for example in the milk of rabbits, cows, goats or sheep (see for example U.S. Pat. No. 6,741,957, U.S. Pat. No. 6,304,489 and U.S. Pat. No. 6,849,992 for general techniques for introducing transgenes into mammals), in plants or parts of plants including but not limited to their leaves, flowers, fruits, seed, roots or turbers (for example in tobacco, maize, soybean or alfalfa) or in for example pupae of the silkworm Bombix mori.

Furthermore, the amino acid sequences, Nanobodies and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, polypeptides of the invention suitable for pharmaceutical use can be prepared using techniques for peptide synthesis.

For production on industrial scale, preferred heterologous hosts for the (industrial) production of Nanobodies or Nanobody-containing protein therapeutics include strains of E. coli, Pichia pastoris, S. cerevisiae that are suitable for large scale expression/production/fermentation, and in particular for large scale pharmaceutical (i.e. GMP grade) expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression. Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast usually leads to a glycosylation pattern that differs from human glycosylation. Nevertheless, it should be understood that all the foregoing host cells and expression systems can be used in the invention, depending on the desired amino acid sequence, Nanobody or polypeptide to be obtained.

Thus, according to one non-limiting aspect of the invention, the amino acid sequence, Nanobody or polypeptide of the invention is glycosylated. According to another non-limiting aspect of the invention, the amino acid sequence, Nanobody or polypeptide of the invention is non-glycosylated.

According to one preferred, but non-limiting aspect of the invention, the amino acid sequence, Nanobody or polypeptide of the invention is produced in a bacterial cell, in particular a bacterial cell suitable for large scale pharmaceutical production, such as cells of the strains mentioned above.

According to another preferred, but non-limiting aspect of the invention, the amino acid sequence, Nanobody or polypeptide of the invention is produced in a yeast cell, in particular a yeast cell suitable for large scale pharmaceutical production, such as cells of the species mentioned above.

According to yet another preferred, but non-limiting aspect of the invention, the amino acid sequence, Nanobody or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

As further described on pages 138 and 139 of WO 08/020,079, when expression in a host cell is used to produce the amino acid sequences, Nanobodies and the polypeptides of the invention, the amino acid sequences, Nanobodies and polypeptides of the invention can be produced either intracellullarly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. Thus, according to one non-limiting aspect of the invention, the amino acid sequence, Nanobody or polypeptide of the invention is an amino acid sequence, Nanobody or polypeptide that has been produced intracellularly and that has been isolated from the host cell, and in particular from a bacterial cell or from an inclusion body in a bacterial cell. According to another non-limiting aspect of the invention, the amino acid sequence, Nanobody or polypeptide of the invention is an amino acid sequence, Nanobody or polypeptide that has been produced extracellularly, and that has been isolated from the medium in which the host cell is cultivated.

Some preferred, but non-limiting promoters for use with these host cells include those mentioned on pages 139 and 140 of WO 08/020,079.

Some preferred, but non-limiting secretory sequences for use with these host cells include those mentioned on page 140 of WO 08/020,079.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence, Nanobody or polypeptide of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence, Nanobody or polypeptide of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence, Nanobody or polypeptide of the invention may (first) be generated in an immature form (as mentioned above), which may then be subjected to post-translational modification, depending on the host cell/host organism used. Also, the amino acid sequence, Nanobody or polypeptide of the invention may be glycosylated, again depending on the host cell/host organism used.

The amino acid sequence, Nanobody or polypeptide of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence, Nanobody or polypeptide of the invention) and/or preparative immunological techniques (i.e. using antibodies against the amino acid sequence to be isolated).

Generally, for pharmaceutical use, the polypeptides of the invention may be formulated as a pharmaceutical preparation or compositions comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one amino acid of the invention, at least one Nanobody of the invention or at least one polypeptide of the invention and at least one suitable carrier, diluent or excipient (i.e. suitable for pharmaceutical use), and optionally one or more further active substances.

Generally, the amino acid sequences, Nanobodies and polypeptides of the invention can be formulated and administered in any suitable manner known per se, for which reference is for example made to the general background art cited above (and in particular to WO 04/041862, WO 04/041863, WO 04/041865, WO 04/041867 and WO 08/020,079) as well as to the standard handbooks, such as Remington□s Pharmaceutical Sciences [symbol] , Mack Publishing Company, USA (1990), Remington, the Science and Practice of Pharmacy, 21st Edition, Lippincott Williams and Wilkins (2005); or the Handbook of Therapeutic Antibodies (S. Dubel, Ed.), Wiley, Weinheim, 2007 (see for example pages 252-255).

For example, the amino acid sequences, Nanobodies and polypeptides of the invention may be formulated and administered in any manner known per se for conventional antibodies and antibody fragments (including ScFv□s and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, those mentioned on page 143 of WO 08/020,079. Usually, aqueous solutions or suspensions will be preferred.

The amino acid sequences, Nanobodies and polypeptides of the invention can also be administered using gene therapy methods of delivery. See, e.g., U.S. Pat. No. 5,399,346, which is incorporated by reference in its entirety. Using a gene therapy method of delivery, primary cells transfected with the gene encoding an amino acid sequence, Nanobody or polypeptide of the invention can additionally be transfected with tissue specific promoters to target specific organs, tissue, grafts, tumors, or cells and can additionally be transfected with signal and stabilization sequences for subcellularly localized expression.

Thus, the amino acid sequences, Nanobodies and polypeptides of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient□s diet. For oral therapeutic administration [symbol] acid sequences, Nanobodies and polypeptides of the invention may be combined with one or more excipients and used in the form of ingestible tablets, buccal tablets, troches, capsules, elixirs, suspensions, syrups, wafers, and the like. Such compositions and preparations should contain at least 0.1% of the amino acid sequence, Nanobody or polypeptide of the invention. Their percentage in the compositions and preparations may, of course, be varied and may conveniently be between about 2 to about 60% of the weight of a given unit dosage form. The amount of the amino acid sequence, Nanobody or polypeptide of the invention in such therapeutically useful compositions is such that an effective dosage level will be obtained.

The tablets, troches, pills, capsules, and the like may also contain binders, excipients, disintegrating agents, lubricants and sweetening or flavouring agents, for example those mentioned on pages 143-144 of WO 08/020,079. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the amino acid sequences, Nanobodies and polypeptides of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the amino acid sequences, Nanobodies and polypeptides of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The amino acid sequences, Nanobodies and polypeptides of the invention may also be administered intravenously or intraperitoneally by infusion or injection, as further described on pages 144 and 145 of WO 08/020,079.

For topical administration, the amino acid sequences, Nanobodies and polypeptides of the invention may be applied in pure form, i.e., when they are liquids. However, it will generally be desirable to administer them to the skin as compositions or formulations, in combination with a dermatologically acceptable carrier, which may be a solid or a liquid, as further described on page 145 of WO 08/020,079.

Generally, the concentration of the amino acid sequences, Nanobodies and polypeptides of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the amino acid sequences, Nanobodies and polypeptides of the invention required for use in treatment will vary not only with the particular amino acid sequence, Nanobody or polypeptide selected but also with the route of administration, the nature of the condition being treated and the age and condition of the patient and will be ultimately at the discretion of the attendant physician or clinician. Also the dosage of the amino acid sequences, Nanobodies and polypeptides of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By □ong-term□ is meant at least two weeks and preferably, several weeks, months, or years of duration. Necessary modifications in this dosage range may be determined by one of ordinary skill in the art using only routine experimentation given the teachings herein. See Remington□s Pharmaceutical Sciences (Martin, E. W., ed. 4), Mack Publishing Co., Easton, Pa. The dosage can also be adjusted by the individual physician in the event of any complication.

In another aspect, the invention relates to a method for the prevention and/or treatment of at least one autoimmune diseases, cancer metastasis and thrombotic vascular diseases, said method comprising administering, to a subject in need thereof, a pharmaceutically active amount of an amino acid sequence of the invention, of a Nanobody of the invention, of a polypeptide of the invention, and/or of a pharmaceutical composition comprising the same.

In the context of the present invention, the term □prevention and/or treatment□not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention relates to a method for the prevention and/or treatment of at least one disease or disorder that is associated with Integrins, with its biological or pharmacological activity, and/or with the biological pathways or signalling in which Integrins is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, the invention relates to a method for the prevention and/or treatment of at least one disease or disorder that can be treated by modulating Integrins, its biological or pharmacological activity, and/or the biological pathways or signalling in which Integrins is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, said pharmaceutically effective amount may be an amount that is sufficient to modulate Integrins, its biological or pharmacological activity, and/or the biological pathways or signalling in which Integrins is involved; and/or an amount that provides a level of the amino acid sequence of the invention, of a Nanobody of the invention, of a polypeptide of the invention in the circulation that is sufficient to modulate Integrins, its biological or pharmacological activity, and/or the biological pathways or signalling in which Integrins is involved.

The invention furthermore relates to a method for the prevention and/or treatment of at least one disease or disorder that can be prevented and/or treated by administering an amino acid sequence of the invention, a Nanobody of the invention or a polypeptide of the invention to a patient, said method comprising administering, to a subject in need thereof, a pharmaceutically active amount of an amino acid sequence of the invention, of a Nanobody of the invention, of a polypeptide of the invention, and/or of a pharmaceutical composition comprising the same.

More in particular, the invention relates to a method for the prevention and/or treatment of at least one disease or disorder chosen from the group consisting of the diseases and disorders listed herein, said method comprising administering, to a subject in need thereof, a pharmaceutically active amount of an amino acid sequence of the invention, of a Nanobody of the invention, of a polypeptide of the invention, and/or of a pharmaceutical composition comprising the same.

In another aspect, the invention relates to a method for immunotherapy, and in particular for passive immunotherapy, which method comprises administering, to a subject suffering from or at risk of the diseases and disorders mentioned herein, a pharmaceutically active amount of an amino acid sequence of the invention, of a Nanobody of the invention, of a polypeptide of the invention, and/or of a pharmaceutical composition comprising the same.

In the above methods, the amino acid sequences, Nanobodies and/or polypeptides of the invention and/or the compositions comprising the same can be administered in any suitable manner, depending on the specific pharmaceutical formulation or composition to be used. Thus, the amino acid sequences, Nanobodies and/or polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

The amino acid sequences, Nanobodies and/or polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific amino acid sequence, Nanobody or polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more amino acid sequences, Nanobodies and/or polypeptides of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency of the specific amino acid sequence, Nanobody and polypeptide of the invention to be used, the specific route of administration and the specific pharmaceutical formulation or composition used, the amino acid sequences, Nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single amino acid sequence, Nanobody or polypeptide of the invention will be used. It is however within the scope of the invention to use two or more amino acid sequences, Nanobodies and/or polypeptides of the invention in combination.

The Nanobodies, amino acid sequences and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

In particular, the amino acid sequences, Nanobodies and polypeptides of the invention may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and on a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of an amino acid sequence, Nanobody or polypeptide of the invention in the preparation of a pharmaceutical composition for prevention and/or treatment of at least one autoimmune diseases, cancer metastasis and thrombotic vascular diseases; and/or for use in one or more of the methods of treatment mentioned herein.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention also relates to the use of an amino acid sequence, Nanobody or polypeptide of the invention in the preparation of a pharmaceutical composition for the prevention and/or treatment of at least one disease or disorder that can be prevented and/or treated by administering an amino acid sequence, Nanobody or polypeptide of the invention to a patient.

More in particular, the invention relates to the use of an amino acid sequence, Nanobody or polypeptide of the invention in the preparation of a pharmaceutical composition for the prevention and/or treatment of autoimmune diseases, cancer metastasis and thrombotic vascular diseases, and in particular for the prevention and treatment of one or more of the diseases and disorders listed herein.

Again, in such a pharmaceutical composition, the one or more amino acid sequences, Nanobodies or polypeptides of the invention may also be suitably combined with one or more other active principles, such as those mentioned herein.

Finally, although the use of the Nanobodies of the invention (as defined herein) and of the polypeptides of the invention is much preferred, it will be clear that on the basis of the description herein, the skilled person will also be able to design and/or generate, in an analogous manner, other amino acid sequences and in particular (single) domain antibodies against Integrins, as well as polypeptides comprising such (single) domain antibodies.

For example, it will also be clear to the skilled person that it may be possible to □graft □ one or more of the CD□s mentioned above for the Nanobodies of the invention ont such (single) domain antibodies or other protein scaffolds, including but not limited to human scaffolds or non-immunoglobulin scaffolds. Suitable scaffolds and techniques for such CDR grafting will be clear to the skilled person and are well known in the art, see for example those mentioned in WO 08/020,079. For example, techniques known per se for grafting mouse or rat CDR□s onto human frameworks and scaffolds can be used in an analogous manner to provide chimeric proteins comprising one or more of the CDR□s of the Nanobodies of the invention and one or more human framework regions or sequences.

It should also be noted that, when the Nanobodies of the inventions contain one or more other CDR sequences than the preferred CDR sequences mentioned above, these CDR sequences can be obtained in any manner known per se, for example using one or more of the techniques described in WO 08/020,079.

Further uses of the amino acid sequences, Nanobodies, polypeptides, nucleic acids, genetic constructs and hosts and host cells of the invention will be clear to the skilled person based on the disclosure herein. For example, and without limitation, the amino acid sequences of the invention can be linked to a suitable carrier or solid support so as to provide a medium than can be used in a manner known per se to purify Integrins from compositions and preparations comprising the same. Derivatives of the amino acid sequences of the invention that comprise a suitable detectable label can also be used as markers to determine (qualitatively or quantitatively) the presence of Integrins in a composition or preparation or as a marker to selectively detect the presence of Integrins on the surface of a cell or tissue (for example, in combination with suitable cell sorting techniques).

The invention will now be further described by means of the following non-limiting experimental part and figures, in which the Figures show:

### FIGURES

FIG. 1: FIG. 1: Schemetic representation of the second round of selection on whole cells. Sub-libraries of VHH-phages enriched on membrane fractions isolated from HeLa cells grown under nomoxic (21% O₂) or hypoxic (1% O₂) conditions from the first round were incubated with live adherent HeLa cells in the corresponding conditions. Phages were preincubated with suspension cells, which were incubated under conditions opposite to the adherent cells (i.e first normoxic than hypoxic and vice versa). The counter selection was maintained during the time of selection. N denotes Normoxia, H denotes Hypoxia.

FIG. 2: Immunoprecipitation of the VHH targets from HeLa cell lysates. VHH-H6 was coupled to ProtA/G beads through the anti-Myc antibody (9E10), and used to pulldown cognate antigens from the lysate of HeLa cells. Bound proteins were analyzed with SDS-PAGE gels stained with SimplyBlue. The expected heavy and light chains of IgGs, BSA (smear) and VHH are indicated on the right. A protein band of approximately 150 kDa (denoted by *) represents the VHH-H6 target antigen. Lane M represents molecular weight marker proteins (in kDa), and are indicated on the left.

FIG. 3: Direct binding of VHH-H6 to recombinant α3β1 integrin. Recombinant VLA-3 (α3β1 integrin) or recombinant VLA-5 (a5β1 integrin) were coated into Maxisorb plates at 0.5 μg/well, and binding with VHH-H6 or no VHH (in which anti-Myc was used) was assayed in ELISA. Binding of the VHHs to the integrins is expressed as absorbance at 450 nm. Data represents the mean±standard deviation of three independent experiments

FIG. 4: DNA sequence of VHH H6, in which the sequences used to design primers for the amplification reaction are as follows: Italic—conserved sequences, in brackets are variations on these sequences to get VHH H6 family members; underlined, italic—sequence unique for CDR3 of VHH H6; and underlined—an additional sequence to improve the hybridisation. In the rest of the sequences there will be sequence variations and by sequencing the PCR products these variations will be determined.

FIG. 5: FACS experiments carried out on K562 and K562 cells expressing VLA-3. This figure clearly demonstrate that only the VLA-3 expressing cells are recognized by VHH H6.

FIG. 6: Inhibition of adhesion of K562 A3A cells by VHH H₆

FIG. 7: Biotinilated-Fibronectin binding to coated aVb6 (1.5 ug/ml) in presence of purified nanobodies: y=Percent biot-fibronectin bound; x=tested clones and controls, i.e. 1=141C1; 2=140A10; 3=140D10; 4=140G10; 5=140F2; 6=no nonobody; 7=140E5; 8=140H5; 9=140G8; 10=140D4; 11=140A5; 12=140B8; 13=222A4 (control)

FIG. 8: a) staining with about 0.03 uM CD49c; b) staining with 0.3 uM SEQ ID NO: 1486, i.e. VHH-5 bihead with 15GS linker; c) staining with SEQ ID NO: 1474, i.e. VHH-5 monohead

### EXPERIMENTAL PART

### Example 1

### Immunizations

Two llamas (169 and 170) were immunized according to standard protocols with 6 boosts of a cocktail of recombinant human proteins (2×40 ug+4×20 ug). Blood was collected from these animals at 6 and 10 days after the 6^{th} boost. The cocktail was a mixture of: Recombinant human alphaLbeta2/LFA-1 carrier free acquired from R&D Systems (cat nr: 3868-AV/CF); recombinant human alphaMbeta2/MAC-1 carrier free acquired from R&D Systems (cat nr: 4047-AM/CF); recombinant human alphavbeta6 carrier free acquired from R&D Systems (cat nr: 3817-AV/CF); recombinant human alpha3betaINLA-3 carrier free acquired from R&D Systems (cat nr: 2840-A3/CF); recombinant human alpha5beta1/VLA-5 carrier free acquired from R&D systems (cat nr: 3230-A5/CF).

### Example 2

### Library Construction

Peripheral blood mononuclear cells were prepared from blood samples using Ficoll-Hypaque according to the manufacturer□s instruction [symbol] tal RNA extracted was extracted from these cells as well as from the lymph node bow cells and used as starting material for RT-PCR to amplify Nanobody encoding gene fragments. These fragments were cloned into phagemid vector pAX50. Phage was prepared according to standard methods (see for example the prior art and applications filed by applicant cited herein) and stored at 4° C. for further use, making phage library 169 and 170.

### Example 3

### Selections

To identify Nanobodies recognizing the chemokines, phage libraries 169 and 170 were used for selections on the integrins that were used for immunization. The integrins were coated independently at 5 ug/ml, 0.5 ug/ml or 0 ug/ml (control) on Nunc Maxisorp ELISA plates (100 ul per wells). Selection was done as usual with the difference that 1 mM MnCl2 and 1 mM MgCl2 was added (or not) with the library during phage binding. Bound phages were eluted from the integrin using trypsine.

In addition to the trypsine elution, competitive elution were done. In this case, bound phages were eluted with either an excess of specific ligands (hICAM in the case of aLb2 and aMb2). Note that other elutions are possible: fibronectin (or RGD containing peptide that bind the ligand binding site of specific integrins) may be used in the case of aVb6 and a5b1; collagen in the case of a3b1) and with EDTA which chelates the bivalent ions bound to the integrin and essential to allow ligand binding.

An alternative to elute site-specific Nanobody is the use of specific antibody which are known to bind at relevant site on the integrin, this include for example the humanized monoclonal antibody Efalizumab that bind and block the aLb2 integrin.

Output of R1 selections were analyzed for enrichment factor (phage present in eluate relative to controls). Based on these parameters the best selections were chosen for further analysis. The polyclonal output was then recloned in PAX51 and individual TG1 colonies were picked and grown in 96 deep well plates (1 ml volume) and induced by adding IPTG for Nanobody expression. Periplasmic extracts (volume: ˜80 ul) were prepared according to standard methods (see for example the prior art and applications filed by applicant cited herein).

Alternatively, to enrich the pool of phage binding at relevant site on the integrin, a second round of selection using the phages (R2 output, see for example the prior art and applications filed by applicant cited herein) may be done in combination to trypsine or specific elution as mentioned above.

To enrich the pool of phage binding to a specific chain (alpha or beta), selection is also done in the presence of a counterselecting protein (during R1, R2 or both). For example to enrich for alpha5 binding phage, selection may be done in the presence of alpha3beta1 integrin.

### Example 4

### Screening for Binding

In order to determine binding specificity of the Nanobodies present in the periplasm fraction, the 10 ul of the periplasm fraction were tested in an ELISA binding assay. In short, 1.5 ug/ml of relevant integrin were coated directly on Maxisorp microtiter plates (Nunc). Free binding sites were blocked using 4% Marvel in PBS. Next, 10 ul of periplasmic extract containing nanobody of the different clones (92 per target) in 100 ul 1% Marvel PBST were allowed to bind to the immobilized antigen. After incubation and a washing step, nanobody binding was revealed using a mouse-anti-myc antibody, which was after a washing step detected with a Alkaline phosphatase (AP)-conjugated goat-anti-mouse antibody. Binding specificity was determined based on OD values compared to controls having received no nanobody. The result are shown in table B-1.

TABLE B-1
Positive clones (binding to the coated integrin) identify
by ELISA. Are depicted the number of positive as well
as the representative percentage of positive.

| | Number positive | Number positive | | |
|---|---|---|---|---|
| Integrin | clone in | clone in | positive | |
| coated | library169 | library170 | clone | Percent |
| (1.5 ug/ml) | (46 clones) | (46 clones) | (/92) | positive |
| aLb2 | 36 | 46 | 82 | 89 |
| aMb2 | 46 | 44 | 90 | 98 |
| aVb6 | 43 | 44 | 87 | 95 |
| a3b1 | 45 | 44 | 89 | 97 |

### Example 5

### Screening for Specificity

Because Integrins are noncovalently associated heterodimeric cell surface adhesion molecules composed of one alpha subunit and one beta subunit, the Nanobodies were tested for their specificity (alpha or beta chain). For this the periplasm from example 3 were tested on direct ELISA on 1) the integrin for which they were selected, 2) integrin sharing similar beta chain and 3) integrin with different alpha and beta chain according to the table B-2. Those results show also the specificity of the periplasm tested.

TABLE B-2

| | | | Coating 3 | | | |
|---|---|---|---|---|---|---|
| | Coating 1 | Coating 2 | (non- | | Alpha | Beta |
| | (selected | (similar beta | relevant | Integrin | chain | chain |
| | target) | chain) | integrin | specific | specific | specific |
| aLb2 | aLb2 | aMb2 | a5b1 | 82% | 18% | 63% |
| aMb2 | aMb2 | aLb2 | aVb6 | 76% | 13% | 63% |
| aVb6 | aVb6 | — | aLb2 | 75% | nt | nt |
| a3b1 | a3b1 | a5b1 | aVb6 | 48% | 16% | 31% |
| a5b1 | a5b1 | a3b1 | aMb2 | 28% | 1% | 27% |

Based on their binding selectivity, Nanobody clones were sequenced and grouped per family or unique non related sequence.

For alphaVbeta6, 23 unique sequences (out of 31 sequenced) were found grouping into: 2 families and 8 unique non-related sequences (see Table B-3).

TABLE B-3

| Name | Specificity | Other members of same family* |
|---|---|---|
| 140-F10 | alphaVbeta6 | 140-H10, 140-B10, 140-B8, 140-C8, |
| | | 140-D8 |
| 140-E5 | alphaVbeta6 | 140-F2, H5 |
| 140-E10 | alphaVbeta6 | No further member |
| 140-D10 | alphaVbeta6 | No further member |
| 140-G8 | alphaVbeta6 | No further member |
| 140-A10 | alphaVbeta6 | No further member |
| 140-A2 | alphaVbeta6 | No further member |
| 140-H8 | alphaVbeta6 | No further member |
| 140-G10 | alphaVbeta6 | No further member |
| 140-E2 | alphaVbeta6 | No further member |
| 140-D4 | alphaVbeta6 | No further member |
| 140-A5 | alphaVbeta6 | No further member |

*Nanobodies from the same family have similar sequences and identical or very similar (only having a few mutation) CDR3. E.g.:
nanobodies having identical CDR3 but having at least 1 mutation in the rest of the sequence (CDR1, CDR2 or in framworks)
nanobodies having few mutation in CDR3
nanobodies having both of the above.
For alphaLBeta2 and alphaMbeta2, 109 unique sequences were found (out of 181 sequenced) and are shown in Table B-4:
50 Beta2 specific (6 families and 6 non related sequences)
29 alphaL specific (8 families and 6 non related sequences)
14 alphaM specific (4 families and 5 non related unique sequence)
4 sequences unknown specificity non related to other families
4 sequences not yet tested (however Nanobodies raised against integrins)

TABLE B-4

| | | Other members of same family that have |
|---|---|---|
| Name | Specificity | same specificity * |
| 153-G1 | Beta2 | 138-C2, 139-C2, 253-E3, 152-C6, 153-G1 |
| 138- | | 152-D4, E4, 152-F2, 138-B2, 153-D4, 153- |
| A2, E5, F5, 139- | | G2, F4, 153-G5, 153-H2, 153-H4, 152-C4, |
| G2, 152-A6, 153- | | 152-F4, 152-E1, 152-H4, 152-F4, 152E1, |
| B5, B6, F5 | Beta2 | 152-H4, 235-C10, D10, 235-C3 |
| | | 153-D7, F7, E8, 152-A8, 236-A11, C5, F5, |
| | | 248-F8, 139-F10, 152-F11, H11, 153-G10, |
| | | 153-B7, E9, 152-H10, 152-B10, 139-D10, |
| | | 153-A10, 153-C7, 153-C9, 152- |
| | | A12, B9, H7, H8, 236-D11, A12, C11, 248- |
| 139-A10, 153- | | B8, E7, 152-G9, 153-F9, 248-G7, 236-B11, |
| D7, F7, E8 | Beta2 | 236-D5, 236-E12, 236-B5, F11, 152, G10 |
| 153-A8, B11 | Beta2 | 152-C9, 236-E5 |
| 138-H2 | Beta2 | 248-C2, 138-B5, 248-H1 |
| 138-A5, 139-E2, | | 152-G2 |
| 235-E6 | Beta2 | |
| 153-E10, D8, G7 | Beta2 | No further member |
| 235-D5, 153-A1 | Beta2 | No further member |
| 138-F2 | Beta2 | No further member |
| 139-H10 | Beta2 | No further member |
| 139-B2, 152-E2, | Beta2 | No further member |
| 235-C9, D9 | | |
| 248-C7 | Beta2 | No further member |
| 138-G2, 235-E10 | alphaL | 138-H5, 235-A6, 243-B9, 235-E9, 138-D2, |
| | | 138-E4 |
| 138-B10 | alphaL | 138-G11 |
| 138-D7 | alphaL | 138-H7, 152-B7, 138-D11, 138-C10 |
| 235-B7 | alphaL | 248-F2 |
| 248-F7, D8 | alphaL | 248-H7 |
| 248-D7 | alphaL | 236-B4, A5, 248-G8, 248-E8 |
| 248-C8 | alphaL | 236-E11 |
| 235-F3 | alphaL | 248-F1, G1, 248-G2 |
| 138-G4, G5 | alphaL | No further member |
| 235-A12, 248- | alphaL | No further member |
| B1, C1, E1, H2 | | |
| 248-B7 | alphaL | No further member |
| 248-A1 | alphaL | No further member |
| 248-D2, D1, E2 | alphaL | No further member |
| 248-A7 | alphaL | No further member |
| 139-B10 | alphaM | 139-B7, 139-C11, 139-C7, 139-H9 |
| 139-C10 | alphaM | 139-H11 |
| 139-D2 | alphaM | 243-G9 |
| 243-C9 | alphaM | 243-A9 |
| 139-E9 | alphaM | No further member |
| 139-H7 | alphaM | No further member |
| 139-H1 | alphaM | No further member |
| 243-H9 | alphaM | No further member |
| 153-D3 | alphaM | No further member |
| 248-A8 | Specificity not tested | No further member |
| | yet | |
| 248-H8 | Specificity not tested | No further member |
| | yet | |
| 248-A2 | Specificity not tested | No further member |
| | yet | |
| 248-B2 | Specificity not tested | No further member |
| | yet | |
| 152-D2 | Unknown specificity | No further member |
| 152-E9 | Unknown specificity | No further member |
| 152-E10 | Unknown specificity | No further member |
| 152-D7 | Unknown specificity | No further member |

* Nanobodies from the same family have similar sequences and identical or very similar (only having a few mutation) CDR3. E.g.:
nanobodies having identical CDR3 but having at least 1 mutation in the rest of the sequence (CDR1, CDR2 or in framworks)
nanobodies having few mutation in CDR3
nanobodies having both of the above.

For alpha3Beta1 and alpha5beta1, 60 unique sequences were found (out of 113 sequenced) and are shown in Table B-5:
24 specific for beta1 (2 families and 5 unique non related sequences)
7 specific for Alpha 5 (3 families)
19 specific for Alpha3 (4 families and 10 unique non related sequences)

TABLE B-5

| | | Other members of same family |
|---|---|---|
| Name | Specificity | that have same specificity * |
| 141-A10 | Beta1 | 141-C10, 142-C11, F7, |
| | | 141-E10, 141-F10, |
| | | 142-F10, 141-A11, 242-A12, |
| | | B12, B4, C4, D12, D4, |
| | | E4, F12, F4, G12, B8, |
| | | D8, 242-E8, 141-C11, |
| | | 242-G4, 142-C7, 142-B10, |
| | | 142-C9, 142-D8, A8, H9, |
| | | 142-E10, 142-E11, G11, |
| | | 141-D11, 141-F11, |
| | | 142-B11, 141-G11, 142-B7 |
| 142-A7 | Beta1 | 142-G7 |
| 141-H10 | Beta1 | No further member |
| 141-E5 | Beta1 | No further member |
| 141-F5 | Beta1 | No further member |
| 141-B6 | Beta1 | No further member |
| 245-D7 | Beta1 | No further member |
| 242-A4, | Alpha5 | No further member |
| A8, C12, C8, D7, | | |
| E12, F5, F8, G7, E11 | | |
| 241-A10, | Alpha5 | 241-D11, 241-C1, C2, |
| A11, B10, B11, | | 241-C10 |
| D10, D8, E10, E11, E4, | | |
| F11, F8, G10, G11, | | |
| H10, C11, C8 | | |
| 241-E1, F10 | Alpha5 | 241-E3 |
| 141-F1 | Alpha3 | 141-B4, 141-F6, G1 |
| 141-B11 | Alpha3 | 141-H11 |
| 141-A5 | Alpha3 | 141-E6 |
| 141-C2 | Alpha3 | 245-G1 |
| 141-D10 | Alpha3 | No further member |
| 141-A4 | Alpha3 | No further member |
| 141-D4, 245-H1 | Alpha3 | No further member |
| 141-B10 | Alpha3 | No further member |
| 245-E7 | Alpha3 | No further member |
| 245-B1 | Alpha3 | No further member |
| 245-D1 | Alpha3 | No further member |
| 245-A1, F1 | Alpha3 | No further member |
| 245-E1 | Alpha3 | No further member |
| 245-F7 | Alpha3 | No further member |

* Nanobodies from the same family have similar sequences and identical or very similar (only having a few mutation) CDR3. E.g.:
nanobodies having identical CDR3 but having at least 1 mutation in the rest of the sequence (CDR1, CDR2 or in framworks)
nanobodies having few mutation in CDR3
nanobodies having both of the above.

### Example 6

### Screening for Nanobody Neutralizing or Activating of aVb6

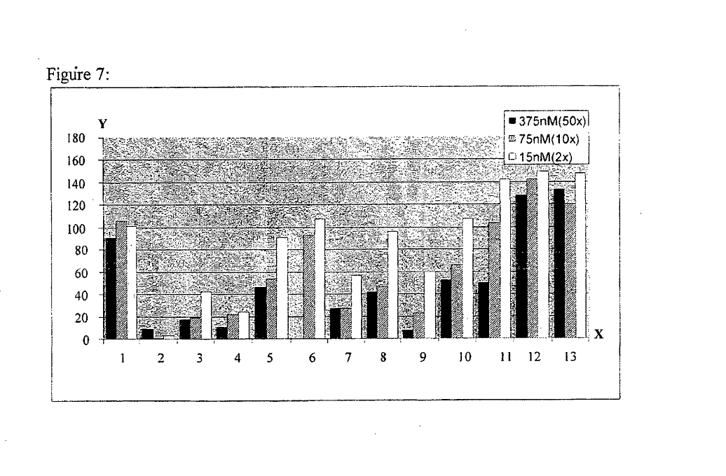

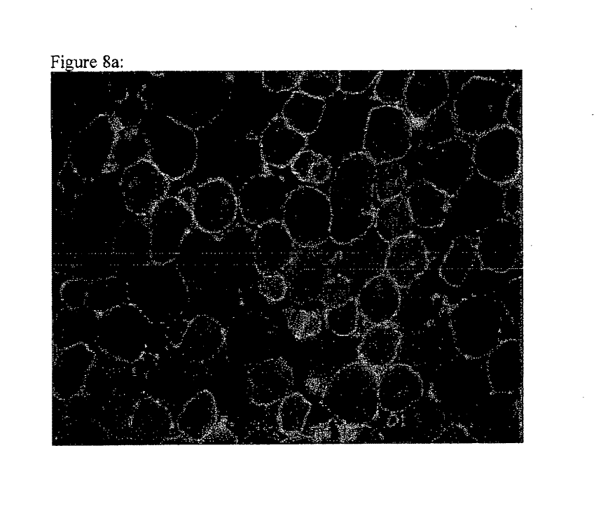

In order to determine neutralizing activity of the Nanobodies against aVb6, the clones were tested in a receptor/ligand binding assay (Competitive ELISA). Shortly, aVb6 was coated in 96 wells (Maxisorp, Nunc). After washing and blocking as usual, aVb6 was incubated with 15 ul periplasmic fraction prepared from example 3 in the presence of 1 mM MgCl2. Finally, biotinylated fibronectin was added. After washing, the presence of integrin bound biotinylated-fibronectin was detected using streptavidine-HRPO antibody. In the case where a Nanobody present in the periplasm neutralizes aVb6 (i.e., compete for ligand binding), no ligand bound was detected (low signal compared to control without any Nanobody). In the case where a Nanobody present in the periplasm activates aVb6 (i.e. favour ligand binding), more bound biotinylated fibronectin was detected (higher signal compared to control without any Nanobody). In all cases, the concentration of protein tested was used to get sub-optimal response. To confirm the function of the neutralizing Nanobody, the latest was further recloned in PAX51, produced in TG1 and purify using the TALON beads (see Table B-6, FIG. 7).

TABLE B-6

| | PERI | PURIFIED |
|---|---|---|
| | □Compete | □Compete |
| Nanobodies | FN binding | FN binding |
| 140-F10 | − | |
| 140-H10 | − | |
| 140-B10 | − | |
| 140-B8 | partial | − |
| 140-C8 | partial | |
| 140-D8 | partial | |
| 140-E5 | + | + |
| 140-F2, H5 | Partial, + | Partial, + |
| 140-E10 | − | |
| 140-D10 | + | + |
| 140-G8 | + | + |
| 140-A10 | + | + |
| 140-A2 | − | |
| 140-H8 | partial | |
| 140-G10 | + | + |
| 140-E2 | − | |
| 140-D4 | partial | partial |
| 140-A5 | partial | partial |

Table 4: The result of competition assay is indicated. (−) no competition, (+) competition. PERI or Purified is for competition assay using periplasm or purified Nanobodies respectively.

### Example 7

### Screening for Nanobody Neutralizing or Activating a5b1

In order to determine neutralizing activity of the Nanobodies against a5b1, the same may be done as in example 5, with the difference that a5b1 is coated instead of aVb6. Alternatively, the binding of alpha5beta1 binding to coated fibronectin may be detected with anti-beta1 antibody (R&D system, cat MAB 1778).

### Example 8

### Screening for Nanobody Neutralizing or Activating a3b1

In order to determine neutralizing activity of the Nanobodies against a3b1, the clones may be tested in a receptor/ligand binding assay (Competitive ELISA). Shortly, rat tail collagen or any specific a3b1 ligand is coated in 96 wells (Maxisorp, Nunc). After washing and blocking as usual, a3b1 is incubated with 15 ul periplasmic fraction prepared from above in the presence of 1 mM MgCl2. Finally, the fraction of a3b1 bound to the collagen is detected using subsequently biotinylated mouse-anti-human beta1 antibody (R&D system, cat MAB1778, biotinylated using Pierce kit according to supplier) and streptavidine-HRPO antibody.

### Example 9

### Screening for Nanobody Neutralizing or Activating aLb2 or aMb2

In order to determine neutralizing or activating activity of the Nanobodies against aLb2 and aMb2, the same may be done as in example 7 except that human-ICAM1-Fc is coated in 96 wells (Maxisorp, Nunc) instead of collagen and that the bound aLb2 and aMb2 is detected using biotinylated mouse-anti-human beta2 antibody (R&D Systems, cat MAB 1530, biotinylated using Pierce kit according to supplier).

### Example 10

### Screening for Nanobody Competing the Binding of Efaluzimab to aLb2

Efaluzimab is a commercial antibody blocking alphaL binding to its ligand and is use to treat psoriasis. In order to identify Nanobodies binding to the same site as Efaluzimab, a competition assay may be performed:

aLb2 is coated in 96 wells (Maxisorp, Nunc). After washing and blocking as usual, aLb2 is incubated with 15 ul periplasmic fraction prepared from above (example 1) in the presence of 1 mM MgCl2 (or 1 mM MnCl2) and Efaluzimab is added. After incubation and washing, the bound Efaluzimab is detected using mouse anti-human Fc-HRPO antibody (Jackson laboratories).

In order to obtain more Efaluzimab competing Nanobodies, new selection (as in example2) may be performed with the difference that an excess of Efaluzimab can be used (in R1, R2 or both) to elute specific phage binding at the same site as Efaluzimab.

### Example 11

### Screening for Nanobody Competing Blocking Integrin on Cells

Because cells rely on integrin to adhere to the substratum, the effect of Nanobodies (purified or as in periplasm fraction) on integrin can be tested directly on their effect on cell adherence on specific substratum. To improve specificity of the assay, a specific integrin can be overexpressed to increase the adhesion depending to this integrin. Increase or decrease in adhesion can be measure by measuring the number of adherent cell at a given time point. IN addition, the same assay can be done and migration can be measured. Alternatively, the binding of labelled ligand directly on cell is also a readout for integrin activity. This can also be achieve in the presence of Nanobodies.

### Example 12

### Selection and Screening of VHHs Recognizing Cell Surface Proteins

### Example 12.1

### Raising Llama Antibodies

Llamas were immunized with the antigens consisting of membrane vesicles of a cell line grown under hypoxia conditions or membrane fractions extracted from cells of a solid tumor in the presence of the adjuvant Stimune by subcutaneous injections. The immunization scheme consisted of a priming immunization (at day 0) followed by 3 boosts (at days 14, 28 and 35). The immune response was measured in the serum taken up at day 28 and compared to day 0. Alternatively, intact cells and tissues were used for immunization. The immunogens were injected subcutaneous in the absence of any adjuvant. The immunization scheme was as described for the membrane vesicles and membrane fractions immonogens.

### Example 12.2

### Construction of Variable Domains of Heavy Chain Llama Antibody Library

When the titer of the heavy chain antibodies increased at day 28, peripheral blood lymphocytes (PBLs) were isolated from 150 ml blood taken up at day 43. Total RNA was isolated from these PBLs using phenol-chloroform-isoamylalcohol method. RNA was converted into cDNA using superscriptIII (invitrogen). IgG binding domains were amplified with PCR using primers annealing at the signal sequence of the IgGs and the hinge region. The ˜700 bp fragment corresponding to the antigen binding domain of the heavy chain antibodies was excised from gel, and the SfiI restriction site was introduced at the 5□ by a nested PCR-step to facilitate cloning into the display vectors.

The purified 700 bp fragment was digested with BstEII (a restriction site found in the hinge region of heavy chain antibodies) and SfiI, and the resulting 400 bp antigen-binding fragment of the heavy chain antibodies were cloned in a phage-display plasmid.

The plasmids were transferred to Escherichia coli strain TG1. A transformation efficiency of 10 exp8, which also represents the diversity in the library, was generally obtained. E. coli TG 1 was used for the production of phages and for the infection by selected phages. Furthermore, E. coli TG1 was used for the production of selected VHH-monohcads and biheads.

### Example 12.3

### Selection of Variable Domains of Heavy Chain Llama Antibodies Recognizing Cell Surface Proteins

### Bacterium Strain and Cultivation Conditions

Escherichia coli strain TG1 was used for the maintenance of the plasmids, infection of the phages and expression of proteins. E. coli TG1 was grown in LB or 2×YT medium supplemented with glucose and antibiotics as indicated. VHH-phages were rescued by incubation of the phages with log-phase E. coli TG1 at 37° C. for 30 min (static conditions), followed by incubation in the presence of selection (ampicillin) overnight at 37° C. (shaking). Phages were produced from E. coli TG I containing phagemids with VHH genes fused to M13 gene3, by infection of log-phase bacteria with the helper phage VCSM13 (Stratagene, La Jolla, Calif., USA) for 30 min at 37° C. (static conditions), followed by incubation in the presence of both ampicillin and kanamycin overnight at 37° C. Produced phages were isolated by PEG precipitation of the culture supernatant.

### Cell Culture

HeLa cells were cultured in DMEM, supplemented with 10% Fetal Calf Serum (Gibco), 100 U/ml penicillin-streptomycin (Gibco) and 100 U/ml L-Glutamine (Gibco) at 5% CO₂, 21% O₂ for normoxia and 1% O₂ for hypoxia in a Invivo² Hypoxia Workstation 1000 (Biotrace International, UK) at 37° C.

Selection of VHH that Differentiate Between Cells Grown Under Hypoxic and Normoxic conditions

A selection procedure was designed to select VHHs against surface markers displayed differentially under hypoxic and normoxic conditions in two rounds. In the 1st round, membrane proteins isolated from HeLa cells grown under hypoxia or normoxia for 24 hrs using the vesicle isolation protocol were coated overnight at 4° C. in 96-wells Nunc Maxisorp plate (NUNC, Roskilde, Denmark). Different amounts of membrane proteins (10 μg, 5 μg, 1 μg and no protein at all) were coated in phosphate buffered saline (PBS) into each well. Coated wells were blocked with 4% Marvel (dried skimmed milk, Premier International Foods, Coolock, UK) in PBS for 1 hour at room temperature prior to the addition of phages. About 10¹¹ phages were added to each well. Phages were pre-incubated in 2% Marvel in PBS for 30 min in the presence of an excess of membrane proteins (15 μg/well) isolated from the condition opposite to the condition used in order to counter select for common antigens between the two conditions. Subsequently, phage/protein mixtures were added to the wells and incubated for 2 h at room temperature. The plate was then washed for 15 times with PBS containing 0.05% tween-20 (PBST) (the 5^{th}, 10^{th} and 15^{th} wash steps were done for 10 min) and 3 times with PBS. Bound phages were eluted from the wells with 100 mM triethylamine (TEA) and neutralized with 1M Tris-HCl pH 7.5. DNA information of the selected phages was rescued by infection of E. coli TG1 strain and subsequent selection for ampicillin

| Membrane | | | | |
|---|---|---|---|---|
| Proteins | 10 μg | 5 μg | 1 μg | PBS |
| Normoxia | 8 × 10⁴ | 1.4 × 10⁴ | 5.7 × 10³ | 1.3 × 10³ |
| Hypoxia | 1 × 10⁵ | 1 × 10⁵ | 6.4 × 10⁴ | 2 × 10² |

resistance.

The number of eluted phages was determined by plating serial dilutions of the different infections. Phages were produced from selections on the highest membrane protein concentrations from both hypoxia and normoxia, which both resulted in a high enrichment factor compared to empty wells.

Table B-5 Number of phages bound to the indicated conditions in the first round of selection.

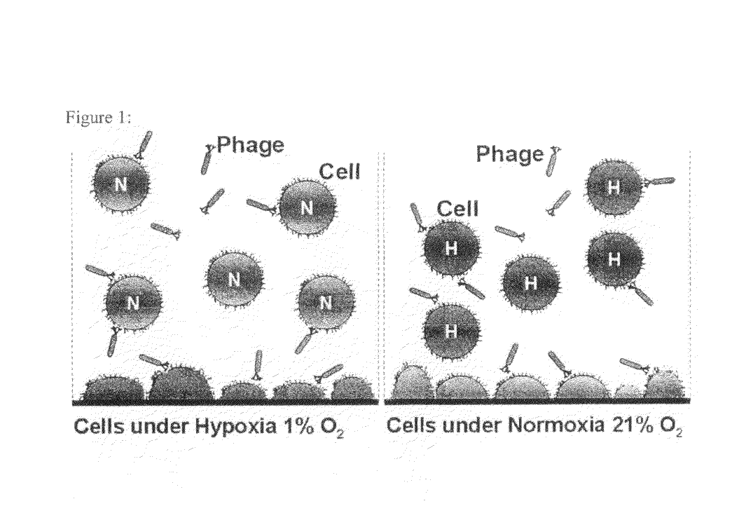

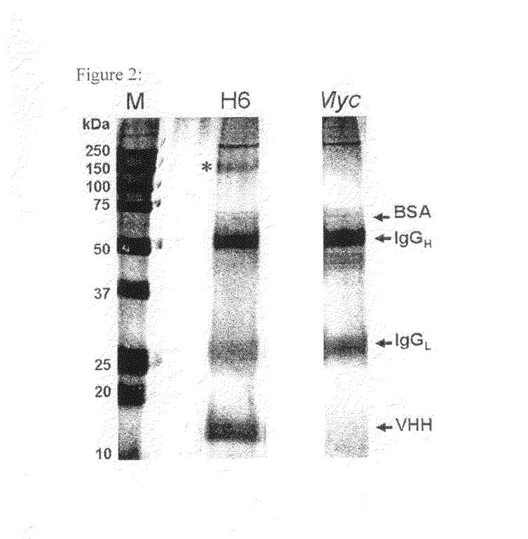

Phagemid containing E. coli TG I were infected with the helper phage VCSM13 and phage particles were produced overnight in medium containing both ampicillin and kanamycin and no glucose. These phages were precipitated with PEG and used in the 2nd round selection. In the second round of selection, live HeLa cells were used as antigen. HeLa cells were cultured in a 6 wells format for 24 hrs under normoxia (21% O₂) or hypoxia (1% O₂) (FIG. 1). Wells contained before the start of the selection procedure 1.1×10⁶ cells grown 60-70% confluence. Prior to hypoxic selections all the buffers used were put overnight in a hypoxic environment. Cells for counter selection were trypsinized and spun down at 1200 rpm in cold DM EM—bicarbonate buffered +10% FCS. The cells were resuspended in cold binding buffer (DMEM-bicarbonate buffered +10% FCS and 25 mM Hepes) and pre-incubated with 10 μl phages/ml (˜10¹⁰ phages/ml) for 30 min. The phage-counter selection cell mixtures (2 ml) were added to adherent cells from the opposite conditions and incubated for 30 min on ice at the indicated conditions. The excess of cells in suspension compared to adherent cells was 5 to 6-fold for hypoxia and normoxia, respectively. Wells with no adherent cells were used as a control.

Unbound phages were washed for 15 times with cold PBS supplemented with 1 mM Ca²⁺ and 1 mM Mg²⁺. Surface bound phages were stripped with three consecutive washes (S1, S2 and S3) of 1 ml of cold glycine buffer (500 mM NaCl; 100 mM glycine pH 2.5) for 5 min on ice. S1, S2 and S3 were neutralized with 0.5 ml 1M Tris-HCl pH 7.4. In a final step remaining phages were eluted by scraping the adherent cells and incubation with 1 ml of cold 100 mM TEA for 4 min (E). E was also neutralized with 0.5 ml 1M Tris-HCl pH 7.4. Phages from the different fractions were rescued by infecting E. coil TG1. Monoclonal phages from successful selections were screened in a 96 well ELISA using HeLa cells that were grown for 24 hrs under normoxia (21% O₂) or hypoxia (1% O₂) and fixed with 3.7% formaldehyde in PBS. Bound phages were detected with an anti-M13 antibody coupled to the enzyme horseradish peroxidase (HRP) (Amersham Pharmacia Biotech, Uppsala, Sweden). This procedure resulted in a number of monoclonal phages. After recloning of the genes encoding VHHs from these monoclonal phages, restriction patterns were determined and from at least two members of each restriction pattern the nucleotide sequences have been determined. These VHHs were screened in a number of additional tests like immunofluorescence, immunoprecipitation and Western blotting, all techniques well known by persons skilled in the art.

This resulted in large number of unique VHHs. From one particular branch of the dendrogramme of all selected VHHs, we deducted an overall amino acid sequence of VHHs that have the desired property: QVQLV(Q)E(D)SGGGLVQAGGSLRLSCA(V,E)ASGRTFSSYAMGWFRQA(P)PGKERE F(L,W)VA(S)T(A)ISRSGSA(T)IYAD(Y)P(S)VKGRFTM(I,V)SDNAKNTVYLEMNSLKP EDTAVF(Y)YCAAARSGV(I) PSSRPTD(N)YDYWGQGTQVTVSS, whereas (X) means that at that postion also the indicated amino acid can be present. These variations can occur in combination with any of the other variations indicated.

One VHH, VHH H6 was further investigated. The a.a. sequences (SEQ ID NO: 1485) of VHH H6 is given below in Table B-6. In italics the CDRs are given.

Table B-6. Amino acid sequence of VHH obtained with the differential hypoxia/normoxia methods described in example 1. The CDRs, defined as described by Lutje Hulsik at all. ( ) are in bold. We compared the amino acid sequence of VHH H6 with the universal VHH framework as proposed by Saerens et al (2005), the differences between that sequence and the sequence of VHH H6 are underscored. Frame work amino acid dat differ from germline (V) genes are in italics. The large number of differences indicate an active maturation process to arrive to these amino acids:

VHH H6:
QVQLQDSGGGLVQAGGSLRLSCEASGRTFSSYAMGWFRQPPGKEREWVST
ISRSGSAIYAYPVKGRFTMSRDNAKNTVYLEMNSLKPEDTAVFYCAAARS
GVPSSRPTDYDYWGQGTQVTVSS

The sequence of VHH H6 is unique in a number of aspects. Uniqueness of the CDRs can be expected, but the large deviations of the frame work residues compared to the consensus sequence as defined by Saerens et al is very surprising. Such a high variation of the frame work indicates an active maturation of these amino acids.

The following changes in the H6-VHH may be of importance (based on analisys of sequence vs germline):
S30
A34
E 23
W 47
S49
T 50
M69
F93

### Example 13

### Reverse Proteomics to Determine the Nature of a Cell Surface Protein Recognized by a Particular VHH Selected According to the Method Described in Example 1 and Proof that Binds to α3β1-Integrin In Vitro and In Vivo.

### Example 13.1

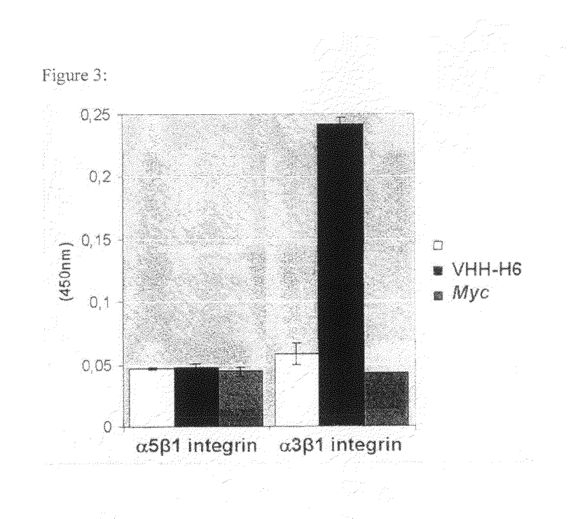

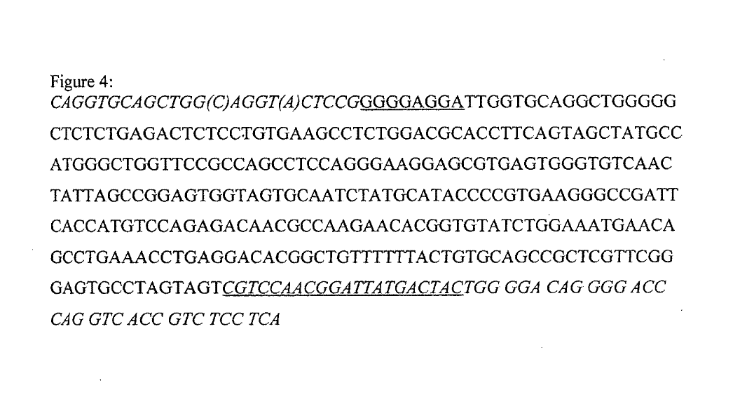

Reverse proteomics was used to identify the antigen(s) of VHH-H6. VHH-H6 clearly immunoprecipitated a single 150 kDa band (FIG. 2). Mass spectrometry analysis assigned the highest scores to integrin 131 for this 150 kDa protein. The highly significant scores strongly suggest VHH-H6 specifically recognizes the α3β1 (VLA-3) integrin complex.

### Example 13.2

To confirm that α3β1 integrin was immunoprecipitated with VHH-H6, lysates from HeLa cells grown under hypoxia or normoxia were immunoprecipitated in the presence or absence of VHH-H6 and the precipitated proteins were analyzed by SDS-PAGE and Western blotting using conventional anti-α3 and anti-β1 integrin antibodies. VHH-H6 immunoprecipitated both α3 light chain and β1. Trace amounts of the integrin α3 light chain were detected independently of the VHH-H6 pulldown. However the amount of α3 light chain subunit increased markedly by VHH-H6 pulldown, suggesting that the α3 subunit was immunoprecipitated in complex with the β1 subunit, probably in the VLA-3 protein complex.

### Example 13.3

Since α3 and β1 proteins are heterodimers in complex with many different proteins, we analyzed the binding of VHH-H6 to purified recombinant VLA-3 in vitro by ELISA to confirm the direct interaction of VHH-H6 with VLA-3. VHH-H6 detected and (VLA-3) integrin but not α5β1 (VLA-5) integrin (Figure) suggesting that VHH-H6 binds directly to the α3β1 integrin. Furthermore, since VLA-5 also contained the β1 subunit, VHH-H6 interacts with the extracellular domain of α3, alone or in complex with β1. Additionally, VHH-B4 did not interact with either recombinant VLA-3 nor VLA-5. The results described in this example showed that VHH H6 recognizes specifically a3β1 integrin.

### Example 14

### Broadening of the number of VHHs Recognizing a Particular Cell Surface Protein Using Part of the Nucleotide Sequence Information of Cdr3

Although the number of selected phages after 2 or 3 rounds of selection was substantial, the affinity and specificity of the VHHs present on these phages was not always as good as necessary for the targets in mind. On the other hand the selected phages were only a fraction of the phages that recognize the antigen of interest. Therefore we have developed a new, versitile method to increase the number of VHHs that recognize the antigen of interest. To that end two sets of DNA primers were designed, one against the rather conserved N terminus of all VHHs and one against the C-terminus. The latter contains the rather conserved C-terminus (Frame work 4 often having the amino acid sequence WGQGTQ VTVSS) and 4-7 amino acids of CDR3, which of course are unique for the VHH recognizing the antigen of interest.

Using PCR technology it is simple to get amplification of just those DNA sequences that encode for VHHs recognizing the antigen of interest. Subsequently the nucleotide sequence of these amplified fragments has been determined and the affinity against α3β1 is determined with a Biocore, in which the antigen is immobilized. In this way variant sequences of a particular sequence could be obtained and a certain fraction of these new VHHs will have either better binding properties or even better properties to modulate certain biological processes.

### Example 15

### Selection of Cells Carrying a Particular Cell Surface Protein Indicative for Tumors

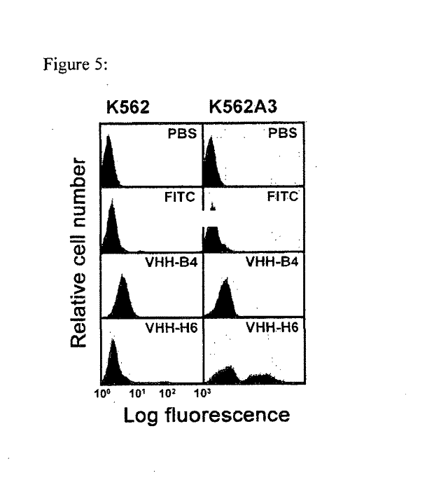

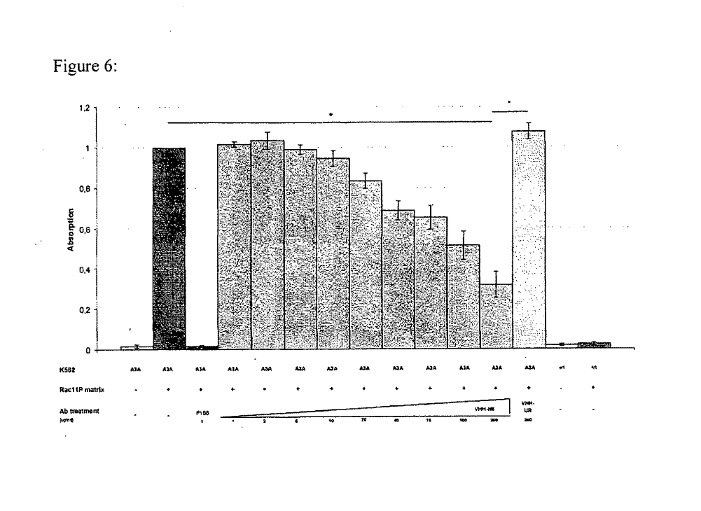

HeLa cells were exposed to normoxic or hypoxic conditions for 24 hrs. Cells were briefly trysinesed and spun down in cold culture medium at 1200 rpm for 5 min. Erythroleukemia K562 and K562A3 cells (a kind gift of Dr A. Sonnenberg) were cultured under normoxic conditions and spun down similar, medium was discarded. Cells were washed with 5 ml ice cold 0.2% BSA in PBS (BP) and divided over different tubes (approximately 500.000 cells per tube). Cells were incubated with VHH-B4 or VHH-H6 at a concentration of 300 μg/ml in BP, or BP alone, for 1 hr on ice. Cells were washed with 5 ml of BP, spun down and incubated with 20 ng/μl of FITC conjugated anti-HIS6 (C-term) (Invitrogen) for 30 min on ice. Next cells were washed again with 5 ml of BP, resuspended in 0.5 ml of BP and transferred into FACS tubes. Additional controls were performed with an anti-α3 integrin antibody (CD49c) (clone C3 II.1, BD Pharmingen, San Diego, Calif., USA), an anti-β1 integrin antibody (TS2/16, a kind gift of Dr. E. H. Danen), and anti GST (Santa Cruz). Antibodies were all used at 1/10 dilution, except for TS2/16 (1/2, supernatant of hybridoma), in combination with rat anti-mouse IgG1 PerCP (Becton Dickinson, San Diego, Calif., USA) diluted 1/50. FACS analysis was performed a flow cytometer (FACSCALIBUL, Becton Dickinson). The relative cell number was plotted against a Log fluorescence scale. To further substantiate the finding that VHH H6 specifically recognizes α3β1 (VLA-3) integrin in their natural context, we performed FACS analysis with K562 cells that lack a3 and compared those with the α3A transfectant line K562A3. Whereas the K562A3 transfectants expresses α3β1 and α5β1 on the surface, K562 cells only express a5β1. FACS analysis demonstrated that VHH-H6 only stained K562A3, indicating that VHH-H6 recognizes a3β1 (FIG. 5).

### Example 16

### Inhibition of Adhesion of K562 A3A Cell to RAC-1 by VHH H16

The linkage of the extracellular matrix to the cell requires transmembrane cell adhesion proteins that act as matrix receptors and tie the matrix to the cell cytoskeleton. Integrins are crucially important in that process. The variety of integrins heterodimers are formed from 9 types of □ubunits and 24 types of -subunits. Also posttranslational processing increase the diversity of integrin on surfaces. Because the same integrin molecule in different cell types can have different ligand-binding specificities, it seems that additional cell-type-specific factors can interact with integrins to modulate their binding activity. So the intrinsic diversity of integrins in the context of additional cell-type-specific factors make these molecules very suitable as diagnostic tool or for targeted delivery of drugs. To achieve that molecules that recognize specific integrins in there natural context are necessary. A subclass of such molecules may bind target integrins in such a way that intracellular signaling events are blocked.

To investigate whether VHH H6 alone or as homo bi-head or as hetero-bi-head can block the interaction of a cell with the extracellular matrix the following experiments were preformed. Rac11P coating: Let confluent cells secrete LN5 (laminin) rich matrix for 24 h 3×PBS wash o/n 20 mM EDTA at 4° C. 3×PBS wash 1 h, block 0.35% BSA in PBS at room temperature, was 2× with PBS.

Adhesion: Wash K562 once with DMEM 25 mM HEPES 0.35% BSA 175 ul 2*10e6 cells/ml K562+xul Ab+xul DMEM 25 mM HEPES 0.35% BSA (total 350 ul) 15 min Ab incubation at 37° C. in triplicate 100 ul/96 well 30 min adhesion at 37° C. 4×wash with DMEM 25 mM HEPES 0.35% BSA fix in 4% PFA 10 min at room temperature 2×H₂O wash stain with 5 mg/ml crystal violet in 2% EtOH 10 min at room temperature 4×H₂O wash 2% SDS 30 min at room temperature RT absorbtion at 655 nm.

### Example 17

### Construction of Homo- and Heterobiheads Recognizing One or Two Cell Surface Proteins Indicative for Solid Tumors

a. PCR was used to amplify the VHH sequences. Different primers sets are designed to amplify the VHH, which will be located at the N terminus and the VHH, which will be located at the C terminus of the bihead. The primers at the 3□ of [symbol] VHH and at the 5□ of [symbol] VHH, may encode a flexible sequence represented by a repeat of the dipeptide Gly-Ser. These same primers contain a unique restriction site (BamHI). After PCR amplification, the generated fragments are digested with a unique N-terminal restriction site (MfeI) and BamHI for the VHH that will be located at the N terminus, and with BamHI and a unique C-terminal restriction site (BstEII) for VHH that will be located at the C terminus. The fragments are ligated into an expression vector, which is digested with MfeI and BstEII. The VHH-bihead constructed in this way will be produced in E. coli after IPTG induction. The formed bihead will be secreted into the periplasm due to the presence of an OmpA-signal sequence.

The VHH-combination described above may consist of the same VHHs (homo-biheads) or of distinct VHHs (hetero-biheads). When the VHH sequence of interest contain an internal BamHI restriction site, this site should be removed beforehand. Alternatively, primers containing different restriction sites (BspEI) were designed.

b. Similar to the example described under a) a hetero-bihead of H6 can be constructed using the nucleotide sequences of H6 and another a3b1 single domain antibody (e.g. the ones described herein). To find the optimal bi-head both will be at the N terminus of the chimeric molecule, which means that H6 may be at the C-terminus resp. Also the nature and the length of the linker has to be optimized for various purposes.

### Example 18

### Fluorescence Microscopy with Biheads

Sample preparation: The human Erythroleukemia cell line K562 was cultivated in RPM1 medium supplemented with 10% FCS+pen+strep at 37° C. in 5% CO₂. K562 cells transfected with the integrin alpha3 (K562-A3) were grown under the same conditions, and the medium was supplemented with 1 mg/ml G418. The medium was exchanged one day before fixing the cells. The cells were fixed by adding equal volume of 4% paraformaldehyde in 0.1 M PHEM buffer (60 mM Pipes, 25 mM Hepes, 2 mM MgCl₂, 10 mM EGTA pH 6.9) to the culture medium, and incubation for 10 min at room temperature. Spin down the cells 5 min at 1200 rpm, and discard supernatant. Resuspend the cells in 20 ml (for a T75 flask) of the 4% paraformaldehyde solution (PFA), and incubate for 2 h at room temperature and overnight at 4° C. The PFA was removed by centrifugation of the cells 5 min at 1200 rpm. The cells were resuspended into 1 ml 0.1 M PHEM pH 6.9. The cells were washed 5-times with the same buffer. The cells were each time spun down for 1 min at 3000 rpm and resuspended into 1.5 ml 0.1 M PHEM pH 6.9. The cells were finally embedded into 12% gelatin in 0.1 M PHEM pH 6.9 by incubation at 37° C. for 5 min. The cells were pelleted in the gelatin solution by 5 min centrifugation at maximal speed. Resuspend the cells in a small volume of 12% gelatin (+/−30 ul). The gelatin was solidified by incubation for 15 min on ice. The cells were recuperated by cutting the tip of the tube and taking out the gelatin block containing the cells. The gelatin block was cut in smaller blocks, and incubated in 2.3 M sucrose in 0.1 M PHEM pH 6.9 overnight at 4° C. Next day, the blocks were put on small pins and frozen in liquid nitrogen.

Immunofluorescence: Make cryo-sections of about 350 to 450 nm thick with a glass knive at −80° C. or −100° C. Pick up the cryo-sections in 2.3 M sucrose in 0.1 M PHEM pH 6.9. Put the sections on silan-coated slide.

To get rid of the gelatin, wash the slides four times with PBS at 37° C. for 5 min each. Subsequently, wash the slides briefly with PBS at room temperature (5-times, 3 min). Incubate the slides with 1 mg/ml of Sodium Borohydride in PBS to quench the free aldehyde groups, followed by washing with PBS (5-times, 2 min), and 20 mM glycin in PBS (2-times, 3 min). The sections were blocked with 1% BSA in PBS (2-times, 5 min). The sections were incubated with the primary anti-integrin antibody (CD49c; Becton Dickinson), or with the anti-integrin nanobodies, for 1 h at room temperature, in 1% BSA in PBS. Different concentrations, ranging from 30 ug/ml up to 1 ug/ml, were used. Wash the sections with 0.1% BSA in PBS (5-times, 2 min).

The sections, which were then incubated with nanobodies (monoheads, biheads, or controls such as a commercial antibody CD49c (from BD Transduction Laboratories, Material number: 611045—see also results below) and were finally incubated with a bridging antibody (rabbit anti-llama heavy chain serum, diluted 1:100) for 1 h at room temperature, in 1% BSA in PBS, after which the sections were washed with 0.1% BSA in PBS (5-times, 2 min).

Finally the slides were incubated with the secondary fluorescent antibodies (Donkey anti-mouse coupled to Cy3, for sections incubated with CD49c, and Donkey anti-rabbit coupled to Cy3, for sections incubated with nanobodies and rabbit anti-llama heavy chain serum). The secondary antibodies were diluted (1:300) in 1% BSA in PBS, and incubated for 45 min at room temperature.

The sections were washed with PBS (5-times, 3 min), incubated with Dapi (1:1000 in PBS, from a stock of 2 mg/ml) for 5 min, and washed for the final time with PBS (5-times, 3 min) and with distilled water (5-times, 3 min).

The sections were embedded in Prolong Gold overnight at room temperature, and the coverslips were sealed the next moring with nailpolish.

The slides were analyzed with an Olympus AX70 fluorescence microscope.

### Sequence of the Biheads:

VHH-5 bihead (GS15):
SEQ ID NO: 1486
EVQLVESGGGLVQAGGSLRLSCAASGGTFRYQNMGWYRQAPGNEREWVASN
WATGATAYADSVKGRFTISRDDAKNVVYLQMNNLKPEDTAVYYCNRLSRPW
GWGQGTQVTVSSGGGGSGGGGSGGGGSEVQLVESGGGLVQAGGSLRLSCAA
SGGTFRYQNMGWYRQAPGNEREWVASNWATGATAYADSVKGRFTISRDDAK
NVVYLQMNNLKPEDTAVYYCNRLSRPWSWGQGTQVTVSS
VHH-5 bihead (GS5):
SEQ ID NO: 1487
EVQLVESGGGLVQAGGSLRLSCAASGGTFRYQNMGWYRQAPGNEREWVASN
WATGATAYADSVKGRFTISRDDAKNVVYLQMNNLKPEDTAVYYCNRLSRPW
GWGQGTQVTVSSGGGGSEVQLVESGGGLVQAGGSLRLSCAASGGTFRYQNM
GWYRQAPGNEREWVASNWATGATAYADSVKGRFTISRDDAKNVVYLQMNNL
KPEDTAVYYCNRLSRPWSWGQGTQVTVSS

### Results of Immunofluorescence:

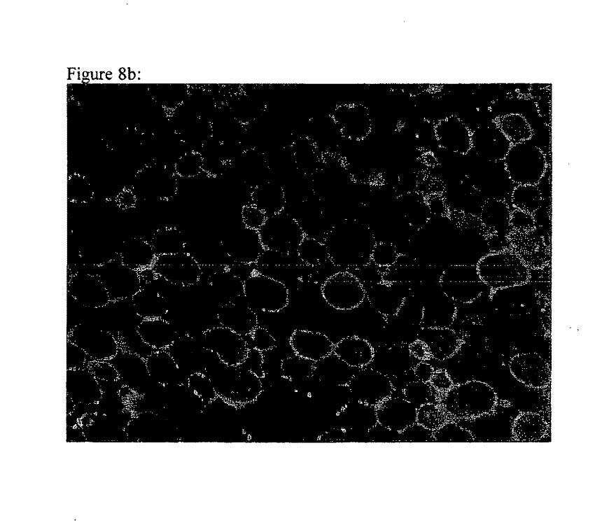

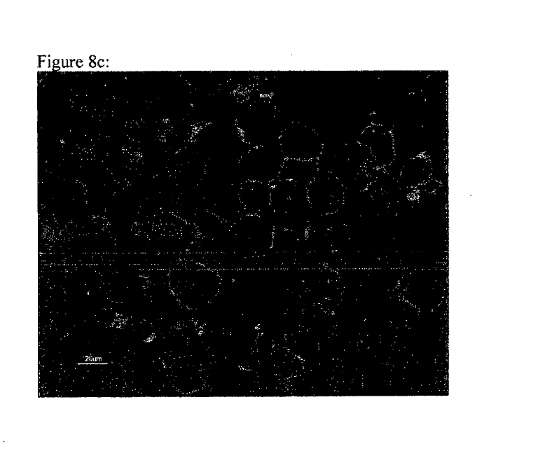

The concentrations used were 10 ug/ml for bihead with SEQ ID NO: 1486, i.e. VHH-5 bihead with GS15 (−0.3 uM), and 5 ug/ml for CD49c (−0.03 uM) and 10 ug/ml for the monhead (SEQ ID NO: 1474). The staining here (see FIGS. 8a and 8b) is better with the bihead compared with CD49c (a 10-fold excess of biheads was used). Bihead was doing also good, when tested at a concentration of 1 ug/ml (the same molarity as for CD49c □data not shown). Quality of labeling by VHH increases by construction of a bihead (SEQ ID NO: 1486) vs monohead (SEQ ID NO: 1474) (FIG. 8b+c).

### Example 18

### Chemotaxis

Wells of 96-well tissue culture plates are coated with various concentrations of fibronectin in PBS overnight at 4° C., blocked with 2% heat-denatured BSA for 2 h at 37° C., and washed once with PBS. Asynchronously growing cells are trypsinized, collected in culture medium, washed once with PBS, resuspended in DME/0.5% BSA, and added to the wells at 2_{—}104 cells per well. After 20 min of incubation at 37° C., unattached cells are removed by rinsing of the plates with PBS, and the remaining attached cells are lysed and stained at 37° C. overnight in 3.75 mM p-nitrophenyl N-acetyl-_-D-glucosamide/0.05 M sodium citrate/0.25% Triton X-100. The OD405 is determined in triplicate wells and related to the OD405 measured in wells in which all 2_{—}104 cells are stained to calculate the percentage of adhered cells (E. Danen et al., 2002, J Cell Biol 159:1071-1086).

Alternatively, cells are plated sparsely (3×10⁴ cells) on 24-mm glass coverslips coated with fibronectin used at a concentration at which cells adhere with high efficiency. 3 h later, coverslips are incubated for 2 h with 10 mg/ml mitomycin-C (Sigma-Aldrich) to inhibit cell division, washed, and incubated overnight in culture medium with or without nanobodies covered with mineral oil at 37° C. and 5% CO₂. A 10×dry lens objective is used and phase-contrast images are taken every 15 min on a Widefield CCD system (Carl Zeiss Microlmaging, Inc.); tracks of individual cells are analyzed using Image) software (National Institutes of Health, Bethesda, Md.). The migration speed is calculated as [total path length (μm)/time (hour)] and the persistence of migration is calculated as [net displacement (μm)/total path length (μm)].

### References to Target Amino Acid Sequence:

| Integrin name referred herein | Protein and nucleotide sequence |
|---|---|
| Human alphaL | GenBank accession number: NM_002209 |
| Human beta2 | GenBank accession number: NM_000211 |
| Human alphaM | GenBank accession number: NM_000632 |
| Human alphaVbeta6 | GenBank accession number: NM_002210 |
| | and NM_000888 |
| Human beta1 | GenBank accession number: NM_002211 |
| Human alpha5 | GenBank accession number: NM_002205 |
| Human alpha3 | GenBank accession number: NM_005501 |

List of targets (and alternative names) within the class (excerpt):

Alpha subunits:

Alpha1/CD49a, alpha2/CD49b, alpha2b/CD41, alpha3/CD49c, alpha4/CD49d, alpha5/CD49e, alpha6/CD49f, alpha7, alpha8, alpha9, alpha10, alpha11, alphaE/CD103, alphaL/CD 11a, alphaM/CD11b, alphaX/CD11c, alphaV/CD51, alphaD/CD11d

### Beta Subunits:

Beta1/CD29, beta2/CD18, beta3/CD61, beta4/CD104, beta5, beta6, beta7, beta8

Example of known heterodimers with other names (not exclusive):

a1b1/VLA-1, a2b1/VLA-2/GPIa, a3b1/VLA-3, a-4b1/VLA-4, a5b1/VLA-5, a6b1/VLA-6/GPIc, αLβ2/LFA-1, αMβ2MAC-1, αXβ2/p150/95/CR4, α4β7/LPAM-1

Alpha Subunits with alphaI Domain:

alphaE/CD103, alphaL/CD11a, alphaM/CD11b, alphaX/CD11c, alphaV/CD51, alphaD/CD11d, alpha1/CD49a, alpha2/CD49b, alpha10

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All references disclosed herein are incorporated by reference, in particular for the teaching that is referenced hereinabove.

### Preferred Aspects:

1. Amino acid sequence that is directed against and/or that can specifically bind to an integrin including human integrin, preferably to a subunit of integrin selected from the group consisting of alpha1, alpha2, alpha2b, alpha3, alpha4, alpha5, alpha6, alpha7, alpha8, alpha9, alpha10, alpha11, alphaE, alphaL, alphaM, alphaX, alphaV, alphaD, beta1, beta2, beta3, beta4, beta5, beta6, beta7, and beta8, more preferably to alpha3, alpha5, alphaL, alphaM, alphaV, beta1, beta2, beta6 and to any of the human forms of said integrins, e.g. for use as a therapeutic, preventative or diagnostic.
2. Amino acid sequence according to aspect 1, that is in essentially isolated form.
3. Amino acid sequence according to aspect 1 or 2, for administration to a subject, wherein said amino acid sequence does not naturally occur in said subject.
4. Amino acid sequence according to any of the preceding aspects, that can specifically bind to an integrin of aspect 1 with a dissociation constant (K_{D}) of 10⁻⁵ to 10⁻¹² moles/litre or less, and preferably 10⁻⁷ to 10⁻¹² moles/litre or less and more preferably 10⁻⁸ to 10⁻¹² moles/litre.
5. Amino acid sequence according to any of the preceding aspects, that can specifically bind to an integrin of aspect 1 with a rate of association (kₒₙ-rate) of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹.
6. Amino acid sequence according to any of the preceding aspects, that can specifically bind to an integrin of aspect 1 with a rate of dissociation (k_{off} rate) between 1s⁻¹ and 10⁻⁶ s⁻¹, preferably between 10⁻² s⁻¹ and 10⁻⁶ s⁻¹, more preferably between 10⁻³ s¹ and 10⁻⁶ s⁻¹, such as between 10⁻⁴ s⁻¹ and 10⁻⁶ s⁻¹.
7. Amino acid sequence according to any of the preceding aspects, that can specifically bind to an integrin of aspect 1 with an affinity less than 500 nM, preferably less than 200 nM, more preferably less than 10 nM, such as less than 500 pM.
8. Amino acid sequence according to any of the preceding aspects, that is a naturally occurring amino acid sequence (from any suitable species) or a synthetic or semi-synthetic amino acid sequence.
9. Amino acid sequence according to any of the preceding aspects, that comprises an immunoglobulin fold or that under suitable conditions is capable of forming an immunoglobulin fold.
10. Amino acid sequence according to any of the preceding aspects, that essentially consists of 4 framework regions (FR1 to FR4 respectively) and 3 complementarity determining regions (CDR1 to CDR3 respectively).
11. Amino acid sequence according to any of the preceding aspects, that is an immunoglobulin sequence.
12. Amino acid sequence according to any of the preceding aspects, that is a naturally occurring immunoglobulin sequence (from any suitable species) or a synthetic or semi-synthetic immunoglobulin sequence.
13. Amino acid sequence according to any of the preceding aspects that is a humanized immunoglobulin sequence, a camelized immunoglobulin sequence or an immunoglobulin sequence that has been obtained by techniques such as affinity maturation.
14. Amino acid sequence according to any of the preceding aspects, that essentially consists of a light chain variable domain sequence (e.g. a V_{L}-sequence); or of a heavy chain variable domain sequence (e.g. a V_{H}-sequence).
15. Amino acid sequence according to any of the preceding aspects, that essentially consists of a heavy chain variable domain sequence that is derived from a conventional four-chain antibody or that essentially consist of a heavy chain variable domain sequence that is derived from heavy chain antibody.
16. Amino acid sequence according to any of the preceding aspects, that essentially consists of a domain antibody (or an amino acid sequence that is suitable for use as a domain antibody), of a single domain antibody (or an amino acid sequence that is suitable for use as a single domain antibody), of a “dAb” (or an amino acid sequence that is suitable for use as a dAb) or of a Nanobody (including but not limited to a V_{HH} sequence).
17. Amino acid sequence according to any of the preceding aspects, that essentially consists of a Nanobody.
18. Amino acid sequence according to any of the preceding aspects, that essentially consists of a Nanobody that
i) has 80% amino acid identity with at least one of the amino acid sequences of SEQ ID NO□s: 1 to 22, in which for the purposes of determining the degree of amino acid identity, the amino acid residues that form the CDR sequences are disregarded;
   and in which:
ii) preferably one or more of the amino acid residues at positions 11, 37, 44, 45, 47, 83, 84, 103, 104 and 108 according to the Kabat numbering are chosen from the Hallmark residues mentioned in Table B-2.
   19. Amino acid sequence according to any of the preceding aspects, that essentially consists of a Nanobody that
i. has 80% amino acid identity with at least one of the amino acid sequences of SEQ ID NO□s 1316 to 1487, in which for the purposes of determining the degree of amino acid identity, the amino acid residues that form the CDR sequences are disregarded; and in which:
ii. preferably one or more of the amino acid residues at positions 11, 37, 44, 45, 47, 83, 84, 103, 104 and 108 according to the Kabat numbering are chosen from the Hallmark residues mentioned in Table B-2.
   20. Amino acid sequence according to any of the preceding aspects, that essentially consists of a humanized Nanobody.
   21. Amino acid sequence according to any of the preceding aspects, that in addition to the at least one binding site for binding against integrin or a subunit of an integrin according to aspect 1, contains one or more further binding sites for binding against other antigens, proteins or targets.
   22. Amino acid sequence directed against an integrin including human integrin, preferably to a subunit of integrin selected from the group consisting of alpha1, alpha2, alpha2b, alpha3, alpha4, alpha5, alpha6, alpha7, alpha8, alpha9, alpha10, alpha11, alphaE, alphaL, alphaM, alphaX, alphaV, alphaD, beta1, beta2, beta3, beta4, beta5, beta6, beta7, and beta8, more preferably to alpha3, alpha5, alphaL, alphaM, alphaV, beta1, beta2, beta6 and to any of the human forms of said integrins, e.g. for use as a therapeutic, preventative or diagnostic (e.g. for correlative imaging), that comprises one or more stretches of amino acid residues chosen from the group consisting of:
j) the amino acid sequences of SEQ ID NO□s 296 to 465;
k) amino acid sequences that have at least 80% amino acid identity with at least one of the amino acid sequences of SEQ ID NO□ 296s to 465;
l) amino acid sequences that have 3, 2, or 1 amino acid difference with at least one of the amino acid sequences of SEQ ID NO□ 296s to 465;
m) the amino acid sequences of SEQ ID NO□636s to 805;
n) amino acid sequences that have at least 80% amino acid identity with at least one of the amino acid sequences of SEQ ID NO□ 636s to 805;
o) amino acid sequences that have 3, 2, or 1 amino acid difference with at least one of the amino acid sequences of SEQ ID NO□ 636s to 805;
p) the amino acid sequences of SEQ ID NO□ 976s to 1145;
q) amino acid sequences that have at least 80% amino acid identity with at least one of the amino acid sequences of SEQ ID NO□ 976s to 1145;
r) amino acid sequences that have 3, 2, or 1 amino acid difference with at least one of the amino acid sequences of SEQ ID NOD: 976 to 1145;
   or any suitable combination thereof.
   23. Amino acid sequence according to aspect 22, in which at least one of said stretches of amino acid residues forms part of the antigen binding site for binding against an integrin.
   24. Amino acid sequence according to aspect 22, that comprises two or more stretches of amino acid residues chosen from the group consisting of:
j) the amino acid sequences of SEQ ID NO□ 296s to 465;
k) amino acid sequences that have at least 80% amino acid identity with at least one of the amino acid sequences of SEQ ID NO□ 296s to 465;
l) amino acid sequences that have 3, 2, or 1 amino acid difference with at least one of the amino acid sequences of SEQ ID NO□ 296s to 465;
m) the amino acid sequences of SEQ ID NO□ 636s to 805;
n) amino acid sequences that have at least 80% amino acid identity with at least one of the amino acid sequences of SEQ ID NO□ 636s to 805;
o) amino acid sequences that have 3, 2, or 1 amino acid difference with at least one of the amino acid sequences of SEQ ID NO□ 636s to 805;
p) the amino acid sequences of SEQ ID NO□ 976s to 1145;
q) amino acid sequences that have at least 80% amino acid identity with at least one of the amino acid sequences of SEQ ID NO□ 976s to 1145;
r) amino acid sequences that have 3, 2, or 1 amino acid difference with at least one of the amino acid sequences of SEQ ID NO□ 976s to 1145;
   such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), e) or f).
   25. Amino acid sequence according to aspect 24, in which the at least two stretches of amino acid residues forms part of the antigen binding site for binding against an integrin.
   26. Amino acid sequence according to any of aspects 24, that comprises three or more stretches of amino acid residues, in which the first stretch of amino acid residues is chosen from the group consisting of:
a) the amino acid sequences of SEQ ID NO□ 296s to 465;
b) amino acid sequences that have at least 80% amino acid identity with at least one of the amino acid sequences of SEQ ID NO□ 296s to 465;
c) amino acid sequences that have 3, 2, or 1 amino acid difference with at least one of the amino acid sequences of SEQ ID NO□ 296s to 465;
   the second stretch of amino acid residues is chosen from the group consisting of:
d) the amino acid sequences of SEQ ID NO□ 636s to 805;
e) amino acid sequences that have at least 80% amino acid identity with at least one of the amino acid sequences of SEQ ID NO□ 636s to 805;
f) amino acid sequences that have 3, 2, or 1 amino acid difference with at least one of the amino acid sequences of SEQ ID NO□ 636s to 805;
   and the third stretch of amino acid residues is chosen from the group consisting of:
g) the amino acid sequences of SEQ ID NO□ 976s to 1145;
h) amino acid sequences that have at least 80% amino acid identity with at least one of the amino acid sequences of SEQ ID NO□ 976s to 1145;
i) amino acid sequences that have 3, 2, or 1 amino acid difference with at least one of the amino acid sequences of SEQ ID NO□ 976s to 1145.
   27. Amino acid sequence according to aspect 24, in which the at least three stretches of amino acid residues forms part of the antigen binding site for binding against integrin.
   28. Amino acid sequence according to any of previous aspects, in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO□ 131s6 to 1487.
   29. Amino acid sequence directed against an integrin that cross-blocks the binding of at least one of the amino acid sequences according to any of aspects 22 to 28.
   30. Amino acid sequence directed against an integrin that is cross-blocked from binding to an integrin by at least one of the amino acid sequences according to any of aspects 22 to 29.
   31. Amino acid sequence according to any of aspects 29 or 30, wherein the ability of said amino acid sequence to cross-block or to be cross-blocked is detected in a Biacore assay.
32. Amino acid sequence according to any of aspects 29 to 31, wherein the ability of said amino acid sequence to cross-block or to be cross-blocked is detected in an ELISA assay.
33. Amino acid sequence according to any of aspects 29 to 31, that can specifically bind to an integrin with a dissociation constant (K_{D}) of 10⁻⁵ to 10⁻¹² moles/litre or less, and preferably 10⁻⁷ to 10⁻¹² moles/litre or less and more preferably 10⁻⁸ to 10⁻¹² moles/litre.
34. Amino acid sequence according to any of aspects 29 to 31, that can specifically bind to an integrin with a rate of association (kₒₙ-rate) of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹.
   35. Amino acid sequence according to any of aspects 29 to 31, that can specifically bind to an integrin with a rate of dissociation (k_{off}-rate) between 1 s⁻¹ and 10⁻⁶ s⁻¹ preferably between 10⁻² s⁻¹ and 10⁻⁶ s⁻¹, more preferably between 10⁻³ s⁻¹ and 10−6 s⁻¹, such as between 10⁻⁴ s⁻¹ and 10⁻⁶ s⁻¹.
   36. Amino acid sequence according to any of aspects 29 to 31, that can specifically bind to an integrin with an affinity less than 500 nM, preferably less than 200 nM, more preferably less than 10 nM, such as less than 500 pM.
   37. Amino acid sequence according to any of aspects 29 to 31, that essentially consists of a heavy chain variable domain sequence that is derived from a conventional four-chain antibody or that essentially consist of a heavy chain variable domain sequence that is derived from heavy chain antibody.
   38. Amino acid sequence according to any of aspects 29 to 31, that essentially consists of a domain antibody (or an amino acid sequence that is suitable for use as a domain antibody), of a single domain antibody (or an amino acid sequence that is suitable for use as a single domain antibody), of a “dAb” (or an amino acid sequence that is suitable for use as a dAb) or of a Nanobody (including but not limited to a V_{HH} sequence).
   39. Amino acid sequence according to any of aspects 22 to 38, that essentially consists of a Nanobody that
i) has 80% amino acid identity with at least one of the amino acid sequences of SEQ ID NOD s: 1 to 22, in which for the purposes of determining the degree of amino acid identity, the amino acid residues that form the CDR sequences are disregarded;
   and in which:
ii) preferably one or more of the amino acid residues at positions 11, 37, 44, 45, 47, 83, 84, 103, 104 and 108 according to the Kabat numbering are chosen from the Hallmark residues mentioned in Table B-2.
   40. Amino acid sequence according to any of aspects 22 to 39, that essentially consists of a Nanobody that
i) has 80% amino acid identity with at least one of the amino acid sequences of SEQ ID-NO□ 131s6 to 1487, in which for the purposes of determining the degree of amino acid identity, the amino acid residues that form the CDR sequences are disregarded;
   and in which:
ii) preferably one or more of the amino acid residues at positions 11, 37, 44, 45, 47, 83, 84, 103, 104 and 108 according to the Kabat numbering are chosen from the Hallmark residues mentioned in Table B-2.
   41. Amino acid sequence according to any of aspects 22 to 40, that essentially consists of a humanized Nanobody.
   42. Amino acid sequence according to any of the preceding aspects, that in addition to the at least one binding site for binding formed by the CDR sequences, contains one or more further binding sites for binding against other antigens, proteins or targets.
   43. Amino acid sequence that essentially consists of 4 framework regions (FR I to FR4, respectively) and 3 complementarity determining regions (CDR1 to CDR3, respectively), in which:
   CDR1 is chosen from the group consisting of:
j) the amino acid sequences of SEQ ID NO□296s to 465;
k) amino acid sequences that have at least 80% amino acid identity with at least one of the amino acid sequences of SEQ ID NO□ 296s to 465;
l) amino acid sequences that have 3, 2, or 1 amino acid difference with at least one of the amino acid sequences of SEQ ID NO□ 296s to 465;
   and/or
   CDR2 is chosen from the group consisting of:
m) the amino acid sequences of SEQ ID NO□ 636s to 805;
n) amino acid sequences that have at least 80% amino acid identity with at least one of the amino acid sequences of SEQ ID NO□ 636s to 805;
o) amino acid sequences that have 3, 2, or 1 amino acid difference with at least one of the amino acid sequences of SEQ ID NO□ 636s to 805;
   and/or
   CDR3 is chosen from the group consisting of:
p) the amino acid sequences of SEQ ID NO□ 976s to 1145;
q) amino acid sequences that have at least 80% amino acid identity with at least one of the amino acid sequences of SEQ ID NO□ 976s to 1145;
r) amino acid sequences that have 3, 2, or 1 amino acid difference with at least one of the amino acid sequences of SEQ ID NO□ 976s to 1145.
   44. Amino acid sequence that essentially consists of 4 framework regions (FR1 to FR4, respectively) and 3 complementarity determining regions (CDR1 to CDR3, respectively), in which:
   CDR1 is chosen from the group consisting of:
j) the amino acid sequences of SEQ ID NO□ 296s to 465;
k) amino acid sequences that have at least 80% amino acid identity with at least one of the amino acid sequences of SEQ ID NO□ 296s to 465;
l) amino acid sequences that have 3, 2, or 1 amino acid difference with at least one of the amino acid sequences of SEQ ID NO□ 296s to 465;
   and
   CDR2 is chosen from the group consisting of:
m) the amino acid sequences of SEQ ID NO□ 636s to 805;
n) amino acid sequences that have at least 80% amino acid identity with at least one of the amino acid sequences of SEQ ID NO□ 636s to 805;
o) amino acid sequences that have 3, 2, or 1 amino acid difference with at least one of the amino acid sequences of SEQ ID NO□ 636s to 805;
   and
   CDR3 is chosen from the group consisting of:
p) the amino acid sequences of SEQ ID NO□ 976s to 1145;
q) amino acid sequences that have at least 80% amino acid identity with at least one of the amino acid sequences of SEQ ID NO□ 976s to 1145;
r) amino acid sequences that have 3, 2, or 1 amino acid difference with at least one of the amino acid sequences of SEQ ID NO□s: 91646 [symbol] .
   45. Amino acid sequence according to any of aspects 43 to 44, in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO□s: 1316 to 1487.
   46. Amino acid sequence directed against an integrin that cross-blocks the binding of at least one of the amino acid sequences according to any of aspects 43 to 45.
   47. Amino acid sequence directed against an integrin that is cross-blocked from binding to an integrin by at least one of the amino acid sequences according to any of aspects 43 to 46.
   48. Amino acid sequence according to any of aspects 43 to 47 wherein the ability of said amino acid sequence to cross-block or to be cross-blocked is detected in a Biacore assay.
   49. Amino acid sequence according to any of aspects 43 to 48 wherein the ability of said amino acid sequence to cross-block or to be cross-blocked is detected in an ELISA assay.
   50. Amino acid sequence according to any of aspects 43 to 49, that is in essentially isolated form.
   51. Amino acid sequence according to any of aspects 43 to 50, for administration to a subject, wherein said amino acid sequence does not naturally occur in said subject.
   52. Amino acid sequence according to any of aspects 43 to 51, that can specifically bind to an integrin with a dissociation constant (K_{D}) of 10⁻⁵ to 10⁻¹² moles/litre or less, and preferably 10⁻⁷ to 10⁻¹² moles/litre or less and more preferably 10⁻⁸ to 10⁻¹² moles/litre.
   53. Amino acid sequence according to any of aspects 43 to 51, that can specifically bind to an integrin with a rate of association (kₒₙ-rate) of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹.
   54. Amino acid sequence according to any of aspects 43 to 51, that can specifically bind to an integrin with a rate of dissociation (k_{off} rate) between 1s⁻¹ and 10⁻⁶ s⁻¹ preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.
   55. Amino acid sequence according to any of aspects 43 to 51, that can specifically bind to an integrin with an affinity less than 500 nM, preferably less than 200 nM, more preferably less than 10 nM, such as less than 500 pM.
   56. Amino acid sequence according to any of aspects 43 to 55, that is a naturally occurring amino acid sequence (from any suitable species) or a synthetic or semi-synthetic amino acid sequence.
   57. Amino acid sequence according to any of aspects 43 to 56, that comprises an immunoglobulin fold or that under suitable conditions is capable of forming an immunoglobulin fold.
   58. Amino acid sequence according to any of aspects 43 to 57, that is an immunoglobulin sequence.
   59. Amino acid sequence according to any of aspects 43 to 58, that is a naturally occurring immunoglobulin sequence (from any suitable species) or a synthetic or semi-synthetic immunoglobulin sequence.
   60. Amino acid sequence according to any of aspects 43 to 59, that is a humanized immunoglobulin sequence, a camelized immunoglobulin sequence or an immunoglobulin sequence that has been obtained by techniques such as affinity maturation.
   61. Amino acid sequence according to any of aspects 43 to 60, that essentially consists of a light chain variable domain sequence (e.g. a V_{L}-sequence); or of a heavy chain variable domain sequence (e.g. a V_{H}-sequence).
   62. Amino acid sequence according to any of aspects 43 to 61, that essentially consists of a heavy chain variable domain sequence that is derived from a conventional four-chain antibody or that essentially consist of a heavy chain variable domain sequence that is derived from heavy chain antibody.
   63. Amino acid sequence according to any of aspects 43 to 62, that essentially consists of a domain antibody (or an amino acid sequence that is suitable for use as a domain antibody), of a single domain antibody (or an amino acid sequence that is suitable for use as a single domain antibody), of a “dAb” (or an amino acid sequence that is suitable for use as a dAb) or of a Nanobody (including but not limited to a V_{HH} sequence).
   64. Amino acid sequence according to any of aspects 43 to 63, that essentially consists of a Nanobody.
   65. Amino acid sequence according to any of aspects 43 to 64, that essentially consists of a Nanobody that
i) has 80% amino acid identity with at least one of the amino acid sequences of SEQ ID NO□s: 1 to 22, in which for the purposes of determining the degree of amino acid identity, the amino acid residues that form the CDR sequences are disregarded;
   and in which:
ii) preferably one or more of the amino acid residues at positions 11, 37, 44, 45, 47, 83, 84, 103, 104 and 108 according to the Kabat numbering are chosen from the Hallmark residues mentioned in Table B-2.
   66. Amino acid sequence according to any of aspects 43 to 65, that essentially consists of a Nanobody that
i) has 80% amino acid identity with at least one of the amino acid sequences of SEQ ID NO□1316 to 1487, in which for the purposes of determining the degree of amino acid identity, the amino acid residues that form the CDR sequences are disregarded;
   and in which:
ii) preferably one or more of the amino acid residues at positions 11, 37, 44, 45, 47, 83, 84, 103, 104 and 108 according to the Kabat numbering are chosen from the Hallmark residues mentioned in Table B-2.
   67. Amino acid sequence according to any of aspects 43 to 66, that essentially consists of a humanized Nanobody.
   68. Amino acid sequence according to any of the preceding aspects, that in addition to the at least one binding site for binding formed by the CDR sequences, contains one or more further binding sites for binding against other antigens, proteins or targets.
   69. Nanobody that is directed against and/or that can specifically bind to an integrin including human integrin, preferably to a subunit of integrin selected from the group consisting of alpha1, alpha2, alpha2b, alpha3, alpha4, alpha5, alpha6, alpha7, alpha8, alpha9, alpha10, alpha11, alphaE, alphaL, alphaM, alphaX, alphaV, alphaD, beta1, beta2, beta3, beta4, beta5, beta6, beta7, and beta8, more preferably to alpha3, alpha5, alphaL, alphaM, alphaV, beta1, beta2, beta6 and to any of the human forms of said integrins, e.g. for use as a therapeutic, preventative or diagnostic (e.g. for correlative imaging).
   70. Nanobody according to aspect 69, that is in essentially isolated form.
   71. Nanobody according to any of aspects 69 to 70, that can specifically bind to an integrin with a dissociation constant (K_{D}) of 10⁻⁵ to 10⁻¹² moles/litre or less, and preferably 10⁻⁷ to 10⁻¹² moles/litre or less and more preferably 10⁻⁸ to 10⁻¹² moles/litre.
   72. Nanobody according to any of aspects 69 to 71, that can specifically bind to an integrin with a rate of association (kₒₙ-rate) of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷M⁻¹s⁻¹, such as between 10⁵ M⁻¹s⁻¹ and 10⁷ M⁻¹s⁻¹.
   73. Nanobody according to any of aspects 69 to 72, that can specifically bind to an integrin with a rate of dissociation (k_{off} rate) between 1 s¹ and 10⁻⁶ s⁻¹ preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.
   74. Nanobody according to any of aspects 69 to 73, that can specifically bind to an integrin with an affinity less than 500 nM, preferably less than 200 nM, more preferably less than 10 nM, such as less than 500 pM.
   75. Nanobody according to any of aspects 69 to 74, that is a naturally occurring Nanobody (from any suitable species) or a synthetic or semi-synthetic Nanobody.
   76. Nanobody according to any of aspects 69 to 75, that is a V_{HH} sequence, a partially humanized VHH sequence, a fully humanized VHH sequence, a camelized heavy chain variable domain or a Nanobody that has been obtained by techniques such as affinity maturation.
   77. Nanobody according to any of aspects 69 to 76, that
iii) has 80% amino acid identity with at least one of the amino acid sequences of SEQ ID NO□s: 1 to 22, in which for the purposes of determining the degree of amino acid identity, the amino acid residues that form the CDR sequences are disregarded;
   and in which:
iv) preferably one or more of the amino acid residues at positions 11, 37, 44, 45, 47, 83, 84, 103, 104 and 108 according to the Kabat numbering are chosen from the Hallmark residues mentioned in Table B-2.
   78. Nanobody according to any of aspects 69 to 76, that
v) has 80% amino acid identity with at least one of the amino acid sequences of SEQ ID NO□ 131s6 to 1487, in which for the purposes of determining the degree of amino acid identity, the amino acid residues that form the CDR sequences are disregarded;
   and in which:
vi) preferably one or more of the amino acid residues at positions 11, 37, 44, 45, 47, 83, 84, 103, 104 and 108 according to the Kabat numbering are chosen from the Hallmark residues mentioned in Table B-2.
   79. Nanobody according to any of aspects 69 to 76, in which:
   CDR1 is chosen from the group consisting of:
a) the amino acid sequences of SEQ ID NO□ 296s to 465;
b) amino acid sequences that have at least 80% amino acid identity with at least one of the amino acid sequences of SEQ ID NO□ 296s to 465;
c) amino acid sequences that have 3, 2, or 1 amino acid difference with at least one of the amino acid sequences of SEQ ID NO□ 296s to 465;
   and/or
   CDR2 is chosen from the group consisting of:
d) the amino acid sequences of SEQ ID NO□ 636s to 805;
e) amino acid sequences that have at least 80% amino acid identity with at least one of the amino acid sequences of SEQ ID NO□636s to 805;
f) amino acid sequences that have 3, 2, or 1 amino acid difference with at least one of the amino acid sequences of SEQ ID NO□ 636s to 805;
   and/or
   CDR3 is chosen from the group consisting of:
g) the amino acid sequences of SEQ ID NO□ 976s to 1145;
h) amino acid sequences that have at least 80% amino acid identity with at least one of the amino acid sequences of SEQ ID NO□s: 976 to 1145;
i) amino acid sequences that have 3, 2, or 1 amino acid difference with at least one of the amino acid sequences of SEQ ID NO□s: 976 to 1145.
   80. Nanobody according to any of aspects 69 to 79, in which:
   CDR1 is chosen from the group consisting of:
a) the amino acid sequences of SEQ ID NO□ 296s to 465;
b) amino acid sequences that have at least 80% amino acid identity with at least one of the amino acid sequences of SEQ ID NO□ 296s to 465;
c) amino acid sequences that have 3, 2, or 1 amino acid difference with at least one of the amino acid sequences of SEQ ID NO□ 296s to 465;
   and
   CDR2 is chosen from the group consisting of:
d) the amino acid sequences of SEQ ID NO□ 636s to 805;
e) amino acid sequences that have at least 80% amino acid identity with at least one of the amino acid sequences of SEQ ID NO□ 636s to 805;
f) amino acid sequences that have 3, 2, or 1 amino acid difference with at least one of the amino acid sequences of SEQ ID NO□ 636s to 805;
   and
   CDR3 is chosen from the group consisting of:
g) the amino acid sequences of SEQ ID NO□ 976s to 1145;
h) amino acid sequences that have at least 80% amino acid identity with at least one of the amino acid sequences of SEQ ID NO□ 976s to 1145;
i) amino acid sequences that have 3, 2, or 1 amino acid difference with at least one of the amino acid sequences of SEQ ID NO□s: 976 to 1145.
   81. Nanobody according to any of aspects 69 to 80, in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO□s: 13 [symbol] .
   82. Nanobody according to any of aspects 69 to 81, which is a partially humanized Nanobody.
   83. Nanobody according to any of aspects, which is a fully humanized Nanobody.
   84. Nanobody according to any of aspects 69 to 83, that is chosen from the group consisting of SEQ ID NO□s: 1316 to 1487 or from the group consisting of from amino acid sequences that have more than 80%, preferably more than 90%, more preferably more than 95%, such as 99% or more sequence identity (as defined herein) with at least one of the amino acid sequences of SEQ ID NO□s: 1316 to 1487.
   85. Nanobody directed against an integrin that cross-blocks the binding of at least one of the amino acid sequences according to any of previous aspects.
   86. Nanobody directed against an integrin that is cross-blocked from binding to an integrin by at least one of the amino acid sequences according to any of the previous aspects.
   87. Nanobody according to any of aspects 85 and 86 wherein the ability of said Nanobody to cross-block or to be cross-blocked is detected in a Biacore assay.
   88. Nanobody according to any of aspects 85 to 87 wherein the ability of said Nanobody to cross-block or to be cross-blocked is detected in an ELISA assay.
   89. Polypeptide that comprises or essentially consists of one or more amino acid sequences according to any of the previous aspects and/or one or more Nanobodies according to any of the previous aspects, and optionally further comprises one or more peptidic moieties.
   90. Polypeptide according to aspect 89, in which said one or more other peptidic moieties are chosen from the group consisting of domain antibodies, amino acid sequences that are suitable for use as a domain antibody, single domain antibodies, amino acid sequences that are suitable for use as a single domain antibody, “dAb”□s, amino acid sequences that are suitable for use as a dAb, or Nanobodies.
   91. Polypeptide according to any of aspects 89 to 90, in which said one or more amino acid sequences of the invention are chosen from the group consisting of domain antibodies, amino acid sequences that are suitable for use as a domain antibody, single domain antibodies, amino acid sequences that are suitable for use as a single domain antibody, “dAb”□s, amino acid sequences that are suitable for use as a dAb, or Nanobodies.
   92. Polypeptide, that comprises or essentially consists of one or more Nanobodies according to any of aspects 89 to 91 and in which said one or more other peptidic moieties are Nanobodies.
   93. Polypeptide according to any of aspects 89 to 91, that is a multivalent construct.
   94. Polypeptide according to any of aspects 89 to 91, that is a multispecific construct.
   95. Polypeptide according to any of aspects 89 to 91, that has an increased half-life.
   96. Polypeptide according to aspects 89 to 91, in which said one or more other peptidic moieties that provide the polypeptide with increased half-life is chosen from the group consisting of serum proteins or fragments thereof, binding units that can bind to serum proteins, an Fc portion, and small proteins or peptides that can bind to serum proteins.
   97. Polypeptide according to aspects 89 to 91, in which said one or more other peptidic moieties that provide the polypeptide with increased half-life is chosen from the group consisting of human serum albumin or fragments thereof.
   98. Polypeptide according to aspect 97, in which said one or more other peptidic moieties that provides the polypeptide with increased half-life are chosen from the group consisting of binding units that can bind to serum albumin (such as human serum albumin) or a serum immunoglobulin (such as IgG).
   99. Compound or construct, that comprises or essentially consists of one or more amino acid sequences according to any of previous aspects and/or one or more Nanobodies according to any previous aspects, and optionally further comprises one or more other groups, residues, moieties or binding units, optionally linked via one or more linkers.
   100. Compound or construct according to aspect 99 in which said one or more other groups, residues, moieties or binding units are amino acid sequences.
   101. Compound or construct according to aspect 99, in which said one or more linkers, if present, are one or more amino acid sequences.
   102. Compound or construct according to any of aspects 99 to 101, in which said one or more other groups, residues, moieties or binding units are immunoglobulin sequences.
   103. Compound or construct according to any of aspects 99 to 102, in which said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”□s, amino acid sequence [symbol] suitable for use as a dAb, or Nanobodies.
   104. Compound or construct according to any of aspects 99 to 103 in which said one or more amino acid sequences of the invention are immunoglobulin sequences.
   105. Compound or construct according to any of aspects 99 to 104, in which said one or more amino acid sequences of the invention are chosen from the group consisting of domain antibodies, amino acid sequences that are suitable for use as a domain antibody, single domain antibodies, amino acid sequences that are suitable for use as a single domain antibody, “dAb”□s, amino acid sequences that are suitable for use as a dAb, or Nanobodies.
   106. Compound or construct, that comprises or essentially consists of one or more Nanobodies according to any of aspects 99 to 105 and in which said one or more other groups, residues, moieties or binding units are Nanobodies.
   107. Compound or construct according to any of aspects 99 to 106, which is a multivalent construct.
   108. Compound or construct according to any of aspects 99 to 106, which is a multispecific construct.
   109. Compound or construct according to aspect 99 to 106, in which said one or more other groups, residues, moieties or binding units provide the compound or construct with an increased half-life and said other groups, residues, moieties or binding units is chosen from the group consisting of serum proteins or fragments thereof, binding units that can bind to serum proteins, an Fc portion, and small proteins or peptides that can bind to serum proteins.
   110. Compound or construct according to aspect 109, in which said one or more other groups, residues, moieties or binding units that provide the compound or construct with increased half-life is chosen from the group consisting of human serum albumin or fragments thereof.
   111. Compound or construct according to aspect 109, in which said one or more other groups, residues, moieties or binding units that provides the compound or construct with increased half-life are chosen from the group consisting of binding units that can bind to serum albumin (such as human serum albumin) or a serum immunoglobulin (such as IgG).
   112. Compound or construct according to aspect 109, in which said one or more other groups, residues, moieties or binding units that provides the compound or construct with increased half-life are chosen from the group consisting of domain antibodies, amino acid sequences that are suitable for use as a domain antibody, single domain antibodies, amino acid sequences that are suitable for use as a single domain antibody, “dAb”□s, amino acid sequences that are suitable for use as a dAb, or Nanobodies that can bind to serum albumin (such as human serum albumin) or a serum immunoglobulin (such as IgG).
   113. Compound or construct according to aspect 109, in which said one or more other groups, residues, moieties or binding units that provides the compound or construct with increased half-life is a Nanobody that can bind to serum albumin (such as human serum albumin) or a serum immunoglobulin (such as IgG).
   114. Compound or construct according to any of aspects 109, that has a serum half-life that is at least 1.5 times, preferably at least 2 times, such as at least 5 times, for example at least 10 times or more than 20 times, greater than the half-life of the corresponding amino acid sequence according without the other groups, residues, moieties or binding units that provides the compound or construct with increased half-life.
   115. Compound or construct according to any of aspects 109 to 114, that has a serum half-life that is increased with more than 1 hours, preferably more than 2 hours, more preferably more than 6 hours, such as more than 12 hours, or even more than 24, 48 or 72 hours than the half-life of the corresponding amino acid sequence according without the other groups, residues, moieties or binding units that provides the compound or construct with increased half-life.
   116. Compound or construct according to any of aspects 109 to 115, that has a serum half-life in human of at least about 12 hours, preferably at least 24 hours, more preferably at least 48 hours, even more preferably at least 72 hours or more; for example, of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).
   117. Monovalent construct, comprising or essentially consisting of one amino acid sequence according to any of previous aspects and/or one Nanobody according to any of previous aspects.
   118. Monovalent construct according to aspect 117 in which said amino acid sequence of the invention is chosen from the group consisting of domain antibodies, amino acid sequences that are suitable for use as a domain antibody, single domain antibodies, amino acid sequences that are suitable for use as a single domain antibody, “dAb”□s, [symbol] amiitb a sequences that are suitable for use as a dAb, or Nanobodies.
   119. Monovalent construct, comprising or essentially consisting of one Nanobody according to any of the previous aspects.
   120. Pharmaceutical composition, comprising at least one amino acid sequence according to any of the previous aspects, Nanobody according to any of the previous aspects, compound or construct according to any of the previous aspects, monovalent construct according to any of the previous aspects.
   121. Composition of aspect 120, that further comprises at least one pharmaceutically acceptable carrier, diluent or excipient and/or adjuvant, and that optionally comprises one or more further pharmaceutically active polypeptides and/or compounds.
   122. Nucleic acid or nucleotide sequence, that encodes an amino acid sequence according to any of the previous aspects, a Nanobody according to any of the previous aspects, a compound or construct according to any of previous aspects that is such that it can be obtained by expression of a nucleic acid or nucleotide sequence encoding the same, or a monovalent construct according to any of the previous aspects.
   123. Nucleic acid or nucleotide sequence according to aspect 122, that is in the form of a genetic construct.
   124. Host or host cell that expresses, or that under suitable circumstances is capable of expressing, an amino acid sequence according to any of previous aspects, a Nanobody according to any of previous aspects, a compound or construct according to any of previous aspects that is such that it can be obtained by expression of a nucleic acid or nucleotide sequence encoding the same, or a monovalent construct according to any of previous aspects and/or that comprises a nucleic acid or nucleotide sequence according to previous aspects, or a genetic construct according to previous aspects.
   125. Method for producing an amino acid sequence according to any of previous aspects, a Nanobody according to any of previous aspects, a compound or construct according to any of aspects, pharmaceutical composition according to previous aspects that is such that it can be obtained by expression of a nucleic acid or nucleotide sequence encoding the same, or a monovalent construct according to any of previous aspects said method at least comprising the steps of:
a) expressing, in a suitable host cell or host organism or in another suitable expression system, a nucleic acid or nucleotide sequence according to previous aspects or a genetic construct according to any of the previous aspects
   optionally followed by:
b) isolating and/or purifying the amino acid sequence according to any of the previous aspects, the Nanobody according to any of the previous aspects, the compound or construct according to any of the previous aspects that is such that it can be obtained by expression of a nucleic acid or nucleotide sequence encoding the same, or the monovalent construct according to any of the previous aspects thus obtained.
   126. Method for producing an amino acid sequence according to any of previous aspects, a Nanobody according to any of previous aspects, a compound or construct according to any of previous aspects, pharmaceutical composition according to any of previous aspects that is such that it can be obtained by expression of a nucleic acid or nucleotide sequence encoding the same, or a monovalent construct according to any of previous aspects, said method at least comprising the steps of:
a) cultivating and/or maintaining a host or host cell according to any of previous aspects under conditions that are such that said host or host cell expresses and/or produces at least one amino acid sequence according to any of previous aspects, Nanobody according to any of previous aspects, compound or construct according to any of previous aspects that is such that it can be obtained by expression of a nucleic acid or nucleotide sequence encoding the same or a monovalent construct according to any of previous aspects,
   optionally followed by:
b) isolating and/or purifying the amino acid sequence according to any of aspects . . . , the Nanobody according to any of previous aspects, the compound or construct according to any of previous aspects that is such that it can be obtained by expression of a nucleic acid or nucleotide sequence encoding the same, or the monovalent construct according to any of previous aspects thus obtained
   127. Method for screening amino acid sequences directed against an integrin as e.g. shown in aspect 1 that comprises at least the steps of:
d) providing a set, collection or library of nucleic acid sequences encoding amino acid sequences;
e) screening said set, collection or library of nucleic acid sequences for nucleic acid sequences that encode an amino acid sequence that can bind to and/or has affinity for an integrin and that is cross-blocked or is cross blocking a Nanobody of the invention, e.g. SEQ ID NO: 1316 to 1487 (table-1); and
f) isolating said nucleic acid sequence, followed by expressing said amino acid sequence.
   128. Method for the prevention and/or treatment of at least one autoimmune diseases, cancer metastasis and thrombotic vascular diseases, said method comprising administering, to a subject in need thereof, a pharmaceutically active amount of at least an agent as previously described.
   129. Method for the prevention and/or treatment of at least one disease or disorder that is associated with an integrin, with its biological or pharmacological activity, and/or with the biological pathways or signalling in which an integrin is involved, said method comprising administering, to a subject in need thereof, a pharmaceutically active amount of an agent of the invention.
   130. Use of an amino acid sequence that can bind to and/or has affinity for an integrin and that is cross-blocked or is cross blocking a Nanobody of the invention, e.g. SEQ ID NO: 1316 to 1487, preferably a polypeptide essentially consisting of two identical or different Nanobodies selected from the group consisting of SEQ ID NO: 1316 to 1484, even more preferably a Nanobody with SEQ ID NO: 1486 and 1487 for diagnostic purposes, i.e. for use in microscopy, e.g. immunofluresence microscopy.
   Even more preferred aspects:

1. A single variable domain that specifically binds to at least one member of the integrins.

2. The single variable domain according to aspect 1, wherein the member of the integrins is selected from the group consisting of the human members of the integrins.

3. The single variable domain according to aspect 1, wherein the member of the integrins is selected from the group consisting of the alpha subunits and beta subunits.

4. The single variable domain according to aspect 1, wherein the member of the integrins is selected from the group consisting of the human alpha subunits and human beta subunits.

5. The single variable domain according to aspect 1, wherein the member of the integrins is selected from the group consisting of alpha1, alpha2, alpha2b, alpha3, alpha4, alpha5, alpha6, alpha7, alpha8, alpha9, alpha10, alpha11, alphaE, alphaL, alphaM, alphaX, alphaV, alphaD, beta1, beta2, beta3, beta4, beta5, beta6, beta7, and beta8.

6. The single variable domain according to aspect 1, wherein the member of the Integrins is selected from the group consisting of the human variant of alpha1, alpha2, alpha2b, alpha3, alpha4, alpha5, alpha6, alpha7, alpha8, alpha9, alpha 10, alpha 11, alphaE, alphaL, alphaM, alphaX, alphaV, alphaD, beta1, beta2, beta3, beta4, beta5, beta6, beta7, and beta8.

7. The single variable domain according to aspect 1, wherein the member of the Integrins is selected from the group consisting of the human variant of alpha3, alpha5, alphaL, alphaM, alphaV, beta1, beta2, beta6.

8. The single variable domain according to aspect 1, wherein the single variable domain additionally blocks the interaction between at least one member of the integrins with at least one other member of the integrins-ligand family.

9. The single variable domain according to aspect 1, wherein the single variable has one of the sequences selected from the group consisting of sequences with SEQ ID NO: 1316 to 1487.

10. The single variable domain according to aspect 1, wherein the single variable domain is selected from the group consisting of a) single variable domains with sequences having SEQ ID NO: 1316 to 1487; and b) single variable domains with 80% sequence identity to at least one sequence selected from the group consisting of single variable domains with sequences having SEQ ID NO: 1316 to 1487.

11. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 10 amino acid residues are replaced by naturally occurring amino acids and wherein said replaced amino acids are located within the framework regions.

12. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 8 amino acid residues are replaced by naturally occurring amino acids and wherein said replaced amino acids are located within the framework regions.

13. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 5 amino acid residues are replaced by naturally occurring amino acids and wherein said replaced amino acids are located within the framework regions.

14. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 3 amino acid residues are replaced by naturally occurring amino acids and wherein said replaced amino acids are located within the framework regions.

15. The single variable domain according to aspect 1, wherein the single variable domain is selected from the group consisting of a) single variable domains with sequences having SEQ ID NO: 1316 to 1487; and b) single variable domains with 80% sequence identity to at least one sequences selected from the group consisting of sequences having SEQ ID NO: 1316 to 1487; and wherein said selected single variable domain from group a) and b) binds to at least one member of the Integrins with a dissociation constant (K_{D}) of 10⁻⁷ to 10⁻¹² moles/liter or less.

16. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 10 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁷ to 10⁻¹² moles/liter or less.

17. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 8 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁷ to 10⁻¹² moles/liter or less.

18. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 5 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁷ to 10⁻¹² moles/liter or less.

19. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 3 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁷ to 10⁻¹² moles/liter or less.

20. The single variable domain according to aspect 1, wherein the single variable domain is selected from the group consisting of a) single variable domains with sequences having SEQ ID NO: 1316 to 1487; and b) single variable domains with 80% sequence identity to at least one sequences selected from the group consisting of sequences having SEQ ID NO: 1316 to 1487; and wherein said selected single variable domain from group a) and b) binds to at least one member of the integrins with a dissociation constant (K_{D}) of 10⁻⁸ to 10⁻¹² moles/liter or less.

21. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 10 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁸ to 10⁻¹² moles/liter or less.

22. The single variable domain according to aspect 1, wherein the single variable domain is selected from the group consisting of a) single variable domains with sequences having SEQ ID NO: 1316 to 1487; and b) single variable domains with sequences SEQ ID NO: 1316 to 1487 wherein up to 8 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁸ to 10⁻¹² moles/liter or less.

23. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 5 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁸ to 10⁻¹² moles/liter or less.

24. The single variable domain according to aspect 1, wherein the single variable domain is selected from the group consisting of a) single variable domains with sequences having SEQ ID NO: 1316 to 1487; and b) single variable domains with sequences having SEQ ID NO: 1316 to 1487, wherein up to 3 amino acid residues are replaced by naturally occurring amino acids and wherein said replaced amino acids are located within the framework regions; and wherein said selected single variable domain from group a) and b) binds to at least one member of the integrins with a dissociation constant (K_{D}) of 10⁻⁸ to 10⁻¹² moles/liter or less.

25. A single variable domain that specifically binds to at least the alpha L.

26. The single variable domain according to aspect 25, binds to at least the human alpha L.

27. The single variable domain according to aspect 25, binds to at least the mouse alpha L.

28. The single variable domain according to aspect 25, wherein the single variable domain additionally blocks the interaction between alpha L with at least one single variable domain with sequences having SEQ ID NO: 1316 to 1344.

29. The single variable domain according to aspect 25, wherein the single variable domain is selected from the group consisting of a) single variable domains with sequences having SEQ ID NO: 1316 to 1344; and b) single variable domains with 80% sequence identity to at least one sequence selected from the group consisting of single variable domains with sequences having SEQ ID NO: 1316 to 1344.

30. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 10 amino acid residues are replaced by naturally occurring amino acids and wherein said replaced amino acids are located within the framework regions.

31. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 8 amino acid residues are replaced by naturally occurring amino acids and wherein said replaced amino acids are located within the framework regions.

32. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 5 amino acid residues are replaced by naturally occurring amino acids and wherein said replaced amino acids are located within the framework regions.

33. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 3 amino acid residues are replaced by naturally occurring amino acids and wherein said replaced amino acids are located within the framework regions.

34. The single variable domain according to aspect 25, wherein the single variable domain is selected from the group consisting of a) single variable domains with sequences having SEQ ID NO: 1316 to 1344; and b) single variable domains with 80% sequence identity to at least one sequences selected from the group consisting of sequences having SEQ ID NO: 1316 to 1344; and wherein said selected single variable domain from group a) and b) binds to at least human alpha L with a dissociation constant (K_{D}) of 10⁻⁷ to 10¹² moles/liter or less.

35. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 10 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁷ to 10¹² moles/liter or less.

36. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 8 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁷ to 10² moles/liter or less.

37. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 5 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁷ to 10⁻¹² moles/liter or less.

38. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 3 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁷ to 10⁻¹² moles/liter or less.

39. The single variable domain according to aspect 25, wherein the single variable domain is selected from the group consisting of a) single variable domains with sequences having SEQ ID NO: 1316 to 1344; and b) single variable domains with 80% sequence identity to at least one sequences selected from the group consisting of sequences having SEQ ID NO: 1316 to 1344; and wherein said selected single variable domain from group a) and b) binds to human alpha L with a dissociation constant (K_{D}) of 10⁻⁸ to 10⁻¹² moles/liter or less.

40. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 10 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁸ to 10⁻¹² moles/liter or less.

41. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 8 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁸ to 10⁻¹² moles/liter or less.

42. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 5 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁸ to 10⁻¹² moles/liter or less.

43. The single variable domain according to aspect 25, wherein the single variable domain is selected from the group consisting of a) single variable domains with sequences having SEQ ID NO: 1316 to 1344; and b) single variable domains with sequences having SEQ ID NO: 1316 to 1344, wherein up to 3 amino acid residues are replaced by naturally occurring amino acids and wherein said replaced amino acids are located within the framework regions; and wherein said selected single variable domain from group a) and b) binds to at least human alpha L with a dissociation constant (K_{D}) of 10⁻⁸ to 10⁻¹² moles/liter or less.

44. A single variable domain that specifically binds to at least beta2.

45. The single variable domain according to aspect 44, wherein the single variable domain that specifically binds to at least human beta2.

46. The single variable domain according to aspect 44, wherein the single variable domain that specifically binds to at least mouse beta2.

47. The single variable domain according to aspect 44, wherein the single variable domain additionally blocks the interaction between at least human beta2 with at least one single variable domain with sequences having SEQ ID NO: 1345 to 1394.

48. The single variable domain according to aspect 44, wherein the single variable domain is selected from the group consisting of a) single variable domains with sequences having SEQ ID NO: 1345 to 1394; and b) single variable domains with 80% sequence identity to at least one sequence selected from the group consisting of single variable domains with sequences having SEQ ID NO: 1345 to 1394.

49. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 10 amino acid residues are replaced by naturally occurring amino acids and wherein said replaced amino acids are located within the framework regions.

50. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 8 amino acid residues are replaced by naturally occurring amino acids and wherein said replaced amino acids are located within the framework regions.

51. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 5 amino acid residues are replaced by naturally occurring amino acids and wherein said replaced amino acids are located within the framework regions.

52. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 3 amino acid residues are replaced by naturally occurring amino acids and wherein said replaced amino acids are located within the framework regions.

53. The single variable domain according to aspect 44, wherein the single variable domain is selected from the group consisting of a) single variable domains with sequences having SEQ ID NO: 1345 to 1394; and b) single variable domains with 80% sequence identity to at least one sequences selected from the group consisting of sequences having SEQ ID NO: 1345 to 1394; and wherein said selected single variable domain from group a) and b) binds to at least human beta2 with a dissociation constant (K_{D}) of 10⁻⁷ to 10⁻¹² moles/liter or less.

54. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 10 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁷ to 10⁻¹² moles/liter or less.

55. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 8 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁷ to 10¹² moles/liter or less.

56. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 5 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁷ to 10⁻¹² moles/liter or less.

57. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 3 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁷ to 10⁻¹² moles/liter or less.

58. The single variable domain according to aspect 44, wherein the single variable domain is selected from the group consisting of a) single variable domains with sequences having SEQ ID NO: 1345 to 1394; and b) single variable domains with 80% sequence identity to at least one sequences selected from the group consisting of sequences having SEQ ID NO: 1345 to 1394; and wherein said selected single variable domain from group a) and b) binds to at least human beta2 with a dissociation constant (K_{D}) of 10⁻⁸ to 10⁻¹² moles/liter or less.

59. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 10 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁸ to 10⁻¹² moles/liter or less.

60. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 8 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁸ to 10⁻¹² moles/liter or less.

61. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 5 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁸ to 10⁻¹² moles/liter or less.

62. The single variable domain according to aspect 44, wherein the single variable domain is selected from the group consisting of a) single variable domains with sequences having SEQ ID NO: 1345 to 1394; and b) single variable domains with sequences having SEQ ID NO: 1345 to 1394, wherein up to 3 amino acid residues are replaced by naturally occurring amino acids and wherein said replaced amino acids are located within the framework regions; and wherein said selected single variable domain from group a) and b) binds to at least human beta2 with a dissociation constant (K_{D}) of 10⁻⁸ to 10⁻² moles/liter or less.

63. A single variable domain that specifically binds to at least alpha M.

64. The single variable domain according to aspect 63, that specifically binds to at least human alpha M.

65. The single variable domain according to aspect 63, that specifically binds to at least mouse alpha M.

66. The single variable domain according to aspect 63, wherein the single variable domain additionally blocks the interaction between human alpha M with at least one single variable domain with sequences having SEQ ID NO: 1395 to 1408.

67. The single variable domain according to aspect 63, wherein the single variable domain is selected from the group consisting of a) single variable domains with sequences having SEQ ID NO: 1395 to 1408; and b) single variable domains with 80% sequence identity to at least one sequence selected from the group consisting of single variable domains with sequences having SEQ ID NO: 1395 to 1408.

68. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 10 amino acid residues are replaced by naturally occurring amino acids and wherein said replaced amino acids are located within the framework regions.

69. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 8 amino acid residues are replaced by naturally occurring amino acids and wherein said replaced amino acids are located within the framework regions.

70. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 5 amino acid residues are replaced by naturally occurring amino acids and wherein said replaced amino acids are located within the framework regions.

71. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 3 amino acid residues are replaced by naturally occurring amino acids and wherein said replaced amino acids are located within the framework regions.

72. The single variable domain according to aspect 63, wherein the single variable domain is selected from the group consisting of a) single variable domains with sequences having SEQ ID NO: 1395 to 1408; and b) single variable domains with 80% sequence identity to at least one sequences selected from the group consisting of sequences having SEQ ID NO: 1395 to 1408; and wherein said selected single variable domain from group a) and b) binds to human alpha M with a dissociation constant (K_{D}) of 10⁻⁷ to 10⁻¹² moles/liter or less.

73. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 10 amino acid residues are replaced by naturally occurring amino acids and wherein said replaced amino acids are located within the framework regions; and wherein said selected single variable domain from group a) and b) binds to human alpha M with a dissociation constant (Kₒ) of 10⁻⁷ to 10⁻¹² moles/liter or less.

74. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 8 amino acid residues are replaced by naturally occurring amino acids and wherein said replaced amino acids are located within the framework regions; and wherein said selected single variable domain from group a) and b) binds to human alpha M with a dissociation constant (K_{D}) of 10⁻⁷ to 10⁻¹² moles/liter or less.

75. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 5 amino acid residues are replaced by naturally occurring amino acids and wherein said replaced amino acids are located within the framework regions; and wherein said selected single variable domain from group a) and b) binds to human alpha M with a dissociation constant (K_{D}) of 10⁻⁷ to 10⁻¹² moles/liter or less.

76. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 3 amino acid residues are replaced by naturally occurring amino acids and wherein said replaced amino acids are located within the framework regions; and wherein said selected single variable domain from group a) and b) binds to human alpha M with a dissociation constant (K_{D}) of 10⁻⁷ to 10⁻¹² moles/liter or less.

77. The single variable domain according to aspect 63, wherein the single variable domain is selected from the group consisting of a) single variable domains with sequences having SEQ ID NO: 1395 to 1408; and b) single variable domains with 80% sequence identity to at least one sequences selected from the group consisting of sequences having SEQ ID NO: 1395 to 1408; and wherein said selected single variable domain from group a) and b) binds to human alpha M with a dissociation constant (K_{D}) of 10⁻⁸ to 10⁻¹² moles/liter or less.

78. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 10 amino acid residues are replaced by naturally occurring amino acids and wherein said replaced amino acids are located within the framework regions; and wherein said selected single variable domain from group a) and b) binds to human alpha M with a dissociation constant (K_{D}) of 10⁻⁸ to 10⁻¹² moles/liter or less.

79. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 8 amino acid residues are replaced by naturally occurring amino acids and wherein said replaced amino acids are located within the framework regions; and wherein said selected single variable domain from group a) and b) binds to human alpha M with a dissociation constant (K_{D}) of 10⁻⁸ to 10⁻¹² moles/liter or less.

80. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 5 amino acid residues are replaced by naturally occurring amino acids and wherein said replaced amino acids are located within the framework regions; and wherein said selected single variable domain from group a) and b) binds to human alpha M with a dissociation constant (K_{D}) of 10⁻⁸ to 10⁻¹² moles/liter or less.

81. The single variable domain according to aspect 63, wherein the single variable domain is selected from the group consisting of a) single variable domains with sequences having SEQ ID NO: 1395 to 1408; and b) single variable domains with sequences having SEQ ID NO: 1395 to 1408, wherein up to 3 amino acid residues are replaced by naturally occurring amino acids and wherein said replaced amino acids are located within the framework regions; and wherein said selected single variable domain from group a) and b) binds to human alpha M with a dissociation constant (Kᵤ) of 10⁻⁸ to 10⁻¹² moles/liter or less.

82. A single variable domain that specifically binds to at least alphaV or beta6.

83. The single variable domain according to aspect 82, that specifically binds to at least human alphaV or human beta6.

84. The single variable domain according to aspect 82, that specifically binds to at least mouse alphaV or mouse beta6.

85. The single variable domain according to aspect 82, wherein the single variable domain additionally blocks the interaction between human alphaV or human beta6 with at least one single variable domain with sequences having SEQ ID NO: 1409 to 1426.

86. The single variable domain according to aspect 82, wherein the single variable domain is selected from the group consisting of a) single variable domains with sequences having SEQ ID NO: 1409 to 1426; and b) single variable domains with 80% sequence identity to at least one sequence selected from the group consisting of single variable domains with sequences having SEQ ID NO: 1409 to 1426.

87. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 10 amino acid residues are replaced by naturally occurring amino acids and wherein said replaced amino acids are located within the framework regions.

88. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 8 amino acid residues are replaced by naturally occurring amino acids and wherein said replaced amino acids are located within the framework regions.

89. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 5 amino acid residues are replaced by naturally occurring amino acids and wherein said replaced amino acids are located within the framework regions.

90. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 3 amino acid residues are replaced by naturally occurring amino acids and wherein said replaced amino acids are located within the framework regions.

91. The single variable domain according to aspect 82, wherein the single variable domain is selected from the group consisting of a) single variable domains with sequences having SEQ ID NO: 1409 to 1426; and b) single variable domains with 80% sequence identity to at least one sequences selected from the group consisting of sequences having SEQ ID NO: 1409 to 1426; and wherein said selected single variable domain from group a) and b) binds to human alphaV or human beta6 with a dissociation constant (K_{D}) of 10⁻⁷ to 10⁻¹² moles/liter or less.

92. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 10 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁷ to 10⁻¹² moles/liter or less.

93. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 8 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁷ to 10¹² moles/liter or less.

94. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 5 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁷ to 10⁻¹² moles/liter or less.

95. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 3 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁷ to 10⁻¹² moles/liter or less.

96. The single variable domain according to aspect 82, wherein the single variable domain is selected from the group consisting of a) single variable domains with sequences having SEQ ID NO: 1409 to 1426; and b) single variable domains with 80% sequence identity to at least one sequences selected from the group consisting of sequences having SEQ ID NO: 1409 to 1426; and wherein said selected single variable domain from group a) and b) binds to human alphaV or human beta6 with a dissociation constant (K_{D}) of 10⁻⁷ to 10⁻¹² moles/liter or less.

97. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 10 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁸ to 10⁻¹² moles/liter or less.

98. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 8 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁸ to 10⁻¹² moles/liter or less.

99. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 5 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁸ to 10⁻¹² moles/liter or less.

100. The single variable domain according to aspect 82, wherein the single variable domain is selected from the group consisting of a) single variable domains with sequences having SEQ ID NO: 1409 to 1426; and b) single variable domains with sequences having SEQ ID NO: 1409 to 1426, wherein up to 3 amino acid residues are replaced by naturally occurring amino acids and wherein said replaced amino acids are located within the framework regions; and wherein said selected single variable domain from group a) and b) binds to human alphaV or human beta6 with a dissociation constant (K_{D}) of 10⁻⁸ to 10⁻¹² moles/liter or less.

101. A single variable domain that specifically binds to at least beta1.

102. The single variable domain according to aspect 101, that specifically binds to at least human beta1.

103. The single variable domain according to aspect 101, that specifically binds to at least mouse beta1.

104. The single variable domain according to aspect 101, wherein the single variable domain additionally blocks the interaction between human Beta1 with at least one single variable domain with sequences having SEQ ID NO: 1427 to 1450.

105. The single variable domain according to aspect 101, wherein the single variable domain is selected from the group consisting of a) single variable domains with sequences having SEQ ID NO: 1427 to 1450; and b) single variable domains with 80% sequence identity to at least one sequence selected from the group consisting of single variable domains with sequences having SEQ ID NO: 1427 to 1450.

106. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 10 amino acid residues are replaced by naturally occurring amino acids and wherein said replaced amino acids are located within the framework regions.

107. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 8 amino acid residues are replaced by naturally occurring amino acids and wherein said replaced amino acids are located within the framework regions.

108. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 5 amino acid residues are replaced by naturally occurring amino acids and wherein said replaced amino acids are located within the framework regions.

109. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 3 amino acid residues are replaced by naturally occurring amino acids and wherein said replaced amino acids are located within the framework regions.

110. The single variable domain according to aspect 101, wherein the single variable domain is selected from the group consisting of a) single variable domains with sequences having SEQ ID NO: 1427 to 1450; and b) single variable domains with 80% sequence identity to at least one sequences selected from the group consisting of sequences having SEQ ID NO: 1427 to 1450; and wherein said selected single variable domain from group a) and b) binds to human beta1 with a dissociation constant (K_{D}) of 10⁻⁷ to 10⁻¹² moles/liter or less.

111. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 10 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁷ to 10⁻¹² moles/liter or less.

112. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 8 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁷ to 10⁻¹² moles/liter or less.

113. The single variable domain according to aspect 101, wherein the single variable domain is selected from the group consisting of a) single variable domains with sequences having SEQ ID NO: 1427 to 1450; and b) single variable domains sequences having SEQ ID NO: 1427 to 1450, wherein up to 5 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁷ to 10⁻¹² moles/liter or less.

114. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 3 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁷ to 10⁻¹² moles/liter or less.

115. The single variable domain according to aspect 101, wherein the single variable domain is selected from the group consisting of a) single variable domains with sequences having SEQ ID NO: 1427 to 1450; and b) single variable domains with 80% sequence identity to at least one sequences selected from the group consisting of sequences having SEQ ID NO: 1427 to 1450; and wherein said selected single variable domain from group a) and b) binds to human beta1 with a dissociation constant (K_{D}) of 10⁻⁸ to 10⁻¹² moles/liter or less.

116. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 10 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁸ to 10⁻¹² moles/liter or less.

117. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 8 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁸ to 10⁻¹² moles/liter or less.

118. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 5 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁸ to 10⁻¹² moles/liter or less.

119. The single variable domain according to aspect 101, wherein the single variable domain is selected from the group consisting of a) single variable domains with sequences having SEQ ID NO: 1427 to 1450; and b) single variable domains with sequences having SEQ ID NO: 1427 to 1450, wherein up to 3 amino acid residues are replaced by naturally occurring amino acids and wherein said replaced amino acids are located within the framework regions; and wherein said selected single variable domain from group a) and b) binds to human beta1 with a dissociation constant (K_{D}) of 10⁻⁸ to 10⁻¹² moles/liter or less.

120. A single variable domain that specifically binds to at least alpha5.

121. The single variable domain according to aspect 120, that specifically binds to at least human alpha5.

122. The single variable domain according to aspect 120, that specifically binds to at least mouse alpha5.

123. The single variable domain according to aspect 120, wherein the single variable domain additionally blocks the interaction between human alpha5 with at least one single variable domain with sequences having SEQ ID NO: 1451 to 1457.

124. The single variable domain according to aspect 120, wherein the single variable domain is selected from the group consisting of a) single variable domains with sequences having SEQ ID NO: 1451 to 1457; and b) single variable domains with 80% sequence identity to at least one sequence selected from the group consisting of single variable domains with sequences having SEQ ID NO: 1451 to 1457.

125. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 10 amino acid residues are replaced by naturally occurring amino acids and wherein said replaced amino acids are located within the framework regions.

126. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 8 amino acid residues are replaced by naturally occurring amino acids and wherein said replaced amino acids are located within the framework regions.

127. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 5 amino acid residues are replaced by naturally occurring amino acids and wherein said replaced amino acids are located within the framework regions.

128. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 3 amino acid residues are replaced by naturally occurring amino acids and wherein said replaced amino acids are located within the framework regions.

129. The single variable domain according to aspect 120, wherein the single variable domain is selected from the group consisting of a) single variable domains with sequences having SEQ ID NO: 1451 to 1457; and b) single variable domains with 80% sequence identity to at least one sequences selected from the group consisting of sequences having SEQ ID NO: 1451 to 1457; and wherein said selected single variable domain from group a) and b) binds to human alpha5 with a dissociation constant (K_{D}) of 10⁻⁷ to 10⁻¹² moles/liter or less.

130. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 10 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁷ to 10⁻¹² moles/liter or less.

131. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 8 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁷ to 10⁻¹² moles/liter or less.

132. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 5 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁷ to 10⁻¹² moles/liter or less.

133. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 3 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁷ to 10⁻¹² moles/liter or less.

134. The single variable domain according to aspect 120, wherein the single variable domain is selected from the group consisting of a) single variable domains with sequences having SEQ ID NO: 1451 to 1457; and b) single variable domains with 80% sequence identity to at least one sequences selected from the group consisting of sequences having SEQ ID NO: 1451 to 1457; and wherein said selected single variable domain from group a) and b) binds to human alpha5 with a dissociation constant (K_{D}) of 10⁻⁸ to 10⁻¹² moles/liter or less.

135. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 10 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁸ to 10⁻¹² moles/liter or less.

136. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 8 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁸ to 10⁻¹² moles/liter or less.

137. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 5 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁸ to 10⁻¹² moles/liter or less.

138. The single variable domain according to aspect 120, wherein the single variable domain is selected from the group consisting of a) single variable domains with sequences having SEQ ID NO: 1451 to 1457; and b) single variable domains with sequences having SEQ ID NO: 1451 to 1457, wherein up to 3 amino acid residues are replaced by naturally occurring amino acids and wherein said replaced amino acids are located within the framework regions; and wherein said selected single variable domain from group a) and b) binds to human alpha5 with a dissociation constant (K_{D}) of 10⁻⁸ to 10⁻¹² moles/liter or less.

139. A single variable domain that specifically binds to at least alpha3.

140. The single variable domain according to aspect 139, that specifically binds to at least human alpha3.

141. The single variable domain according to aspect 139, that specifically binds to at least mouse alpha3.

142. The single variable domain according to aspect 139, wherein the single variable domain additionally blocks the interaction between human alpha3 with at least one single variable domain with sequences having SEQ ID NO: 1458 to 1476, and SEQ ID NO: 1485, 1486, and 1487.

143. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with 80% sequence identity to at least one sequence selected from the group consisting of single variable domains with sequences having SEQ ID NO: 1458 to 1476, and SEQ ID NO: 1485, 1486, and 1487.

144. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 10 amino acid residues are replaced by naturally occurring amino acids and wherein said replaced amino acids are located within the framework regions.

145. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 8 amino acid residues are replaced by naturally occurring amino acids and wherein said replaced amino acids are located within the framework regions.

146. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 5 amino acid residues are replaced by naturally occurring amino acids and wherein said replaced amino acids are located within the framework regions.

147. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 3 amino acid residues are replaced by naturally occurring amino acids and wherein said replaced amino acids are located within the framework regions.

148. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with 80% sequence identity to at least one sequences selected from the group consisting of sequences having SEQ ID NO: 1458 to 1476, and SEQ ID NO: 1485, 1486, and 1487; and wherein said selected single variable domain from group a) and b) binds to human alpha3 with a dissociation constant (K_{D}) of 10⁻⁷ to 10⁻¹² moles/liter or less.

149. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 10 amino acid residues are replaced by naturally occurring amino acids and wherein said replaced amino acids are located within the framework regions; and wherein said selected single variable domain from group a) and b) binds to human alpha3 with a dissociation constant (K_{D}) of 10⁻⁷ to 10⁻¹² moles/liter or less.

150. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 8 amino acid residues are replaced by naturally occurring amino acids and wherein said replaced amino acids are located within the framework regions; and wherein said selected single variable domain from group a) and b) binds to human alpha3 with a dissociation constant (K_{D}) of 10⁻⁷ to 10⁻¹² moles/liter or less.

151. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 5 amino acid residues are replaced by naturally occurring amino acids and wherein said replaced amino acids are located within the framework regions; and wherein said selected single variable domain from group a) and b) binds to human alpha3 with a dissociation constant (K_{D}) of 10⁻⁷ to 10⁻¹² moles/liter or less.

152. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 3 amino acid residues are replaced by naturally occurring amino acids and wherein said replaced amino acids are located within the framework regions; and wherein said selected single variable domain from group a) and b) binds to human alpha3 with a dissociation constant (K_{D}) of 10⁻⁷ to 10⁻¹² moles/liter or less.

153. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with 80% sequence identity to at least one sequences selected from the group consisting of sequences having SEQ ID NO: 1458 to 1476, and SEQ ID NO: 1485, 1486, and 1487; and wherein said selected single variable domain from group a) and b) binds to human alpha3 with a dissociation constant (K_{D}) of 10⁻⁸ to 10⁻¹² moles/liter or less.

154. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 10 amino acid residues are replaced by naturally occurring amino acids and wherein said replaced amino acids are located within the framework regions; and wherein said selected single variable domain from group a) and b) binds to human alpha3 with a dissociation constant (IC_{D}) of 10⁻⁸ to 10⁻¹² moles/liter or less.

155. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 8 amino acid residues are replaced by naturally occurring amino acids and wherein said replaced amino acids are located within the framework regions; and wherein said selected single variable domain from group a) and b) binds to human alpha3 with a dissociation constant (K_{D}) of 10⁻⁸ to 10⁻¹² moles/liter or less.

156. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 5 amino acid residues are replaced by naturally occurring amino acids and wherein said replaced amino acids are located within the framework regions; and wherein said selected single variable domain from group a) and b) binds to human alpha3 with a dissociation constant (K_{D}) of 10⁻⁸ to 10⁻¹² moles/liter or less.

157. The single variable domain according to aspect 139, wherein the single variable domain is selected from the group consisting of a) single variable domains with sequences having SEQ ID NO: 1458 to 1476, and SEQ ID NO: 1485, 1486, and 1487; and b) single variable domains with sequences having SEQ ID NO: 1458 to 1476, and SEQ ID NO: 1485, 1486, and 1487, wherein up to 3 amino acid residues are replaced by naturally occurring amino acids and wherein said replaced amino acids are located within the framework regions; and wherein said selected single variable domain from group a) and b) binds to human alpha3 with a dissociation constant (K_{D}) of 10⁻⁸ to 10⁻¹² moles/liter or less.

158. A single variable domain that specifically binds to at least human alpha3.

159. The single variable domain according to aspect 158, wherein the single variable domain additionally blocks the interaction between human alpha3 with at least one single variable domain with sequences having SEQ ID NO: 1485 to 1487.

160. The single variable domain according to aspect 158, wherein the single variable domain is selected from the group consisting of a) single variable domains with sequences having SEQ ID NO: 1485 to 1487; and b) single variable domains with 80% sequence identity to at least one sequence selected from the group consisting of single variable domains with sequences having SEQ ID NO: 1485 to 1487.

161. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 10 amino acid residues are replaced by naturally occurring amino acids and wherein said replaced amino acids are located within the framework regions.

162. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 8 amino acid residues are replaced by naturally occurring amino acids and wherein said replaced amino acids are located within the framework regions.

163. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 5 amino acid residues are replaced by naturally occurring amino acids and wherein said replaced amino acids are located within the framework regions.

164. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 3 amino acid residues are replaced by naturally occurring amino acids and wherein said replaced amino acids are located within the framework regions.

165. The single variable domain according to aspect 158, wherein the single variable domain is selected from the group consisting of a) single variable domains with sequences having SEQ ID NO: 1485 to 1487; and b) single variable domains with 80% sequence identity to at least one sequences selected from the group consisting of sequences having SEQ ID NO: 1485 to 1487; and wherein said selected single variable domain from group a) and b) binds to human alpha3 or human beta1 with a dissociation constant (K_{D}) of 10⁻⁷ to 10⁻¹² moles/liter or less.

166. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 10 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁷ to 10⁻¹² moles/liter or less.

167. The single variable domain according to aspect 158, wherein the single variable domain is selected from the group consisting of a) single variable domains with sequences having SEQ ID NO: 1485 to 1487; and b) single variable domains with, sequences having SEQ ID NO: 1485 to 1487, wherein up to 8 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁷ to 10¹² moles/liter or less.

168. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 5 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁷ to 10⁻¹² moles/liter or less.

169. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 3 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁷ to 10⁻¹² moles/liter or less.

170. The single variable domain according to aspect 158, wherein the single variable domain is selected from the group consisting of a) single variable domains with sequences having SEQ ID NO: 1485 to 1487; and b) single variable domains with 80% sequence identity to at least one sequences selected from the group consisting of sequences having SEQ ID NO: 1485 to 1487; and wherein said selected single variable domain from group a) and b) binds to human alpha3 or human beta1 with a dissociation constant (K_{D}) of 10⁻⁸ to 10⁻¹² moles/liter or less.

171. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 10 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁸ to 10⁻¹² moles/liter or less.

172. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 8 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁸ to 10⁻¹² moles/liter or less.

173. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 5 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁸ to 10⁻¹² moles/liter or less.

174. The single variable domain according to aspect 158, wherein the single variable domain is selected from the group consisting of a) single variable domains with sequences having SEQ ID NO: 1485 to 1487; and b) single variable domains with sequences having SEQ ID NO: 1485 to 1487, wherein up to 3 amino acid residues are replaced by naturally occurring amino acids and wherein said replaced amino acids are located within the framework regions; and wherein said selected single variable domain from group a) and b) binds to human alpha3 or human beta1 with a dissociation constant (K_{D}) of 10⁻⁸ to 10⁻¹² moles/liter or less.

175. A construct comprising at least one single variable domain of aspects 1 to 24.

176. A construct comprising at least one single variable domain according to aspects 25 to 43.

177. A construct comprising at least one single variable domain of aspects 44 to 62.

178. A construct comprising at least one single variable domain of aspects 63 to 81.

179. A construct comprising at least one single variable domain of aspects 82 to 100.

180. A construct comprising at least one single variable domain of aspects 101 to 119.

181. A construct comprising at least one single variable domain of aspects 120 to 138.

182. A construct comprising at least one single variable domain of aspects 139 to 157.

183. A construct comprising at least one single variable domain of aspects 158 to 174.

184. A pharmaceutical composition comprising a single variable domain of aspects 1 to 174 and/or a construct of aspects 175 to 183.

185. A nucleotide sequence encoding for a single variable domain of aspects 1 to 174 and/or a construct of aspects 175 to 183.

186. A host cell able to express and/or comprising a single variable domain of aspects 1 to 174 and/or a construct of aspects 175 to 183, and/or a nucleotide sequence of aspect 185.

187. Method of making a single variable domain of any aspects 1 to 174, method of making a construct of aspects 175 to 183; method of making a pharmaceutical composition of aspect 184; method of making a nucleotide sequence of aspect 185; and/or making a host cell of aspect 186.

188. Method of screening a molecule specifically binding to a member of the Integrins using a single variable domain of aspects 1 to 174 or a construct of aspects 175 to 183, a pharmaceutical composition of aspect 184, a nucleotide sequence of aspect 185, and/or a host cell of aspect 186.

189. Method for the prevention and/or treatment of at least one disease or disorder in which a member selected from the group consisting of the Integrins plays a role or is implicated, said method comprising administering, to a subject in need thereof, a pharmaceutically active amount of at least one single variable domain of aspects 1 to 174, one construct of aspects 175 to 183, or one pharmaceutical composition of aspect 184.

190. Method for the prevention and/or treatment of at least one disease or disorder selected from the group of diseases consisting of cancers, an autoimmune diseases or neurodegenerative diseases, said method comprising administering, to a subject in need thereof, a pharmaceutically active amount of at least one single variable domain of aspects 1 to 174, one construct of aspects 175 to 183, or one pharmaceutical composition of aspect 184.

191. Use of a pharmaceutically active amount of at least one single variable domain of aspects 1 to 174, one construct of aspects 175 to 183, or one pharmaceutical composition of aspect 184 for the prevention and/or treatment of at least one disease or disorder in which a member selected from the group consisting of the integrins plays a role or is implicated.

192. Use of a pharmaceutically active amount of at least one single variable domain of aspects 1 to 174, one construct of aspects 175 to 183, or one pharmaceutical composition of aspect 184 for the prevention and/or treatment of at least one disease or disorder selected from the group of diseases consisting of cancers, an autoimmune diseases or neurodegenerative diseases.

193. Use of a pharmaceutically active amount of at least one single variable domain of aspects 1 to 174, one construct of aspects 175 to 183, or one pharmaceutical composition of aspect 184 for the manufacture of a medicament for the prevention and/or treatment of at least one disease or disorder in which a member selected from the group consisting of the integrins plays a role or is implicated.

194. Use of a pharmaceutically active amount of at least one single variable domain of aspects 1 to 174, one construct of aspects 175 to 183, or one pharmaceutical composition of aspect 184 for the manufacture of a medicament for the prevention and/or treatment of at least one disease or disorder selected from the group of diseases consisting of cancers, an autoimmune diseases or neurodegenerative diseases.

195. Diagnostic kit comprising at least one single variable domain of aspects 1 to 174.

1. A single variable domain that specifically binds to at least one member of the integrins.

2. The single variable domain according to claim 1, wherein the member of the integrins is selected from the group consisting of the human members of the integrins.

3. The single variable domain according to claim 1, wherein the member of the integrins is selected from the group consisting of the alpha subunits and beta subunits.

4. The single variable domain according to claim 1, wherein the member of the integrins is selected from the group consisting of the human alpha subunits and human beta subunits.

5. The single variable domain according to claim 1, wherein the member of the integrins is selected from the group consisting of alpha1, alpha2, alpha2b, alpha3, alpha4, alpha5, alpha6, alpha7, alpha8, alpha9, alpha10, alpha11, alphaE, alphaL, alphaM, alphaX, alphaV, alphaD, beta1, beta2, beta3, beta4, beta5, beta6, beta7, and beta8.

6. The single variable domain according to claim 1, wherein the member of the Integrins is selected from the group consisting of the human variant of alpha1, alpha2, alpha2b, alpha3, alpha4, alpha5, alpha6, alpha7, alpha8, alpha9, alpha10, alpha11, alphaE, alphaL, alphaM, alphaX, alphaV, alphaD, beta1, beta2, beta3, beta4, beta5, beta6, beta7, and beta8.

7. The single variable domain according to claim 1, wherein the member of the Integrins is selected from the group consisting of the human variant of alpha3, alpha5, alphaL, alphaM, alphaV, beta1, beta2, beta6.

8. The single variable domain according to claim 1, wherein the single variable domain additionally blocks the interaction between at least one member of the integrins with at least one other member of the integrins-ligand family.

9. The single variable domain according to claim 1, wherein the single variable has one of the sequences selected from the group consisting of sequences with SEQ ID NO: 1316 to 1487.

10. The single variable domain according to claim 1, wherein the single variable domain is selected from the group consisting of a) single variable domains with sequences having SEQ ID NO: 1316 to 1487; and b) single variable domains with 80% sequence identity to at least one sequence selected from the group consisting of single variable domains with sequences having SEQ ID NO: 1316 to 1487.

11. The single variable domain according to claim 1, wherein the single variable domain is selected from the group consisting of a) single variable domains with sequences having SEQ ID NO: 1316 to 1487; and b) single variable domains with sequences having SEQ ID NO: 1316 to 1487, wherein up to 10 amino acid residues are replaced by naturally occurring amino acids and wherein said replaced amino acids are located within the framework regions.

12. The single variable domain according to claim 1, wherein the single variable domain is selected from the group consisting of a) single variable domains with sequences having SEQ ID NO: 1316 to 1487; and b) single variable domains with sequences having SEQ ID NO: 1316 to 1487, wherein up to 8 amino acid residues are replaced by naturally occurring amino acids and wherein said replaced amino acids are located within the framework regions.

13. The single variable domain according to claim 1, wherein the single variable domain is selected from the group consisting of a) single variable domains with sequences having SEQ ID NO: 1316 to 1487; and b) single variable domains with sequences having SEQ ID NO: 1316 to 1487, wherein up to 5 amino acid residues are replaced by naturally occurring amino acids and wherein said replaced amino acids are located within the framework regions.

14. The single variable domain according to claim 1, wherein the single variable domain is selected from the group consisting of a) single variable domains with sequences having SEQ ID NO: 1316 to 1487; and b) single variable domains with sequences having SEQ ID NO: 1316 to 1487, wherein up to 3 amino acid residues are replaced by naturally occurring amino acids and wherein said replaced amino acids are located within the framework regions.

15. The single variable domain according to claim 1, wherein the single variable domain is selected from the group consisting of a) single variable domains with sequences having SEQ ID NO: 1316 to 1487; and b) single variable domains with 80% sequence identity to at least one sequences selected from the group consisting of sequences having SEQ ID NO: 1316 to 1487; and wherein said selected single variable domain from group a) and b) binds to at least one member of the Integrins with a dissociation constant (K_{D}) of 10⁻⁷ to 10⁻¹² moles/liter or less.
